(19) United States
(12) Patent Application Publication
(10) Pub. No.: US 2020/0138827 A1
(43) Pub. Date: May 7, 2020

(54) SMALL MOLECULE MODULATORS OF HUMAN STING

(71) Applicant: Curadev Pharma Limited, Sandwich, Kent (GB)

(72) Inventors: Monali Banerjee, Noida, Uttar Pradesh (IN); Sandip Middya, Noida, Uttar Pradesh (IN); Sourav Basu, Noida, Uttar Pradesh (IN); Rajib Ghosh, Noida, Uttar Pradesh (IN); David Pryde, Sandwich, Kent (GB); Dharmendra Yadav, Noida, Uttar Pradesh (IN); Ritesh Shrivastava, Noida, Uttar Pradesh (IN); Arjun Surya, Noida, Uttar Pradesh (IN)

(21) Appl. No.: 16/625,032

(22) PCT Filed: Jun. 21, 2018
(86) PCT No.: PCT/GB18/51727

§ 371 Date: Dec. 20, 2019

(30) Foreign Application Priority Data
Jun. 22, 2017 (GB) 1709959.9
Jun. 22, 2017 (IN) 201711021858

(51) Int. Cl. A61K 31/5415; C07D 417/12; C07D 417/14; C07D 279/16; C07D 513/04; C07D 413/12; C07D 265/36; C07D 215/227; C07D 405/12; C07D 491/048; C07D 471/04; C07D 279/02; A61P 35/00; A61K 47/51; A61K 45/06; A61K 31/542; A61K 31/536; A61K 31/5365; A61K 31/438; A61K 31/4375

(52) U.S. Cl. CPC A61K 31/5415; A61K 31/4375; C07D 417/14; C07D 279/16; C07D 513/04; C07D 413/12; C07D 265/36; C07D 215/227; C07D 405/12; C07D 491/048; C07D 471/04; C07D 279/02; A61P 35/00; A61K 47/51; A61K 45/06; A61K 31/542; A61K 31/536; A61K 31/5365; A61K 31/438; C07D 417/12

(57) ABSTRACT

The present invention relates to compounds of formula (I). The compounds may be used to modulate the Stimulator of Interferon Genes (STING) protein and thereby treat diseases such as cancer and microbial infections.

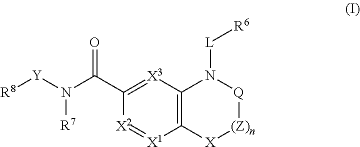

The present invention relates to small molecules for use in modulating the Stimulator of Interferon Genes (STING) protein. Accordingly, the small molecules may be for use in the treatment of diseases, such as cancer and microbial infections, and so on. The invention extends to the compounds per se pharmaceutical compositions, methods of making the compounds and methods of modulating the STING protein.

The human immune system may generally be divided into two arms, referred to as the ‘innate immune system’ and the ‘adaptive immune system’. The innate arm is mainly responsible for an initial inflammatory response via a number of factors such as cytokines, chemokines and complement factors. These factors act upon a number of different cell types including mast cells, macrophages, dendritic cells and natural killer cells. The adaptive arm involves a delayed and longer lasting response to challenge via antibody production together with CD8+ and CD4+ T-cell responses that are critical for immunological memory.

Research has been conducted for many years on how the immune system can recognise and eliminate malignant tumors (Parish et. al., Immunol and Cell Biol, 2003, 81, 106-113). One of the pioneers in this area is William Coley, who in the late 1800's noted that a cancer patient had a complete remission of their cancer after acute infection with the bacteria Streptococcus pyogenes. Subsequent studies with Coley's toxin and with bacille Calmette-Guerin (BCG) for cancer immunotherapy provided some clinical success but by no means offered a panacea for tumor treatment (Coley, Am J Med Sci., 1893, 105, 487-511). Through the 1900's, opinions fluctuated on the benefits of immunotherapy, with theories of acquired immunological tolerance (Burnet, Lancet, 1967, 1, 1171-1174 and Matzinger, Ann. Rev. Immunol., 1994, 12, 991-1045 and Smyth et. al., Nat Immunol., 2001, 2, 293-299) and tumor-associated antigens (Rosenberg et. al., Immunity, 1999, 10, 281-287) gaining support with the emergence of the innate immune system as an important mediator of immunity (Lanier, Nat Med. 2001, 2, 1178-1180 and Mayardomo et al., Nat Med. 1995, 1, 1297-1302 and Medzhitov et al., Trends Microbiol., 2000, 8, 452-456 and Akira et. al., Nat. Immunol., 2001, 2, 675-680). The detection of pathogen-associated molecular patterns (PAMPs) such as nucleic acids is now recognized as a central strategy by which the innate immune system senses microbes and tumor-associated antigens to then initiate protective responses (Barbalat et. al., Annu. Rev. Imunol., 2011, 29, 185-214).

As described above, innate immunity is initiated when PAMPs or damage-associated molecular patterns (DAMPs) are detected by pattern recognition receptors which include TLRs, NOD-like receptors and RIG-I-like receptors. These pattern recognition receptors respond to DAMPs and PAMPs by up-regulating Type-1 interferons and cytokines. Cytosolic nucleic acids are known PAMPs/DAMPs and engage the STING protein to stimulate the innate immune system and promote an antitumor response. Binding of dsDNA by cyclic GMP-AMP (cGAMP) synthase (cGAS) triggers formation of cyclic dinucleotides (CDNs). CDNs are second messenger signalling molecules produced by diverse bacteria and consist of two ribonucleotides that are connected via phosphodiester bonds to make a cyclic structure. CDNs Cyclo-di(GMP), cyclo-di(AMP) and hybrid cyclo-(AMP/GMP) derivatives all bind to STING with subsequent activation of the interferon pathway (Gao et. al., Cell, 2013, 153, 1094-1107; Zhang et. al., Mol. Cell, 2013, 51, 226-235). The canonical 5′-3′ phosphodiester linkage is recognised along with various other linkage isomers (notably the 5′-2′ linkage, e.g. c[G(2′,5′)pA(3′,5′)p]) which all bind to STING with various affinities (Shi et. al., PNAS, 2015, 112, 1947-8952). These observations have been corroborated by structural studies (Gao et. al., Cell, 2013, 154, 748-762) of various linkage isomers of CDNs bound to the human and mouse STING proteins.

One possible mechanism by which traditional vaccine adjuvants, such as alum, potentiate an immune response is through the release of DAMPs. Adjuvants, such as alum, trigger the release of host cell DNA, which can promote a Th2 response, induce T cell responses and the production of IgG1 and IgE. Ideally, adjuvants should be molecularly defined and able to enhance the magnitude and timeframe of a specific immune response to an antigen that offers protection against intracellular pathogens and/or reduce tumor burden.

Activation of the STING protein can create an activated or primed immune system, similarly to that generated by an adjuvant. This may produce a protective or prophylactic state upon challenge or re-challenge by intracellular pathogens or by tumors which inhibits the growth or propagation of intracellular pathogens or tumors.

It can also be appreciated that when a STING activator is administered therapeutically to a system in which tumors/pathogens are present it can act beneficially in two different, but related, ways. Firstly, by direct shrinkage of tumors/pathogen eradication through up-regulation of Type-I interferons and cytokines to act directly upon the tumor/pathogens, as described above. Secondly, a STING activator will also induce a lasting immune response, such that re-challenge or re-inoculation with a pathogen or tumors will be resisted both through a general activation of the immune system and through a latent antigen-specific response to said pathogen or tumor.

Tumor immunosurveillance does occur with, for example, thriving tumors having been immunoselected to evade immune elimination and indeed, the crucial role that the innate immune system plays in tumor clearance puts Coley's original findings in a new light. It is now clear that fragments of cyclic nucleotides, oligonucleotides and double stranded motifs can all activate the innate immune system through toll-like receptors (Horscroft, J. Antimicrob. Ther., 2012, 67(4), 789-801 and Diebold et al., Science, 2004, 303, 1529-1531), RIG-I like receptors (Pichlmair et. al., Science, 2006, 314, 997-1001) and stimulator of IFN genes (STING) adaptor proteins (Burdette et. al., Nat. Immunol., 2013, 14(1), 19-26).

This developing knowledge has stimulated considerable research into possible therapeutic applications of immunomodulation via some of these target classes.

STING has emerged more recently as a critical signalling molecule in the innate response to cytosolic nucleic acid molecules (Burdette and Vance, Nat. Immunol, 2013, 14, 19-26). STING plays a role in the transcriptional induction of Type I interferons and coregulated genes in response to nucleic acids in the cytosol. Studies in STING-deficient mice have confirmed the role of STING in innate responses to cytosolic nucleic-acid ligands, particularly double stranded DNA and bacterial nucleic acids based on a cyclic dinucleotide structure (Ishikawa et. al., Nature, 2009, 461, 788-792). STING has a critical role in the innate response to many bacterial, viral and eukaryotic pathogens (Watson et. al., Cell, 2012, 0.50, 803-815; de Almeida et. al., PLoS One, 2011, 6, e23135; Holm et. al, Nat. Immunol, 2012, 13, 737-743; Stein et. al., J. Virol., 2012, 86, 4527-4537; Sharma et. al., Immunity, 2011, 35, 194-207).

STING is broadly expressed throughout the body in both immune cells and non-immune cells, for example in the spleen, heart, thymus, placenta, lung and peripheral leukocytes, indicating a role in triggering the innate immune system in response to PAMPs/DAMPs (Sun et. al., PNAS, 2009, 106, 8653-8658). Its expression in immune cells leads to rapid amplification of the initial immune signal and maturation of APCs.

It is expressed in several transformed cell lines including HEK293 human embryonic kidney cells, A549 adenocarcinomic human alveolar basal epithelial cells, THP-1 monocytic cells and U937 leukemic monocytic lymphoma cells.

STING also has a central role in certain autoimmune disorders initiated by inappropriate recognition of self DNA (Gall et. al., Immunity, 2012, 36, 120-131) and has been proposed to sense membrane-fusing events associated with viral entry, in a manner independent of the sensing of nucleic acids (Holm et. al., Nat. Immunol., 2012, 13, 737-743).

STING is comprised of an N-terminal transmembrane domain, a central globular domain and a C-terminal tail. The protein forms a symmetrical dimer in the ligand bound state, with the cyclic dinucleotides binding at a dimer interface binding pocket. Binding of CDNs to STING activates a cascade of events whereby the protein recruits and activates IκB kinase (IKK) and TANK-binding kinase (TBK1), which following their phosphorylation activate nuclear transcription factors (NFκB) and interferon regulatory factor 3 (IRF3), respectively. These activated proteins translocate to the nucleus to induce transcription of the genes that encode Type I interferon and cytokines for promoting intercellular immune system defense. Sequence variations are known between human and mouse STING proteins, and between STING proteins within the human population. Several naturally occurring variant alleles have been identified.

Derivatives of the CDN class are currently being developed as antitumor agents upon intratumoral injection (Corrales et. al., Cell Rep., 2015, 19, 1018-1030). The xanthene-based small molecule 5,6-dimethyl-xanthenone acetic acid (DMXAA) was initially identified as a small molecule exhibiting immune modulatory activities through induction of cytokines and disrupting tumor vascularization in mouse xenograft models (Baguley and Ching, Int. J. Radiat. Oncol. Biol. Phys., 2002, 54, 1503-1511). This promising efficacy led to its investigation in a Phase II clinical trial against non-small cell lung carcinoma but subsequently failed its endpoints. The mechanism of DMXAA's activity against murine tumors was eventually ascribed to its activity as a murine STING activator. Its failure in human clinical trials was due to the fact that DMXAA was only capable of activating mouse STING and not human STING (Lara et. al., J. Clin. Oncol., 2011, 29, 2965-2971; Conlon et. al., J. Immunol., 2013, 190, 5216-5225). This lack of human activity has hampered all further attempts to develop this agent as a tumor therapy. Recently, a related small molecule 10-carboxymethyl-9-acridanone (CMA) (Cavlar et. al., EMBO J., 2013, 32, 1440-1450) has been found to bind to mouse STING, but also not to human STING. Both DMXAA and CMA have been shown to bind two molecules of each ligand to the STING dimer at a region close to the dimer interface.

Accordingly, there remains a need in the art for improved therapies for treating diseases, such as cancer, which can be refractory to traditional therapeutic approaches.

Immunologic strategies show promise for the treatment of cancer, and there is a need to develop improved compositions and methods in this field. In particular, there is a need for compounds that modulate the human STING protein, as well as methods for treating diseases that can benefit from such modulation.

The present invention has arisen from the inventors work in attempting to identify STING protein modulators.

Hence, in a first aspect of the invention, there is provided a compound of formula (I):

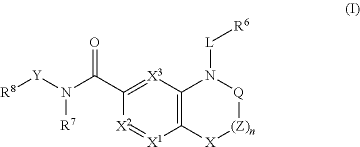

or a pharmaceutically acceptable salt or prodrug thereof, wherein:

X is CR⁹R¹⁰, O, S, S═O or SO₂;

X¹ is CR¹ or N;

X² is CR² or N;

X³ is CR³ or N;

the or each Z is CR¹¹R¹²;

n is 1 or 2;

Q is C═O, S═O, SO₂, C═S or CR⁴R⁵;

L is optionally substituted C₁-C₆ alkyl, C₁-C₃ polyfluoroalkyl, optionally substituted C₃-C₆ cycloalkyl, optionally substituted C₂-C₆ alkenyl, optionally substituted C₂-C₆ alkynyl, C═O, S═O, SO₂, —CH₂C(O)—, —CH₂CONH—, or —CONH—;

Y is an optionally substituted C₁-C₆ alkyl, C₁-C₃ polyfluoroalkyl, an optionally substituted C₂-C₆ alkenyl, an optionally substituted C₂-C₆ alkynyl, an optionally substituted C₃-C₆ cycloalkyl, or an optionally substituted mono or bicyclic 3 to 8 membered heterocycle;

R¹, R² and R³ are each independently selected from the group consisting of H, halogen, CN, hydroxyl, COOH, CONR¹R², NR¹R², NHCOR¹, optionally substituted C₁-C₆ alkyl, C₁-C₃ polyfluoroalkyl, optionally substituted C₁-C₆ alkylsulfonyl, optionally substituted mono or bicyclic C₃-C₆ cycloalkyl, optionally substituted C₂-C₆ alkenyl, optionally substituted C₂-C₆ alkynyl, optionally substituted C₁-C₆ alkoxy, optionally substituted C₁-C₆ alkoxycarbonyl group, mono or bicyclic optionally substituted C₅-C₁₀ aryl, mono or bicyclic optionally substituted 5 to 10 membered heteroaryl, optionally substituted mono or bicyclic 3 to 8 membered heterocycle, optionally substituted aryloxy, optionally substituted heteroaryloxy, and optionally substituted heterocyclyloxy;

R⁴ and R⁵ are each independently selected from the group consisting of H, halogen, optionally substituted C₁-C₆ alkyl and optionally substituted C₃-C₆ cycloalkyl; or R⁴ and

R⁵ together with the atom to which they are attached form a spirocyclic ring;

R⁶ is a mono or bicyclic optionally substituted C₅-C₁₀ aryl, mono or bicyclic optionally substituted 5 to 10 membered heteroaryl, optionally substituted C₃-C₆ cycloalkyl or an optionally substituted mono or bicyclic 3 to 8 membered heterocycle;

R⁷ is H, optionally substituted C₁-C₆ alkyl, optionally substituted sulfonyl, optionally substituted C₁-C₆ alkylsulfonyl, optionally substituted C₃-C₆ cycloalkyl, optionally substituted C₂-C₆ alkenyl and optionally substituted C₂-C₆ alkynyl;

R⁸ is a mono or bicyclic optionally substituted C₅-C₁₀ aryl, mono or bicyclic optionally substituted 5 to 10 membered heteroaryl, optionally substituted mono or bicyclic C₃-C₆ cycloalkyl or an optionally substituted mono or bicyclic 3 to 8 membered heterocycle;

R⁹ and R¹⁰ are each independently selected from the group consisting of optionally substituted C₁-C₆ alkyl, H, halogen, CN, hydroxyl, CO₂H, CONR¹R², azido, sulfonyl, NR¹R², NHCOR¹, C₁-C₃ polyfluoroalkyl, optionally substituted C₁-C₆ thioalkyl, optionally substituted C₁-C₆ alkylsulfonyl, optionally substituted C₃-C₆ cycloalkyl, optionally substituted C₂-C₆ alkenyl, optionally substituted C₂-C₆ alkynyl, optionally substituted C₁-C₆ alkoxy, optionally substituted C₁-C₆ alkoxycarbonyl, mono or bicyclic optionally substituted C₅-C₁₀ aryl, mono or bicyclic optionally substituted 5 to 10 membered heteroaryl, optionally substituted heterocycle, optionally substituted aryloxy, and an optionally substituted heteroaryloxy; or R⁹ and R¹⁰ together with the C atom to which they are attached can combine to form an optionally substituted spirocyclic ring; and

R¹¹ and R¹² are each independently selected from the group consisting of optionally substituted C₁-C₆ alkyl, H, halogen, CN, hydroxyl, CO₂H, CONR¹R², azido, sulfonyl, NR¹R², NHCOR¹, C₁-C₃ polyfluoroalkyl, optionally substituted C₁-C₆ thioalkyl, optionally substituted C₁-C₆ alkylsulfonyl, optionally substituted C₃-C₆ cycloalkyl, optionally substituted C₂-C₆ alkenyl, optionally substituted C₂-C₆ alkynyl, optionally substituted C₁-C₆ alkoxy, optionally substituted C₁-C₆ alkoxycarbonyl, mono or bicyclic optionally substituted C₅-C₁₀ aryl, mono or bicyclic optionally substituted 5 to 10 membered heteroaryl, optionally substituted heterocycle, optionally substituted aryloxy, and an optionally substituted heteroaryloxy; or R¹¹ and R¹² together with the C atom to which they are attached can combine to form an optionally substituted spirocyclic ring;

with the proviso that when X is S; X¹, X² and X³ are CH; n is 1; Z is CH₂; Q is C═O; L is —CH₂—; Y is —CH₂—; R⁷ is H; and R⁶ is unsubstituted phenyl

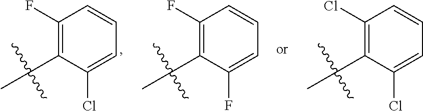 [SMILES: *c1c(F)cccc1Cl ; *c1c(F)cccc1F ; *c1c(Cl)cccc1Cl]

then R⁸ is not unsubstituted furanyl; and

when X is S; X¹, X² and X³ are CH; n is 1; Z is CH₂; Q is C═O; L is —CH₂—; Y is —CH₂—;

R⁷ is H; and R⁶ is

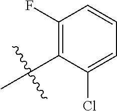 [SMILES: *c1c(F)cccc1Cl]

then R⁸ is not unsubstituted phenyl;

or a pharmaceutically acceptable complex, salt, solvate, tautomeric form or polymorphic form thereof, for use in therapy.

Preferably, when X is S; X¹, X² and X³ are CH; n is 1; Z is CH₂; Q is C═O; L is —CH₂—; Y is —CH₂—; R⁷ is H; and R⁶ is an optionally substituted phenyl then R⁸ is not an optionally substituted 5 membered heteroaryl. Optionally, when X is S; X¹, X² and X³ are CH; n is 1; Z is CH₂; Q is C═O; L is —CH₂—; Y is —CH₂—; R⁷ is H; and R⁶ is an optionally substituted phenyl then R⁸ is not an optionally substituted 5 or 6 membered heteroaryl.

Preferably, when X is S; X¹, X² and X³ are CH; n is 1; Z is CH₂; Q is C═O; L is —CH₂—; Y is —CH₂—; R⁷ is H; and R⁶ is unsubstituted phenyl,

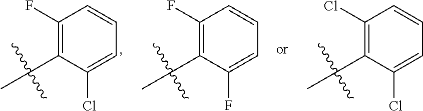 [SMILES: *c1c(F)cccc1Cl ; *c1c(F)cccc1F ; *c1c(Cl)cccc1Cl]

then R⁸ is not unsubstituted phenyl.

Preferably, when X is S; X¹, X² and X³ are CH; n is 1; Z is CH₂; Q is C═O; L is —CH₂—; Y is —CH₂—; R⁷ is H; and R⁶ is an optionally substituted phenyl then R⁸ is not an unsubstituted phenyl.

The inventors have also found that compounds of formula (I) are useful in modulating the Stimulator of Interferon Genes (STING) protein.

Hence, in a second aspect, there is provided a compound of formula (I) or a pharmaceutically acceptable complex, salt, solvate, tautomeric form or polymorphic form thereof, for use in modulating the Stimulator of Interferon Genes (STING) protein.

Preferably, the compound of formula (I) is for use in activating, or agonising, the STING protein.

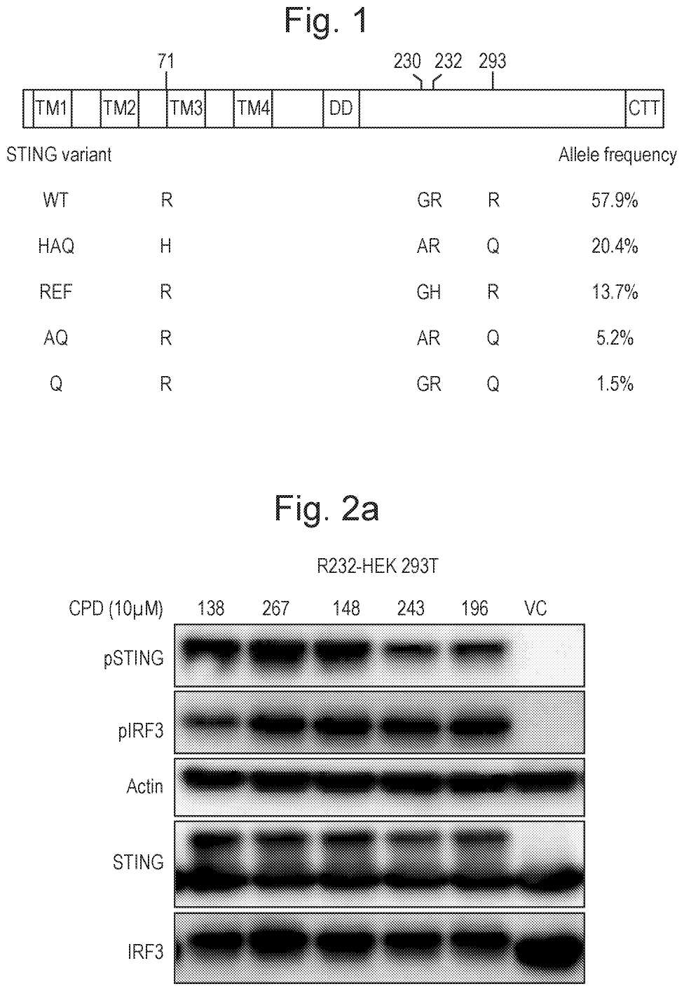

Advantageously, the compounds of the invention modulate the major polymorphs of the human STING protein. There are many STING polymorphs reported, but the 5 polymorphs listed below are the major ones which comprise 99% of the total human population. Accordingly, the STING protein may be a wild type polymorph (WT/R232), a HAQ polymorph, a REF polymorph (H232), an AQ polymorph or a Q polymorph. As shown in FIG. 1, the wild type polymorph has arginines at the 71, 232 and 293 positions and a glycine at the 230 position, the HAQ polymorph has a histidine at the 71 position, an alanine at the 230 position, an arginine at the 232 position and a glutamine at the 293 position, the REF polymorph has arginines at the 71 and 293 positions, a glycine at the 230 position and a histidine at the 232 position, the AQ polymorph has arginines at the 71 and 232 positions, an alanine at the 230 position and a glutamine at the 293 position, and the Q polymorph has arginines at the 71 and 232 positions, a glycine at the 230 position and a glutamine at the 293 position.

By modulating the STING protein, it is possible to treat, ameliorate or prevent cancer, bacterial infection, viral infection, parasitic infection, fungal infection, immune-mediated disorder, central nervous system disease, peripheral nervous system disease, neurodegenerative disease, mood disorder, sleep disorder, cerebrovascular disease, peripheral artery disease or cardiovascular disease.

Accordingly, in a third aspect there is provided a compound of formula (I) or a pharmaceutically acceptable complex, salt, solvate, tautomeric form or polymorphic form thereof, for use in treating, ameliorating or preventing a disease selected from cancer, bacterial infection, viral infection, parasitic infection, fungal infection, immune-mediated disorder, central nervous system disease, peripheral nervous system disease, neurodegenerative disease, mood disorder, sleep disorder, cerebrovascular disease, peripheral artery disease or cardiovascular disease.

Preferably, the disease is cancer.

In a fourth aspect, there is provided a method of modulating the Stimulator of Interferon Genes (STING) protein in a subject, the method comprising administering, to a subject in need of such treatment, a therapeutically effective amount of a compound of formula (I) or a pharmaceutically acceptable complex, salt, solvate, tautomeric form or polymorphic form thereof.

Preferably, the method comprises activating the STING protein.

The STING protein may be a wild type polymorph, a HAQ polymorph, a REF polymorph, an AQ polymorph or a Q polymorph.

In a fifth aspect, there is provided a method of treating, ameliorating or preventing a disease selected from cancer, bacterial infection, viral infection, parasitic infection, fungal infection, immune-mediated disorder, central nervous system disease, peripheral nervous system disease, neurodegenerative disease, mood disorder, sleep disorder, cerebrovascular disease, peripheral artery disease or cardiovascular disease, the method comprising administering, to a subject in need of such treatment, a therapeutically effective amount of a compound of formula (I) or a pharmaceutically acceptable complex, salt, solvate, tautomeric form or polymorphic form thereof.

Preferably, the disease is cancer.

It may be appreciated that the term “preventing” can mean “reducing the likelihood of”.

The neurodegenerative disease may be Alzheimer's disease or dementia. The viral disease may be Hepatitis. The parasitic infection may be malaria. The mood disorder may be depression. The sleep disorder may be insomnia.

In one preferred embodiment, the disease is cancer. The cancer may be selected from the group consisting of colorectal cancer, aero-digestive squamous cancer, lung cancer, brain cancer, liver cancer, stomach cancer, sarcoma, leukaemia, lymphoma, multiple myeloma, ovarian cancer, uterine cancer, breast cancer, melanoma, prostate cancer, bladder cancer, pancreatic carcinoma or renal carcinoma.

In an alternative preferred embodiment, the disease is a viral infection. The viral infection may be a hepatitis C virus (HCV) infection.

The inventors believe that a number of the compounds which fall within the scope of formula (I) are novel and inventive per se.

Hence, in a sixth aspect, there is provided a compound of formula (I):

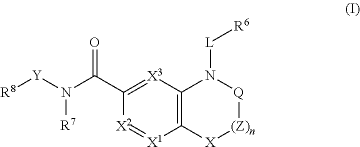

or a pharmaceutically acceptable salt or prodrug thereof, wherein:

X is CR⁹R¹⁰, O, S, S═O or SO₂;

X¹ is CR¹ or N;

X² is CR² or N;

X³ is CR³ or N;

the or each Z is CR¹¹R¹²;

n is 1 or 2;

Q is C═O, S═O, SO₂, C═S or CR⁴R⁵;

L is optionally substituted C₁-C₆ alkyl, C₁-C₃ polyfluoroalkyl, optionally substituted C₃-C₆ cycloalkyl, optionally substituted C₂-C₆ alkenyl, optionally substituted C₂-C₆ alkynyl, C═O, S═O, SO₂, —CH₂C(O)—, —CH₂CONH—, or —CONH—;

Y is an optionally substituted C₁-C₆ alkyl, C₁-C₃ polyfluoroalkyl, an optionally substituted C₂-C₆ alkenyl, an optionally substituted C₂-C₆ alkynyl, an optionally substituted C₃-C₆ cycloalkyl, or an optionally substituted mono or bicyclic 3 to 8 membered heterocycle;

R¹, R² and R³ are each independently selected from the group consisting of H, halogen, CN, hydroxyl, COOH, CONR¹R², NR¹R², NHCOR¹, optionally substituted C₁-C₆ alkyl, C₁-C₃ polyfluoroalkyl, optionally substituted C₁-C₆ alkylsulfonyl, optionally substituted mono or bicyclic C₃-C₆ cycloalkyl, optionally substituted C₂-C₆ alkenyl, optionally substituted C₂-C₆ alkynyl, optionally substituted C₁-C₆ alkoxy, optionally substituted C₁-C₆ alkoxycarbonyl group, mono or bicyclic optionally substituted C₅-C₁₀ aryl, mono or bicyclic optionally substituted 5 to 10 membered heteroaryl, optionally substituted mono or bicyclic 3 to 8 membered heterocycle, optionally substituted aryloxy, optionally substituted heteroaryloxy, and optionally substituted heterocyclyloxy;

R⁴ and R⁵ are each independently selected from the group consisting of H, halogen, optionally substituted C₁-C₆ alkyl, optionally substituted (C₃-C₆) cycloalkyl or R⁴ and R⁵ together with the atom to which they are attached form a spirocyclic ring;

R⁶ is mono or bicyclic optionally substituted C₅-C₁₀ aryl, mono or bicyclic optionally substituted 5 to 10 membered heteroaryl, optionally substituted C₃-C₆ cycloalkyl or an optionally substituted mono or bicyclic 3 to 8 membered heterocycle;

R⁷ is H, optionally substituted C₁-C₆ alkyl, optionally substituted sulfonyl, optionally substituted C₁-C₆ alkylsulfonyl, optionally substituted C₃-C₆ cycloalkyl, optionally substituted C₂-C₆ alkenyl and optionally substituted C₂-C₆ alkynyl;

R⁸ is mono or bicyclic optionally substituted C₅-C₁₀ aryl, mono or bicyclic optionally substituted 5 to 10 membered heteroaryl, optionally substituted mono or bicyclic C₃-C₆ cycloalkyl or an optionally substituted mono or bicyclic 3 to 8 membered heterocycle;

R⁹ and R¹⁰ are each independently selected from the group consisting of optionally substituted C₁-C₆ alkyl, H, halogen, CN, hydroxyl, CO₂H, CONR¹R², azido, sulfonyl, NR¹R², NHCOR, C₁-C₃ polyfluoroalkyl, optionally substituted C₁-C₆ thioalkyl, optionally substituted C₁-C₆ alkylsulfonyl, optionally substituted C₃-C₆ cycloalkyl, optionally substituted C₂-C₆ alkenyl, optionally substituted C₂-C₆ alkynyl, optionally substituted C₁-C₆ alkoxy, optionally substituted C₁-C₆ alkoxycarbonyl, mono or bicyclic optionally substituted C₅-C₁₀ aryl, mono or bicyclic optionally substituted 5 to 10 membered heteroaryl, optionally substituted heterocycle, optionally substituted aryloxy, and an optionally substituted heteroaryloxy; or R⁹ and R¹⁰ together with the C atom to which they are attached can combine to form an optionally substituted spirocyclic ring; and

R¹¹ and R¹² are each independently selected from the group consisting of optionally substituted C₁-C₆ alkyl, H, halogen, CN, hydroxyl, CO₂H, CONR¹R², azido, sulfonyl, NR¹R², NHCOR¹, C₁-C₃ polyfluoroalkyl, optionally substituted C₁-C₆ thioalkyl, optionally substituted C₁-C₆ alkylsulfonyl, optionally substituted C₃-C₆ cycloalkyl, optionally substituted C₂-C₆ alkenyl, optionally substituted C₂-C₆ alkynyl, optionally substituted C₁-C₆ alkoxy, optionally substituted C₁-C₆ alkoxycarbonyl, mono or bicyclic optionally substituted C₅-C₁₀ aryl, mono or bicyclic optionally substituted 5 to 10 membered heteroaryl, optionally substituted heterocycle, optionally substituted aryloxy, and an optionally substituted heteroaryloxy; or R¹¹ and R¹² together with the C atom to which they are attached can combine to form an optionally substituted spirocyclic ring;

with the proviso that when X is S; X¹, X² and X³ are CH; n is 1; Z is CH₂; Q is C═O; L is —CH₂—; Y is —CH₂—; R⁷ is H; and R⁶ is

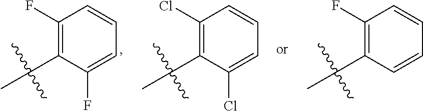 [SMILES: *c1c(F)cccc1F ; *c1c(Cl)cccc1Cl ; *c1ccccc1F]

then R⁸ is not unsubstituted furanyl;

when X is S; X¹, X² and X³ are CH; n is 1; Z is CH₂; Q is C═O; L is —CH₂—; Y is —CH₂—;

R⁷ is H; and R⁶ is

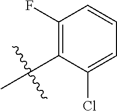 [SMILES: *c1c(F)cccc1Cl]

then R⁸ is not unsubstituted phenyl, unsubstituted thiophenyl, unsubstituted pyridinyl, unsubstituted furanyl, unsubstituted tetrahydrofuranyl,

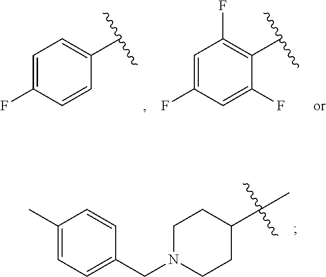 [SMILES: *c1ccc(F)cc1 ; *c1c(F)cc(F)cc1F ; *C1CCN(Cc2ccc(C)cc2)CC1]

when X is S; X¹, X² and X³ are CH; n is 1; Z is CH₂; Q is C═O; L is —CH₂—; Y is —CH₂CH₂—; R⁷ is H; and R⁶ is

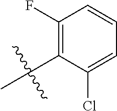 [SMILES: *c1c(F)cccc1Cl]

then R⁸ is not unsubstituted phenyl;

when X is S; X¹, X² and X³ are CH; n is 1; Z is CH₂; Q is C═O; L is —CH₂—; Y is

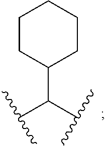 [SMILES: *C(*)C1CCCCC1]

R⁷ is H; and R⁶ is

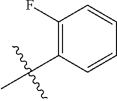 [SMILES: *c1ccccc1F]

then R⁸ is not unsubstituted phenyl;

when X is S; X¹, X² and X³ are CH; n is 1; Z is CH₂; Q is C═O; L is —CH₂—; Y is —CH₂—;

R⁷ is H; and R⁶ is unsubstituted phenyl then R⁸ is not unsubstituted furanyl, unsubstituted phenyl,

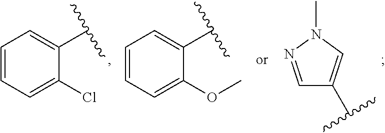

when X is S; X¹, X² and X³ are CH; n is 1; Z is CH₂; Q is C═O or CH₂; L is —CH₂—; Y is —CH₂—; R⁷ is H; and R⁶ is

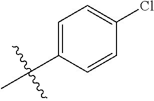 [SMILES: *c1ccc(Cl)cc1]

then R⁸ is not unsubstituted furanyl;

when X is S; X¹, X² and X³ are CH; n is 1; Z is CH₂; Q is C═O; L is —CH₂—; Y is —CH₂—;

R⁷ is H; and R⁶ is

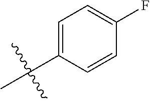 [SMILES: *c1ccc(F)cc1]

then R⁸ is not unsubstituted furanyl;

when X is S; X¹, X² and X³ are CH; n is 1; Z is CH₂; Q is C═O; L is —CH₂—; Y is —CH₂—;

R⁷ is H; and R⁶ is

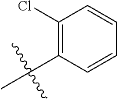 [SMILES: *c1ccccc1Cl]

then R⁸ is not unsubstituted furanyl,

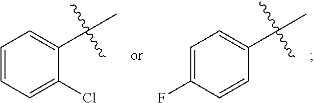 [SMILES: *c1ccccc1Cl ; *c1ccc(F)cc1]

when X is S; X¹, X² and X³ are CH; n is 1; Z is CH₂; Q is C═O; L is —CH₂—; Y is —CH₂—;

R⁷ is H; and R⁶ is

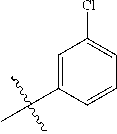 [SMILES: *c1cccc(Cl)c1]

then R⁸ is not unsubstituted furanyl or

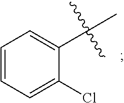 [SMILES: *c1ccccc1Cl]

and

when X is S; X, X² and X³ are CH; n is 1; Z is CH₂; Q is C═O; L is —CH₂—; Y is

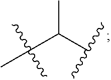 [SMILES: *C(*)C]

R⁷ is H; and R⁶ is

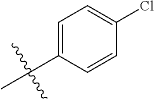 [SMILES: *c1ccc(Cl)cc1]

then R⁸ is not unsubstituted phenyl;

or a pharmaceutically acceptable complex, salt, solvate, tautomeric form or polymorphic form thereof.

Preferably, when X is S; X¹, X² and X³ are CH; n is 1; Z is CH₂; Q is C═O; L is —CH₂—; Y is —CH₂—; R⁷ is H; and R⁶ is an optionally substituted phenyl then R⁸ is not an optionally substituted 5 or 6 membered heteroaryl or tetrahydrofuranyl.

Preferably, when X is S; X¹, X² and X³ are CH; n is 1; Z is CH₂; Q is C═O; L is —CH₂—; R⁷ is H; and R⁶ is

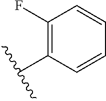 [SMILES: *c1ccccc1F]

then R⁸ is not an unsubstituted phenyl or unsubstituted cyclohexane.

Preferably, when X is S; X¹, X² and X³ are CH; n is 1; Z is CH₂; Q is C═O; L is —CH₂—; Y is an optionally substituted C₁-C₂ alkyl; R⁷ is H; and R⁶ is an optionally substituted phenyl then R⁸ is not an optionally substituted 5 or 6 membered heteroaryl, an optionally substituted phenyl or tetrahydrofuranyl.

The following definitions are used in connection with the compounds of the present invention unless the context indicates otherwise.

Throughout the description and the claims of this specification the word “comprise” and other forms of the word, such as “comprising” and “comprises,” means including but not limited to, and is not intended to exclude for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions.

“Optional” or “optionally” means that the subsequently described event, operation or circumstances can or cannot occur, and that the description includes instances where the event, operation or circumstance occurs and instances where it does not.

The term “alkyl,” as used herein, unless otherwise specified, refers to a saturated straight or branched hydrocarbon. In certain embodiments, the alkyl group is a primary, secondary, or tertiary hydrocarbon. In certain embodiments, the alkyl group includes one to six carbon atoms, i.e. C₁-C₆ alkyl. C₁-C₆ alkyl includes for example methyl, ethyl, n-propyl (1-propyl) and isopropyl (2-propyl, 1-methylethyl), butyl, pentyl, hexyl, isobutyl, sec-butyl, tert-butyl, isopentyl, neopentyl, and isohexyl. An alkyl group can be unsubstituted or substituted with one or more of halogen, OH, O(P)O(OH)₂, C₁-C₆ alkoxy, NR¹R², CONR¹R², CN, COOH, C₅-C₁₀ aryl, 5 to 10 membered heteroaryl, C₃-C₆ cycloalkyl and 3 to 8 membered heterocycle. Accordingly, it will be appreciated that an optionally substituted C₁-C₆ alkyl may be an optionally substituted C₁-C₆ haloalkyl, i.e. a C₁-C₆ alkyl substituted with at least one halogen, and optionally further substituted with one or more of OH, C₁-C₆ alkoxy, NR¹R², CONR¹R², CN, COOH, C₅-C₁₀ aryl, 5 to 10 membered heteroaryl, C₃-C₆ cycloalkyl and 3 to 8 membered heterocycle. R¹ and R² may each independently be selected from the group consisting of H, halogen and optionally substituted C₁-C₆ alkyl.

The term “halo” includes fluoro (—F), chloro (—C₁), bromo (—Br) and iodo (—I).

The term “polyfluoroalkyl” may denote a C₁-C₃ alkyl group in which two or more hydrogen atoms are replaced by fluorine atoms. The term may include perfluoroalkyl groups, i.e. a C₁-C₃ alkyl group in which all the hydrogen atoms are replaced by fluorine atoms. Accordingly, the term C₁-C₃ polyfluoroalkyl includes, but is not limited to, difluoromethyl, trifluoromethyl, 2,2,2-trifluoroethyl, pentafluoroethyl, 3,3,3-trifluoropropyl, 2,2,3,3,3-pentafluoropropyl, and 2,2,2-trifluoro-1-(trifluoromethyl)ethyl.

“Alkoxy” refers to the group R¹³—O— where R¹³ is an optionally substituted C₁-C₆ alkyl group, an optionally substituted C₁-C₆ alkenyl group, an optionally substituted C₂-C₆ alkynyl or an optionally substituted C₃-C₆ cycloalkyl group. Exemplary C₁-C₆ alkoxy groups include but are not limited to methoxy, ethoxy, n-propoxy (1-propoxy), n-butoxy and tert-butoxy. An alkoxy group can be unsubstituted or substituted with one or more of halogen, OH, O(P)O(OH)₂, alkoxy, NR¹R², CONR¹R², CN, COOH, aryl, heteroaryl, cycloalkyl and heterocycle. R¹ and R² may each independently be selected from the group consisting of H, halogen and optionally substituted C₁-C₆ alkyl.

“Thioalkyl” refers to the group R¹³—S— where R¹³ is an optionally substituted C₁-C₆ alkyl group or an optionally substituted C₃-C₆ cycloalkyl group. A thioalkyl group can be unsubstituted or substituted with one or more of halogen, OH, O(P)O(OH)₂, alkoxy, NR¹R², CONR¹R², CN, COOH, aryl, heteroaryl, cycloalkyl and heterocycle. R¹ and R² may each independently be selected from the group consisting of H, halogen and optionally substituted C₁-C₆ alkyl.

“Aryl” refers to an aromatic 5 to 10 membered hydrocarbon group. Examples of a C₅-C₁₀ aryl group include, but are not limited to, phenyl, α-naphthyl, β-naphthyl, biphenyl, tetrahydronaphthyl and indanyl. An aryl group can be unsubstituted or substituted with one or more of optionally substituted C₁-C₆ alkyl, halogen, OH, O(P)O(OH)₂, optionally substituted C₁-C₆ alkoxy, NR¹R², CONR¹R², CO₂R¹, OC(O)OR¹, OC(O)NR¹R², CN, COOH, NO₂, azido, C₁-C₃ polyfluoroalkyl, aryloxy, heteroaryloxy, 5 to 10 membered heteroaryl, 3 to 8 membered heterocycle, SO₂R¹ and NHCOR¹. R¹ and R² may each independently be selected from the group consisting of H, halogen and optionally substituted C₁-C₆ alkyl.

The term “bicycle” or “bicyclic” as used herein refers to a molecule that features two fused rings, which rings are a cycloalkyl, heterocyclyl, or heteroaryl. In one embodiment, the rings are fused across a bond between two atoms. The bicyclic moiety formed therefrom shares a bond between the rings. In another embodiment, the bicyclic moiety is formed by the fusion of two rings across a sequence of atoms of the rings to form a bridgehead. Similarly, a “bridge” is an unbranched chain of one or more atoms connecting two bridgeheads in a polycyclic compound. In another embodiment, the bicyclic molecule is a “spiro” or “spirocyclic” moiety. The spirocyclic group may be a C₃-C₆ cycloalkyl or a mono or bicyclic 3 to 8 membered heterocycle which is bound through a single carbon atom of the spirocyclic moiety to a single carbon atom of a carbocyclic or heterocyclic moiety. In one embodiment, the spirocyclic group is a cycloalkyl and is bound to another cycloalkyl. In another embodiment, the spirocyclic group is a cycloalkyl and is bound to a heterocyclyl. In a further embodiment, the spirocyclic group is a heterocyclyl and is bound to another heterocyclyl. In still another embodiment, the spirocyclic group is a heterocyclyl and is bound to a cycloalkyl. A spirocyclic group can be unsubstituted or substituted with one or more of an optionally substituted C₁-C₆ alkyl, halogen, OH, optionally substituted C₁-C₆ alkoxy, NR¹R², CONR¹R², CN, COOH, NO₂, azido, C₁-C₃ polyfluoroalkyl and NHCOR¹. R¹ and R² may each independently be selected from the group consisting of H, halogen and optionally substituted C₁-C₆ alkyl.

“Alkoxycarbonyl” refers to the group alkyl-O—C(O)—, where alkyl is a C₁-C₆ alkyl. An alkoxycarbonyl group can be unsubstituted or substituted with one or more of halogen, OH, NR¹R², CN, C₁-C₆ alkoxy, COOH, C₅-C₁₀ aryl, 5 to 10 membered heteroaryl or C₃-C₆ cycloalkyl. R¹ and R² may each independently be selected from the group consisting of H, halogen and optionally substituted C₁-C₆ alkyl.

“Aryloxy” refers to the group Ar—O— where Ar is a mono or bicyclic optionally substituted C₅-C₁₀ aryl group, as defined above.

“Cycloalkyl” refers to a non-aromatic, saturated, partially saturated, monocyclic, bicyclic or polycyclic hydrocarbon 3 to 6 membered ring system. Representative examples of a C₃-C₆ cycloalkyl include, but are not limited to, cyclopropyl, cyclobutyl, cyclopentyl, cyclohexyl. A cycloalkyl group can be unsubstituted or substituted with one or more of optionally substituted C₁-C₆ alkyl, halogen, CN, hydroxyl, COOH, CONR¹R², NR¹R², NHCOR¹, optionally substituted C₁-C₆ alkoxy, azido, C₁-C₃ polyfluoroalkyl, aryloxy, heteroaryloxy, SO₂R¹, mono or bicyclic optionally substituted C₅-C₁₀ aryl, mono or bicyclic optionally substituted 5 to 10 membered heteroaryl, or optionally substituted mono or bicyclic 3 to 8 membered heterocycle, C₃-C₆ cycloalkyl. R¹ and R² may each independently be selected from the group consisting of H, halogen and optionally substituted C₁-C₆ alkyl.

“Heteroaryl” refers to a monocyclic or bicyclic aromatic 5 to 10 membered ring system in which at least one ring atom is a heteroatom. The or each heteroatom may be independently selected from the group consisting of oxygen, sulfur and nitrogen. Examples of 5 to 10 membered heteroaryl groups include furan, thiophene, indole, azaindole, oxazole, thiazole, isoxazole, isothiazole, imidazole, N-methylimidazole, pyridine, pyrimidine, pyrazine, pyrrole, N-methylpyrrole, pyrazole, N-methylpyrazole, 1,3,4-oxadiazole, 1,2,4-triazole, 1-methyl-1,2,4-triazole, 1H-tetrazole, 1-methyltetrazole, benzoxazole, benzothiazole, benzofuran, benzisoxazole, benzimidazole, N-methylbenzimidazole, azabenzimidazole, indazole, quinazoline, quinoline, and isoquinoline. Bicyclic 5 to 10 membered heteroaryl groups include those where a phenyl, pyridine, pyrimidine, pyrazine or pyridazine ring is fused to a 5 or 6-membered monocyclic heteroaryl ring. A heteroaryl group can be unsubstituted or substituted with one or more of C₁-C₆ alkyl, halogen, OH, CN, NR¹R², azido, COOH, C₁-C₆ alkoxycarbonyl, C₁-C₃ polyfluoroalkyl, CONR¹R², CO₂R¹, OC(O)OR¹, OC(O)NR¹R², O(P)O(OH)₂, NO₂, NHCOR¹ and SO₂R¹. R¹ and R² may each independently be selected from the group consisting of H, halogen and optionally substituted C₁-C₆ alkyl.

“Heteroaryl” refers to a monocyclic or bicyclic aromatic 5 to 10 membered ring system in which at least one ring atom is a heteroatom. The or each heteroatom may be independently selected from the group consisting of oxygen, sulfur and nitrogen.

Examples of 5 to 10 membered heteroaryl groups include furan, thiophene, indole, azaindole, oxazole, thiazole, isoxazole, isothiazole, imidazole, N-methylimidazole, pyridine, pyrimidine, pyrazine, pyrrole, N-methylpyrrole, pyrazole, N-methylpyrazole, 1,3,4-oxadiazole, 1,2,4-triazole, 1-methyl-1,2,4-triazole, 1H-tetrazole, 1-methyltetrazole, benzoxazole, benzothiazole, benzofuran, benzisoxazole, benzimidazole, N-methylbenzimidazole, azabenzimidazole, indazole, quinazoline, quinoline, and isoquinoline. Bicyclic 5 to 10 membered heteroaryl groups include those where a phenyl, pyridine, pyrimidine, pyrazine or pyridazine ring is fused to a 5 or 6-membered monocyclic heteroaryl ring. A heteroaryl group can be unsubstituted or substituted with one or more of optionally substituted C₁-C₆ alkyl, halogen, optionally substituted C₁-C₆ alkoxy, OH, CN, NR¹R², azido, COOH, C₁-C₆ alkoxycarbonyl, C₁-C₃ polyfluoroalkyl, CONR¹R², NO₂, NHCOR¹ and SO₂R¹. R¹ and R² may each independently be selected from the group consisting of H, halogen and optionally substituted C₁-C₆ alkyl.

“Heterocycle” or “heterocyclyl” refers to 3 to 8 membered monocyclic, bicyclic or bridged molecules in which at least one ring atom is a heteroatom. The or each heteroatom may be independently selected from the group consisting of oxygen, sulfur and nitrogen. A heterocycle may be saturated or partially saturated. Exemplary 3 to 8 membered heterocyclyl groups include but are not limited to aziridine, oxirane, oxirene, thiirane, pyrroline, pyrrolidine, dihydrofuran, tetrahydrofuran, dihydrothiophene, tetrahydrothiophene, dithiolane, piperidine, 1,2,3,6-tetrahydropyridine-1-yl, tetrahydropyran, pyran, morpholine, piperazine, thiane, thiine, piperazine, azepane, diazepane, oxazine. A heterocyclyl group can be unsubstituted or substituted with one or more of optionally substituted C₁-C₆ alkyl, halogen, optionally substituted C₁-C₆ alkoxy, OH, NR¹R², COOH, C₁-C₆ alkoxycarbonyl, CONR¹R², NO₂, NHCOR, mono or bicyclic optionally substituted C₅-C₁₀ aryl and SO₂R¹. R¹ and R² may each independently be selected from the group consisting of H, halogen and optionally substituted C₁-C₆ alkyl.

“Alkenyl” refers to olefinically unsaturated hydrocarbon groups which can be unbranched or branched. In certain embodiments, the alkenyl group has 2 to 6 carbons, i.e. it is a C₂-C₆ alkenyl. C₂-C₆ alkenyl includes for example vinyl, allyl, propenyl, butenyl, pentenyl and hexenyl. An alkenyl group can be unsubstituted or substituted with one or more of C₁-C₆ alkyl, halogen, OH, C₁-C₆ alkoxy, C₁-C₃ polyfluoroalkyl, NR¹R², CONR¹R², SO₂R¹, NHCOR¹, CN, COOH, C₅-C₁₀ aryl, 5 to 10 membered heteroaryl, C₃-C₆ cycloalkyl, aryloxy, heteroaryloxy, and 3 to 8 membered heterocycle. R¹ and R² may each independently be selected from the group consisting of H, halogen and optionally substituted C₁-C₆ alkyl.

“Alkynyl” refers to acetylenically unsaturated hydrocarbon groups which can be unbranched or branched. In certain embodiments, the alkynyl group has 2 to 6 carbons, i.e. it is a C₂-C₆ alkynyl. C₂-C₆ alkynyl includes for example propargyl, propynyl, butynyl, pentynyl and hexynyl. An alkynyl group can be unsubstituted or substituted with one or more of C₁-C₆ alkyl, halogen, OH, C₁-C₆ alkoxy, C₁-C₃ polyfluoroalkyl, NR¹R², CONR¹R², SO₂R¹, NHCOR¹, CN, COOH, C₅-C₁₀ aryl, 5 to 10 membered heteroaryl, C₃-C₆ cycloalkyl, aryloxy, heteroaryloxy, and 3 to 8 membered heterocycle. R¹ and R² may each independently be selected from the group consisting of H, halogen and optionally substituted C₁-C₆ alkyl.

“Alkylsulfonyl” refers to the group alkyl-SO₂— where alkyl is an optionally substituted C₁-C₆ alkyl, and is as defined as above.

“Heteroaryloxy” refers to the group heteroaryl-O— where the heteroaryl is a mono or bicyclic optionally substituted 5 to 10 membered heteroaryl, and is as defined above.

“Heterocyclyloxy” refers to the group heterocycle-O— where heterocycle is an optionally substituted mono or bicyclic 3 to 8 membered heterocycle, and is as defined as above.

A complex of the compound of formula (I) may be understood to be a multi-component complex, wherein the drug and at least one other component are present in stoichiometric or non-stoichiometric amounts. The complex may be other than a salt or solvate. Complexes of this type include clathrates (drug-host inclusion complexes) and co-crystals. The latter are typically defined as crystalline complexes of neutral molecular constituents which are bound together through non-covalent interactions, but could also be a complex of a neutral molecule with a salt. Co-crystals may be prepared by melt crystallisation, by recrystallisation from solvents, or by physically grinding the components together—see Chem Commun, 1, 1889-1896, by O. Almarsson and M. J. Zaworotko (2004), incorporated herein by reference. For a general review of multi-component complexes, see J Pharm Sci, 64 (8), 1269-1288, by Haleblian (August 1975), incorporated herein by reference.

The term “pharmaceutically acceptable salt” may be understood to refer to any salt of a compound provided herein which retains its biological properties and which is not toxic or otherwise undesirable for pharmaceutical use. Such salts may be derived from a variety of organic and inorganic counter-ions well known in the art. Such salts include, but are not limited to: (1) acid addition salts formed with organic or inorganic acids such as hydrochloric, hydrobromic, sulfuric, nitric, phosphoric, sulfamic, acetic, adepic, aspartic, trifluoroacetic, trichloroacetic, propionic, hexanoic, cyclopentylpropionic, glycolic, glutaric, pyruvic, lactic, malonic, succinic, sorbic, ascorbic, malic, maleic, fumaric, tartaric, citric, benzoic, 3-(4-hydroxybenzoyl)benzoic, picric, cinnamic, mandelic, phthalic, lauric, methanesulfonic, ethanesulfonic, 1,2-ethane-disulfonic, 2-hydroxyethanesulfonic, benzenesulfonic, 4-chlorobenzenesulfonic, 2-naphthalenesulfonic, 4-toluenesulfonic, camphoric, camphorsulfonic, 4-methylbicyclo[2.2.2]-oct-2-ene-1-carboxylic, glucoheptonic, 3-phenylpropionic, trimethylacetic, tert-butylacetic, lauryl sulfuric, gluconic, benzoic, glutamic, hydroxynaphthoic, salicylic, stearic, cyclohexylsulfamic, quinic, muconic acid and the like acids; or (2) base addition salts formed when an acidic proton present in the parent compound either (a) is replaced by a metal ion, e.g., an alkali metal ion, an alkaline earth ion or an aluminium ion, or alkali metal or alkaline earth metal hydroxides, such as sodium, potassium, calcium, magnesium, aluminium, lithium, zinc, and barium hydroxide, ammonia or (b) coordinates with an organic base, such as aliphatic, alicyclic, or aromatic organic amines, such as ammonia, methylamine, dimethylamine, diethylamine, picoline, ethanolamine, diethanolamine, triethanolamine, ethylenediamine, lysine, arginine, ornithine, choline, N,N′-dibenzylethylene-diamine, chloroprocaine, diethanolamine, procaine, N-benzylphenethylamine, N-methylglucamine piperazine, tris(hydroxymethyl)-aminomethane, tetramethylammonium hydroxide, and the like.

Pharmaceutically acceptable salts may include, sodium, potassium, calcium, magnesium, ammonium, tetraalkylammonium and the like, and when the compound contains a basic functionality, salts of non-toxic organic or inorganic acids, such as hydrohalides, e.g. hydrochloride, hydrobromide and hydroiodide, carbonate or bicarbonate, sulfate or bisulfate, borate, phosphate, hydrogen phosphate, dihydrogen phosphate, pyroglutamate, saccharate, stearate, sulfamate, nitrate, orotate, oxalate, palmitate, pamoate, acetate, trifluoroacetate, trichloroacetate, propionate, hexanoate, cyclopentylpropionate, glycolate, glutarate, pyruvate, lactate, malonate, succinate, tannate, tartrate, tosylate, sorbate, ascorbate, malate, maleate, fumarate, tartarate, camsylate, citrate, cyclamate, benzoate, isethionate, esylate, formate, 3-(4-hydroxybenzoyl)benzoate, picrate, cinnamate, mandelate, phthalate, laurate, methanesulfonate (mesylate), methylsulphate, naphthylate, 2-napsylate, nicotinate, ethanesulfonate, 1,2-ethane-disulfonate, 2-hydroxyethanesulfonate, benzenesulfonate (besylate), 4-chlorobenzenesulfonate, 2-naphthalenesulfonate, 4-toluenesulfonate, camphorate, camphorsulfonate, 4-methylbicyclo[2.2.2]-oct-2-ene-1-carboxylate, glucoheptonate, 3-phenylpropionate, trimethylacetate, tert-butylacetate, lauryl sulfate, gluceptate, gluconate, glucoronate, hexafluorophosphate, hibenzate, benzoate, glutamate, hydroxynaphthoate, salicylate, stearate, cyclohexylsulfamate, quinate, muconate, xinofoate and the like.

Hemisalts of acids and bases may also be formed, for example, hemisulphate salts.

The skilled person will appreciate that the aforementioned salts include ones wherein the counterion is optically active, for example D-lactate, or racemic, for example DL-tartrate.

For a review on suitable salts, see “Handbook of Pharmaceutical Salts: Properties, Selection, and Use” by Stahl and Wermuth (Wiley-VCH, Weinheim, Germany, 2002).

Pharmaceutically acceptable salts of compounds of formula (I) may be prepared by one or more of three methods:
(i) by reacting the compound of formula (I) with the desired acid or base;
(ii) by removing an acid- or base-labile protecting group from a suitable precursor of the compound of formula (I) using the desired acid or base; or
(iii) by converting one salt of the compound of formula (I) to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionisation in the resulting salt may vary from completely ionised to almost non-ionised.

The term “solvate” may be understood to refer to a compound provided herein or a salt thereof, which further includes a stoichiometric or non-stoichiometric amount of solvent bound by non-covalent intermolecular forces. Where the solvent is water, the solvate is a hydrate. Pharmaceutically acceptable solvates in accordance with the invention include those wherein the solvent of crystallization may be isotopically substituted, e.g. D₂O, d₆-acetone and d₆-DMSO.

A currently accepted classification system for organic hydrates is one that defines isolated site, channel, or metal-ion coordinated hydrates—see Polymorphism in Pharmaceutical Solids by K. R. Morris (Ed. H. G. Brittain, Marcel Dekker, 1995), incorporated herein by reference. Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm.

The compounds of the invention may exist in a continuum of solid states ranging from fully amorphous to fully crystalline, including polymorphs of said crystalline material. The term ‘amorphous’ refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterised by a change of state, typically second order (‘glass transition’).

The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterised by a phase change, typically first order (‘melting point’).

The compounds of the invention may also exist in a mesomorphic state (mesophase or liquid crystal) when subjected to suitable conditions. The mesomorphic state is intermediate between the true crystalline state and the true liquid state (either melt or solution). Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO—Na⁺, —COO—K⁺, or —SO₃ Na⁺) or non-ionic (such as —N⁻N⁺ (CH₃)3) polar head group. For more information, see Crystals and the Polarizing Microscope by N. H. Hartshorne and A. Stuart, 4^{th} Edition (Edward Arnold, 1970), incorporated herein by reference.

In one embodiment, n is 1. Accordingly, the compound may have formula (I_{A}):

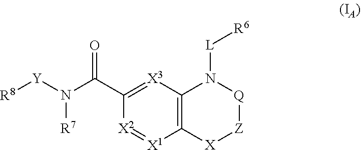

In an alternative embodiment, n is 2. Accordingly, the compound may have formula (I_{B}):

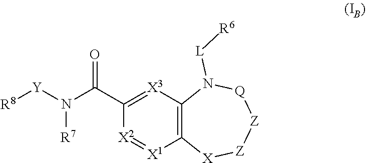

X may be CR⁹R¹⁰. X may be S. X may be O. X may be SO₂. X may be S═O.

Hence compounds of the invention may also be represented by the below structures (I_{A}-I)-(I_{A}-V):

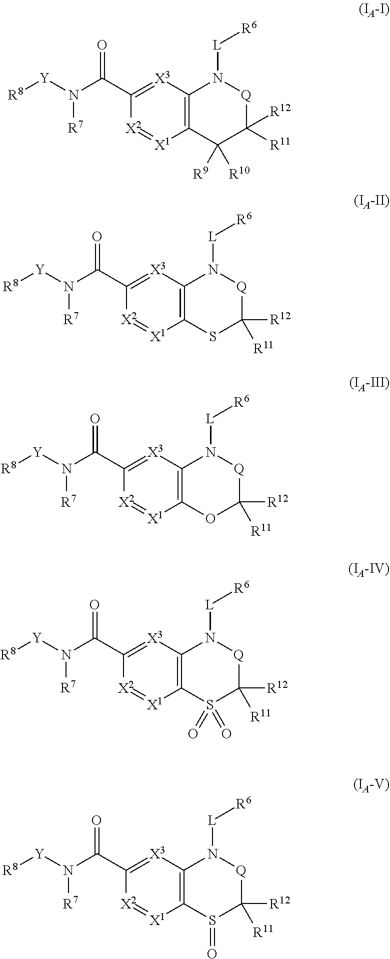

It will be appreciated that analogous structures of (I_{B}) are also within the scope of the present invention and are also incorporated herein.

L may be CH₂, C═O or SO₂. Accordingly, compounds of the invention may also be represented by the below structures (I-I) to (I-III);

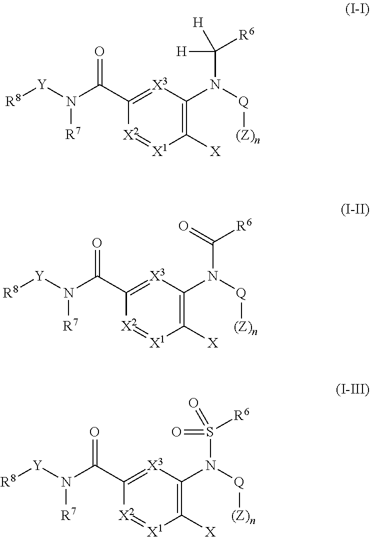

Q may be C═O, SO₂, S═O, CR⁴R⁵ or C═S. Accordingly, using (I_{A}-I) as an exemplar, compounds of the invention may also be represented by the below structures (I_{A}-I-I) to (I_{A}-I-V);

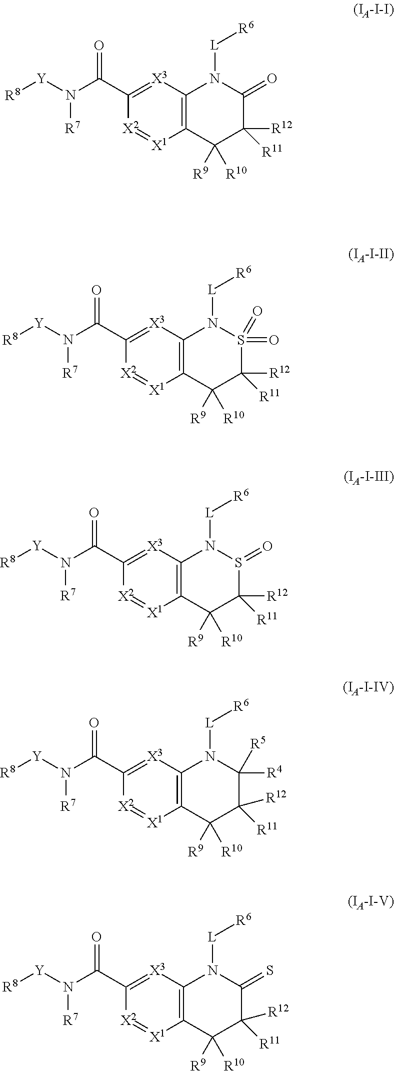

It will be appreciated that analogous structures of (I_{A}-II) to (I_{A}-VI) and (I_{B}) are also within the scope of the present invention and are also incorporated herein.

In one embodiment X¹ is CR¹, X² is CR² and X³ is CR³. R¹, R² and R³ may each independently be selected from the group consisting of H, halogen, and optionally substituted C₁-C₆ alkyl. Preferably, R¹, R² and R³ are each independently selected from the group consisting of H, halogen, and C₁-C₃ alkyl. More preferably, R¹, R² and R³ are each independently selected from the group consisting of H, halogen, and methyl. Most preferably, R¹, R² and R³ are each H.

In an alternative embodiment, one or two of X¹, X² and X³ is N. Accordingly, X¹ may be N, X² may be CR² and X³ may be CR³, X¹ may be CR¹, X² may be N and X³ may be CR³ or X¹ may be CR¹, X² may be CR² and X³ may be N.

Hence the compounds of the invention may also be represented by the below structures (I_{A}-I-I-I) to (I_{A}-I-I-III);

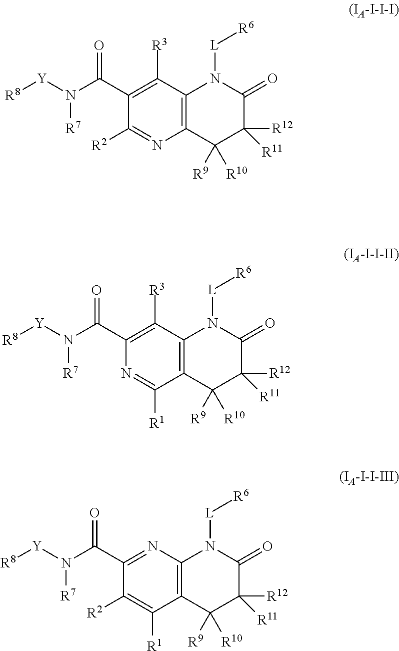

Preferably X² is CR². Accordingly, X¹ may be CR¹ or N and X³ may be CR³ or N. X¹ may be N, X² may be CR² and X³ may be CR³, or X¹ may be CR¹, X² may be CR² and X³ may be N, or X¹ may be N, X² may be CR² and X³ may be N. Preferably, R² is H, halogen or C₁-C₃ alkyl. More preferably, R² is H, halogen or methyl. Most preferably, R² is each H.

Preferably, R¹ and/or R³, in embodiments where they are present, are independently H, halogen or C₁-C₃ alkyl. More preferably, R¹ and/or R³, in embodiments where they are present, are independently H, halogen or methyl. Most preferably, R¹ and/or R³, in embodiments where they are present, are H.

Compounds of formula (I) may include one or more stereogenic centers and so may exist as optical isomers, such as enantiomers and diastereomers. All such isomers and mixtures thereof are included within the scope of the present invention.

In a preferred embodiment, X is CR⁹R¹⁰. Accordingly, the compound may be a compound of formula (I)-ent 1 or (I)-ent 2:

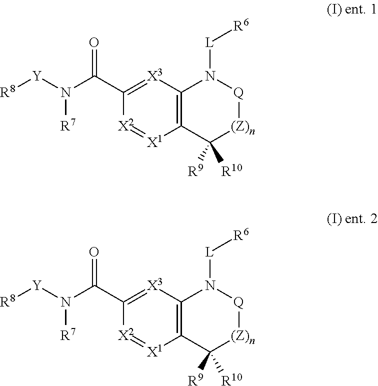

In an alternative embodiment, Q is CR⁴R⁵. Accordingly, the compound may be a compound of formula (I)-ent 3 or (I)-ent 4:

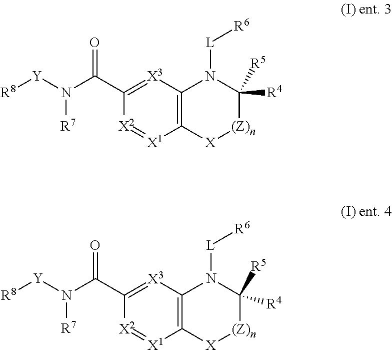

As mentioned above, Z is CR¹¹R¹², and the compound may possess a chiral centre at this location. Accordingly, the compound may be a compound of formula (I)-ent 5 or (I)-ent 6:

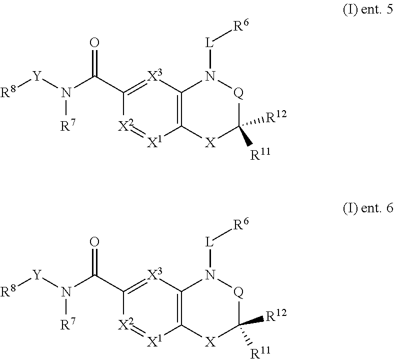

In yet another embodiment, where X is CR⁹R¹⁰ and n is 1, the compound could possess two chiral centres, and could be a compound of formula (I_{A}-I)-ent 1, formula (I_{A}-I)-ent 2, formula (I_{A}-I)-ent 3 or formula (I_{A}-I)-ent 4:

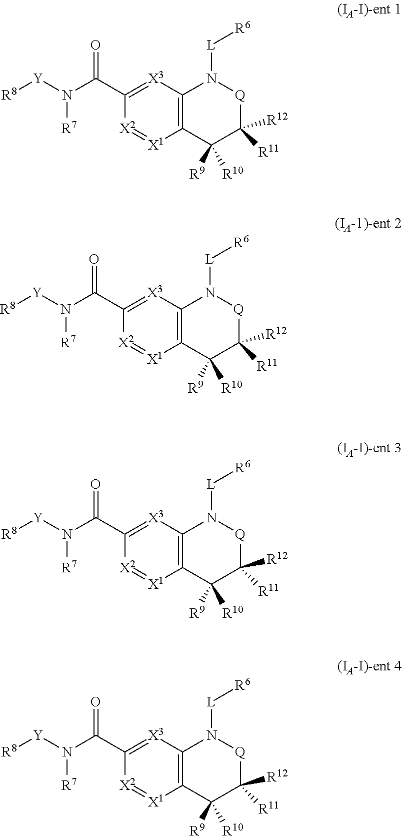

In an embodiment, where Q is CR⁴R⁵, X is CR⁹R¹⁰ and n is 2, the compound could possess four chiral centres, and could be any one of 16 possible enantiomers.

It will be understood that the above compounds may exist as enantiomers and as diastereoisomeric pairs. These isomers also represent further embodiments of the invention.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of formula (I) contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and from 0 to 5% by volume of an alkylamine, typically 0.1% diethylamine.

Concentration of the eluate affords the enriched mixture.

Mixtures of stereoisomers may be separated by conventional techniques known to those skilled in the art; see, for example, “Stereochemistry of Organic Compounds” by E. L. Eliel and S. H. Wilen (Wiley, New York, 1994).

In a preferred embodiment, X is O, S or CR⁹R¹⁰. More preferably, X is S or CR⁹R¹⁰.

Preferably, at least one of R⁹ and R¹⁰ is an optionally substituted C₁-C₆ alkyl, halogen, H, a C₃-C₆ cycloalkyl or C₁-C₃ polyfluoroalkyl. More preferably, at least one of R⁹ and R¹⁰ is a C₁-C₆ alkyl, H or a C₃-C₆ cycloalkyl, even more preferably a C₁-C₃ alkyl, H or a C₃-C₆ cycloalkyl, and most preferably at least one of R⁹ and R¹⁰ is H, methyl, ethyl, isopropyl or cyclopropyl. In one embodiment, both R⁹ and R¹⁰ are an optionally substituted C₁-C₆ alkyl or H. In one embodiment, both R⁹ and R¹⁰ are a C₁-C₆ alkyl, more preferably a C₁-C₃ alkyl, even more preferably methyl, ethyl or isopropyl, and most both R⁹ and R¹⁰ are methyl. In a most preferred embodiment, both R⁹ and R¹⁰ are H.

Preferably, n is 1.

Preferably, at least one of R¹¹ and R¹² is H. In one embodiment, both R¹ and R¹² is H.

Preferably, at least one of R¹ and R¹² is an optionally substituted C₁-C₆ alkyl, an optionally substituted C₂-C₆ alkenyl, a C₃-C₆ cycloalkyl or C₁-C₃ polyfluoroalkyl. More preferably, at least one of R¹ and R¹² is a C₁-C₆ alkyl, a C₂-C₆ alkenyl, or a C₃-C₆ cycloalkyl, even more preferably a C₁-C₃ alkyl, a C₂-C₃ alkenyl or a C₃-C₆ cycloalkyl, and most preferably at least one of R¹¹ and R¹² is methyl, ethyl, isopropyl or cyclopropyl.

In one embodiment, one of R¹¹ and R¹² is H and the other is an optionally substituted C₁-C₆ alkyl or an optionally substituted C₂-C₆ alkenyl. More preferably, one or R¹ and R¹² is H and the other is a C₁-C₃ alkyl or a C₂-C₃ alkenyl. Even more preferably, one or R¹¹ and R¹² is H and the other is methyl, ethyl, propyl or CH═CHCH₂—. Most preferably, one or R¹¹ and R¹² is H and the other is methyl.

In a preferred embodiment, Q is C═O, SO₂ or CR⁴R⁵. Preferably, Q is C═O or CR⁴R⁵. Preferably, R⁴ and R⁵ are each independently selected from the group consisting of H, halogen, optionally substituted C₁-C₆ alkyl, optionally substituted C₃-C₆ cycloalkyl or R⁴ and R⁵ together with the atom to which they are attached form a spirocyclic ring. Accordingly, R⁴ and R⁵ may both be H. Alternatively, R⁴ and R⁵ may both be Me or R⁴ may be Me and R⁵ may be H.

Most preferably, Q is C═O.

L may be C═O or SO₂. However, in a preferred embodiment, L is optionally substituted C₁-C₆ alkyl, —CH₂C(O)— or —CH₂CONH—. Preferably, L is optionally substituted C₁-C₃ alkyl, more preferably —CH₂—, —CH₂CH₂—, —CH₂CH₂CH₂—, C(Me)H, CF₂ or C(H)F and most preferably —CH₂—.

Preferably, R⁶ is a mono or bicyclic optionally substituted C₅-C₁₀ aryl, a mono or bicyclic optionally substituted 5 to 10 membered heteroaryl, an optionally substituted C₃-C₆ cycloalkyl or an optionally substituted C₃-C₆ heterocyclyl. More preferably, R⁶ is a mono or bicyclic optionally substituted C₅-C₁₀ aryl or a mono or bicyclic optionally substituted 5 to 10 membered heteroaryl. Most preferably, R⁶ is a mono or bicyclic optionally substituted C₅-C₁₀ aryl.

R⁶ may comprise between 1 and 5 substituents. The or each substituent may be independently selected from the list consisting of halogen, C₁-C₆ alkyl, CN, C₁-C₆ alkoxy, C₁-C₃ polyfluoroalkyl, azido, CONR¹R² and —OH. Preferably, the or each substituent is selected from the list consisting of halogen, C₁-C₆ alkyl, CN, OMe, OEt, OCF₃, CF₃, azido, CONH₂ and —OH.

Preferably, R⁶ is an optionally substituted C₅-C₁₀ aryl, wherein the C₅-C₁₀ aryl is a phenyl or a naphthyl. Most preferably, the C₅-C₁₀ aryl is phenyl. Preferably, C₅-C₁₀ aryl is substituted with methyl, ethyl, propyl, azido or halogen. More preferably, the C₅-C₁₀ aryl is substituted with at least one halogen. Accordingly, the C₅-C₁₀ aryl may be substituted by 1 or 2 halogens. Preferably, the or each halogen is fluorine or chlorine.

In some embodiments, when X¹ is CH, X² is CH and X³ is CH then R⁶ may not be an unsubstituted phenyl.

Alternatively, R⁶ may comprise an optionally substituted pyridine, an optionally substituted pyrazole, an optionally substituted thiazole or an optionally substituted isoxazole.

R⁷ is preferably H or an optionally substituted C₁-C₆ alkyl, more preferably H or a C₁-C₃ alkyl, and most preferably R⁷ is H. Preferably, Y is an optionally substituted C₁-C₆ alkyl, more preferably a C₁-C₃ alkyl, even more preferably —CH₂—, —CH₂CH₂— or —CH₂CH₂CH₂—, and most preferably —CH₂—.

Preferably, Y is an optionally substituted C₁-C₆ alkyl, more preferably a C₁-C₃ alkyl, even more preferably —CH₂—, —CH₂CH₂—, —CH₂CH₂CH₂—, —CH(CH₃)—, —CH(F)— and —CF₂— and most preferably —CH₂—.

Preferably, R⁸ is a mono or bicyclic optionally substituted C₅-C₁₀ aryl, a mono or bicyclic optionally substituted 5 to 10 membered heteroaryl, an optionally substituted C₃-C₆ cycloalkyl or an optionally substituted C₃-C₆ heterocyclyl.

In some embodiments, R⁸ may be an optionally substituted an optionally substituted C₃-C₆ cycloalkyl or C₃-C₆ heterocyclyl. R⁸ may comprise a C₆ cycloalkyl or a 6 membered heterocycle. The C₆ cycloalkyl or 6 membered heterocycle may be substituted with an optionally substituted C₁-C₆ alkyl or a mono or bicyclic optionally substituted C₅-C₁₀ aryl. Preferably, the C₆ cycloalkyl or 6 membered heterocycle is substituted with a phenyl or a C₁-C₃ alkyl substituted with a phenyl, more preferably the C₆ cycloalkyl or 6 membered heterocycle is substituted with a phenyl or —CH₂-phenyl.

Preferably, R⁸ is optionally substituted cyclohexane, optionally substituted piperidine or optionally substituted piperazine. More, preferably, R⁸ is substituted piperidine.

Accordingly, R⁸ may be:

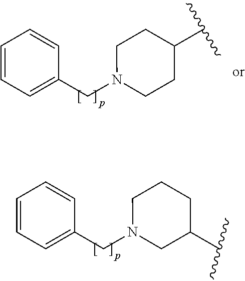

wherein p is 0 or 1.

However, in a preferred embodiment, R⁸ is a mono or bicyclic optionally substituted C₅-C₁₀ aryl or a mono or bicyclic optionally substituted 5 to 10 membered heteroaryl. R⁸ may be an optionally substituted phenyl, an optionally substituted pyridine, an optionally substituted naphthyl, an optionally substituted furanyl, an optionally substituted benzofuranyl, an optionally substituted thiophene, an optionally substituted pyridofuran, an optionally substituted benzoxazole or an optionally substituted benzothiazole. The mono or bicyclic C₅-C₁₀ aryl or the mono or bicyclic 5 to 10 membered heteroaryl may be substituted with between 1 and 5 substituents. The or each substituent may independently be selected from the list consisting of C₁-C₆ alkyl, halogen, OH, C₁-C₆ alkoxy, C₁-C₃ polyfluoroalkyl, CONR¹R², CN and azido. Preferably, the or each substituent is independently selected from the list consisting of C₁-C₆ alkyl, halogen, OH, OMe, OEt, OCF₃, CF₃, CONH₂, CN and azido. More preferably, the mono or bicyclic C₅-C₁₀ aryl or the mono or bicyclic 5 to 10 membered heteroaryl may be substituted with at least one C₁-C₆ alkyl or halogen, even more preferably at least one C₁-C₃ alkyl or halogen, and most preferably at least one methyl or fluorine.

In a preferred embodiment, R⁸ is an optionally substituted benzofuranyl. Preferably, R⁸ is an unsubstituted benzofuranyl.

In an alternative preferred embodiment, R⁸ is an optionally substituted furanyl. The furanyl may be an unsubstituted furanyl. Alternatively, the furanyl may be substituted.

Preferably, the furanyl is substituted with at least one of C₁-C₃ alkyl or halogen, more preferably at least one of methyl or fluorine and most preferably with one methyl group.

In an alternative preferred embodiment, R⁸ is an optionally substituted phenyl. The phenyl may be unsubstituted. Alternatively, the phenyl may be substituted. Preferably, the phenyl is substituted with at least one of C₁-C₃ alkyl or halogen, more preferably at least one of methyl or fluorine and most preferably with 1, 2 or 3 fluorines.

In a preferred embodiment, X¹ is CR¹; X² is CR²; X³ is CR³; n is 1; Q is CO; L is —CH₂—; Y is —CH₂—; and R⁷ is H.

In a further preferred embodiment, X¹ is CR¹; X² is CR²; X³ is N; n is 1; Q is CO; L is —CH₂—; Y is —CH₂—; and R⁷ is H.

In a further preferred embodiment, X¹ is CR¹; X² is CR²; X³ is CR³; n is 1; Q is CR⁴R⁵; L is C═O; Y is —CH₂—; and R⁷ is H.

In a further preferred embodiment, X¹ is CR¹; X² is CR²; X³ is CR³; n is 1; Q is CR⁴R⁵; L is SO₂; Y is —CH₂—; and R⁷ is H.

In a further preferred embodiment X is S, O or CR⁹R¹⁰. Preferably, X² is CR².

Preferably, n is 1. Preferably, Q is C═O or CR⁴R⁵. Preferably, L is optionally substituted C₁-C₃ alkyl or C₁-C₃ polyfluoroalkyl. L is most preferably C₁-C₂ alkyl. Preferably, Y is an optionally substituted C₁-C₆ alkyl, more preferably a C₁-C₃ alkyl, and most preferably a C₁-C₂ alkyl. Preferably, R¹, R² and R³ are each independently selected from the group consisting of H, halogen, CN, optionally substituted C₁-C₆ alkyl, C₁-C₃ polyfluoroalkyl, and optionally substituted mono or bicyclic C₃-C₆ cycloalkyl. Preferably, R⁴ and R⁵ are each independently selected from the group consisting of H and C₁-C₆ alkyl. Preferably, R⁶ is a mono or bicyclic substituted C₅-C₁₀ aryl, a mono or bicyclic optionally substituted 5 to 10 membered heteroaryl or an optionally substituted C₃-C₆ cycloalkyl. Preferably, R⁷ is H. Preferably, R⁸ is a mono or bicyclic optionally substituted C₅-C₁₀ aryl, a mono or bicyclic optionally substituted 5 to 10 membered heteroaryl or an optionally substituted C₃-C₆ cycloalkyl. Preferably, R⁹ and R¹⁰ are each independently selected from the group consisting of optionally substituted C₁-C₆ alkyl, H, halogen, CN, hydroxyl, azido, NR¹R², C₁-C₃ polyfluoroalkyl, optionally substituted C₃-C₆ cycloalkyl, optionally substituted C₁-C₆ alkoxy or optionally substituted C₂-C₆ alkenyl. Preferably, R¹¹ and R¹² are each independently selected from the group consisting of optionally substituted C₁-C₆ alkyl, H, halogen, CN, hydroxyl, azido, NR¹R², C₁-C₃ polyfluoroalkyl, optionally substituted C₃-C₆ cycloalkyl, optionally substituted C₁-C₆ alkoxy or optionally substituted C₂-C₆ alkenyl.

In a more preferred embodiment X is S or CR⁹R¹⁰. Preferably, X¹ is CH or N. Preferably, X² is CH. Preferably, X³ is CH or N. Preferably, n is 1. Preferably, Q is C═O. Preferably, L is a C₁-C₂ alkyl. More preferably, L is —CH₂—. Preferably, Y is a C₁-C₂ alkyl. More preferably, Y is —CH₂—. Preferably, R⁶ is a mono or bicyclic optionally substituted C₅-C₁₀ aryl, more preferably an optionally substituted phenyl ring.

Preferably, R⁶ is substituted with at least one halogen. Most preferably, R⁶ is substituted with one or two halogens. The or each halogen is preferably independently chlorine or fluorine. Preferably, R⁷ is H. Preferably, R⁸ is a mono or bicyclic optionally substituted C₅-C₁₀ aryl or a mono or bicyclic optionally substituted 5 to 10 membered heteroaryl. Most preferably, R⁸ is a substituted phenyl ring. Preferably, R⁸ is substituted with at least one halogen. Preferably, R⁸ is substituted with 1, 2 or 3 halogens, more preferably 2 or 3 halogens. Preferably, the or each halogen is fluorine. Preferably, R⁹ and R¹⁰ are each independently selected from the group consisting of C₁-C₆ alkyl, H, halogen, CN and azido. More preferably, R⁹ and R¹⁰ are each independently selected from the group consisting of C₁-C₃ alkyl and H. More preferably, R⁹ and R¹⁰ are each independently selected from the group consisting of CH₃ and H. Preferably, R¹¹ and R¹² are each independently selected from the group consisting of C₁-C₆ alkyl, H, halogen, CN and azido. More preferably, R¹¹ and R¹² are each independently selected from the group consisting of C₁-C₃ alkyl and H. More preferably, R¹¹ and R¹² are each independently selected from the group consisting of CH₃ and H.

It will be appreciated that an ‘agonist’, an ‘effector’ or an activator, as it relates to a ligand and STING, comprises a molecule, combination of molecules, or a complex, that stimulates STING. Conversely, an ‘antagonist’, as it relates to a ligand and STING, comprises a molecule, combination of molecules, or a complex, that inhibits, counteracts, downregulates, and/or desensitizes STING. ‘Antagonist’ encompasses any reagent that inhibits a constitutive activity of STING. A constitutive activity is one that is manifest in the absence of a ligand/STING interaction. ‘Antagonist’ also encompasses any reagent that inhibits or prevents a stimulated (or regulated) activity of STING.

Preferably, the compound of formula (I) is an activator of the STING protein.

It will be appreciated that the compounds described herein or a pharmaceutically acceptable salt, solvate, tautomeric form or polymorphic form thereof may be used in a medicament which may be used in a monotherapy (i.e. use of the compound alone), for modulating the STING protein and/or treating, ameliorating or preventing a disease.

Alternatively, the compounds or a pharmaceutically acceptable salt, solvate, tautomeric form or polymorphic form thereof may be used as an adjunct to, or in combination with, known therapies for modulating the STING protein and/or treating, ameliorating or preventing a disease.

Accordingly, in one aspect, a second therapeutic agent may be administered with a compound of Formula (I). The compound of Formula (I) may be administered before, after, and/or together with the second therapeutic agent. The second therapeutic agent may comprise an antiviral agent, an anti-inflammation agent, conventional chemotherapy, an anti-cancer vaccine and/or hormonal therapy. Alternatively, or additionally, the second therapeutic agent may comprise a B7 costimulatory molecule, interleukin-2, interferon-g, GM-CSF, a CTLA-4 antagonist (such as Ipilimumab and tremilimumab), an IDO inhibitor or IDO/TDO inhibitor (such as Epacadostat and GDC-o919), a PD-1 inhibitor (such as Nivolumab, Pembrolizumab, Pidilizumab, AMP-224, and MDX-1106), a PD-L1 inhibitor (such as Durvalumab, Avelumab and Atezolizumab), an OX-40 ligand, a LAG3 inhibitor, a CD40 ligand, a 41BB/CD137 ligand, a CD27 ligand, Bacille Calmette-Guerin (BCG), liposomes, alum, Freund's complete or incomplete adjuvant, a TLR agonist (such as Poly I:C, MPL, LPS, bacterial flagellin, imiquimod, resiquimod, loxoribine and a CpG dinucleotide) and/or detoxified endotoxins.

Methods for co-administration with an additional therapeutic agent are well known in the art (Hardman et. al. (eds.), Goodman and Gilman's The Pharmacological Basis of Therapeutics, 10^{th} ed., 2001, McGraw-Hill New York, N.Y.; Poole and Peterson (eds.), Pharmacotherapeutics for Advanced Practice: A Practical Approach, 2001, Lippincott, Williams and Wilkins, Philadelphia, Pa.; Chabner and Longo (eds.), Cancer Chemotherapy and Biotherapy, 2001, Lippincott, Williams and Wilkins, Philadelphia, Pa.).

In one aspect, the disease is cancer and a chemotherapeutic agent may be administered with a compound of Formula (I). The chemotherapeutic agent may be selected from a group further consisting of a cancer vaccine, a targeted drug, a targeted antibody, an antibody fragment, an antimetabolite, an antineoplastic, an antifolate, a toxin, an alkylating agent, a DNA strand breaking agent, a DNA minor groove binding agent, a pyrimidine analogue, a ribonucleotide reductase inhibitor, a tubulin interactive agent, an anti-hormonal agent, an immunomodulator, an anti-adrenal agent, a cytokine, radiation therapy, a cell therapy, cell depletion therapy such as B-cell depletion therapy and a hormone therapy. Alternatively or additionally, the chemotherapeutic agent may comprise abiraterone, altretamine, anhydrovinblastine, auristatin, bexarotene, bicalutamide, bleomycin, cachectin, cemadotin, chlorambucil, cyclophosphamide, docetaxol, doxetaxel, carboplatin, cysplatin, cytarabine, dactinomycin, daunorubicin, decitabine, doxorubicin, etoposide, 5-fluorouracil, finasteride, flutamide, hydroxyurea, streptozocin, mitomycin, methotrexate, taxanes, tamoxifen, vinblastine, vincristine and/or vindesine.

The compound of Formula (I) may be combined in compositions having a number of different forms depending, in particular, on the manner in which the composition is to be used. Thus, for example, the composition may be in the form of a powder, tablet, capsule, liquid, ointment, cream, gel, hydrogel, aerosol, spray, micellar solution, transdermal patch, liposome suspension or any other suitable form that may be administered to a person or animal in need of treatment. It will be appreciated that the vehicle of medicaments according to the invention should be one which is well-tolerated by the subject to whom it is given.

Medicaments comprising the compounds described herein may be used in a number of ways. Suitable modes of administration include oral, intra-tumoral, parenteral, topical, inhaled/intranasal, rectal/intravaginal, and ocular/aural administration.

Formulations suitable for the aforementioned modes of administration may be formulated to be immediate and/or modified release. Modified release formulations include delayed-, sustained-, pulsed-, controlled-, targeted and programmed release.

The compounds of the invention may be administered orally. Oral administration may involve swallowing, so that the compound enters the gastrointestinal tract, or buccal or sublingual administration may be employed by which the compound enters the blood stream directly from the mouth. Formulations suitable for oral administration include solid formulations such as tablets, capsules containing particulates, liquids, or powders, lozenges (including liquid-filled), chews, multi- and nano-particulates, gels, solid solution, liposome, films, ovules, sprays, liquid formulations and buccal/mucoadhesive patches.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986, by Liang and Chen (2001).

For tablet dosage forms, depending on dose, the drug may make up from 1 weight % to 80 weight % of the dosage form, more typically from 5 weight % to 60 weight % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet.

Other possible ingredients include anti-oxidants, colourants, flavouring agents, preservatives and taste-masking agents.

Exemplary tablets contain up to about 80% drug, from about 10 weight % to about 90 weight % binder, from about 0 weight % to about 85 weight % diluent, from about 2 weight % to about 10 weight % disintegrant, and from about 0.25 weight % to about 10 weight % lubricant. Tablet blends may be compressed directly or by roller to form tablets. Tablet blends or portions of blends may alternatively be wet-, dry-, or melt-granulated, melt congealed, or extruded before tableting. The final formulation may comprise one or more layers and may be coated or uncoated; it may even be encapsulated. The formulation of tablets is discussed in “Pharmaceutical Dosage Forms: Tablets”, Vol. 1, by H. Lieberman and L. Lachman (Marcel Dekker, New York, 1980).

Suitable modified release formulations for the purposes of the invention are described in U.S. Pat. No. 6,106,864. Details of other suitable release technologies such as high energy dispersions and osmotic and coated particles are to be found in “Pharmaceutical Technology On-line”, 25(2), 1-14, by Verma et al (2001). The use of chewing gum to achieve controlled release is described in WO 00/35298.

The compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of compounds of formula (I) used in the preparation of parenteral solutions may be increased by the use of appropriate formulation techniques, such as the incorporation of solubility-enhancing agents. Formulations for parenteral administration may be formulated to be immediate and/or modified release. Modified release formulations include delayed-, sustained-, pulsed-, controlled-, targeted and programmed release. Thus compounds of the invention may be formulated as a solid, semi-solid, or thixotropic liquid for administration as an implanted depot providing modified release of the active compound. Examples of such formulations include drug-coated stents and poly(dl-lactic-coglycolic)acid (PGLA) microspheres.

The compounds of the invention may also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958, by Finnin and Morgan (October 1999).

Other means of topical administration include delivery by electroporation, iontophoresis, phonophoresis, sonophoresis and microneedle or needle-free (e.g. Powderject™, Bioject™, etc.) injection.

The compounds of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

The pressurised container, pump, spray, atomizer, or nebuliser contains a solution or suspension of the compound(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose), blisters and cartridges for use in an inhaler or insufflator may be formulated to contain a powder mix of the compound of the invention, a suitable powder base such as lactose or starch and a performance modifier such as L-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound of the invention per actuation and the actuation volume may vary from 1 μl to 100 μl. A typical formulation may comprise a compound of formula (I), propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

In the case of dry powder inhalers and aerosols, the dosage unit is determined by means of a valve which delivers a metered amount. Units in accordance with the invention are typically arranged to administer a metered dose or “puff” containing from 1 μg to 100 mg of the compound of formula (I). The overall daily dose will typically be in the range 1 μg to 200 mg which may be administered in a single dose or, more usually, as divided doses throughout the day.

The compounds of the invention may be administered rectally or vaginally, for example, in the form of a suppository, pessary, microbicide, vaginal ring or enema. Cocoa butter is a traditional suppository base, but various alternatives may be used as appropriate.

The compounds of the invention may also be administered directly to the eye or ear, typically in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

The compounds of the invention may also be administered directly to a site of interest by injection of a solution or suspension containing the active drug substance. The site of interest may be a tumour and the compound may by administer via intratumoral injection. Typical injection solutions are comprised of propylene glycol, sterile water, ethanol and sodium chloride. Alternative solvents which may be used instead of propylene glycol include glycerol and polyethylene glycol.

The compounds of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

It will be appreciated that the amount of the compound that is required is determined by its biological activity and bioavailability, which in turn depends on the mode of administration, the physiochemical properties of the compound, and whether it is being used as a mono-therapy, or in a combined therapy. The frequency of administration will also be influenced by the half-life of the compound within the subject being treated. Optimal dosages to be administered may be determined by those skilled in the art, and will vary with the particular compound in use, the strength of the pharmaceutical composition, the mode of administration, and the advancement of the disease. Additional factors depending on the particular subject being treated will result in a need to adjust dosages, including subject age, weight, gender, diet, and time of administration.

Generally, for administration to a human, the total daily dose of the compounds of the invention is typically in the range 100 μg to 10 g, such as 1 mg to 1 g, for example 10 mg to 500 mg. For example, oral administration may require a total daily dose of from 25 mg to 250 mg. The total daily dose may be administered in single or divided doses and may, at the physician's discretion, fall outside of the typical range given herein. These dosages are based on an average human subject having a weight of about 60 kg to 70 kg. The physician will readily be able to determine doses for subjects whose weight falls outside this range, such as infants and the elderly.

However, it is appreciated by those skilled in the art that for agents that modulate the immune system, both the dose and the frequency of administration may be different to those of more traditional therapies. In particular, for agents that stimulate the immune system, for example through modulation of STING, they may be administered in small doses, and quite infrequently, for example twice weekly, weekly or monthly. Smaller doses may also be effective when administered topically to a small area of skin.

The compound may be administered before, during or after onset of the disease to be treated.

Known procedures, such as those conventionally employed by the pharmaceutical industry (e.g. in vivo experimentation, clinical trials, etc.), may be used to form specific formulations comprising the compounds according to the invention and precise therapeutic regimes (such as daily doses of the compounds and the frequency of administration). The inventors believe that they are the first to describe a pharmaceutical composition for treating a disease, based on the use of the compounds of the invention.

Hence, in a seventh aspect of the invention, there is provided a pharmaceutical composition comprising a compound according to the first aspect, or a pharmaceutically acceptable salt, solvate, tautomeric form or polymorphic form thereof, and a pharmaceutically acceptable vehicle.

The invention also provides, in an eighth aspect, a process for making the composition according to the seventh aspect, the process comprising contacting a therapeutically effective amount of a compound of the first aspect, or a pharmaceutically acceptable salt, solvate, tautomeric form or polymorphic form thereof, and a pharmaceutically acceptable vehicle.

A “subject” may be a vertebrate, mammal, or domestic animal. Hence, compounds, compositions and medicaments according to the invention may be used to treat any mammal, for example livestock (e.g. a horse), pets, or may be used in other veterinary applications. Most preferably, however, the subject is a human being.

A “therapeutically effective amount” of compound is any amount which, when administered to a subject, is the amount of drug that is needed to treat the target disease, or produce the desired effect, i.e. modulate the STING protein.

For example, the therapeutically effective amount of compound used may be from about 0.01 mg to about 800 mg, and preferably from about 0.01 mg to about 500 mg. It is preferred that the amount of compound is an amount from about 0.1 mg to about 250 mg, and most preferably from about 0.1 mg to about 20 mg.

A “pharmaceutically acceptable vehicle” as referred to herein, is any known compound or combination of known compounds that are known to those skilled in the art to be useful in formulating pharmaceutical compositions.

In one embodiment, the pharmaceutically acceptable vehicle may be a solid, and the composition may be in the form of a powder or tablet. A solid pharmaceutically acceptable vehicle may include one or more substances which may also act as flavouring agents, lubricants, solubilisers, suspending agents, dyes, fillers, glidants, compression aids, inert binders, sweeteners, preservatives, dyes, coatings, or tablet-disintegrating agents. The vehicle may also be an encapsulating material. In powders, the vehicle is a finely divided solid that is in admixture with the finely divided active agents (i.e. the compound according to the first, second and third aspects) according to the invention. In tablets, the active compound may be mixed with a vehicle having the necessary compression properties in suitable proportions and compacted in the shape and size desired. The powders and tablets preferably contain up to 99% of the active compound. Suitable solid vehicles include, for example calcium phosphate, magnesium stearate, talc, sugars, lactose, dextrin, starch, gelatin, cellulose, polyvinylpyrrolidine, low melting waxes and ion exchange resins. In another embodiment, the pharmaceutical vehicle may be a gel and the composition may be in the form of a cream or the like.

However, the pharmaceutical vehicle may be a liquid, and the pharmaceutical composition is in the form of a solution. Liquid vehicles are used in preparing solutions, suspensions, emulsions, syrups, elixirs and pressurized compositions. The compound according to the invention may be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilisers, emulsifiers, buffers, preservatives, sweeteners, flavouring agents, suspending agents, thickening agents, colours, viscosity regulators, stabilizers or osmo-regulators. Suitable examples of liquid vehicles for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can also be an oily ester such as ethyl oleate and isopropyl myristate. Sterile liquid vehicles are useful in sterile liquid form compositions for parenteral administration. The liquid vehicle for pressurized compositions can be a halogenated hydrocarbon or other pharmaceutically acceptable propellant.

Liquid pharmaceutical compositions, which are sterile solutions or suspensions, can be utilized by, for example, intramuscular, intrathecal, epidural, intraperitoneal, intravenous and particularly subcutaneous injection. The compound may be prepared as a sterile solid composition that may be dissolved or suspended at the time of administration using sterile water, saline, or other appropriate sterile injectable medium.

The compound and compositions of the invention may be administered in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like. The compounds used according to the invention can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

It will be known to those skilled in the art that active drug ingredients may be converted into a prodrug, which is a metabolically labile derivative that is converted within the body into the active drug substance. Also included within the scope of the invention are prodrugs which are compounds of formula (I) which contain metabolically or hydrolytically labile moieties which in vivo are converted into the active drug of formula (I). The processes by which the prodrug is converted into the active drug substance include, but are not limited to, ester hydrolysis, phosphate ester hydrolysis, S-oxidation, N-oxidation, dealkylation and metabolic oxidation as described in Beaumont et. al., Curr. Drug Metab., 2003, 4, 461-485 and Huttenen et. al., Pharmacol. Revs., 2011, 63, 750-771. Such prodrug derivatives may offer improved solubility, stability or permeability compared to the parent drug substance, or may better allow the drug substance to be administered by an alternative route of administration, for example as an intravenous solution.

Also included within the scope of the invention are soft drugs or antedrugs which are compounds of formula (I) which contain metabolically or hydrolytically labile moieties which in vivo are converted into inactive derivatives. The processes by which the active drug substance is converted into an inactive derivative include, but are not limited to, ester hydrolysis, S-oxidation, N-oxidation, dealkylation and metabolic oxidation as described for example in Pearce et al., Drug Metab. Dispos., 2006, 34, 1035-1040 and B. Testa, Prodrug and Soft Drug Design, in Comprehensive Medicinal Chemistry II, Volume 5, Elsevier, Oxford, 2007, pp. 1009-1041 and Bodor, N. Chem. Tech. 1984, 14, 28-38.

The invention also extends to a conjugate of a compound of formula (I).

Accordingly, in a further aspect of the invention, there is provided a conjugate of formula (IV):

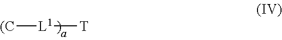

wherein, C is a compound of formula (I);

L¹ is a linker;

T is a targeting moiety; and

a is an integer between 1 and 10.

Such conjugates may be designed to specifically target certain cell types or tumor types via the targeting moiety, which directs the compound of formula (I) to just those cells or tumors and deliver the STING activator in a cell-specific manner. The principle of this targeted delivery will be known to those skilled in the art as being closely related to ADC (antibody-drug conjugate) technology, for example as described in Polakis, P., Pharmacol. Revs., 2016, 68, 3-19. The linker will then be designed to cleave and the active compound would then diffuse into the cell and contact the STING protein.

T may comprise an antibody, an antibody fragment, a nucleic acid based molecule, a carbohydrate, a peptide or a modified peptide.

In one embodiment, T comprises an antibody or antibody fragment. The antibody or antibody fragment may be designed to target the Human Epidermal Growth Factor Receptor (EGFR), a plasminogen activator, a cytotoxic T-lymphocyte associated antigen (CTLA) such as CTLA-4, vascular endothelial growth factor (VEGF), neurotrophic factors such as BDNF, a nerve growth factor, platelet-derived growth factor (PDGF), transforming growth factor (TGF), EpCAM, FLT3, PSMA, PSCA, STEAP, CEA, folate receptor, the CD33/CD30/CD79/CD22 receptors, the SLC34A2 gene product, the mesothelin protein, the EphA2 tyrosine kinase, the Muci/Muc16 cell-surface antigens, ALK, AFP, brc-abl, caspase-8, CD20, CD40, CD123, CDK4, c-kit, cMET, ErbB2/Her2, ErbB3/Her3, ErbB4/Her4, Her2, OX40, p53, PAP, PAX3, PAX5, Ras, Rho or any other tumor antigen known to those skilled in the art.

The invention extends to both whole antibodies, as well as to antigen-binding fragments or regions of the corresponding full-length antibody.

The antibody or antigen-binding fragment thereof may be monovalent, divalent or polyvalent. Monovalent antibodies are dimers (HL) comprising a heavy (H) chain associated by a disulphide bridge with a light chain (L). Divalent antibodies are tetramer (H2L2) comprising two dimers associated by at least one disulphide bridge. Polyvalent antibodies may also be produced, for example by linking multiple dimers. The basic structure of an antibody molecule consists of two identical light chains and two identical heavy chains which associate non-covalently and can be linked by disulphide bonds. Each heavy and light chain contains an amino-terminal variable region of about 110 amino acids, and constant sequences in the remainder of the chain. The variable region includes several hypervariable regions, or Complementarity Determining Regions (CDRs), that form the antigen-binding site of the antibody molecule and determine its specificity for the antigen or variant or fragment thereof (e.g. an epitope). On either side of the CDRs of the heavy and light chains is a framework region, a relatively conserved sequence of amino acids that anchors and orients the CDRs. Antibody fragments may include a bi-specific antibody (BsAb) or a chimeric antigen receptor (CAR).

The constant region consists of one of five heavy chain sequences (μ, γ, ζ, α, or ε) and one of two light chain sequences (κ or λ). The heavy chain constant region sequences determine the isotype of the antibody and the effector functions of the molecule.

Preferably, the antibody or antigen-binding fragment thereof is isolated or purified.

In one preferred embodiment, the antibody or antigen-binding fragment thereof comprises a polyclonal antibody, or an antigen-binding fragment thereof. The antibody or antigen-binding fragment thereof may be generated in a rabbit, mouse or rat.

In another preferred embodiment, the antibody or antigen-binding fragment thereof comprises a monoclonal antibody or an antigen-binding fragment thereof. Preferably, the antibody is a human antibody. As used herein, the term “human antibody” can mean an antibody, such as a monoclonal antibody, which comprises substantially the same heavy and light chain CDR amino acid sequences as found in a particular human antibody exhibiting immunospecificity. An amino acid sequence, which is substantially the same as a heavy or light chain CDR, exhibits a considerable amount of sequence identity when compared to a reference sequence. Such identity is definitively known or recognizable as representing the amino acid sequence of the particular human antibody. Substantially the same heavy and light chain CDR amino acid sequence can have, for example, minor modifications or conservative substitutions of amino acids.

The term “human monoclonal antibody” can include a monoclonal antibody with substantially or entirely human CDR amino acid sequences produced, for example by recombinant methods such as production by a phage library, by lymphocytes or by hybridoma cells.

The term “humanised antibody” can mean an antibody from a non-human species (e.g. mouse or rabbit) whose protein sequences have been modified to increase their similarity to antibodies produced naturally in humans.

The antibody may be a recombinant antibody. The term “recombinant human antibody” can include a human antibody produced using recombinant DNA technology.

The term “antigen-binding region” can mean a region of the antibody having specific binding affinity for its target antigen or a variant or fragment thereof. Preferably, the fragment is an epitope. The binding region may be a hypervariable CDR or a functional portion thereof. The term “functional portion” of a CDR can mean a sequence within the CDR which shows specific affinity for the target antigen. The functional portion of a CDR may comprise a ligand which specifically binds to the target antigen or a fragment thereof.

The term “CDR” can mean a hypervariable region in the heavy and light variable chains. There may be one, two, three or more CDRs in each of the heavy and light chains of the antibody. Normally, there are at least three CDRs on each chain which, when configured together, form the antigen-binding site, i.e. the three-dimensional combining site with which the antigen binds or specifically reacts. It has however been postulated that there may be four CDRs in the heavy chains of some antibodies.

The definition of CDR also includes overlapping or subsets of amino acid residues when compared against each other. The exact residue numbers which encompass a particular CDR or a functional portion thereof will vary depending on the sequence and size of the CDR. Those skilled in the art can routinely determine which residues comprise a particular CDR given the variable region amino acid sequence of the antibody.

The term “functional fragment” of an antibody can mean a portion of the antibody which retains a functional activity. A functional activity can be, for example antigen binding activity or specificity. A functional activity can also be, for example, an effector function provided by an antibody constant region. The term “functional fragment” is also intended to include, for example, fragments produced by protease digestion or reduction of a human monoclonal antibody and by recombinant DNA methods known to those skilled in the art. Human monoclonal antibody functional fragments include, for example individual heavy or light chains and fragments thereof, such as VL, VH and Fd; monovalent fragments, such as Fv, Fab, and Fab′; bivalent fragments such as F(ab′)₂; single chain Fv (scFv); and Fc fragments.

The term “VL fragment” can mean a fragment of the light chain of a human monoclonal antibody which includes all or part of the light chain variable region, including the CDRs. A VL fragment can further include light chain constant region sequences.

The term “VH fragment” can means a fragment of the heavy chain of a human monoclonal antibody which includes all or part of the heavy chain variable region, including the CDRs.

The term “Fd fragment” can mean the heavy chain variable region coupled to the first heavy chain constant region, i.e. VH and CH-1. The “Fd fragment” does not include the light chain, or the second and third constant regions of the heavy chain.

The term “Fv fragment” can mean a monovalent antigen-binding fragment of a human monoclonal antibody, including all or part of the variable regions of the heavy and light chains, and absent of the constant regions of the heavy and light chains. The variable regions of the heavy and light chains include, for example, the CDRs. For example, an Fv fragment includes all or part of the amino terminal variable region of about 110 amino acids of both the heavy and light chains.

The term “Fab fragment” can mean a monovalent antigen-binding fragment of a human monoclonal antibody that is larger than an Fv fragment. For example, a Fab fragment includes the variable regions, and all or part of the first constant domain of the heavy and light chains. Thus, a Fab fragment additionally includes, for example, amino acid residues from about 110 to about 220 of the heavy and light chains.

The term “Fab′ fragment” can mean a monovalent antigen-binding fragment of a human monoclonal antibody that is larger than a Fab fragment. For example, a Fab′ fragment includes all of the light chain, all of the variable region of the heavy chain, and all or part of the first and second constant domains of the heavy chain. For example, a Fab′ fragment can additionally include some or all of amino acid residues 220 to 330 of the heavy chain.

The term “F(ab′)₂ fragment” can mean a bivalent antigen-binding fragment of a human monoclonal antibody. An F(ab′)₂ fragment includes, for example, all or part of the variable regions of two heavy chains-and two light chains, and can further include all or part of the first constant domains of two heavy chains and two light chains.

The term “single chain Fv (scFv)” can mean a fusion of the variable regions of the heavy (VH) and light chains (VL) connected with a short linker peptide.

The term “bispecific antibody (BsAb)” can mean a bispecific antibody comprising two scFv linked to each other by a shorter linked peptide.

One skilled in the art knows that the exact boundaries of a fragment of an antibody are not important, so long as the fragment maintains a functional activity. Using well-known recombinant methods, one skilled in the art can engineer a polynucleotide sequence to express a functional fragment with any endpoints desired for a particular application. A functional fragment of the antibody may comprise or consist of a fragment with substantially the same heavy and light chain variable regions as the human antibody.

The antigen-binding fragment thereof may comprise or consist of any of the fragments selected from a group consisting of VH, VL, Fd, Fv, Fab, Fab′, scFv, F (ab′)₂ and Fc fragment.

The antigen-binding fragment thereof may comprise or consist of any one of the antigen binding region sequences of the VL, any one of the antigen binding region sequences of the VH, or a combination of VL and VH antigen binding regions of a human antibody. The appropriate number and combination of VH and VL antigen binding region sequences may be determined by those skilled in the art depending on the desired affinity and specificity and the intended use of the antigen-binding fragment. Functional fragments or antigen-binding fragments of antibodies may be readily produced and isolated using methods well known to those skilled in the art. Such methods include, for example, proteolytic methods, recombinant methods and chemical synthesis. Proteolytic methods for the isolation of functional fragments comprise using human antibodies as a starting material. Enzymes suitable for proteolysis of human immunoglobulins may include, for example, papain, and pepsin. The appropriate enzyme may be readily chosen by one skilled in the art, depending on, for example, whether monovalent or bivalent fragments are required. For example, papain cleavage results in two monovalent Fab′ fragments that bind antigen and an Fc fragment. Pepsin cleavage, for example, results in a bivalent F (ab′) fragment. An F (ab′)₂ fragment of the invention may be further reduced using, for example, DTT or 2-mercaptoethanol to produce two monovalent Fab′ fragments.

Functional or antigen-binding fragments of antibodies produced by proteolysis may be purified by affinity and column chromatographic procedures. For example, undigested antibodies and Fc fragments may be removed by binding to protein A. Additionally, functional fragments may be purified by virtue of their charge and size, using, for example, ion exchange and gel filtration chromatography. Such methods are well known to those skilled in the art.

The antibody or antigen-binding fragment thereof may be produced by recombinant methodology. Preferably, one initially isolates a polynucleotide encoding desired regions of the antibody heavy and light chains. Such regions may include, for example, all or part of the variable region of the heavy and light chains. Preferably, such regions can particularly include the antigen binding regions of the heavy and light chains, preferably the antigen binding sites, most preferably the CDRs.

The polynucleotide encoding the antibody or antigen-binding fragment thereof according to the invention may be produced using methods known to those skilled in the art. The polynucleotide encoding the antibody or antigen-binding fragment thereof may be directly synthesized by methods of oligonucleotide synthesis known in the art. Alternatively, smaller fragments may be synthesized and joined to form a larger functional fragment using recombinant methods known in the art.

As used herein, the term “immunospecificity” can mean the binding region is capable of immunoreacting with the target antigen, or a variant or fragment thereof, by specifically binding therewith. The antibody or antigen-binding fragment thereof can selectively interact with an antigen with an affinity constant of approximately 10⁻⁵ to 10⁻¹³ M⁻¹, preferably 10⁻⁶ to 10⁻⁹ M⁻¹, even more preferably, 10⁻¹⁰ to 10⁻¹² M⁻¹.

The term “immunoreact” can mean the binding region is capable of eliciting an immune response upon binding with SEQ ID No:3, or an epitope thereof.

The term “epitope” can mean any region of an antigen with the ability to elicit, and combine with, a binding region of the antibody or antigen-binding fragment thereof.

In one embodiment, T comprises a nucleic acid based molecule. The nucleic acid base molecule may be an aptamer. The nucleic acid based molecule may target the CD33/CD34 or PSMA tumor antigens, or any other tumor antigen known to those skilled in the art, for example as described in Orava, E., Biochem. Biophys. Acta, 2010, 1798, 2190-2200.

Aptamers are nucleic acid or peptide molecules that assume a specific, sequence-dependent shape and bind to specific target ligands based on a lock-and-key fit between the aptamer and ligand. Typically, aptamers may comprise either single- or double-stranded DNA molecules (ssDNA or dsDNA) or single-stranded RNA molecules (ssRNA). Peptide aptamers consist of a short variable peptide domain, attached at both ends to a protein scaffold. Aptamers may be used to bind both nucleic acid and non-nucleic acid targets.

Suitable aptamers may be selected from random sequence pools, from which specific aptamers may be identified which bind to the selected antigen with high affinity. Methods for the production and selection of aptamers having desired specificity are well known to those skilled in the art, and include the SELEX (systematic evolution of ligands by exponential enrichment) process. Briefly, large libraries of oligonucleotides are produced, allowing the isolation of large amounts of functional nucleic acids by an iterative process of in vitro selection and subsequent amplification through polymerase chain reaction. Preferred methodologies for producing aptamers include those disclosed in WO 2004/042083.

In an alternative embodiment, T comprises a peptide or a modified peptide. The peptide or modified peptide may comprise the RGD sequence motif, as described in Mousavizadeh, A., Colloids Surfaces B., 2017, 158, 507-517.

L¹ may comprise a carbonate, a carbamate, an ester, an amide, a urea and/or a lactam functional group (Beck, A. et. al., Nat. Revs. Drug Disc., 2017, 16, 315-337). Said linkers will be known to those skilled in the art as either ‘stable’ linkers which are resistant to degradation in cells and in the systemic circulation or ‘conditionally labile’ linkers which are designed to degrade in cells and/or in the systemic circulation following a defined trigger event, which may be a change in pH or a metabolic process such as ester or amide hydrolysis. Specific hydrolysis processes have been described, such as the peptidase cleavage of a dipeptide e.g. the valine-citrulline dipeptide moiety contained in the clinically precedented ADC brentuximab vedotin or the hydrolysis of a labile hydrazone moiety in gemtuzumab ozogamicin. Non-cleavable linkers include that contained in the clinically precedented ADC trastuzumab emtansine.

a may be 1, 2, 3, 4, 5, 6, 7, 8, 9 or 10.

L¹ may comprise an extended chain of carbon atoms or heteroatoms, for example a linear or branched polyethylene glycol (PEG) chain, an optionally substituted natural or unnatural sequence of amino acids or a linear or branchedoptionally substituted alkyl chain. The linked may be viewed as comprising an optionally substituted backbone, and the backbone of carbon atoms and/or heteroatoms. The backbone may consist of between 2 and 100 atoms, more preferably between 10 and 80 atoms or between 20 and 600 atoms. The backbone atoms may define one or more optionally substituted C₅-C₁₀ aryl, optionally substituted 5 to 10 membered heteroaryl, optionally substituted C₃-C₆ cycloalkyl and/or optionally substituted 3 to 8 membered heterocycle rings within the backbone. The backbone atoms may consist of carbon, nitrogen and/or oxygen atoms. The backbone atoms may be substituted with H, OH, ═O, halogen, optionally substituted C₁-C₆ alkyl, optionally substituted C₃-C₆ cycloalkyl and/or optionally substituted C₁-C₆ alkoxy. L¹ may also contain a functional group handle that allows the STING modulator to be chemically combined with the targeting moiety via a covalent bond. For example thiol groups, or cysteine residues may be bonded to the linker or spacer group via a maleimide group. Alternative conjugation chemistries include lysine reactive groups, such as succinyl esters, pentafluorophenyl esters, β-lactam amides, isocyanates, and isothiocyanates; azide reactive groups, such as alkynes and strained alkynes; cysteine reactive groups, such as maleimides, α-haloacetamides, pyridyl disulfides and vinyl sulfoxides; and ketone reactive groups, such as hydroxylamines, hydrazines and acyl hydrazides.

Linkers may be joined to a compound of formula (I) through a C atom, an O atom, a N atom or a S atom and may be functionalised with groups that include, but are not limited to, the following;

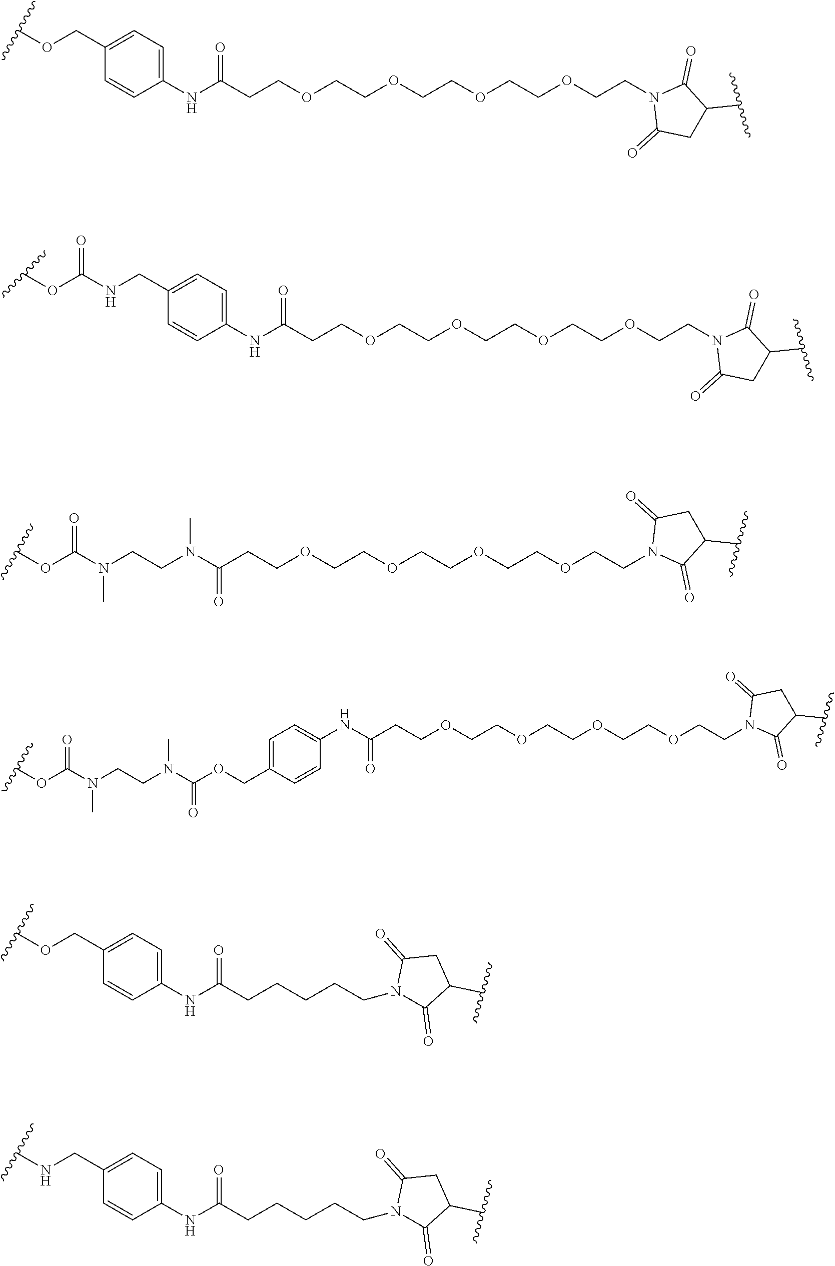 [SMILES: *OCc1ccc(NC(=O)CCOCCOCCOCCOCCN2C(=O)CC(*)C2=O)cc1 ; *OC(=O)NCc1ccc(NC(=O)CCOCCOCCOCCOCCN2C(=O)CC(*)C2=O)cc1 ; *OC(=O)N(C)CCN(C)C(=O)CCOCCOCCOCCOCCN1C(=O)CC(*)C1=O ; *OC(=O)N(C)CCN(C)C(=O)OCc1ccc(NC(=O)CCOCCOCCOCCOCCN2C(=O)CC(*)C2=O)cc1 ; *OCc1ccc(NC(=O)CCCCCN2C(=O)CC(*)C2=O)cc1 ; *NCc1ccc(NC(=O)CCCCCN2C(=O)CC(*)C2=O)cc1]

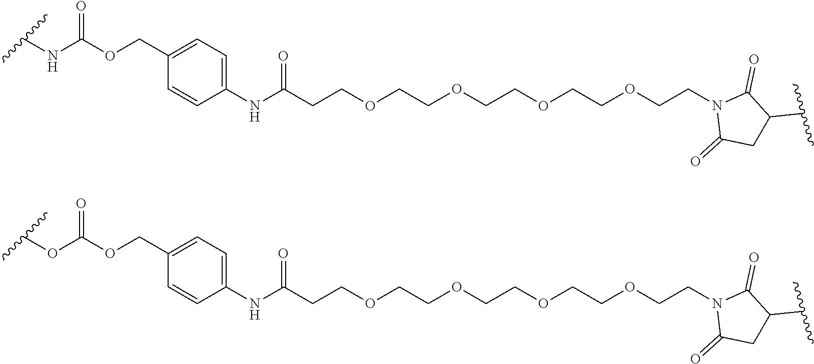 [SMILES: *NC(=O)OCc1ccc(NC(=O)CCOCCOCCOCCOCCN2C(=O)CC(*)C2=O)cc1 ; *OC(=O)OCc1ccc(NC(=O)CCOCCOCCOCCOCCN2C(=O)CC(*)C2=O)cc1]

Linkers may be cleavable, non-cleavable, hydrophilic or hydrophobic. A cleavable linker can be sensitive to enzymes and may be cleaved by enzymes such as proteases. For example, a cleavable linker can be a valine-citrulline linker or a valine-alanine linker.

For example;

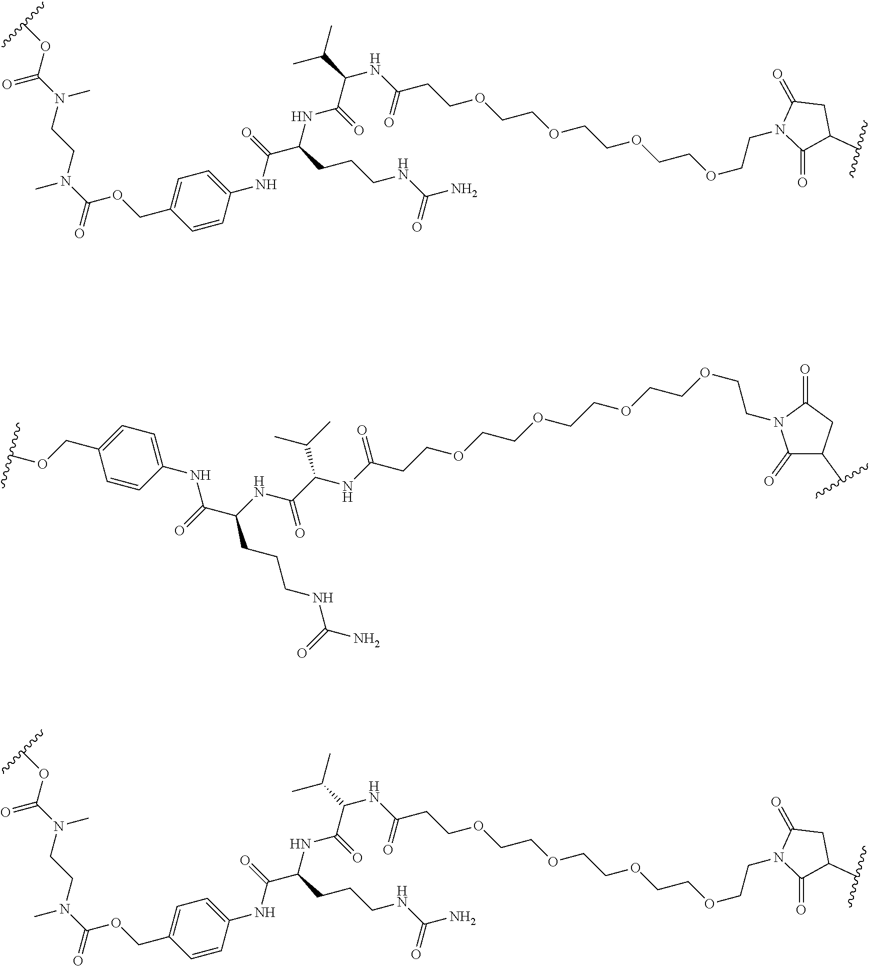

A non-cleavable linker may be protease insensitive.

L¹ may include alkyl chains (for example n-hexyl, n-pentyl, n-butyl, n-propyl), heteroatom containing chains (for example ethyloxy, propyloxy, butyloxy, pentyloxy, hexyoxy, ethylene dioxy, polyethylene glycol (PEG)), amino acids (gycinyl, alaninyl, aminopropanoic acid, aminobutanoic acid, aminopentanoic acid, aminohexanoic acid) and peptide units.

The inventors have found that compounds of the current invention may be functionalised in various locations with a variety of linkers and spacers to provide conjugate molecules. Said linkers may include self-immolating groups (for example a p-aminobenzyl ether or amine and/or a valine-citrulline unit) that are designed to release the parent STING modulator upon a hydrolytic event, for example following amide, peptide or carbamate hydrolysis.

The scope of the invention includes all pharmaceutically acceptable isotopically-labelled compounds of the invention wherein one or more atoms are replaced by atoms having the same atomic number, but an atomic mass or mass number different from the atomic mass or mass number which predominates in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of the invention, for example those incorporating a radioactive isotope, are useful in drug and/or substrate tissue distribution studies. The radioactive isotopes tritium, i.e. 3H, and carbon-14, i.e. ¹⁴C, are particularly useful for this purpose in view of their ease of incorporation and ready means of detection. Substitution with isotopes such as deuterium, i.e. ²H, may afford certain therapeutic advantages resulting from greater metabolic stability, for example, increased in vivo half-life or reduced dosage requirements, and hence may be preferred in some circumstances. Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled compounds of formula (I) can generally be prepared by conventional techniques known to those skilled in the art or by processes analogous to those described in the accompanying Examples and Preparations using an appropriate isotopically-labeled reagent in place of the non-labeled reagent previously employed.

In accordance with a further aspect of the invention, there is provided a compound of the formula (II) or (III):

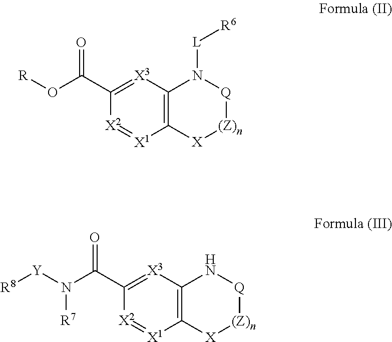

wherein, X, X¹, X², X³, n, Z, Q, L, Y, R⁶, R⁷ and R⁸ are as defined in the first aspect; and R is H or a C₁-C₆ alkyl,

or a pharmaceutically acceptable complex, salt, solvate, tautomeric form or polymorphic form thereof.

It will be appreciated that compounds of formula (II) and (III) may be used to synthesise compounds of formula (I).

Preferably, X is CR⁹R¹⁰, O or S.

When X is CR⁹R¹⁰, R⁹ and R¹⁰ are preferably independently C₁-C₆ alkyl, hydroxyl, halogen or CN. More preferably, R⁹ and R¹⁰ are independently hydrogen, methyl, hydroxyl, halogen or CN. In one embodiment, at least one of R⁹ and R¹⁰ is methyl.

Preferably, one of R⁹ and R¹⁰ is methyl and the other is hydrogen or methyl. In a preferred embodiment, at least one of R⁹ and R¹⁰ is hydrogen, and preferably both R⁹ and R¹⁰ are hydrogen.

Preferably, X² is CH.

Preferably, Q is C═O, SO₂ or CR⁴R⁵. More preferably, Q is C═O.

Preferably, L is C₁-C₆ alkyl, more preferably C₁-C₃ alkyl, and most preferably —CH₂—.

Preferably, R⁶ is optionally substituted C₅-C₁₀ aryl. More preferably, R⁶ is substituted phenyl. Even more preferably, R⁶ is phenyl substituted with at least one halogen. Most preferably, R⁶ is phenyl substituted with one or two halogens. Preferably, the or each halogen is chlorine or fluorine.

Preferably, R is H or methyl, ethyl, benzyl or tert-butyl. More preferably, R is H or methyl.

The compound of formula (II) may be selected from:

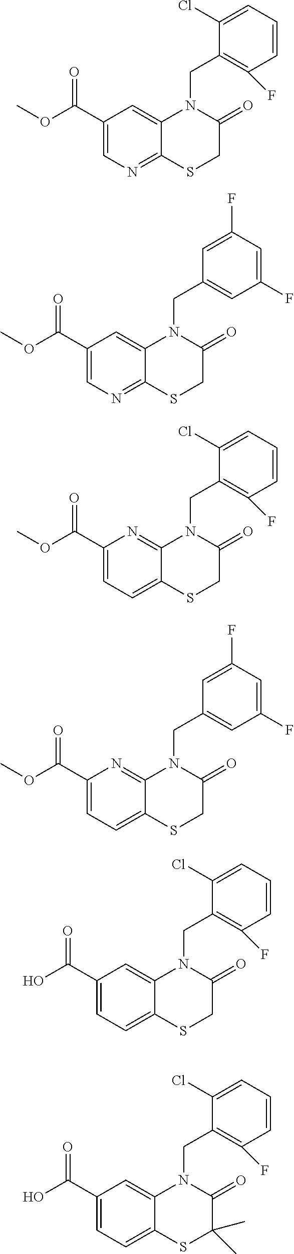

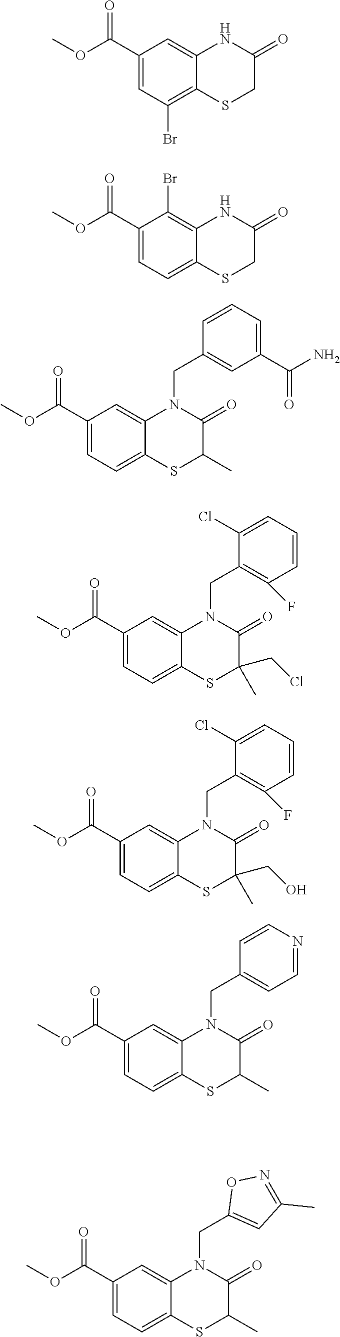

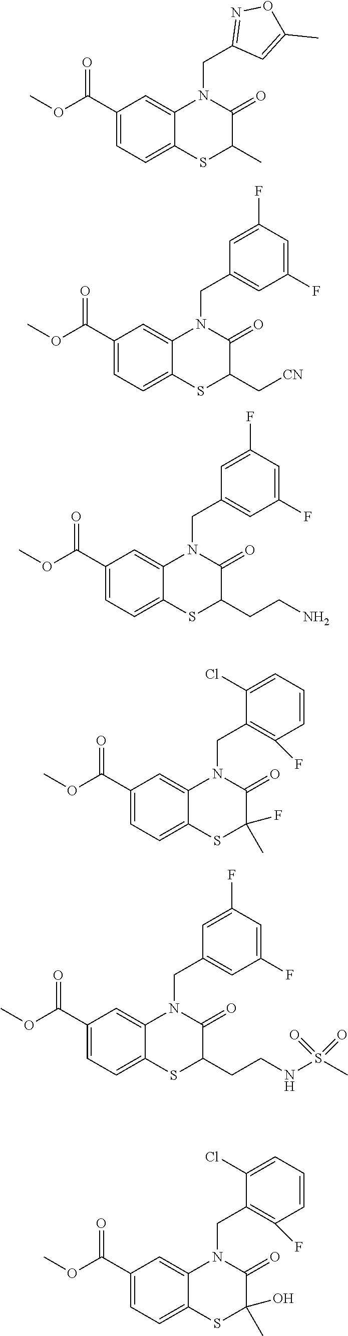

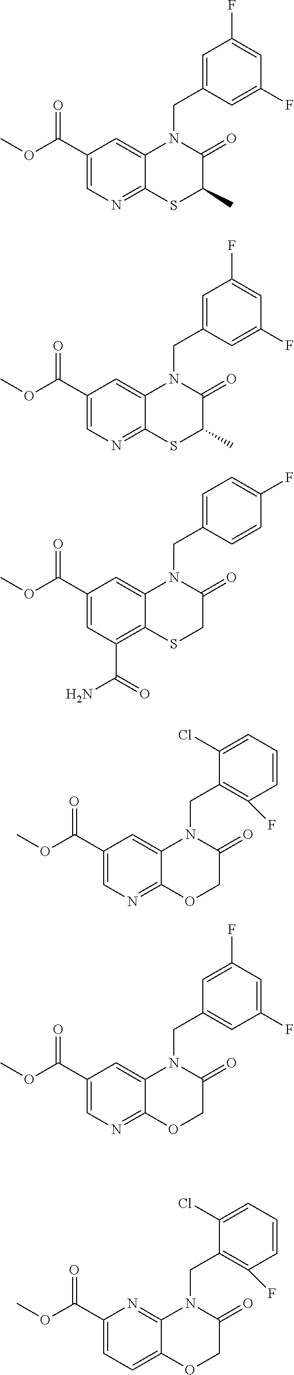

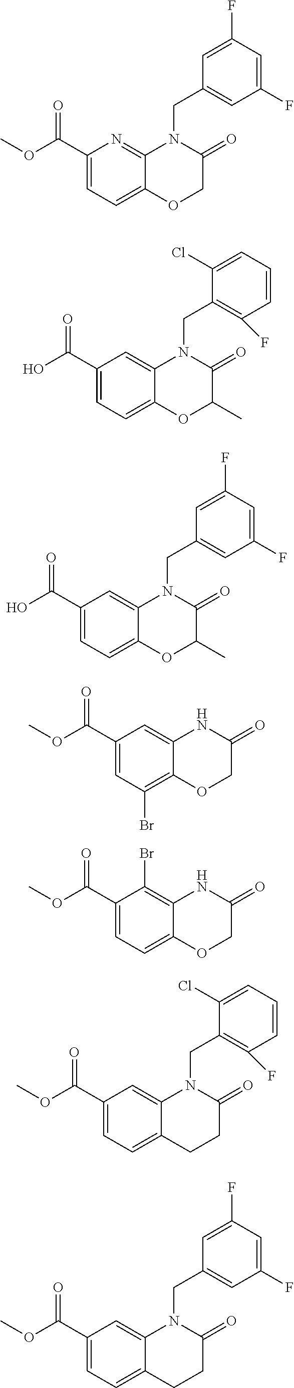

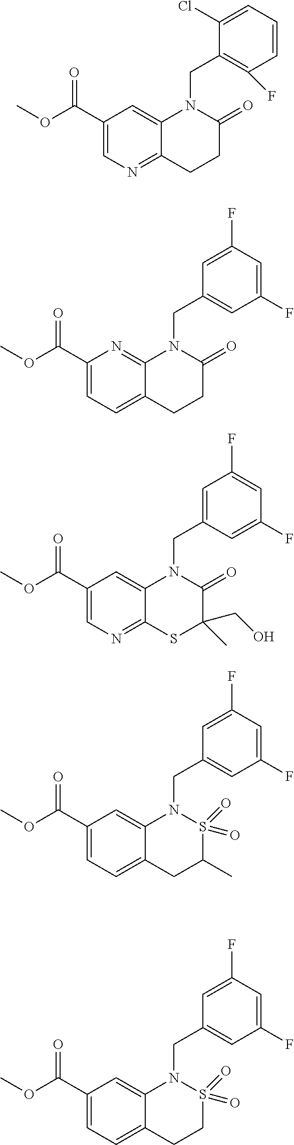

Preferably, Y is C₁-C₆ alkyl, more preferably C₁-C₃ alkyl, and most preferably —CH₂—.

Preferably, R⁷ is H.

Preferably, R⁸ is a mono or bicyclic optionally substituted C₅-C₁₀ aryl, a mono or bicyclic optionally substituted 5 to 10 membered heteroaryl, an optionally substituted C₃-C₆ cycloalkyl or an optionally substituted C₃-C₆ heterocyclyl. Preferably, R⁸ is a mono or bicyclic C₅-C₁₀ aryl or a mono or bicyclic 5 to 10 membered heteroaryl substituted with between 1 and 5 substituents, and the or each substituent is independently selected from the list consisting of C₁-C₆ alkyl, halogen, OH, C₁-C₆ alkoxy, C₁-C₃ polyfluoroalkyl, CONR¹R², CN and azido. More preferably, R⁸ may be an optionally substituted phenyl, an optionally substituted pyridine, an optionally substituted naphthyl, an optionally substituted furanyl, an optionally substituted benzofuranyl, an optionally substituted thiophene, an optionally substituted pyridofuran, an optionally substituted benzoxazole or an optionally substituted benzothiazole.

The compound of formula (III) may be selected from:

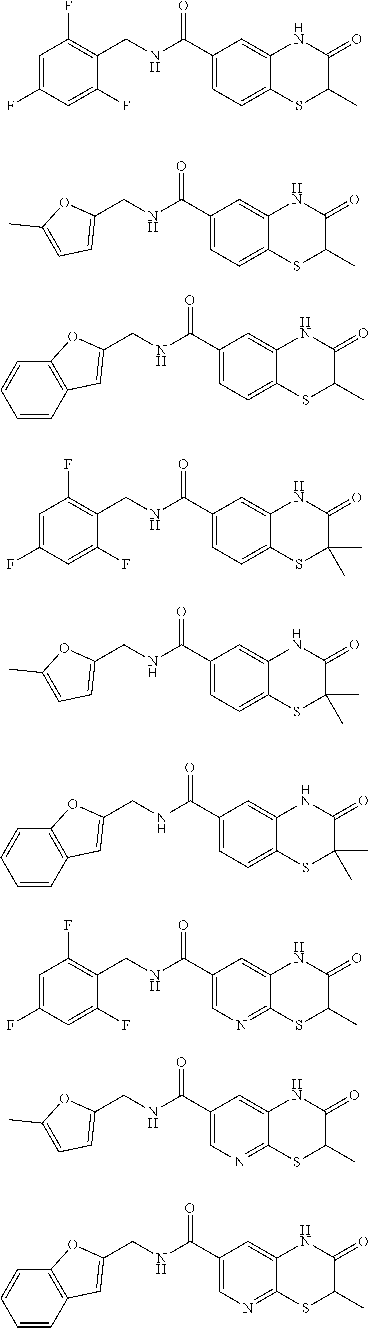

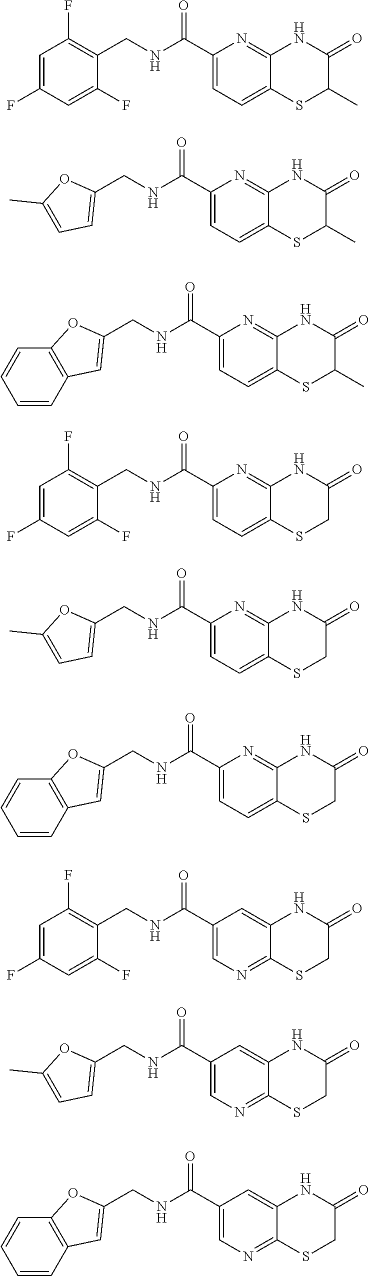

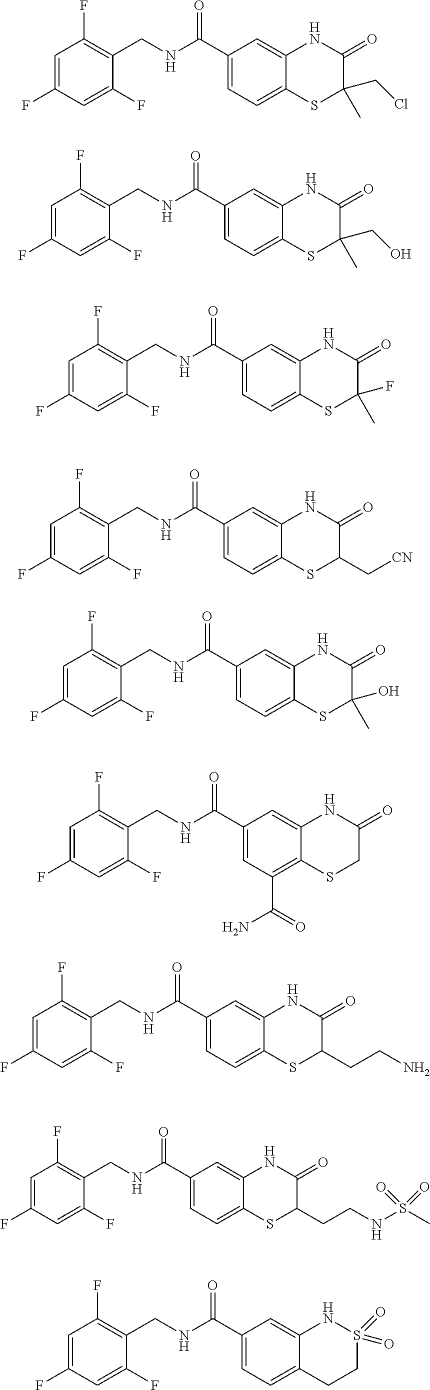

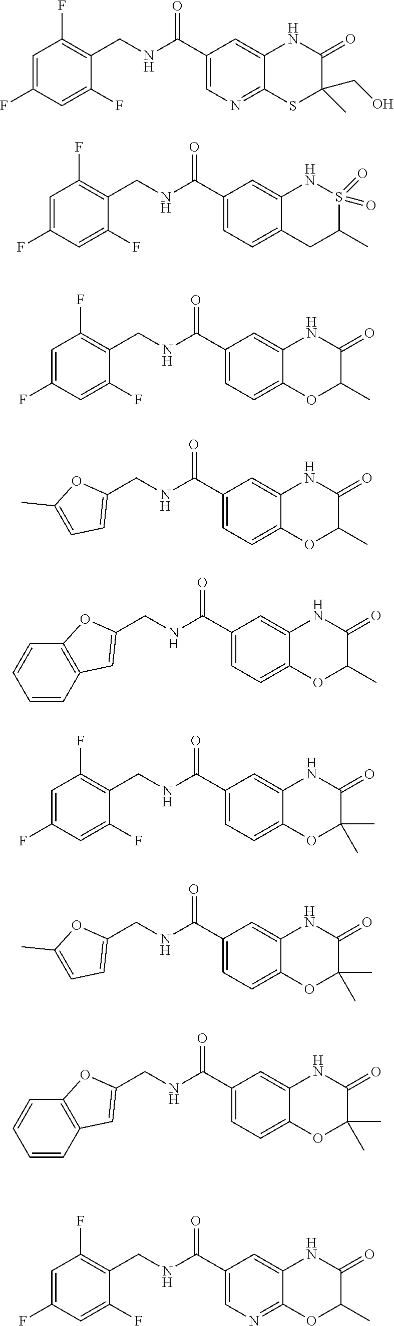

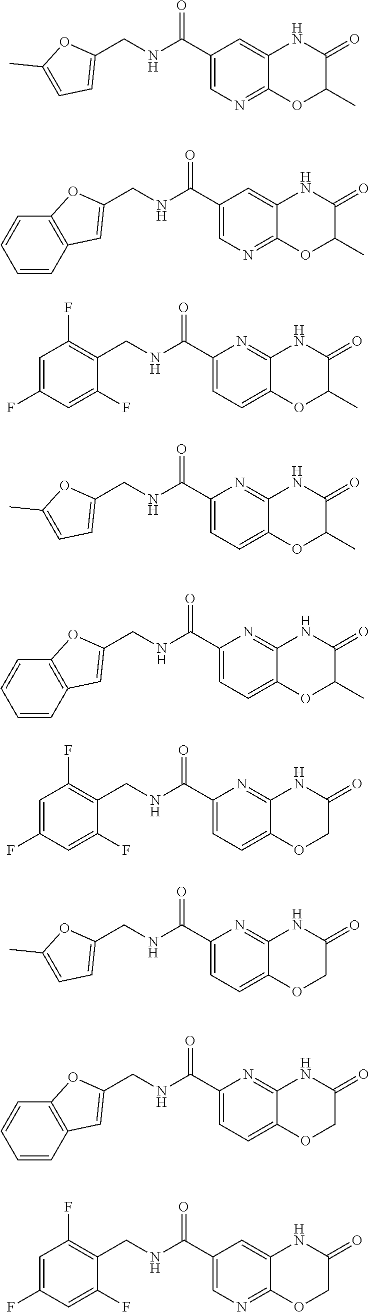

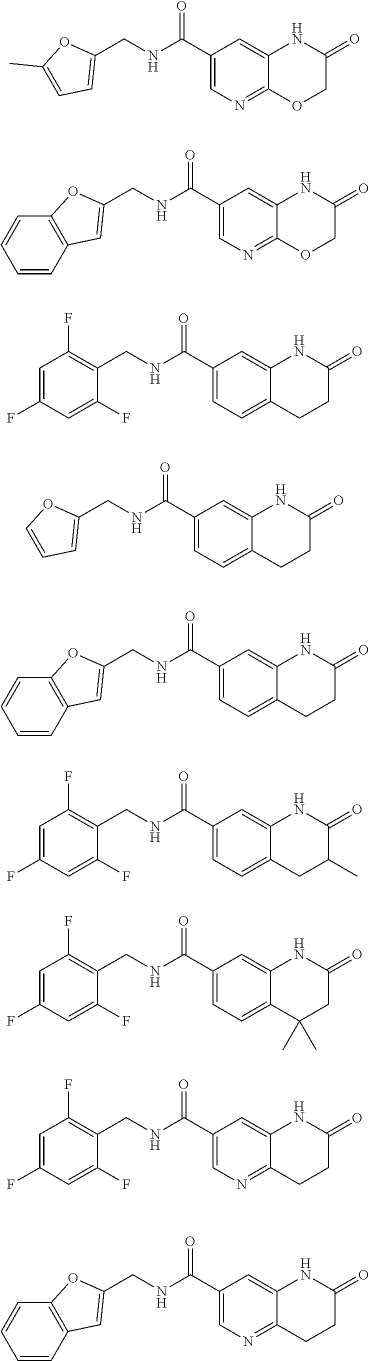

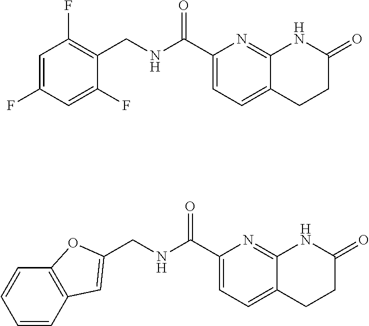 [SMILES: O=C1CCc2ccc(C(=O)NCc3c(F)cc(F)cc3F)nc2N1 ; O=C1CCc2ccc(C(=O)NCc3cc4ccccc4o3)nc2N1]

The disclosure also describes a compound of formula (V):

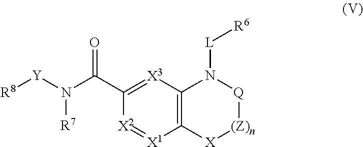

or a pharmaceutically acceptable salt or prodrug thereof, wherein:

X is CR⁹R¹⁰, O, S, S═O or SO₂;

X¹ is CR¹ or N;

X² is CR² or N;

X³ is CR³ or N;

the or each Z is independently CR¹¹R¹² or NR¹¹;

n is 1 or 2;

Q is C═O, S═O, SO₂, C═S or CR⁴R⁵;

L is optionally substituted C₁-C₆ alkyl, C₁-C₃ polyfluoroalkyl, optionally substituted C₃-C₆ cycloalkyl, optionally substituted C₂-C₆ alkenyl, optionally substituted C₂-C₆ alkynyl, C═O, S═O, SO₂, —CH₂C(O)—, —CH₂CONH—, or —CONH—;

Y is an optionally substituted C₁-C₆ alkyl, C₁-C₃ polyfluoroalkyl, an optionally substituted C₂-C₆ alkenyl, an optionally substituted C₂-C₆ alkynyl, an optionally substituted C₃-C₆ cycloalkyl, an optionally substituted mono or bicyclic 3 to 8 membered heterocycle;

R¹, R² and R³ are each independently selected from the group consisting of H, halogen, CN, hydroxyl, COOH, CONR¹R², NR¹R², NHCOR, optionally substituted C₁-C₆ alkyl, C₁-C₃ polyfluoroalkyl, optionally substituted C₁-C₆ alkylsulfonyl, optionally substituted mono or bicyclic C₃-C₆ cycloalkyl, optionally substituted C₂-C₆ alkenyl, optionally substituted C₂-C₆ alkynyl, optionally substituted C₁-C₆ alkoxy, optionally substituted C₁-C₆ alkoxycarbonyl group, mono or bicyclic optionally substituted C₅-C₁₀ aryl, mono or bicyclic optionally substituted 5 to 10 membered heteroaryl, optionally substituted mono or bicyclic 3 to 8 membered heterocycle, optionally substituted aryloxy, optionally substituted heteroaryloxy, and optionally substituted heterocyclyloxy;

R⁴ and R⁵ are each independently selected from the group consisting of H, halogen, optionally substituted C₁-C₆ alkyl, optionally substituted (C₃-C₆) cycloalkyl or R⁴ and R⁵ together with the atom to which they are attached form a spirocyclic ring;

R⁶ is a mono or bicyclic optionally substituted C₅-C₁₀ aryl, mono or bicyclic optionally substituted 5 to 10 membered heteroaryl, optionally substituted C₃-C₆ cycloalkyl or an optionally substituted mono or bicyclic 3 to 8 membered heterocycle;

R⁷ is H, optionally substituted C₁-C₆ alkyl, optionally substituted sulfonyl, optionally substituted C₁-C₆ alkylsulfonyl, optionally substituted C₃-C₆ cycloalkyl, optionally substituted C₂-C₆ alkenyl and optionally substituted C₂-C₆ alkynyl;

R⁸ is a mono or bicyclic optionally substituted C₅-C₁₀ aryl, mono or bicyclic optionally substituted 5 to 10 membered heteroaryl, optionally substituted mono or bicyclic C₃-C₆ cycloalkyl or an optionally substituted mono or bicyclic 3 to 8 membered heterocycle;

R⁹ and R¹⁰ are each independently selected from the group consisting of optionally substituted C₁-C₆ alkyl, H, halogen, CN, hydroxyl, CO₂H, CONR¹R², azido, sulfonyl, NR¹R², NHCOR, C₁-C₃ polyfluoroalkyl, optionally substituted C₁-C₆ thioalkyl, optionally substituted C₁-C₆ alkylsulfonyl, optionally substituted C₃-C₆ cycloalkyl, optionally substituted C₂-C₆ alkenyl, optionally substituted C₂-C₆ alkynyl, optionally substituted C₁-C₆ alkoxy, optionally substituted C₁-C₆ alkoxycarbonyl, mono or bicyclic optionally substituted C₅-C₁₀ aryl, mono or bicyclic optionally substituted 5 to 10 membered heteroaryl, optionally substituted heterocycle, optionally substituted aryloxy, and an optionally substituted heteroaryloxy; or R⁹ and R¹⁰ together with the C atom to which they are attached can combine to form an optionally substituted spirocyclic ring; and

R¹¹ and R¹² are each independently selected from the group consisting of optionally substituted C₁-C₆ alkyl, H, halogen, CN, hydroxyl, CO₂H, CONR¹R², azido, sulfonyl, NR¹R², NHCOR¹, C₁-C₃ polyfluoroalkyl, optionally substituted C₁-C₆ thioalkyl, optionally substituted C₁-C₆ alkylsulfonyl, optionally substituted C₃-C₆ cycloalkyl, optionally substituted C₂-C₆ alkenyl, optionally substituted C₂-C₆ alkynyl, optionally substituted C₁-C₆ alkoxy, optionally substituted C₁-C₆ alkoxycarbonyl, mono or bicyclic optionally substituted C₅-C₁₀ aryl, mono or bicyclic optionally substituted 5 to 10 membered heteroaryl, optionally substituted heterocycle, optionally substituted aryloxy, and an optionally substituted heteroaryloxy; or R¹ and R¹² together with the C atom to which they are attached can combine to form an optionally substituted spirocyclic ring;

with the proviso that when X is S; X, X² and X³ are CH; n is 1; Z is CH₂; Q is C═O; L is —CH₂—; Y is —CH₂—; R⁷ is H; and R⁶ is unsubstituted phenyl,

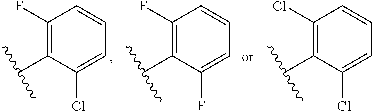

then R⁸ is not unsubstituted furanyl; and

when X is S; X¹, X² and X³ are CH; n is 1; Z is CH₂; Q is C═O; L is —CH₂—; Y is —CH₂—;

R⁷ is H; and R⁶ is

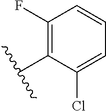 [SMILES: *c1c(F)cccc1Cl]

then R⁸ is not unsubstituted phenyl;

or a pharmaceutically acceptable complex, salt, solvate, tautomeric form or polymorphic form thereof, for use in therapy.

Preferably, when X is S; X¹, X² and X³ are CH; n is 1; Z is CH₂; Q is C═O; L is —CH₂—; Y is —CH₂—; R⁷ is H; and R⁶ is an optionally substituted phenyl then R⁸ is not an optionally substituted 5 membered heteroaryl. Optionally, when X is S; X¹, X² and X³ are CH; n is 1; Z is CH₂; Q is C═O; L is —CH₂—; Y is —CH₂—; R⁷ is H; and R⁶ is an optionally substituted phenyl then R⁸ is not an optionally substituted 5 or 6 membered heteroaryl.

The disclosure also describes a compound of formula (VI):

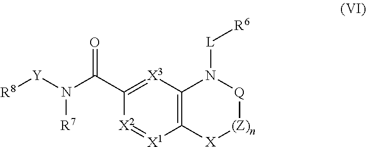

or a pharmaceutically acceptable salt or prodrug thereof, wherein:

X is CR⁹R¹⁰, O, S, S═O or SO₂;

X¹ is CR¹ or N;

X² is CR² or N;

X³ is CR³ or N;

the or each Z is independently CR¹¹R¹² or NR¹¹;

n is 1 or 2;

Q is C═O, S═O, SO₂, C═S or CR⁴R⁵;

L is optionally substituted C₁-C₆ alkyl, C₁-C₃ polyfluoroalkyl, optionally substituted C₃-C₆ cycloalkyl, optionally substituted C₂-C₆ alkenyl, optionally substituted C₂-C₆ alkynyl, C═O, S═O, SO₂, —CH₂C(O)—, —CH₂CONH—, or —CONH—;

Y is an optionally substituted C₁-C₆ alkyl, C₁-C₃ polyfluoroalkyl, an optionally substituted C₂-C₆ alkenyl, an optionally substituted C₂-C₆ alkynyl, an optionally substituted C₃-C₆ cycloalkyl;

R¹, R² and R³ are each independently selected from the group consisting of H, halogen, CN, hydroxyl, COOH, CONR¹R², NR¹R², NHCOR, optionally substituted C₁-C₆ alkyl, C₁-C₃ polyfluoroalkyl, optionally substituted C₁-C₆ alkylsulfonyl, optionally substituted mono or bicyclic C₃-C₆ cycloalkyl, optionally substituted C₂-C₆ alkenyl, optionally substituted C₂-C₆ alkynyl, optionally substituted C₁-C₆ alkoxy, optionally substituted C₁-C₆ alkoxycarbonyl group, mono or bicyclic optionally substituted C₅-C₁₀ aryl, mono or bicyclic optionally substituted 5 to 10 membered heteroaryl, optionally substituted mono or bicyclic 3 to 8 membered heterocycle, optionally substituted aryloxy, optionally substituted heteroaryloxy, and optionally substituted heterocyclyloxy;

R⁴ and R⁵ are each independently selected from the group consisting of H, halogen, optionally substituted C₁-C₆ alkyl, optionally substituted (C₃-C₆) cycloalkyl or R⁴ and R⁵ together with the atom to which they are attached form a spirocyclic ring;

R⁶ is mono or bicyclic optionally substituted C₅-C₁₀ aryl, mono or bicyclic optionally substituted 5 to 10 membered heteroaryl, optionally substituted C₃-C₆ cycloalkyl or an optionally substituted mono or bicyclic 3 to 8 membered heterocycle;

R⁷ is H, optionally substituted C₁-C₆ alkyl, optionally substituted sulfonyl, optionally substituted C₁-C₆ alkylsulfonyl, optionally substituted C₃-C₆ cycloalkyl, optionally substituted C₂-C₆ alkenyl and optionally substituted C₂-C₆ alkynyl;

R⁸ is mono or bicyclic optionally substituted C₅-C₁₀ aryl, mono or bicyclic optionally substituted 5 to 10 membered heteroaryl, optionally substituted mono or bicyclic C₃-C₆ cycloalkyl or an optionally substituted mono or bicyclic 3 to 8 membered heterocycle;

R⁹ and R¹⁰ are each independently selected from the group consisting of optionally substituted C₁-C₆ alkyl, H, halogen, CN, hydroxyl, CO₂H, CONR¹R², azido, sulfonyl, NR¹R², NHCOR¹, C₁-C₃ polyfluoroalkyl, optionally substituted C₁-C₆ thioalkyl, optionally substituted C₁-C₆ alkylsulfonyl, optionally substituted C₃-C₆ cycloalkyl, optionally substituted C₂-C₆ alkenyl, optionally substituted C₂-C₆ alkynyl, optionally substituted C₁-C₆ alkoxy, optionally substituted C₁-C₆ alkoxycarbonyl, mono or bicyclic optionally substituted C₅-C₁₀ aryl, mono or bicyclic optionally substituted 5 to 10 membered heteroaryl, optionally substituted heterocycle, optionally substituted aryloxy, and an optionally substituted heteroaryloxy; or R⁹ and R¹⁰ together with the C atom to which they are attached can combine to form an optionally substituted spirocyclic ring; and

R¹¹ and R¹² are each independently selected from the group consisting of optionally substituted C₁-C₆ alkyl, H, halogen, CN, hydroxyl, CO₂H, CONR¹R², azido, sulfonyl, NR¹R², NHCOR¹, C₁-C₃ polyfluoroalkyl, optionally substituted C₁-C₆ thioalkyl, optionally substituted C₁-C₆ alkylsulfonyl, optionally substituted C₃-C₆ cycloalkyl, optionally substituted C₂-C₆ alkenyl, optionally substituted C₂-C₆ alkynyl, optionally substituted C₁-C₆ alkoxy, optionally substituted C₁-C₆ alkoxycarbonyl, mono or bicyclic optionally substituted C₅-C₁₀ aryl, mono or bicyclic optionally substituted 5 to 10 membered heteroaryl, optionally substituted heterocycle, optionally substituted aryloxy, and an optionally substituted heteroaryloxy; or R¹¹ and R¹² together with the C atom to which they are attached can combine to form an optionally substituted spirocyclic ring;

with the proviso that when X is S; X¹, X² and X³ are CH; n is 1; Z is CH₂; Q is C═O; L is —CH₂—; Y is —CH₂—; R⁷ is H; and R⁶ is

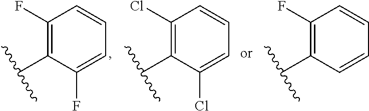

then R^{S} is not unsubstituted furanyl;

when X is S; X¹, X² and X³ are CH; n is 1; Z is CH₂; Q is C═O; L is —CH₂—; Y is —CH₂—;

R⁷ is H; and R⁶ is

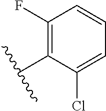 [SMILES: *c1c(F)cccc1Cl]

then R⁸ is not unsubstituted phenyl, unsubstituted thiophenyl, unsubstituted pyridinyl, unsubstituted furanyl, unsubstituted tetrahydrofuranyl,

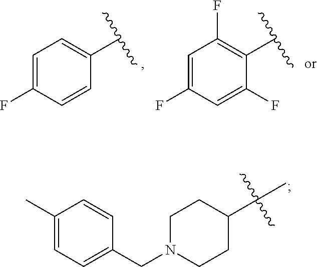 [SMILES: *c1ccc(F)cc1 ; *c1c(F)cc(F)cc1F ; *C1CCN(Cc2ccc(C)cc2)CC1]

when X is S; X¹, X² and X³ are CH; n is 1; Z is CH₂; Q is C═O; L is —CH₂—; Y is —CH₂CH₂—; R⁷ is H; and R⁶ is

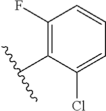 [SMILES: *c1c(F)cccc1Cl]

then R⁸ is not unsubstituted phenyl;

when X is S; X¹, X² and X³ are CH; n is 1; Z is CH₂; Q is C═O; L is —CH₂—; Y is;

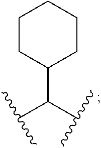 [SMILES: *C(*)C1CCCCC1]

R⁷ is H; and R⁶ is

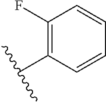 [SMILES: *c1ccccc1F]

then R⁸ is not unsubstituted phenyl;

when X is S; X¹, X² and X³ are CH; n is 1; Z is CH₂; Q is C═O; L is —CH₂—; Y is —CH₂—;

R⁷ is H; and R⁶ is unsubstituted phenyl then R⁸ is not unsubstituted furanyl, unsubstituted phenyl,

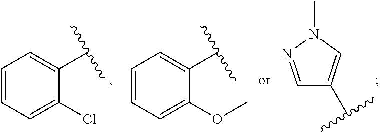

when X is S; X¹, X² and X³ are CH; n is 1; Z is CH₂; Q is C═O or CH₂; L is —CH₂—; Y is —CH₂—; R⁷ is H; and R⁶ is

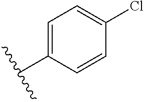 [SMILES: *c1ccc(Cl)cc1]

then R⁸ is not unsubstituted furanyl;

when X is S; X¹, X² and X³ are CH; n is 1; Z is CH₂; Q is C═O; L is —CH₂—; Y is —CH₂—;

R⁷ is H; and R⁶ is

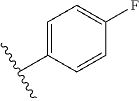 [SMILES: *c1ccc(F)cc1]

then R⁸ is not unsubstituted furanyl;

when X is S; X¹, X² and X³ are CH; n is 1; Z is CH₂; Q is C═O; L is —CH₂—; Y is —CH₂—;

R⁷ is H; and R⁶ is

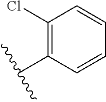 [SMILES: *c1ccccc1Cl]

then R⁸ is not unsubstituted furanyl,

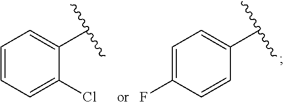 [SMILES: *c1ccccc1Cl ; *c1ccc(F)cc1]

when X is S; X, X² and X³ are CH; n is 1; Z is CH₂; Q is C═O; L is —CH₂—; Y is —CH₂—;

R⁷ is H; and R⁶ is

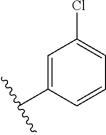 [SMILES: *c1cccc(Cl)c1]

then R⁸ is not unsubstituted furanyl or

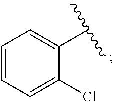 [SMILES: *c1ccccc1Cl]

and

when X is S; X¹, X² and X³ are CH; n is 1; Z is CH₂; Q is C═O; L is —CH₂—; Y is

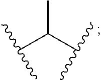 [SMILES: *C(*)C]

R⁷ is H; and R⁶ is

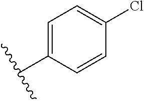 [SMILES: *c1ccc(Cl)cc1]

then R⁸ is not unsubstituted phenyl;

or a pharmaceutically acceptable complex, salt, solvate, tautomeric form or polymorphic form thereof.

All features described herein (including any accompanying claims and abstract), and/or all of the steps of any method or process so disclosed, may be combined with any of the above aspects in any combination, except combinations where at least some of such features and/or steps are mutually exclusive.

For a better understanding of the invention, and to show embodiments of the same may be carried into effect, reference will now be made, by way of example, to the accompanying Figures, in which:—

FIG. 1 shows allele frequency of the major polymorphisms of human STING derived from the 1000 Genome Project database;

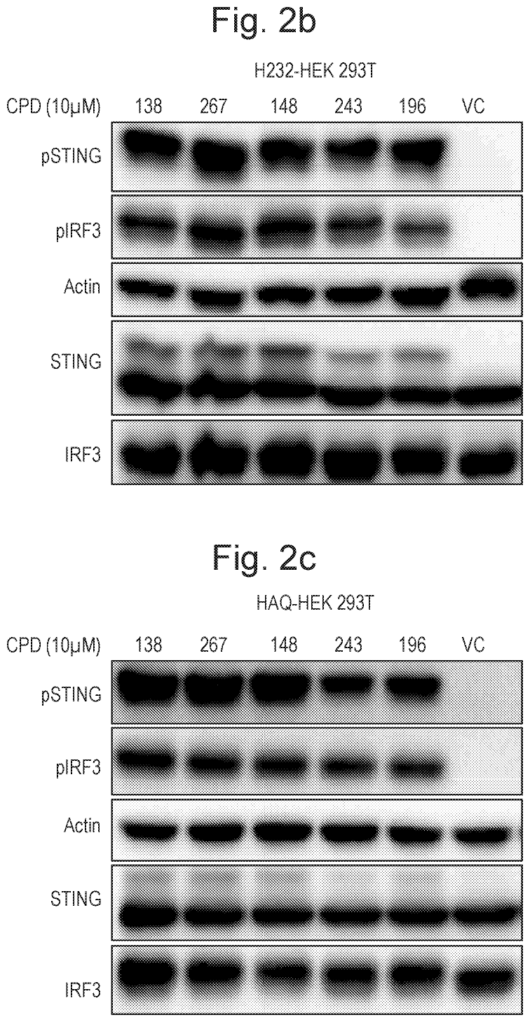

FIG. 2 are Western blots of human STING proteins combined with compounds of the invention or a vehicle control (VC) and incubated with antibodies specific for phosphorylated STING (pSTING), phosphorylated IRF3 (pIRF3), ACTIN, total STING (STING), and IRF3;

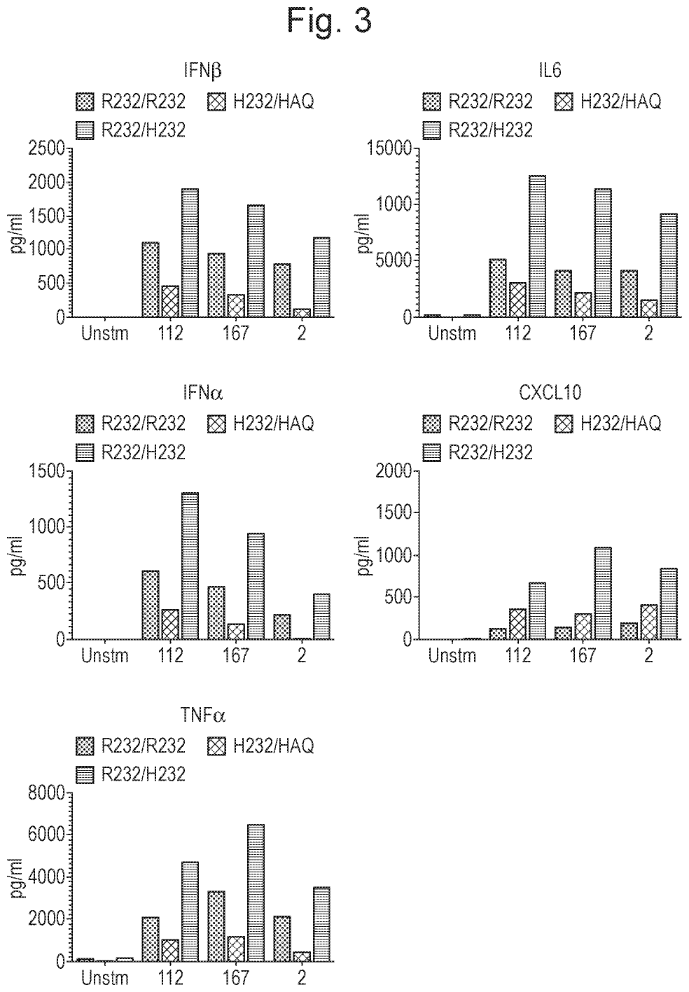

FIG. 3 shows the results of cytokines measured by an ELISA assay of human PBMCs stimulated with compounds of the invention compared to an unstimulated control (Unstm); and

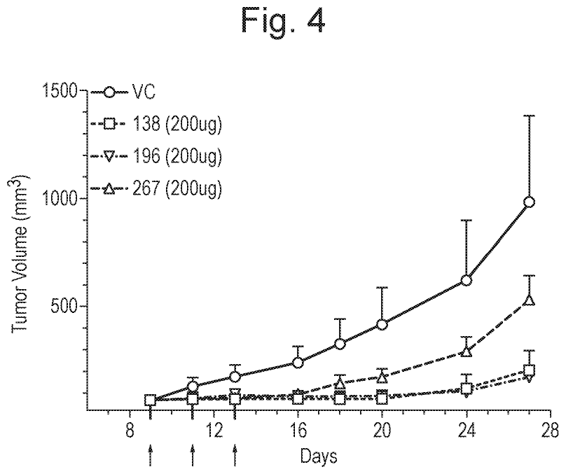

FIG. 4 shows tumour growth against time (in days) in mice dosed intra-tumorally with compounds of the invention or a VC.

### GENERAL SCHEMES

### General Scheme 1

Compounds of formula (I) may be prepared from compounds of formula (II) and (III) using an amide bond forming reaction, as shown below.

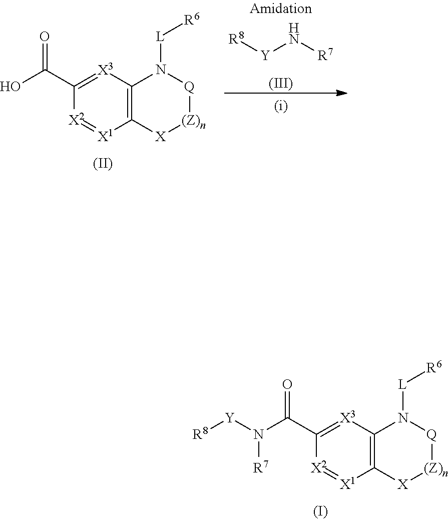

Typical conditions employ activation of the carboxylic acid of the compound of formula (II) using a suitable organic base and a suitable coupling agent. Preferred coupling agents are either EDCI with HOBt, T₃P, HATU, HBTU or BOP. Preferred organic bases comprise either DIPEA or TEA in a suitable organic solvent such as DCM, DMF, DMA or MeCN. The reaction may be shaken or stirred at room temperature.

Compounds of formula (II) and (III) are commercially available or may be synthesized by those skilled in the art. In particular, methods of synthesising compounds of formula (II) are described in General Schemes 2 to 4 (below).

### General Scheme 2

Compounds of formula (II) may be synthesized from esters of formula (IV), where R is methyl, ethyl, benzyl or tert-butyl, by a hydrolysis reaction.

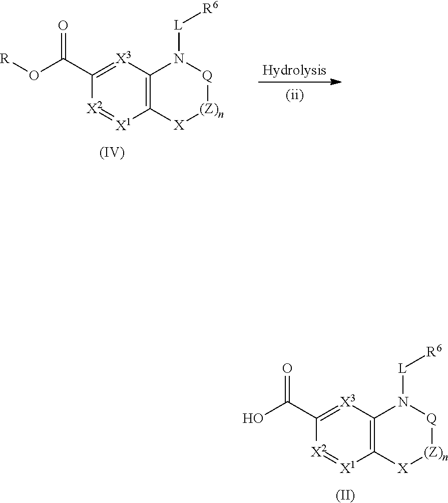

The compound of Formula (IV) may be reacted with a suitable alkali or base to cause it to undergo hydrolysis and provide a compound of formula (II). The suitable alkali or base may be LiOH, KOH, NaOH or K₂CO₃, and the reaction may be conducted in an aqueous solution.

### General Scheme 3

Alternatively, compounds of formula (II) can be obtained from a halide of formula (V) as shown in the general scheme below.

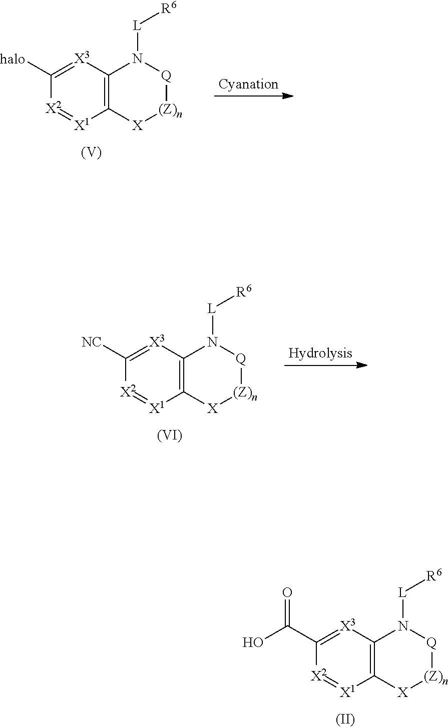

First the compound of formula (V) undergoes a cyanation reaction to give a compound of formula (VI). This could be conducted in the using CuCN or ZnCN₂ in a polar solvent at elevated temperatures with a suitable catalyst. The polar solvent could be NMP, DMF, DMA or MeCN and catalyst could be tetrakistriphenylphosphine palladium(0).

The compound of formula (VI) may then undergo hydrolysis to give the compound of formula (II). In particular, the compound of formula (II) may be hydrolysed using an aqueous solution of an alkali, such as NaOH, LiOH and KOH, or an acid, such as HCl, at an elevated temperature.

### General Scheme 4

In a further alternative process, the compound of formula (V) may undergo a direct carbonylation reaction to produce a compound of formula (II), as shown below.

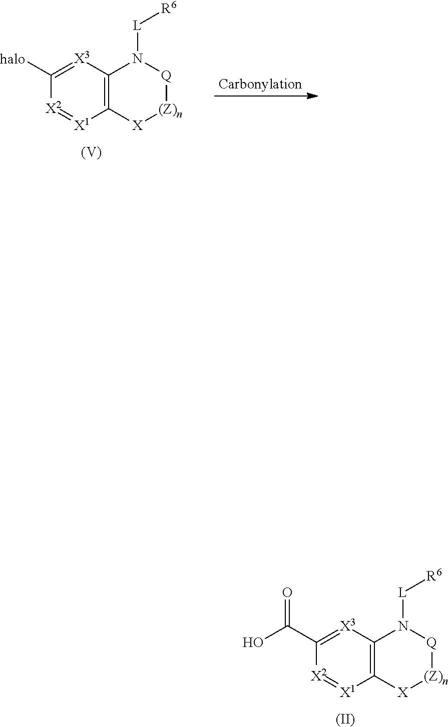

The reaction could be conducted using CO gas in the presence of a suitable catalyst in an appropriate polar solvent. The catalyst may be a Pd, Rh, Ir or Fe catalyst, and the solvent may be NMP, DMF, DMA or MeCN with the reaction carried out in the presence of a suitable nucleophile such as water or alcohols (to prepare the corresponding esters).

### General Scheme 5

Compounds of formulae (IV), (V) and (VI) may be synthesized by those skilled in the art via an alkylation/acylation/sulfonylation reaction with a compound of formula (VII), where G is a leaving group such as an optionally substituted alkylaryl(het), alkyl, aryl(het), cycloalkyl, alkyleyloalkyl halide, triflate or tosylate.

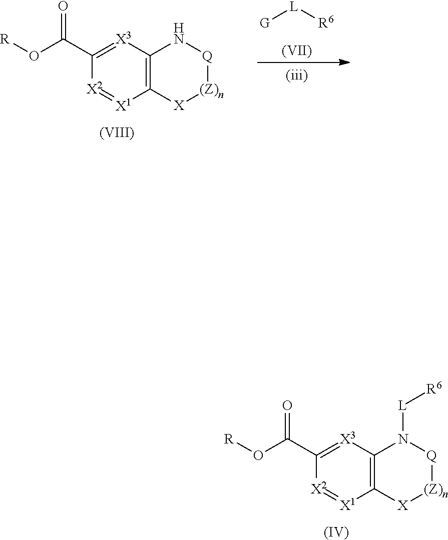

### General Scheme 6

Alternatively, a compound of formula (IX) may be prepared in a five-step process, as shown below, from a compound of formula (XIV), where R is methyl, ethyl, benzyl or tert-butyl.

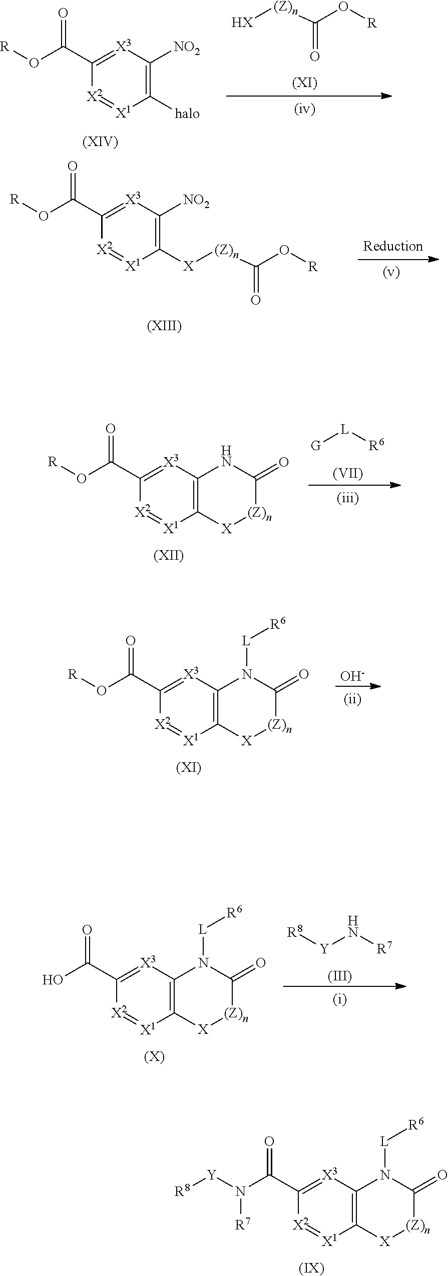

First, the compound of formula (XIV) undergoes a nucleophilic substitution reaction with a compound of formula (XI), where R is methyl, ethyl, benzyl or tert-butyl, to produce a compound of formula (XIII). The nucleation substitution reaction may be conducted in the presence of a mild base, such as DBU, NaH, TEA, DIPEA, K₂CO₃, Cs₂CO₃ or KHCO₃. The solvent used may be 1,4-dioxane, acetone, MeCN, THF or DMF.

The nitro group on the compound of formula (XIII) may then be reduced to an amino group by using a reducing agent, such as Fe/AcOH, Zn/HCl, Zn/NH₄Cl, Zn/HCOONH₄, SnCl₂/HCl or Pd/C/H₂, in a suitable solvent, such as EtOH, MeOH or THF. The ensuing amino compounds undergo in-situ cyclization resulting in the formation of a compound of formula (XII).

The compound of formula (XII) may then undergo an alkylation/acylation/sulfonylation reaction, as described in General Scheme 5, to give a compound of formula (XI). This compound may undergo a hydrolysis reaction, as described in General Scheme 2, to give a compound of formula (X). Finally, this compound may be reacted with a compound of formula (III), as described in General Scheme 1, to give a compound of formula (IX).

It will be appreciated that the compound of formula (IX) is a compound of formula (I) where Q is C═O.

### General Scheme 7

Alternatively, a compound of formula (XV) may be prepared in a five-step process, as shown below, from a compound of formula (XII), where R is methyl, ethyl, benzyl or tert-butyl.

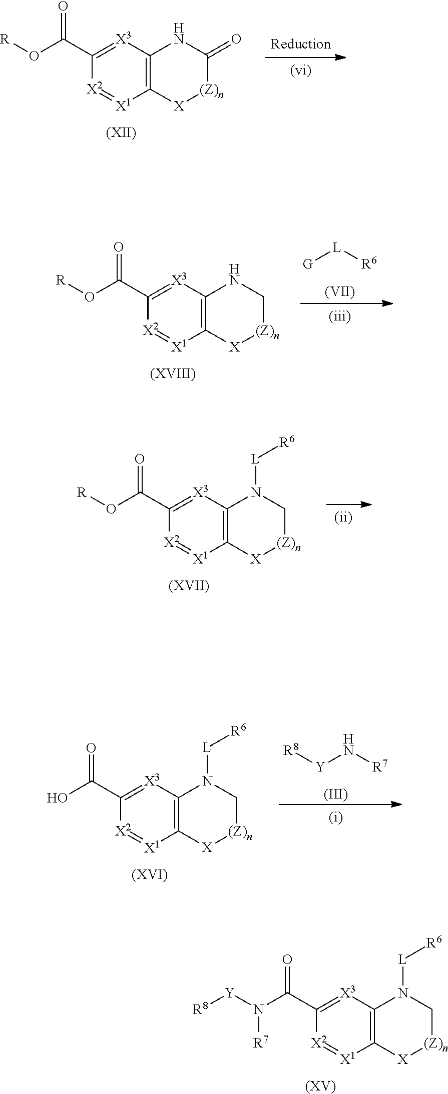

Firstly, the carbonyl group of the compound of formula (XII) may be reduced to the corresponding methylene group to give a compound of formula (XVIII). This can be achieved by using a suitable reducing agent, such as borane-THF or borane-DMSO, in a solvent such as THF or DMSO.

The compound of formula (XVIII) may then undergo an alkylation/acylation/sulfonylation reaction, as described in General Scheme 5, to give a compound of formula (XVII). This compound may undergo a hydrolysis reaction, as described in General Scheme 2, to give a compound of formula (XVI). Finally, this compound may be reacted with a compound of formula (III), as described in General Scheme 1, to give a compound of formula (XV).

It will be appreciated that the compound of formula (IX) is a compound of formula (I) where Q is CH₂

### General Scheme 8

A compound of formula (XIX) may be prepared in a six-step process, as shown below, from a compound of formula (XXV), where R is methyl, ethyl, benzyl or tert-butyl.

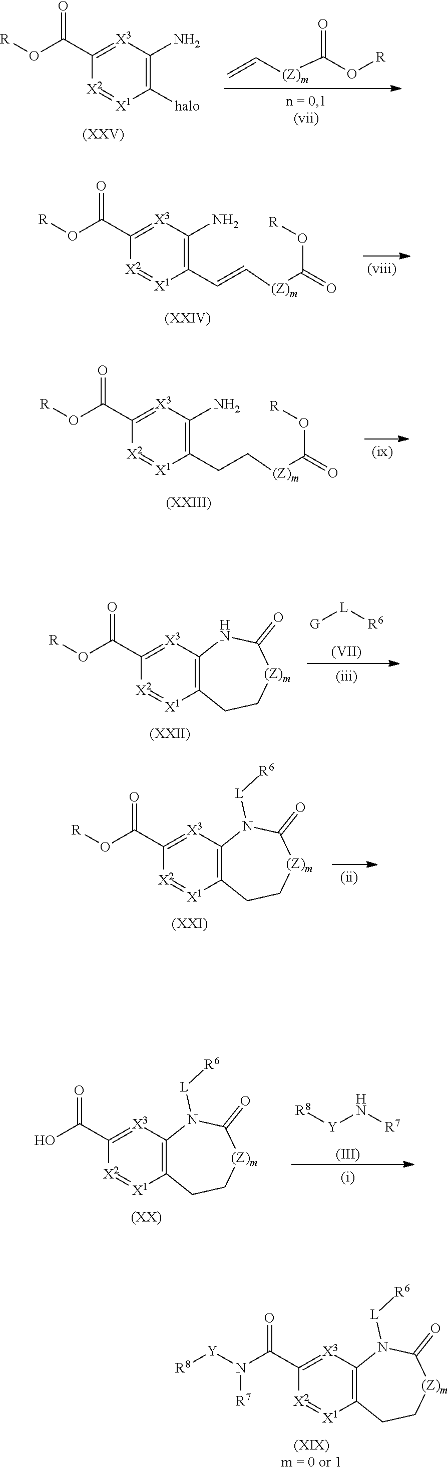

First the compound of formula (XXV) may react with an active olefin-containing compound via a Heck alkenylation reaction to give a compound of formula (XXIV). This reaction will be conducted in a sealed tube at an elevated temperature using a suitable transition metal catalyst and a suitable base in a suitable solvent. The transition metal catalyst might be a palladium catalyst, such as tetrakistriphenylphosphine palladium (0) or Ataphose, the base may be TEA or DIPEA and the solvent may be NMP, DMF, DMA or MeCN.

The compound of formula (XXIV) may then be reduced using a hydrogenation reaction with hydrogen gas and a suitable catalyst in a suitable solvent to provide a compound of formula (XXIII). The catalyst may be palladised charcoal and the solvent may EtOH or MeOH.

The compound of formula (XXIII) may then undergo a cyclization reaction to provide a compound of formula (XXII). The reaction may be conducted in an acidic medium at an elevated temperature in a suitable solvent, such as toluene, DMF, DMA or MeCN.

The compound of formula (XXII) may then undergo an alkylation/acylation/sulfonylation reaction, as described in General Scheme 5, to give a compound of formula (XXI). This compound may undergo a hydrolysis reaction, as described in General Scheme 2, to give a compound of formula (XX). Finally, this compound may be reacted with a compound of formula (III), as described in General Scheme 1, to give a compound of formula (XIX).

It will be appreciated that when m is 0 the compound of formula (XIX) is a compound of formula (I) where X is CH₂ and Z is CH₂, and when m is 1 the compound of formula (XIX) is a compound of formula (I) where X is CH₂, one Z is CH₂ and the other may be any one of the groups defined in claim 1.

### General Scheme 9

A compound of formula (XXVI) may be prepared in a five-step process, as shown below, from a compound of formula (XXVI), where R is methyl, ethyl, benzyl or tert-butyl.

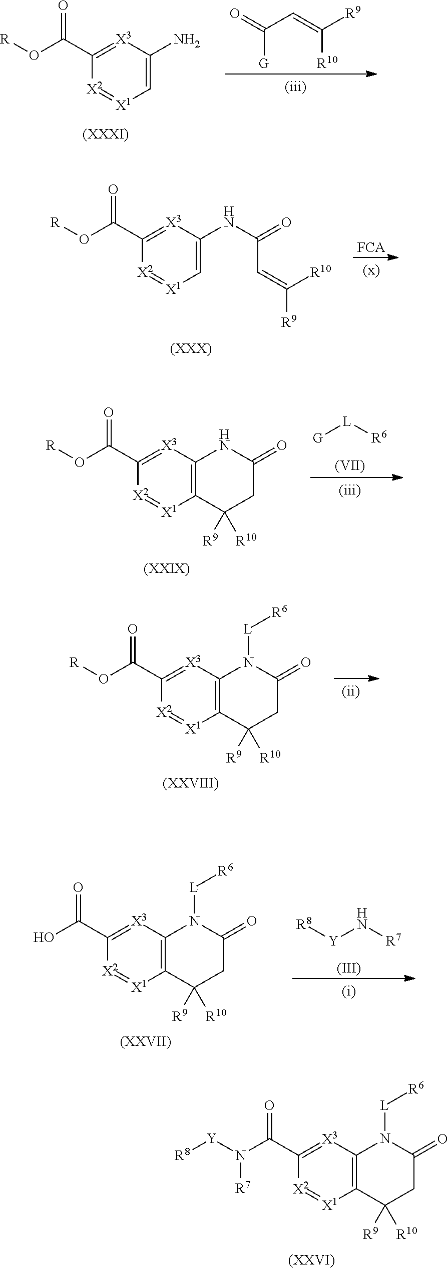

First, the compound of formula (XXXI) may undergo an amide formation to provide a compound of formula (XXX). The conditions used may be similar to those described under General Scheme 5.

The compound of formula (XXX) may then be cyclized to give a compound of formula (XXIX). For instance, a Friedel-Crafts cyclisation reaction could be used. This reaction would use a suitable Lewis acid reagent at elevated temperatures and in a suitable solvent. The Lewis acid reagent could be AlCl₃ or FeCl₃, and the solvent could be MeCN, toluene, EDC or MeOAc. Alternatively, Bronsted acids, such as TfOH, may be used to cause the compound of formula (XXX) to cyclise.

The compound of formula (XXIX) may then undergo an alkylation/acylation/sulfonylation reaction, as described in General Scheme 5, to give a compound of formula (XXVIII). This compound may undergo a hydrolysis reaction, as described in General Scheme 2, to give a compound of formula (XXVII). Finally, this compound may be reacted with a compound of formula (III), as described in General Scheme 1, to give a compound of formula (XXVI).

It will be appreciated that the compound of formula (XXVI) is a compound of formula (I) where X is R⁹R¹⁰, Q is C═O, n is 1 and Z is CH₂.

### General Scheme 10

A compound of formula (XXXII) may be prepared in a four- or five-step process, as shown below, from a compound of formula (XXXV), where R is methyl, ethyl, benzyl or tert-butyl.

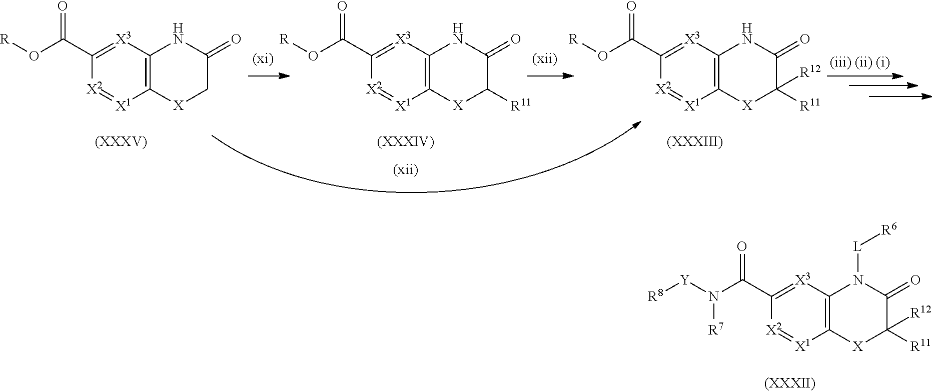

The compound of formula (XXXV) is reacted with a suitable base and a suitable electrophile to cause an alkylation reaction and provide the compound of formula (XXXIII). The base may be a mild base, such as TEA, DIPEA, K₂CO₃, Cs₂CO₃ or KHCO₃, or a stronger base, such as NaH or LiHMDS. The electrophile may be R⁹-G and/or R¹⁰-G where G is a suitable leaving group. The process may comprise sequential alkylation reactions, analogous to steps (xi) and (xii) shown above, or a double alkylation, analogous to step (xii).

The compound of formula (XXXIII may then undergo an alkylation/acylation/sulfonylation reaction, as described in General Scheme 5, a hydrolysis reaction, as described in General Scheme 2, and then the resulting compound may be reacted with a compound of formula (III), as described in General Scheme 1, to give a compound of formula (XXXII).

It will be appreciated that the compound of formula (XXXII) is a compound of formula (I) where Q is C═O and n is 1.

### General Scheme 11

Compounds of formula (XXXVII) and (XXXVI) can be synthesized by S-oxidation of compounds of formula (XXXVIII) with a suitable oxidizing agent, such as mCPBA, hydrogen peroxide or oxygen, in a solvent such as DCM, CHCl₃, CCl₄ or EDC.

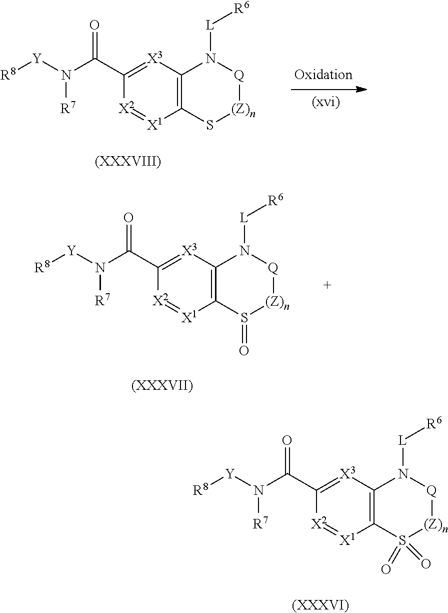

It will be appreciated that the compound of formula (XXXVII) is a compound of formula (I) where X is SO and the compound of formula (XXXVI) is a compound of formula (I) where X is SO₂.

### General Synthetic Procedures

### General Procedure 1

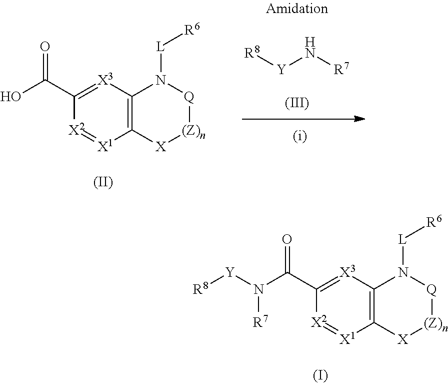

To a stirred solution of a carboxylic acid (II) (1.277 mmol) in a suitable solvent, such as DCM, DMF, DMA or MeCN, (10 mL) was added amine (III) (1.2 eq.) and a coupling reagent, such as T₃P, HATU, EDCl, HOBT, BOP or HBTU (1.5 eq.) followed by addition of an organic base, such as DIPEA or TEA (2.0 eq.) drop wise to the solution and the mixture allowed to stir at RT for 2-3 h. When UPLC or TLC showed completion of the reaction, the reaction mixture was diluted with water and extracted with EtOAc. The combined organic layers were washed with aqueous NaHCO₃ solution followed by dilute aqueous HCl and finally with brine, and then dried over anhydrous Na₂SO₄. The solvent was evaporated under reduced pressure to obtain the crude material which was purified by Combi-flash using mixtures of EtOAc in hexanes as eluent to afford a compound of formula (I) (70-80% yield) as a pale yellow solid. A similar procedure can be followed to synthesize all amides of formula (I).

Compounds of formula (I) were also prepared in parallel using library or array techniques according to one of the following methods.

### Library General Procedure 1

### Method 1

To a stirred solution of carboxylic acid (0.084 mmol, 1 eq.) in DMF (1.0 mL) was added DIPEA (0.209 mmol, 2.5 eq.) and T₃P (0.251 mmol, 3.0 eq.) at RT and stirred for 5 min. The corresponding amine (1.5 eq.) was added and the whole stirred for 16 h at RT. The progress of the reaction was monitored by LCMS. After completion, the reaction mixture was purified by prep-HPLC.

### Method 2

To a stirred solution of carboxylic acid (0.084 mmol, 1.0 eq.) in DMF (1.0 mL) was added DIPEA (0.209 mmol, 2.5 eq.) and the corresponding amine (1.5 eq.) followed by HATU (0.125 mmol, 1.5 eq.) and stirred for 16 h at RT. The progress of the reaction was monitored by LCMS. After completion, the reaction mixture was purified by prep-HPLC.

### Method 3

To a stirred solution of carboxylic acid (0.084 mmol, 1.0 eq.) in DMF (1.0 mL) was added DIPEA (0.209 mmol, 2.5 eq.), HOBT (0.1 mmol, 1.2 eq.) and EDC.HCl (0.167 mmol, 2.0 eq.) followed by corresponding amine (1.5 eq.) and stirred for 16 h at room temperature. The progress of the reaction was monitored by LCMS. After completion, the reaction mixture was purified by prep-HPLC.

### General Purification and Analytical Methods

All final compounds were purified by either Combi-flash or prep-HPLC purification, and analysed for purity and product identity by UPLC or LCMS according to one of the below conditions.

### Prep-HPLC

Preparative HPLC was carried out on a Waters auto purification instrument using either a YMC Triart C18 column (250×20 mm, 5 μm) or a Phenyl Hexyl column (250×21.2 mm, 5 μm) operating at between ambient temperature and 50° C. with a flow rate of 16.0-50.0 mL/min.

Mobile phase 1: A=20 mM Ammonium Bicarbonate in water, B=Acetonitrile; Gradient Profile: Mobile phase initial composition of 80% A and 20% B, then to 600% A and 40% B after 3 min., then to 30% A and 70% B after 20 min., then to 5% A and 95% B after 21 min., held at this composition for 1 min. for column washing, then returned to initial composition for 3 min.

Mobile phase 2: A=10 mM Ammonium Acetate in water, B=Acetonitrile; Gradient Profile: Mobile phase initial composition of 90% A and 10% B, then to 70% A and 30% B after 2 min., then to 20% A and 80% B after 20 min., then to 5% A and 95% B after 21 min., held at this composition for 1 min. for column washing, then returned to initial composition for 3 min.

LCMS method General 5 min method: Zorbax Extend C₁₈ column (500×4.6 mm, 5 μm) operating at ambient temperature and a flow rate of 1.2 mL/min. Mobile phase: A=10 mM Ammonium Acetate in water, B=Acetonitrile; Gradient profile: from 90% A and 10% B to 70% A and B in 1.5 min, and then to 10% A and 90% B in 3.0 min, held at this composition for 1.0 min, and finally back to initial composition for 2.0 min.

### UPLC Method

UPLC was carried out on a Waters auto purification instrument using a Zorbax Extend C₁₈ column (50×4.6 mm, 5 μm) at ambient temperature and a flow rate of 1.5 ml/min. Mobile phase 1: A=5 mM Ammonium Acetate in water, B=5 mM Ammonium Acetate in 90:10 Acetonitrile/water; Gradient profile from 95% A and 5% B to 65% A and 35% B in 2 min., then to % A and 90% B in 3.0 min., held at this composition for 4.0 min. and finally back to the initial composition for 5.0 min.

Mobile phase 2: A=0.05% formic acid in water, B=Acetonitrile; Gradient profile from 98% A and 2% B over 1 min., then 90% A and 10% B for 1 min., then 2% A and 98% B for 2 min. and then back to the initial composition for 3 min.

### General Procedure 2

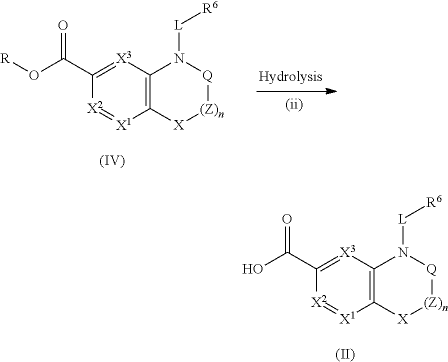

To a stirred solution of ester (IV) (1.49 mmol) in a mixture of MeOH or THF (10 mL) and water (5 mL) was added LiOH, NaOH or KOH (2.0 eq.) at RT and the resulting reaction mixture was stirred at RT for 2-16 h. TLC showed complete consumption of the ester (IV), upon which the solvent was evaporated under reduced pressure and the resulting residue was washed with ether. The residue was then acidified with 1N HCl to pH 2-4, which resulted in the formation of a precipitate, which was filtered and washed with water and then dried under reduced pressure at 50-60° C. to afford the desired carboxylic acid of formula (II) (70-85% yield) as an off white solid.

### General Procedure 3

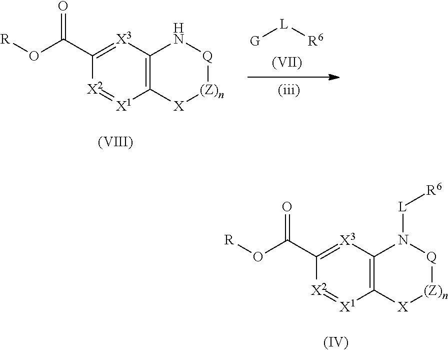

### Option 1

To a stirred solution of a compound of formula (VIII) (2.77 mmol, 1.0 eq.) in DMF or THF (4 mL/mmol) was added K₂CO₃, Cs₂CO₃, Na₂CO₃, NaOH or NaH (2.0 eq.)—in the case where NaOH was used, TBAB (0.1 eq.) was also added as a phase transfer catalyst followed by addition of a compound of formula (VII) (1.5 eq.) and the mixture allowed to stir at RT for 0.5-1 h. The reaction was monitored by TLC. After completion of the reaction; the reaction mixture was diluted with water, extracted with EtOAc, and the organic layers were washed with brine and dried over anhydrous Na₂SO₄. The organics were evaporated under reduced pressure to obtain the crude product which was purified by Combi-flash using mixtures of EtOAc in hexanes as eluent to afford compounds of formula (IV) (80-90% yield) as colourless oils.

### Option 2

Alternatively, to a stirred solution of a compound of formula (VIII) (2.77 mmol) in DCM or MeCN or THF (4 mL/mmol) was added TEA or DIPEA (2.0 eq.) followed by addition of a compound of formula (VII) (1.5 eq.) and the whole allowed to stir at RT for 0.5 to 1 h. The progress of the reaction was monitored by TLC. After completion of the reaction, the mixture was diluted with water, extracted with EtOAc, and the combined organic layers were washed with brine and dried over anhydrous Na₂SO₄. The organic layers were evaporated under reduced pressure to obtain the crude product which was purified by Combi-flash using mixtures of EtOAc in hexanes as eluent to afford a compound of formula (IV) (80-90% yield) as colourless oil.

Compounds of formula (I) were prepared using a modification of General Procedure 3 in parallel using library or array techniques starting from the corresponding amide version of the compound of formula (VIII) according to one of the following methods.

### Library General Procedure 2

### Method 1

To a stirred suspension of lactam (0.104 mmol, 1.0 eq.) in EDC (1.5 mL/mmol) was added NaOH (2.0 eq., dissolved in 1.5 mL water) and TBAB (5 mg). The resulting reaction mixture was stirred for 1 h at RT, then corresponding aryl halide (1.5 eq.) was added to it. The reaction mixture was further stirred for 3-4 h at RT. The progress of the reactions was monitored by LCMS and after completion of reaction; the reaction mixture was diluted with DCM and washed with water. The organic layers were dried over anhydrous Na₂SO₄ and evaporated to dryness to obtain the crude product, which was purified by prep-HPLC.

### Method 2

To a stirred solution of the lactam (0.104 mmol, 1.0 eq.) in DMF (3 mL/mmol) was added Cs₂CO₃ (1.5 eq.) at RT. After 10-15 min stirring, the corresponding aryl halides (1.5 eq.) were added and the whole stirred at 60° C. for 12 h. Progress of the reactions was monitored by LCMS. After completion, the reaction mixture was purified by prep-HPLC.

### General Procedure 4

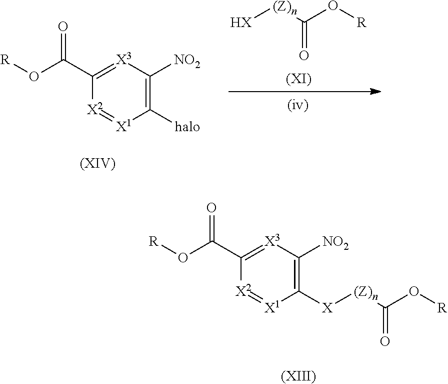

To a stirred solution of a compound of formula (XIV) (50.2 mmol, 1.0 eq.) and an appropriate nucleophile (XI) (1.25 eq.) in a suitable solvent, such as 1,4-dioxane, MeCN, DMF or THF, (3 mL/mmol) was added drop wise or portion wise a suitable base, such as TEA, DBU, NaH, K₂CO₃ (1.5 eq.) with ice bath cooling and allowed to stir at 0-25° C. for 1-16 h. The progress of the reaction was monitored by TLC or LCMS and on completion of the reaction; the reaction mixture was quenched with NH₄Cl solution and extracted with EtOAc. The combined organic layers were washed with brine, dried over anhydrous Na₂SO₄ and evaporated under reduced pressure to dryness. The crude compound of formula (XIII) (60-95% yield) obtained as a yellow solid which were pure enough to be used directly in the next step without further purification.

### General Procedure 5

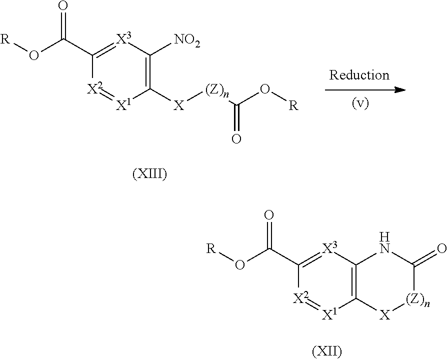

To a stirred solution of a compound of formula (XIII) (46.7 mmol, 1.0 eq.) in a suitable acid, such as AcOH or aq. HCl, (3 mL/mmol) was added iron powder or zinc powder (4.0 eq.) at RT. The reaction mixture was stirred at 75-85° C. for 2-5 h. The reaction was monitored by TLC or LCMS and on completion of the reaction, cooled to room temperature and poured into 1N HCl and then stirred for 1-2 h. The white precipitate was filtered off and washed with excess water. The residue obtained was re-dissolved in 5% MeOH in DCM and filtered through a bed of celite. The filtrate was evaporated under reduced pressure to obtain a compound of formula (XII) (80-95% yield) as a white solid which were pure enough to proceed in to the next step.

General procedure 6

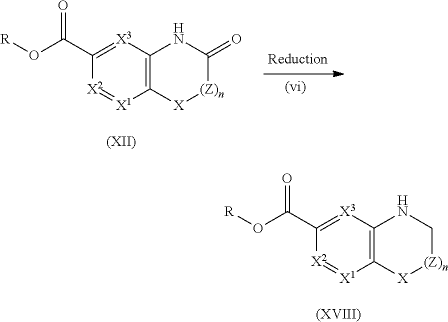

To a stirred solution of ester (XII) (2.24 mmol, 1.0 eq.) in a suitable solvent, such as THF or DMSO, was added borane-THF or borane-DMSO (5.0 eq.; 1M solution) at 0-5° C. The reaction mixture was allowed to stir at room temperature for 12-16 h. The progress of the reaction was monitored by TLC or LCMS and upon completion the reaction was quenched by drop wise addition of an appropriate amount of MeOH with ice cooling. The solvent was evaporated under reduced pressure. The residue obtained was partitioned between EtOAc and water; the organic layer was washed with brine, dried over anhydrous Na₂SO₄ and evaporated under reduced pressure to dryness to provide the crude products (XVIII) (85-96% yield) which were used without further purification.

### General Procedure 7

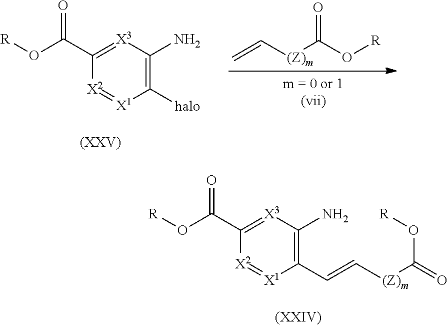

To a stirred solution of a compound of formula (XXV) (8.65 mmol, 1.0 eq.) in a suitable dry solvent, such as DMF, NMP or DMA, (1.5 mL/mmol) was added an olefin derivative (3.8 eq.) and a base, such as TEA or DIPEA, in a sealed tube reaction container at RT and the resulting reaction mixture degassed by bubbling N₂ gas through the solution for 5-10 min. A metal catalyst, such as bis(di-tert-butyl(4-dimethylaminophenyl)-phosphine)dichloropalladium (II), (0.03 eq.) was added and the solution was further degassed for 5-10 min. The tube was then sealed and heated at 130-140° C. for 10-16 h. After complete consumption of the starting material (monitored by TLC), the reaction mixture was filtered through a small pad of celite and then thoroughly washed with EtOAc. The filtrate was diluted with water and extracted with EtOAc. The organic layer was dried over anhydrous Na₂SO₄ and concentrated under reduced pressure to obtain the crude product, which was purified by silica gel column chromatography by using mixtures of EtOAc in hexanes as eluent to afford a compound of formula (XXIV) (20-50% yield).

### General Procedure 8

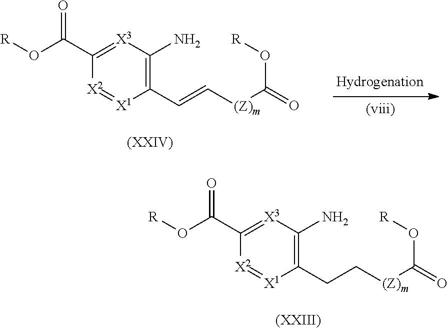

To a stirred solution of a compound of formula (XXIV) (2.32 mmol, 1.0 eq.) in a suitable solvent, for example MeOH or EtOAc, (5 mL/mmol) was added an appropriate amount of Pd—C (10% w/w) under a N₂ gas atmosphere at 0-25° C. The resulting reaction mixture was stirred at RT under H₂ gas balloon pressure for 2-4 h. On completion of the reaction (monitored by TLC), the reaction mixture was filtered through a celite bed under a N₂ gas atmosphere. The filtrate was concentrated under reduced pressure to obtain the crude product which was purified by column chromatography on silica gel using mixtures of EtOAc in hexanes as eluent to afford the reduced alkane derivative (XXIII) (80-85% yield).

### General Procedure 9

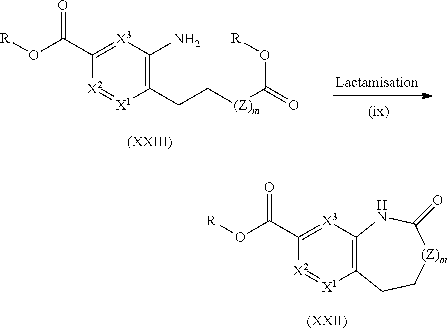

To a stirred solution of a compound of formula (XXIII) (1.9 mmol, 1.0 eq.) in a suitable solvent, such as toluene, DMF, DMA or MeCN, (5 mL/mmol) was added formic acid (1.5 mL/mmol) and the resulting reaction mixture was heated to reflux (100-115° C.) for 1-12 h. After completion of the reaction the mixture was concentrated under reduced pressure to obtain a residue which was diluted with water and extracted with EtOAc. The combined organic layers were washed with brine solution, dried over anhydrous Na₂SO₄ and concentrated under reduced pressure to afford the crude product which was purified by column chromatography to yield the cyclized compound of formula (XXII) in 60-70% yield.

### General Procedure 10

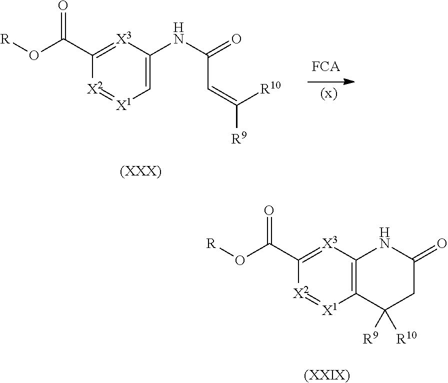

To a stirred solution of a compound of formula (XXX) (0.86 mmol, 1.0 eq.) in a suitable solvent such as EDC or MeCN under an inert atmosphere was added anhydrous AiCl₃ or FeCl₃ (4.0 eq.) portionwise and the whole heated at reflux temperature for 1-2 h. After completion of the reaction (TLC/LCMS), the reaction mixture was diluted with EDC and washed with dilute HCl followed by brine solution. The organic layer was dried over anhydrous Na₂SO₄ and evaporated under reduced pressure to dryness to obtain the crude product which was purified by column chromatography on silica gel using mixtures of EtOAc in hexanes as eluent to afford the desired compound of formula (XXIX) (50-55% yield) as a brown solid.

### General Procedure 11

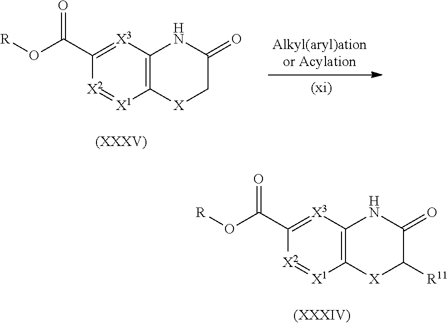

To a stirred solution of a compound of formula (XXXV) (26.16 mmol, 1.0 eq.) in DMF or THF (150 mL) at −65 to −78° C. was added dropwise a suitable base such as LiHMDS, LDA or NaH (1.0 eq.) and the mixture allowed to stir for 15-30 min. A suitable alkyl/aryl(het) halide (R⁹-L-G) (1.0 eq.) was added at the same temperature. The whole was allowed to stir at 0 to −10° C. for 0.5 to 1 h. The progress of the reaction was monitored by TLC. After completion of the reaction, the mixture was quenched with an aqueous solution of NH₄Cl, extracted with EtOAc, and the combined organic layers were washed with brine and dried over anhydrous Na₂SO₄. The dried organics were evaporated under reduced pressure to obtain a crude residue which was purified by Combi-flash using EtOAc in hexanes as eluent to afford a compound of formula (XXXIV) (25-30% yield) as a light orange to faint pink solid.

### General Procedure 12

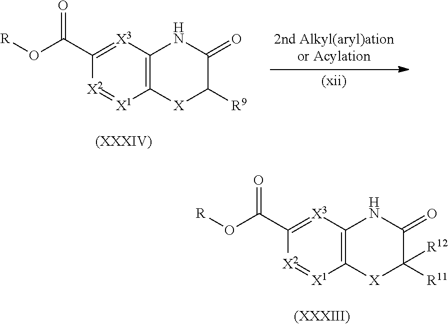

To a stirred solution of (XXXIV) (26.16 mmol, 1.0 eq.) in DMF or THF (150 mL) at −65 to −78° C. was added dropwise a suitable base, such as LiHMDS, LDA or NaH, (1.0 eq.) and the whole allowed to stir for 15-30 min., then a suitable alkyl/aryl(het) halide (R¹⁰-L-G) (1.0 eq.) was added at the same temperature. The whole was allowed to stir at 0 to −10° C. for 0.5 to 1 h. After completion of the reaction, the mixture was quenched with an aqueous solution of NH₄Cl, extracted with EtOAc, and the combined organic layers were washed with brine and dried over anhydrous Na₂SO₄. The dried organics were evaporated under reduced pressure to obtain a crude residue which was purified by Combi-flash using 35-50% EtOAc in hexanes as eluent to afford a compound of formula (XXXIII) (65-70% yield) as a light orange to faint pink solid.

### General Procedure 13

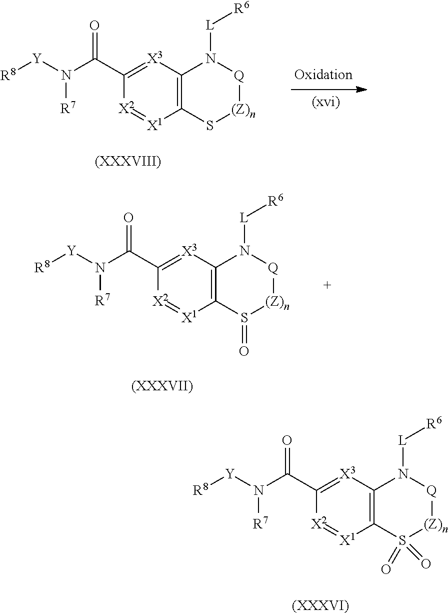

To a stirred solution of a compound of formula (XXXVIII) (1.0 eq.) in suitable solvents, such as DCM, CHCl₃, CCl₄ or EDC, (2 mL) was added mCPBA (1.0-3.0 eq.) at RT. The reaction mixture was further stirred at the same temperature for 2-16 h. The progress of the reaction was monitored by TLC and after completion of the reaction the mixture was diluted with more solvent and washed with a saturated solution of aqueous NaHCO₃ followed by brine. The organic layers were dried over anhydrous Na₂SO₄ and evaporated under reduced pressure. The crude product was purified by prep-HPLC to afford sulfoxide (XXXVII) and sulfone (XXXVI) as off white solids.

### EXAMPLES

Nuclear magnetic resonance (NMR) spectra were in all cases consistent with the proposed structures. Characteristic chemical shifts (6) are given in parts-per-million (ppm) downfield from tetramethylsilane (for ¹H-NMR) and upfield from trichloro-fluoro-methane (for ¹⁹F NMR) using conventional abbreviations for designation of major peaks: e.g. s, singlet; d, doublet; t, triplet; q, quartet; m, multiplet; br, broad. The following abbreviations have been used for common solvents: CDCl₃, deuterochloroform; d₆-DMSO, deuterodimethylsulphoxide; and CD₃OD, deuteromethanol.

Mass spectra, MS (m/z), were recorded using electrospray ionisation (ESI). Where relevant and unless otherwise stated the m/z data provided are for isotopes ¹⁹F, 35Cl, ⁷⁹Br and ¹²⁷I.

All chemicals, reagents and solvents were purchased from commercial sources and used without further purification. All reactions were performed under an atmosphere of nitrogen unless otherwise noted.

Flash column chromatography was carried out using pre-packed silica gel cartridges in a Combi-Flash platform. Prep-HPLC purification was carried out according to the General purification and analytical methods described above. Thin layer chromatography (TLC) was carried out on Merck silica gel 60 plates (5729). All final compounds were >95% pure as judged by the LCMS or UPLC analysis methods described in the General purification and analytical methods above unless otherwise stated.

### Example 1: 4-(2-chlorobenzyl)-N-(furan-2-ylmethyl)-3-oxo-3,4-dihydro-2H-benzo[b-1,4]thiazine-6-carboxamide

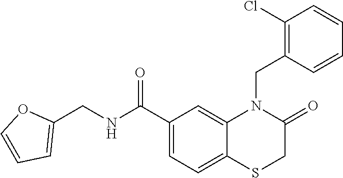 [SMILES: O=C(NCc1ccco1)c1ccc2c(c1)N(Cc1ccccc1Cl)C(=O)CS2]

Example 1 was prepared according to the methods described in General Procedures 1-3, and the methods described below.

### Preparation 1: Methyl 3-oxo-3,4-dihydro-2H-1,4-benzothiazine-6-carboxylate

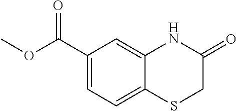

### Step 1: Methyl 4-((2-ethoxy-2-oxoethyl)thio)-3-nitrobenzoate

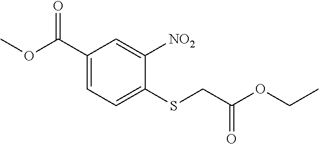 [SMILES: CCOC(=O)CSc1ccc(C(=O)OC)cc1[N+](=O)[O-]]

Methyl 4-fluoro-3-nitrobenzoate (10.0 g, 50.2 mmol) was taken up in MeCN (2.0 L) and TEA (7.61 g, 75.38 mmol) was added to it. The reaction mixture was cooled to 0° C. and ethyl thioglycolate (7.25 g, 62.7 mmol) was added drop wise. The reaction mixture was stirred for 30 min. at ice cold temperature. It was then diluted with EtOAc and washed with saturated solution of NH₄Cl and brine. The organic layer was dried over anhydrous Na₂SO₄ and evaporated under reduced pressure to dryness to give methyl 4-((2-ethoxy-2-oxoethyl)thio)-3-nitrobenzoate (14.0 g, 46.82 mmol, 93% yield) as a yellow solid, which was pure enough to be used in the next step without any further purification. LCMS m/z: 300.06 [M+H].

### Step 2: Methyl 3-oxo-3,4-dihydro-2H-benzo[b-1,4]thiazine-6-carboxylate

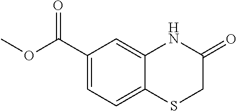

To a stirred solution of methyl 4-((2-ethoxy-2-oxoethyl)thio)-3-nitrobenzoate (Step 1) (5.0 g, 16.7 mmol) in acetic acid (50 mL) was added iron powder (3.73 g, 66.8 mmol). The resulting reaction mixture was stirred at 80° C. for 3 h. On completion (monitored by TLC), it was cooled to room temperature and poured onto 1N HCl (250 mL) and then stirred for 1 h. The white precipitate was filtered off and washed with water. The residue obtained was re-dissolved in 5% MeOH in DCM (50 mL) and filtered through a bed of celite. The filtrate was evaporated to dryness to afford the methyl 3-oxo-3,4-dihydro-2H-benzo[b-1,4]thiazine-6-carboxylate (3.5 g, 15.6 mmol, 91% yield) as a golden white solid. LCMS m/z: 222.05 [M−H].

### Preparation 4: Methyl 4-(2-chlorobenzyl)-3-oxo-3,4-dihydro-2H-benzo[b-1,4]thiazine-6 carboxylate

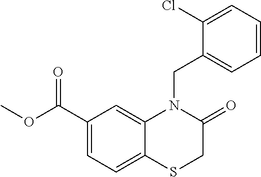 [SMILES: COC(=O)c1ccc2c(c1)N(Cc1ccccc1Cl)C(=O)CS2]

To a stirred solution of methyl 3-oxo-3,4-dihydro-2H-benzo[b-1,4]thiazine-6-carboxylate (Preparation 1) (0.3 g, 1.34 mmol) in EDC (5 mL) was added NaOH solution (0.107 g, 2.69 mmol in 5 mL H₂O) portion wise followed by TBAB (0.05 g, 0.15 mmol) at RT. The whole was stirred for 30 min at RT and then 2-chloro-benzyl bromide (0.209 mL, 1.61 mmol) was added drop wise. The reaction mixture was then stirred at room temperature for 2 h. The progress of the reaction was monitored by TLC and after completion; the reaction mixture was diluted with DCM, washed with water and brine solution. The organic layer was dried over anhydrous Na₂SO₄ and evaporated under reduced pressure to dryness to give 4-(2-chlorobenzyl)-3-oxo-3,4-dihydro-2H-benzo[b-1,4]thiazine-6-carboxylate (0.32 g, 0.92 mmol, 69% yield) which was used directly in Preparation 5. LCMS m/z: 348.19 [M+H].

### Preparation 5: 4-(2-Chlorobenzoyl)-3-oxo-3,4-dihydro-2H-benzo[b-1,4]thiazine-6-carboxylic acid

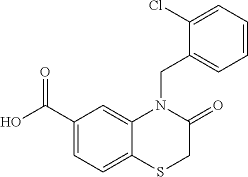 [SMILES: O=C(O)c1ccc2c(c1)N(Cc1ccccc1Cl)C(=O)CS2]

To a stirred solution of methyl 4-(2-chlorobenzyl)-3-oxo-3,4-dihydro-2H-benzo[b-1,4]thiazine-6-carboxylate (Preparation 4) (0.32 g, 0.92 mmol) in a mixture of MeOH, water and THF (1:1:2; 12 mL) was added LiOH.H₂O (0.077 g, 1.84 mmol) at RT. The reaction was allowed to stir at RT for 2 h. Upon completion of the reaction, the solvents were evaporated under reduced pressure and the residue was dissolved in water, washed with EtOAc and then acidified to ˜pH 5. The acidified aqueous solution was then extracted with EtOAc. The combined organic extracts were washed with brine, dried over anhydrous Na₂SO₄ and evaporated to dryness to give 4-(2-chlorobenzyl)-3-oxo-3,4-dihydro-2H-benzo[b-1,4]thiazine-6-carboxylic acid (0.22 g. 0.66 mmol, 72% yield) as white solid which was used in the next step without any further purification. LCMS m/z: 332.12 [M−H].

### Preparation 6: 4-(2-chlorobenzyl)-N-(furan-2-ylmethyl)-3-oxo-, 4-dihydro-2H-benzo[b-1,4]thiazine-6-carboxamide

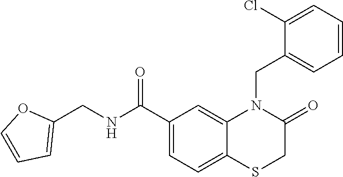 [SMILES: O=C(NCc1ccco1)c1ccc2c(c1)N(Cc1ccccc1Cl)C(=O)CS2]

To a stirred solution of 4-(2-chlorobenzyl)-3-oxo-3,4-dihydro-2H-benzo[b-1,4]thiazine-6-carboxylic acid (Preparation 5) (0.1 g, 0.3 mmol) in DMF (5 mL) was added HATU (0.136 g, 0.36 mmol) followed by TEA (0.104 mL, 1.03 mmol) at RT. The resulting reaction mixture was stirred at RT for 30 min. then 1-(furan-2-yl)-methanamine (0.026 mL, 0.27 mmol) was added and stirring continued at RT for 2 h. After this time, the reaction mixture was diluted with EtOAc and washed with chilled water and brine. The organic layer was dried over anhydrous Na₂SO₄ and evaporated under reduced pressure to dryness. The crude material obtained was purified by prep-HPLC to afford 4-(2-chlorobenzyl)-N-(furan-2-ylmethyl)-3-oxo-3,4-dihydro-2H-benzo[b-1,4]thiazine-6-carboxamide (Example 1) (0.04 g, 0.097 mmol, 32% yield) as a white solid. LCMS m/z: 413.18 [M+H]; ¹H NMR (500 MHz; DMSO-d₆): δ 3.76 (s, 2H), 4.41 (d, J=5.6 Hz, 2H), 5.24 (s, 2H), 6.20 (d, J=2.7 Hz, 1H), 6.37-6.38 (dd, J₁=1.85 Hz, J₂=2.9 Hz, 1H), 7.07 (d, J=7.55 Hz, 1H), 7.26-7.31 (m, 2H), 7.49-7.52 (m, 2H), 7.55-7.57 (m, 3H), 9.00 (t, J=5.65 Hz, 1H).

### Examples 2-49

Examples 2-49 were prepared according to the above methods used to make Example 1 as described in General Procedures 1-3 using the appropriate amines and acids.

Purification was as stated in the aforementioned methods.

| | | IUPAC | | LCMS |
|---|---|---|---|---|
| Example | Structure | Name | ¹H-NMR | [M + H] |
| 2 | 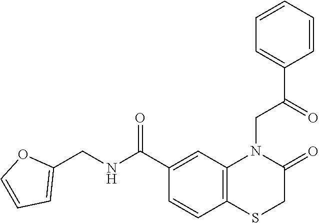 [SMILES: O=C(CN1C(=O)CSc2ccc(C(=O)NCc3ccco3)cc21)c1ccccc1] | N-(furan-2- ylmethyl)-3- oxo-4-(2-oxo- 2- phenylethyl)- 3,4-dihydro- 2H- benzo[b][1,4] thiazine-6- carboxamide | (500 MHz; DMSO-d₆): δ 3.36 (s, 2H), 4.42 (d, J = 5.55 Hz, 2H), 5.57 (s, 2H), 6.21 (d, J = 2.55 Hz, 1H), 6.36 (s, 1H), 7.40 (s, 1H), 7.54 (s, 1H), 7.57 (s, 2H), 7.63 (t, J = 7.65 Hz, 2H), 7.75 (t, J = 7.45 Hz, 1H), 8.13 (d, J = 7.75 Hz, 2H), 9.01 (t, J = 5.5 Hz, 1H). | 407.26 |
| 3 | 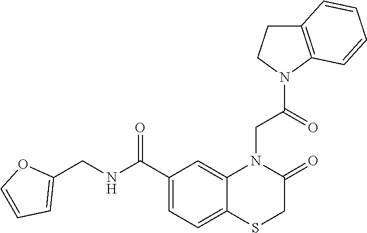 [SMILES: O=C(NCc1ccco1)c1ccc2c(c1)N(CC(=O)N1CCc3ccccc31)C(=O)CS2] | N-(furan-2- ylmethyl)-4- (2-(indolin-1- yl)-2- oxoethyl)-3- oxo-3,4- dihyrdo-2H- benzo[b- 1,4]thiazine- 6- carboxamide | (500 MHz; DMSO-d₆): 3.25 (m, 2H), 3.63 (s, 2H), 4.28 (t, J = 8.4 Hz, 2H), 4.44 (d, J = 5.55 Hz, 2H), 4.93 (s, 2H), 6.22 (d, J = 2.8 Hz, 1H), 6.34 (bs, 1H), 7.04 (t, J = 7.35 Hz, 1H), 7.17 (t, J = 7.5 Hz, 1H), 7.30 (d, J = 7.25 Hz, 1H), 7.52- 7.54 (m, 2H), 7.55-7.57 (m 2H), 8.01 (d, J = 8.05 Hz, 1H) 9.01-9.03 (m, 1H). | 448.20 |
| 4 | 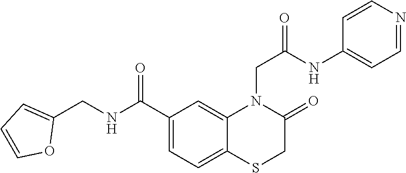 [SMILES: O=C(CN1C(=O)CSc2ccc(C(=O)NCc3ccco3)cc21)Nc1ccncc1] | N-(furan-2- ylmethyl)-3- oxo-4-(2-oxo- 2-(pyridin-4- ylamino)ethyl)- 3,4-dihydro- 2H-benzo[b- 1,4]-thiazine- 6- carboxamide | (500 MHz; DMSO-d₆): δ 3.65 (s, 2H), 4.50 (d, J = 5.5 Hz, 2H), 4.95 (s, 2H), 6.24 (d, J = 2.7 Hz, 1H), 6.37 (s, 1H), 7.55- 7.63 (m, 4H), 8.00 (d, J = 5.8 Hz, 2H), 8.68 (d, J = 5.85 Hz, 2H), 9.10 (t, J = 5.45 Hz, 1H), 11.84 (bs, 1H). | 423.19 |
| 5 | 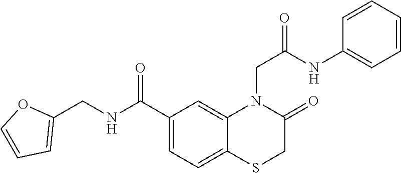 [SMILES: O=C(CN1C(=O)CSc2ccc(C(=O)NCc3ccco3)cc21)Nc1ccccc1] | N-(furan-2- ylmethyl)-3- oxo-4-(2-oxo- 2- (phenylamino) ethyl)-3,4- dihydro-2H- benzo[b- 1,4]thiazine- 6-carboxamide | (500 MHz; DMSO-d₆): δ 3.63 (s, 2H), 4.45 (d, J = 5.55 Hz, 2H), 4.79 (s, 2H), 6.24 (d, J = 2.5 Hz, 1H), 6.37 (s, 1H), 7.08 (t, J = 7.3 Hz, 1H), 7.33 (t, J = 7.75 Hz, 2H), 7.53-7.61 (m, 6H), 9.07 (t, J = 5.45 Hz, 1H), 10.34 (s, 1H). | 422.22 |
| 6 | 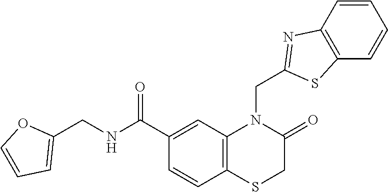 [SMILES: O=C(NCc1ccco1)c1ccc2c(c1)N(Cc1nc3ccccc3s1)C(=O)CS2] | 4- (benzo[d] thiazol-2- ylmethyl)-N- (furan-2- ylmethyl)-3- oxo-3,4- dihydro-2H- benzo[b- 1,4]thiazine- 6-carboxamide | (500 MHz; DMSO-d₆): δ 3.73 (s, 2H), 4.43 (d, J = 5.55 Hz, 2H), 5.68 (s, 2H), 6.23 (d, J = 2.55 Hz, 1H), 6.37 (s, 1H), 7.43 (t, J = 7.55 Hz, 1H), 7.51 (t, J = 7.4 Hz, 1H), 7.56-7.58 (m, 3H), 7.88 (s, 1H), 7.96 (d, J = 8.1 Hz, 1H), 8.07 (d, J = 8.0 Hz, 1H), 9.03 (t, J = 5.4 Hz, 1H). | 436.19 |
| 7 | 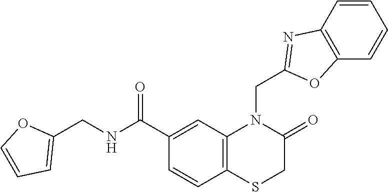 [SMILES: O=C(NCc1ccco1)c1ccc2c(c1)N(Cc1nc3ccccc3o1)C(=O)CS2] | 4-(benzo[d] oxazol-2- ylmethyl)-N- (furan-2- oxo-3,4- dihydro-2H- benzo[b- 1,4]thiazine- 6-carboxamide | (500 MHz; DMSO-d₆): δ 3.70 (s, 2H), 4.43 (d, J = 5.55 Hz, 2H), 5.52 (s, 2H), 6.21 (d, J = 2.45 Hz, 1H), 6.35 (s, 1H), 7.36-7.42 (m, 2H), 7.54 (s, 1H), 7.57-7.61 (m, 2H), 7.70-7.74 (m, 2H), 7.79 (s, 1H), 9.01 (t, J = 5.35 Hz, 1H). | 420.20 |
| 8 | 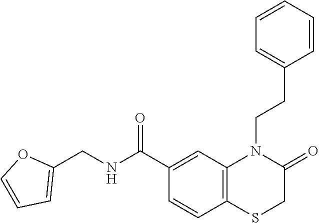 [SMILES: O=C(NCc1ccco1)c1ccc2c(c1)N(CCc1ccccc1)C(=O)CS2] | N-(furan-2- ylmethyl)-3- oxo-4- phenethyl- 3,4-dihydro- 2H-benzo[b- 1,4]thiazine- 6-carboxamide | (500 MHz; DMSO-d₆): δ 2.83-2.86 (m, 2H), 3.54 (s, 2H), 4.23-4.26 (m, 2H), 4.51 (d, J = 5.4 Hz, 2H), 6.32 (s, 1H), 6.43 (s, 1H), 7.19-7.20 (m, 3H), 7.26-7.29 (m, 2H), 7.51-7.62 (m, 3H), 7.79 (s, 1H), 9.09 (t, J = 5.2 Hz, 1H). | 393.24 |
| 9 | 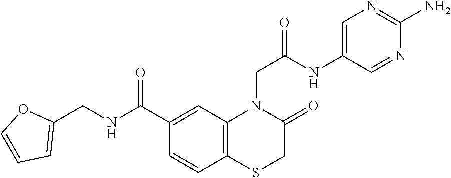 [SMILES: Nc1ncc(NC(=O)CN2C(=O)CSc3ccc(C(=O)NCc4ccco4)cc32)cn1] | 4-(2-((2- amino- pyrimidin-5- yl)amino)-2- oxoethyl)-N- (furan-2- ylmethyl)-3- oxo-3,4- dihydro-2H- benzo[b- 1,4]thiazine- | (500 MHz; DMSO-d₆): δ 3.62 (s, 2H), 4.46 (d, J = 5.55 Hz, 2H), 4.75 (s, 2H), 6.26 (d, J = 2.6 Hz, 1H), 6.38 (s, 1H), 6.57 (bs, 2H), 7.54 (d, J = 8.0 Hz, 1H), 7.57-7.60 (m, 3H), 8.38 (s, 2H), 9.07 (t, J = 5.5 Hz, 1H), 10.13 (s, 1H). | 439.21 |
| | | 6-carboxamide | | |
| 10 | 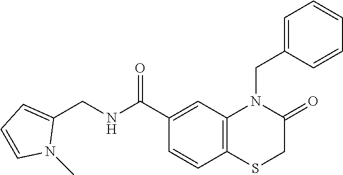 [SMILES: Cn1cccc1CNC(=O)c1ccc2c(c1)N(Cc1ccccc1)C(=O)CS2] | 4-benzyl-N- ((1-methyl- 1H-pyrrol-2- yl)methyl)-3- oxo-3,4- dihydro-2H- benzo[b- 1,4]thiazine- 6-carboxamide | (500 MHz; DMSO-d₆): δ 3.50 (s, 3H), 3.72 (s, 2H), 4.39 (d, J = 5.25 Hz, 2H), 5.28 (s, 2H), 5.88-5.91 (m, 2H), 6.66 (s, 1H), 7.19-7.24 (m, 3H), 7.31 (t, J = 7.45 Hz, 2H), 7.49-7.53 (m, 2H), 7.63 (s, 1H), 8.77 (t, J = 5.1 Hz, 1H). | 392.24 |
| 11 | 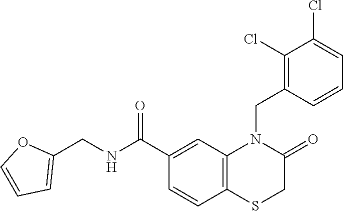 [SMILES: O=C(NCc1ccco1)c1ccc2c(c1)N(Cc1cccc(Cl)c1Cl)C(=O)CS2] | 4-(2,3- dichloro- benzyl)-N- (furan-2- ylmethyl)-3- oxo-3,4- dihydro-2H- benzo[b- 1,4]thiazine- 6-carboxamide | (500 MHz; DMSO-d₆): δ 3.77 (s, 2H), 4.41 (d, J = 5.6 Hz, 2H), 5.25 (s, 2H), 6.20 (d, J = 2.85 Hz, 1H), 6.37 (s, 1H), 7.07 (d, J = 7.8 Hz, 1H), 7.30 (t, J = 7.9 Hz, 1H), 7.45 (s, 1H), 7.53-7.60 (m, 4H), 9.02 (t, J = 5.55 Hz, 1H). | 447.14 |
| 12 | 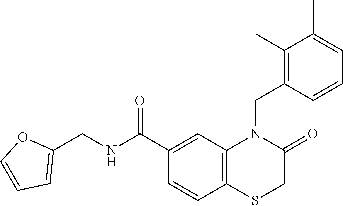 [SMILES: Cc1cccc(CN2C(=O)CSc3ccc(C(=O)NCc4ccco4)cc32)c1C] | 4-(2,3- dimethyl- benzyl)-N- (furan-2- ylmethyl)- 3-oxo-3,4- dihydro-2H- benzo[b- 1,4]thiazine- 6-caroxamide | (400 MHz; DMSO-d₆): δ 2.20 (s, 3H), 2.24 (s, 3H), 3.70 (s, 2H), 4.37 (d, J = 5.56 Hz, 2H), 5.16 (s, 2H), 6.16 (d, J = 2.96 Hz, 1H), 6.34-6.35 (m, 1H), 6.73 (d, J = 7.6 Hz, 1H), 6.92 (t, J = 7.64 Hz, 1H), 7.01 (d, J = 7.36 Hz, 1H), 7.47 (s, 1H), 7.51-7.52 (m, 3H), 8.94 (t, J = 5.44 Hz, 1H). | 407.25 |
| 13 | 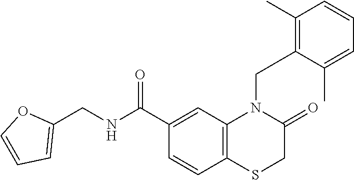 [SMILES: Cc1cccc(C)c1CN1C(=O)CSc2ccc(C(=O)NCc3ccco3)cc21] | 4-(2,6- dimethyl- benzyl)-N- (furan-2- ylmethyl)- 3-oxo-3,4- dihydro-2H- benzo[b- 1,4]thiazine- 6-carboxamide | (400 MHz; DMSO-d₆): δ 2.27 (s, 6H), 3.56 (s, 2H), 4.41 (d, Hz, 2H), 5.25 (s, 2H), 6.21 (d, J = 2.84 Hz, 1H), 6.38-6.39 (m, 1H), 6.84 (d, J = 7.4 Hz, 2H), 6.89-6.93 (m, 1H), 7.41- 7.46 (m, 2H), 7.58 (d, J = 12.52 Hz, 2H), 8.87 (t, J = 5.40 Hz, 1H). | 407.27 |
| 14 | 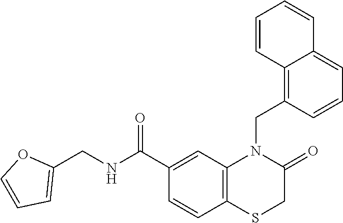 [SMILES: O=C(NCc1ccco1)c1ccc2c(c1)N(Cc1cccc3ccccc13)C(=O)CS2] | N-(furan-2- ylmethyl)-4- (naphthalen- 1-ylmethyl)-3- oxo-3,4- dihydro-2H- benzo[b- 1,4]thiazine- 6-carboxamide | (400 MHz; DMSO-d₆): δ 3.78 (s, 2H), 4.31 (d, J = 5.6 Hz, 2H), 5.67 (s, 2H), 6.09 (d, J = 3.04 Hz, 1H), 6.29-6.31 (m, 1H), 7.14 (d, J = 7.08 Hz, 1H), 7.36 (d, J = 7.48 Hz, 1H), 7.48 (bs, 1H), 7.50-7.55 (m, 3H), 7.57-7.61 (m, 2H), 7.81 (d, J = 8.16 Hz, 1H), 8.95-8.97 (m, 1H), 8.16 (d, J = 7.96 Hz, 1H), 8.91 (t, J = 5.64 Hz, 1H). | 429.26 |
| 15 | 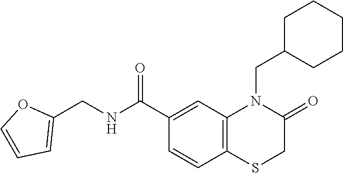 [SMILES: O=C(NCc1ccco1)c1ccc2c(c1)N(CC1CCCCC1)C(=O)CS2] | 4- (cyclohexyl- methyl)-N- (furan-2- ylmethyl)-3- oxo-3,4- dihydro-2H- benzo[b- 1,4]thiazine- 6-carboxamide | (400 MHz; DMSO-d₆): δ 0.88-0.90 (m, 2H), 1.04 (bs, 3H), 1.50-1.59 (m, 6H), 3.50 (s, 2H), 3.94 (s, 2H), 4.46 (s, 2H), 6.25 (s, 1H), 6.38 (s, 1H), 7.48-7.56 (m, 3H), 7.78 (s, 1H), 9.02 (bs, 1H). | 385.26 |
| 16 | 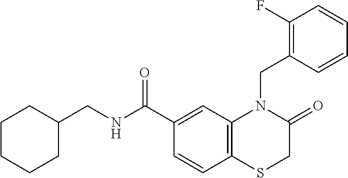 [SMILES: O=C(NCC1CCCCC1)c1ccc2c(c1)N(Cc1ccccc1F)C(=O)CS2] | N- (cyclo- hexylmethyl)-4- (2-fluoro- benzyl)- 3-oxo-3,4- dihydro-2H- benzo[b- 1,4]thiazine- 6-carboxamide | (500 MHz; DMSO-d₆): δ 0.82-0.88 (m, 2H), 1.09-1.18 (m, 3H), 1.47 (bs, 1H), 1.61-1.64 (m, 5H), 3.04 (t, J = 6.15 Hz, 2H), 3.73 (s, 2H), 5.29 (s, 2H), 7.10 (d, J = 5.75 Hz, 2H), 7.22 (t, J = 9.9 Hz, 1H), 7.30-7.31 (m, 1H), 7.51-7.54 (m, 3H), 8.47 (t, J = 5.4 Hz, 1H). | 413.33 |
| 17 | 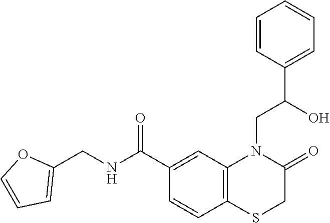 [SMILES: O=C(NCc1ccco1)c1ccc2c(c1)N(CC(O)c1ccccc1)C(=O)CS2] | N-(furan-2- ylmethyl)-4- (2-hydroxy-2- phenylethyl)- 3-oxo-3,4- dihydro-2H- benzo[b- 1,4]thiazine- 6-carboxamide | 500 MHz; DMSO-d₆): δ 3.48-3.56 (q, J = 14.4 Hz, 2H), 4.06-4.10 (m, 1H), 4.18-4.21 (m, 1H), 4.45-4.54 (m, 2H), 4.83- 4.86 (m, 1H), 5.61 (d, J = 4.2 Hz, 1H), 6.32 (d, J = 2.75 Hz, 1H), 6.43 (s, 1H), 7.21-7.24 (m, 1H), 7.28-7.35 (m, 4H), 7.46 (d, J = 8.05 Hz, 1H), 7.51 (d, J = 8.0 Hz, 1H), 7.62 (s, 1H), 8.04 (s, 1H), 9.03 (t, J = 5.5 Hz, 1H). | 409.25 |
| 18 | 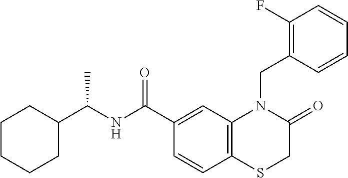 [SMILES: C[C@H](NC(=O)c1ccc2c(c1)N(Cc1ccccc1F)C(=O)CS2)C1CCCCC1] | (S)-N-(1- cyclohexyl- ethyl)-4-(2- fluorobenzyl)- 3-oxo-3,4- dihydro-2H- benzo[b- 1,4]thiazine- 6-carboxamide | (500 MHz; DMSO-d₆): δ 0.81-0.92 (m, 2H), 1.05- 1.14 (m, 6H), 1.33-1.35 (m, 1H), 1.58-1.69 (m, 5H), 3.69-3.77 (m, 3H), 5.24 (d, J = 16.75 Hz, 1H), 5.35 (d, J = 16.85 Hz, 1H), 7.10 (d, J = 4.7 Hz, 2H), 7.21 (t, J = 10.0 Hz, 1H), 7.30-7.32 (m, 1H), 7.52 (s, 3H), 8.12 | 427.32 |
| | | | (d, J = 8.7 Hz, 1H). | |
| 19 | 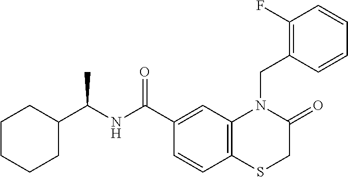 [SMILES: C[C@@H](NC(=O)c1ccc2c(c1)N(Cc1ccccc1F)C(=O)CS2)C1CCCCC1] | (R)-N-(1- cyclohexyl- ethyl)-4-(2- flurobenzyl)- 3-oxo-3,4- dihydro-2H- benzo[b- 1,4]thiazine- 6-carboxamide | (500 MHz; DMSO-d₆): δ 0.83-0.92 (m, 2H), 1.05-1.16 (m, 6H), 1.33- 1.35 (m, 1H), 1.58-1.69 (m, 5H), 3.69-3.77 (m, 3H), 5.24 (d, J = 16.9 Hz, 1H), 5.35 (d, J = 16.75 Hz, 1H), 7.10 (d, J = 4.65 Hz, 2H), 7.22 (t, J = 9.7 Hz, 1H), 7.30- 7.31 (m, 1H), 7.52 (s, | 427.33 |
| | | | 3H), 8.12 (d, J = 8.6 Hz, 1H). | |
| 20 | 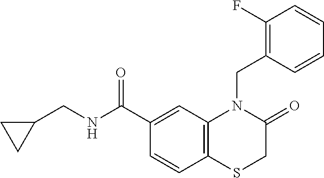 [SMILES: O=C(NCC1CC1)c1ccc2c(c1)N(Cc1ccccc1F)C(=O)CS2] | N- (cyclopropyl- methyl)-4-(2- fluorobenzyl)- 3-oxo-3,4- dihydro-2H- benzo[b- 1,4]thiazine- 6-carboxamide | (500 MHz; DMSO-d₆): δ 0.18 (d, J = 4.55 Hz, 2H), 0.40 (d, J = 7.8 Hz, 2H), 0.95-1.10 (m, 1H), 3.08-3.10 (m, 2H), 3.73 (s, 2H), 5.28 (s, 2H), 7.08-7.13 (m, 2H), 7.23 (t, J = 9.85 Hz, 1H), 7.29-7.32 (m, 1H), 7.54 (s, 2H), 7.58 (s, 1H), 8.60 (t, J = 5.15 Hz, 1H). | 371.22 |
| 21 | 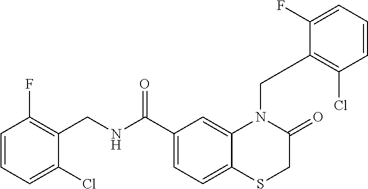 [SMILES: O=C(NCc1c(F)cccc1Cl)c1ccc2c(c1)N(Cc1c(F)cccc1Cl)C(=O)CS2] | N,4-bis(2- chloro-6- fluorobenzyl)- 3-oxo-3,4- dihydro-2H- benzo[b- 1,4]thiazine- carboxamide | (500 MHz; DMSO-d₆): δ 3.58 (s, 2H), 4.47 (d, J = 4.5 Hz, 2H), 5.37 (s, 2H), 7.11 (t, J = 9.4 Hz, 1H), 7.22-7.31 (m, 3H), 7.35 (d, J = 7.9 Hz, 1H), 7.38-7.41 (m, 1H), 7.45 (s, 2H), 7.74 (s, 1H), 8.72 (t, J = 4.75 Hz, 1H). | 493.20 |
| 22 | 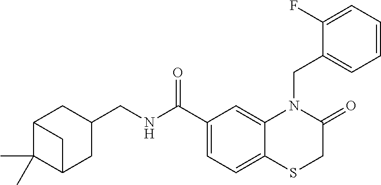 [SMILES: CC1(C)C2CC(CNC(=O)c3ccc4c(c3)N(Cc3ccccc3F)C(=O)CS4)CC1C2] | N-((6,6- dimethyl- bicyclo[3.1.1] heptan-3- yl)methyl)-4- (2- fluorobenzyl)- 3-oxo-3,4- dihydro-2H- benzo[b- 1,4]thiazine 6-carboxamide | (500 MHz; DMSO-d₆): δ 0.81-0.83 (m, 1H), 1.03 (s, 3H), 1.14 (s, 3H), 1.42-1.48 (m, 1H), 1.74- 1.90 (m, 5H), 2.21-2.24 (m, 1H), 2.28-2.31 (m, 1H), 3.20 (t, J = 6.95 Hz, 2H), 3.73 (s, 2H), 5.25-5.32 (m, 2H), 7.08- 7.12 (m, 2H), 7.22 (t, J = 10.15 Hz, 1H), 7.29-7.32 (m, 1H), 7.48-7.54 (m, | 453.31 |
| | | | 3H), 8.47 (t, J = 5.4 Hz, | |
| | | | 1H). | |
| 23 | 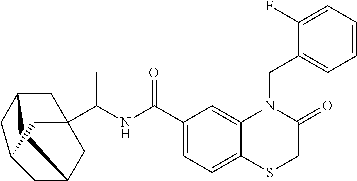 [SMILES: CC(NC(=O)c1ccc2c(c1)N(Cc1ccccc1F)C(=O)CS2)C12C[C@H]3C[C@@H](C1)C[C@@H](C2)C3] | N-(1- ((3r,5r,7r)- adamantan-1- yl)ethyl)-4-(2- fluorobenzyl)- 3-oxo-3,4- dihydro-2H- benzo[b- 1,4]thiazine- 6-carboxamide | (500 MHz; DMSO-d₆): δ 1.00 (d, J = 6.9 Hz, 3H), 1.38-1.52 (m, 9H), 1.61- 1.63 (s, 3H), 1.88 (bs, 3H), 3.68-3.75 (m, 2H), 3.77-3.80 (m, 1H), 5.23 (d, J = 17.0 Hz, 1H), 5.40 (d, J = 16.9 Hz, 1H), 7.10-7.11 (m, 2H), 7.20 (t, J = 9.8 Hz, 1H), 7.29-7.30 (m, 1H), 7.48- | 479.35 |
| | | | 7.53 (m, 3H), 7.93 (d, J = | |
| | | | 9.25 Hz, 1H), 8.47 (t, J = | |
| | | | 5.4 Hz, 1H). | |
| 24 | 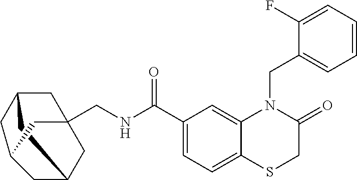 [SMILES: O=C(NCC12C[C@H]3C[C@@H](C1)C[C@@H](C2)C3)c1ccc2c(c1)N(Cc1ccccc1F)C(=O)CS2] | N-((3r,5r,7r)- adamantan-1- ylmethyl)-4- (2- fluorobenzyl)- 3-oxo-3,4- dihydro-2H- benzo[b- 1,4]thiazine- 6-carboxamide | (500 MHz; DMSO-d₆): δ 1.39 (s, 6H), 1.53 (d, J = 11.7 Hz, 3H), 1.63 (d, J = 11.85 Hz, 3H), 1.88 (bs, 3H), 2.92 (d, J = 6.25 Hz, 2H), 3.74 (s, 2H), 5.30 (s, 2H), 7.10-7.11 (m, 2H), 7.21 (t, J = 9.8 Hz, 1H), 7.29-7.30 (m, 1H), 7.49-7.54 (m, 3H), 8.31 (bs, 1H). | 465.34 |
| 25 | 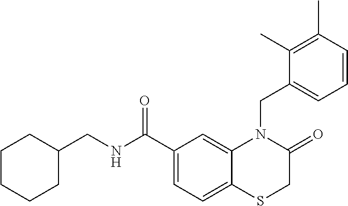 [SMILES: Cc1cccc(CN2C(=O)CSc3ccc(C(=O)NCC4CCCCC4)cc32)c1C] | N- (cyclohexyl- methyl)-4-(2,3- dimethyl- benzyl)- 3-oxo-3,4- dihydro-2H- benzo[b- 1,4]thiazine- 6-carboxamide | 500 MHz; DMSO-d₆): δ 0.81-0.88 (m, 2H), 1.08-1.15 (m, 3H), 1.45 (s, 1H), 1.60-1.63 (m, 5H), 2.25 (d, J = 13.15 Hz, 6H), 3.02 (t, J = 6.2 Hz, 2H), 3.74 (s, 2H), 5.18 (s, 2H), 6.76 (d, J = 7.7 Hz, 1H), 6.95 (t, J = 7.6 Hz, 1H), 7.01 (d, J = 7.35 Hz, 1H), 7.43 (s, 1H), 7.47-7.53 (m, 2H), 8.43 (t, J = 5.7 Hz, 1H). | 423.31 |
| 26 | 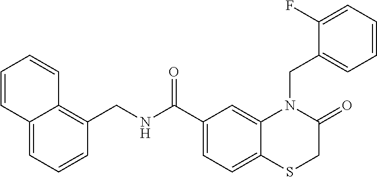 [SMILES: O=C(NCc1cccc2ccccc12)c1ccc2c(c1)N(Cc1ccccc1F)C(=O)CS2] | 4-(2- fluorobenzyl)- N- (naphthalen- 1-ylmethyl)-3- oxo-3,4- dihydro-2H- benzo[b- 1,4]thiazine- 6-carboxamide | 500 MHz; DMSO-d₆): δ 3.74 (s, 2H), 4.91 (d, J = 5.45 Hz, 2H), 5.29 (s, 2H), 7.09-7.10 (m, 2H), 7.22 (t, J = 10.0 Hz, 1H), 7.30-7.31 (m, 1H), 7.40 (d, J = 6.95 Hz, 1H), 7.46 (t, J = 7.30 Hz, 1H), 7.53-7.60 (m, 4H), 7.66 (s, 1H), 7.85 (d, J = 8.1 Hz, 1H), 7.95-7.97 (m, | 456.98 |
| | | | 1H), 8.11-8.12 (m, 1H), | |
| | | | 9.12 (t, J = 5.25 Hz, 1H). | |
| 27 | 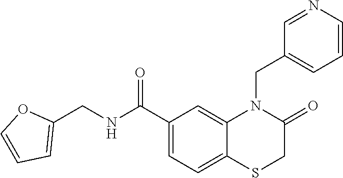 [SMILES: O=C(NCc1ccco1)c1ccc2c(c1)N(Cc1cccnc1)C(=O)CS2] | N-(furan-2- ylmethyl)-3- oxo-4- (pyridin-3- ylmethyl)-3,4- dihydro-2H- benzo[b- 1,4]thiazine- 6-carboxamide | (500 MHz; DMSO-d₆): δ 3.74 (s, 2H), 4.43 (d, J = 5.55 Hz, 2H), 5.32 (s, 2H), 6.22 (d, J = 2.8 Hz, 1H), 6.39 (m, 1H), 7.34- 7.36 (m, 1H), 7.53-7.59 (m, 4H), 7.64 (s 1H), 8.45-8.47 (m, 2H), 9.02 (t, J = 5.5 Hz, 1H). | 379.93 |
| 28 | 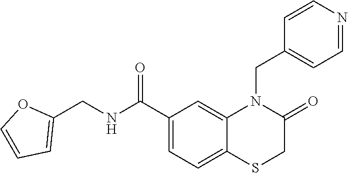 [SMILES: O=C(NCc1ccco1)c1ccc2c(c1)N(Cc1ccncc1)C(=O)CS2] | N-(furan-2- ylmethyl)-3- oxo-4- (pyridin-4- ylmethyl)-3,4- dihydro-2H- benzo[b- 1,4]thiazine- 6-carboxamide | (500 MHz; DMSO-d₆): δ 3.76 (s, 2H), 4.41 (d, J = 5.40 Hz, 2H), 5.29 (s, 2H), 6.21 (d, J = 1.95 Hz, 1H), 6.38 (s, 1H), 7.22 (d, J = 4.75 Hz, 2H), 7.51-7.57 (m, 4H), 8.50 (d, J = 4.8 Hz, 2H), 9.01 (t, J = 5.2 Hz, 1H). | 379.92 |
| 29 | 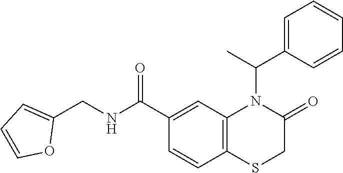 [SMILES: CC(c1ccccc1)N1C(=O)CSc2ccc(C(=O)NCc3ccco3)cc21] | N-(furan-2- ylmethyl)-3- oxo-4-(1- phenylethyl)- 3,4-dihydro- 2H-benzo[b- 1,4]thiazine- 6-carboxamide | (500 MHz; DMSO-d₆): δ 1.73 (d, J = 7.0 Hz, 3H), 3.55 (d, J = 14.5 Hz, 1H), 3.66 (d, J = 14.45 Hz, 1H), 4.39-4.40 (m, 2H), 5.98-6.02 (m, 1H), 6.21 (d, J = 2.9 Hz, 1H), 6.39 (s, 1H), 7.23-7.35 (m, 5H), 7.48 (s, 1H), 7.52-7.57 (m, 3H), 8.98 (t, J = 5.5 Hz, 1H). | 393.26 |
| 30 | 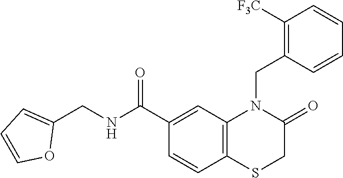 [SMILES: O=C(NCc1ccco1)c1ccc2c(c1)N(Cc1ccccc1C(F)(F)F)C(=O)CS2] | N-(furan-2- ylmethyl)-3- oxo-4-(2- (trifluoro- methyl)benzyl)- 3,4-dihydro- 2H-benzo[b- 1,4]thiazine- 6-carboxamide | (500 MHz; DMSO-d₆): δ 3.80 (s, 2H), 4.39 (d, J = 5.6 Hz, 2H), 5.34 (s, 2H), 6.20 (d, J = 2.95 Hz, 1H), 6.36-6.37 (m, 1H), 7.20 (d, J = 7.8 Hz, 1H), 7.45-7.61 (m, 6H), 7.81 (d, J = 7.7 Hz, 1H), 8.01 (t, J = 5.65 Hz, 1H). | 446.97 |
| 31 | 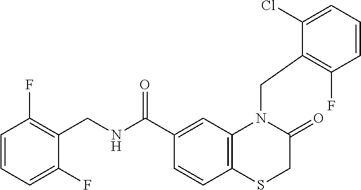 [SMILES: O=C(NCc1c(F)cccc1F)c1ccc2c(c1)N(Cc1c(F)cccc1Cl)C(=O)CS2] | 4-(2-chloro-6- fluorobenzyl)- N-(2,6- difluorobenzyl)- 3-oxo-3,4- dihydro-2H- benzo[b- 1,4]thiazine 6-carboxamide | (500 MHz; DMSO-d₆): δ 3.58 (s, 2H), 4.50 (d, J = 5.05 Hz, 2H), 5.36 (s, 2H), 7.08-7.13 (m, 3H), 7.22-7.23 (m, 1H), 7.26- 7.31 (m, 1H), 7.38-7.47 (m, 3H), 7.74 (s, 1H), 8.84 (t, J = 5.5 Hz, 1H). | 477.25 |
| 32 | 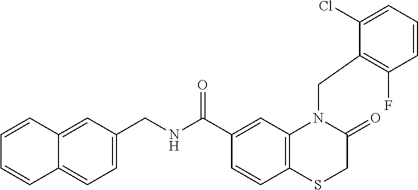 [SMILES: O=C(NCc1ccc2ccccc2c1)c1ccc2c(c1)N(Cc1c(F)cccc1Cl)C(=O)CS2] | 4-(2-chloro-6- fluorobenzyl)- N- (naphthalen- 2-ylmethyl)- 3-oxo-3,4- dihydro-2H- benzo[b- 1,4]thiazine 6-carboxamide | (500 MHz; DMSO-d₆): δ 3.60 (s, 2H), 4.64 (d, J = 5.8 Hz, 2H), 5.40 (s, 2H), 7.11 (t, J = 9.45 Hz, 1H), 7.24 (d, J = 7.85 Hz, 1H), 7.27-7.31 (m, 1H), 7.46-7.51 (m, 5H), 7.79 (s, 1H), 7.83 (s, 1H), 7.87-7.92 (m, 3H), 9.14 (t, J = 5.8 Hz, 1H). | 491.27 |
| 33 | 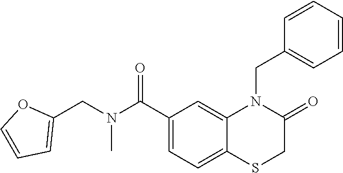 [SMILES: CN(Cc1ccco1)C(=O)c1ccc2c(c1)N(Cc1ccccc1)C(=O)CS2] | 4-benzyl-N- (furan-2- ylmethyl)-N- methyl-3-oxo- 3,4-dihydro- 2H- benzo[b][1,4] thiazine-6- carboxamide | (400 MHz; DMSO-d₆): δ 2.59-2.80 (m, 3H), 3.72 (s, 2H), 4.20 (bs, 1H), 4.56 (bs, 1H), 5.25 (s, 2H), 6.21-6.30 (m, 1H), 6.41 (bs, 1H), 7.03- 7.21 (m, 5H), 7.26-7.29 (m, 2H), 7.48 (d, J = 7.8 Hz, 1H), 7.55-7.59 (m, 1H). | 393.2 |
| 34 | 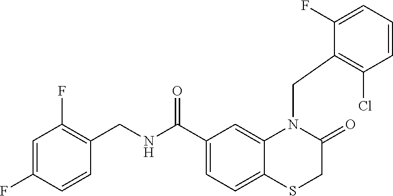 [SMILES: O=C(NCc1ccc(F)cc1F)c1ccc2c(c1)N(Cc1c(F)cccc1Cl)C(=O)CS2] | 4-(2-chloro-6- fluorobenzyl)- N-(2,4- difluorobenzyl)- 3-oxo-3,4- dihydro-2H- benzo[b- 1,4]thiazine- 6-carboxamide | (500 MHz; DMSO-d₆): δ 3.60 (s, 2H), 4.46 (d, J = 5.5 Hz, 2H), 5.39 (s, 2H), 7.08-7.14 (m, 2H), 7.24-7.32 (m, 3H), 7.35- 7.40 (m, 1H), 7.49 (s, 2H), 7.78 (s, 1H), 9.00 (t, J = 5.65 Hz, 1H). | 477.35 |
| 35 | 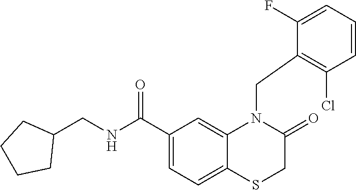 [SMILES: O=C(NCC1CCCC1)c1ccc2c(c1)N(Cc1c(F)cccc1Cl)C(=O)CS2] | 4-(2-chloro-6- fluorobenzyl)- N- (cyclopentyl- methyl)-3-oxo- 3,4-dihydro- 2H- benzo[b][1,4] thiazine-6- carboxamide | (500 MHz; DMSO-d₆): δ 1.20-126 (m, 2H), 1.49- 1.53 (m, 2H), 1.57-1.58 (m, 2H), 1.61-1.68 (m, 2H), 2.09-2.14 (m, 1H), 3.16 (t, J = 6.2 Hz, 2H), 3.59 (s, 2H), 5.39 (s, 2H), 7.11 (t, J = 9.75 Hz, 1H), 7.23-7.31 (m, 2H), 7.43-7.48 (m, 2H), 7.31 (s, 1H), 8.44 (t, J = 5.45 Hz, 1H). | 433.28 |
| 36 | 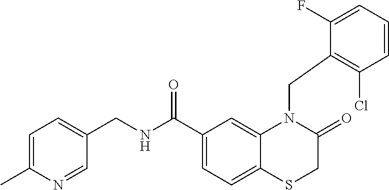 [SMILES: Cc1ccc(CNC(=O)c2ccc3c(c2)N(Cc2c(F)cccc2Cl)C(=O)CS3)cn1] | 4-(2-chloro-6- fluorobenzyl)- N-((6- methyl- pyridin- 3-yl)methyl)- 3-oxo-3,4- dihydro-2H- benzo[b- 1,4]thiazine- 6-carboxamide | (500 MHz; DMSO-d₆): δ 2.45 (s, 3H), 3.59 (s, 2H), 4.43 (d, J = 5.7 Hz, 2H), 5.38 (s, 2H), 7.12 (t, J = 9.45 Hz, 1H), 7.22-7.25 (m, 2H), 7.27- 7.31 (m, 1H), 7.46-7.50 (m, 2H), 7.57 (d, J = 7.95 Hz, 1H), 7.77 (s, 1H), 8.39 (bs, 1H), 9.03 (t, J = 5.65 Hz, 1H). | 456.30 |
| 37 | 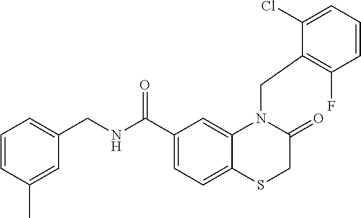 [SMILES: Cc1cccc(CNC(=O)c2ccc3c(c2)N(Cc2c(F)cccc2Cl)C(=O)CS3)c1] | 4-(2-chloro-6- fluorobenzyl)- N-(3- methylbenzyl)- 3-oxo-3,4- dihydro-2H- benzo[b- 1,4]thiazine- 6-carboxamide | (500 MHz; DMSO-d₆): δ 2.30 (s, 3H), 3.60 (s, 2H), 4.43 (d, J = 5.85 Hz, 2H), 5.39 (s, 2H), 7.06-7.13 (m, 4H), 7.21- 7.32 (m, 3H), 7.48-7.52 (m, 2H), 7.81 (s, 1H), 9.00 (t, J = 5.85 Hz, 1H). | 455.28 |
| 38 | 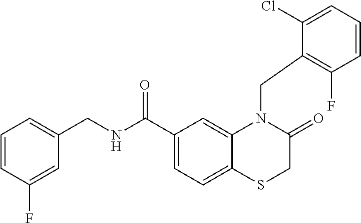 [SMILES: O=C(NCc1cccc(F)c1)c1ccc2c(c1)N(Cc1c(F)cccc1Cl)C(=O)CS2] | 4-(2-chloro-6- fluorobenzyl)- N-(3- fluorobenzyl)- 3-oxo-3,4- dihydro-2H- benzo[b- 1,4]thiazine- 6-carboxamide | (500 MHz; DMSO-d₆): δ 3.60 (s, 2H), 4.48 (d, J = 5.75 Hz, 2H), 5.40 (s, 2H), 7.08-7.14 (m, 4H), 7.24 (d, J = 8.0 Hz, 1H), 7.27-7.32 (m, 1H), 7.37- 7.42 (m, 1H), 7.501 (s, 2H), 7.80 (s, 1H), 9.08 (t, J = 5.80 Hz, 1H). | 459.22 |
| 39 | 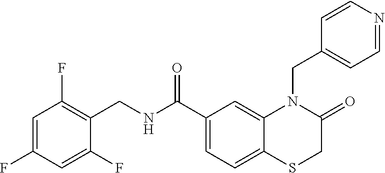 [SMILES: O=C(NCc1c(F)cc(F)cc1F)c1ccc2c(c1)N(Cc1ccncc1)C(=O)CS2] | 3-oxo-4- (pyridin-4- ylmethyl)-N- (2,4,6- trifluoro- benzyl)-3,4- dihydro-2H- benzo[b- 1,4]thiazine- 6-carboxamide | (500 MHz; DMSO-d₆): δ 3.75 (s, 2H), 4.40 (d, J = 3.85 Hz, 2H), 5.28 (s, 2H), 7.16-7.20 (m, 4H), 7.47-7.55 (m, 3H), 8.50 (bs, 2H), 8.93 (bs, 1H). | 444.28 |
| 40 | 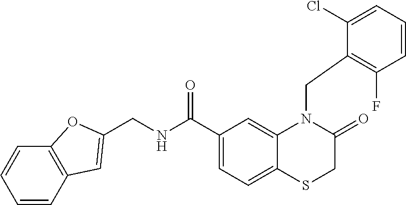 [SMILES: O=C(NCc1cc2ccccc2o1)c1ccc2c(c1)N(Cc1c(F)cccc1Cl)C(=O)CS2] | N- (benzofuran- 2-ylmethyl)- 4-(2-chloro-6- fluorobenzyl)- 3-oxo-3,4- dihydro-2H- benzo[b- 1,4]thiazine- 6-carboxamide | (500 MHz; DMSO-d₆): δ 3.59 (s, 2H), 4.63 (d, J = 5.5 Hz, 2H), 5.39 (s, 2H), 6.74 (s, 1H), 7.10 (t, J = 9.55 Hz, 1H), 6.21-7.31 (m, 4H), 7.49- 7.55 (m, 3H), 7.60 (d, J = 7.5 Hz, 1H), 7.81 (s, 1H), 9.13 (t, J = 5.5 Hz, 1H). | 481.25 |
| 41 | 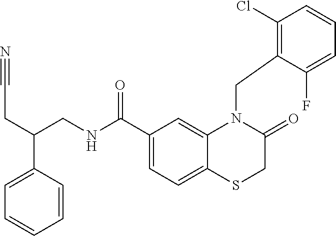 [SMILES: N#CCC(CNC(=O)c1ccc2c(c1)N(Cc1c(F)cccc1Cl)C(=O)CS2)c1ccccc1] | 4-(2-chloro-6- fluorobenzyl)- N-(3-cyano-2- phenylpropyl)- 3-oxo-3,4- dihydro-2H- benzo[b- 1,4]thiazine- 6-carboxamide | (400 MHz; DMSO-d₆): δ 2.89-2.92 (m, 2H), 3.26-3.28 (m, 1H), 3.49- 3.52 (m, 2H), 3.57 (s, 2H), 5.36 (d, J = 4.12 Hz, 2H), 7.08-7.13 (m, 1H), 7.22-7.38 (m, 8H), 7.45 (d, J = 8.08 Hz, 1H), 7.68 (s, 1H), 8.52- 8.53 (m, 1H). | 494.1 |
| 42 | 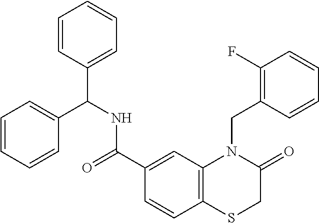 [SMILES: O=C(NC(c1ccccc1)c1ccccc1)c1ccc2c(c1)N(Cc1ccccc1F)C(=O)CS2] | N- benzhydryl-4- (2- fluorobenzyl)- 3-oxo-3,4- dihydro-2H- benzo[b][1,4] thiazine-6- carboxamide | (500 MHz; DMSO-d₆): δ 1.45-1.53 (m, 3H), 1.64- 1.66 (m, 2H), 1.81-1.85 (m, 2H), 3.73-3.75 (m, 2H), 4.13-4.17 (m, 1H), 5.28-5.32 (m, 2H), 7.07- 7.13 (m, 2H), 7.21-7.35 (m, 5H), 7.5.-7.62 (m, 3H), 8.28-8.30 (m, 1H). | 483.26 |
| 43 | 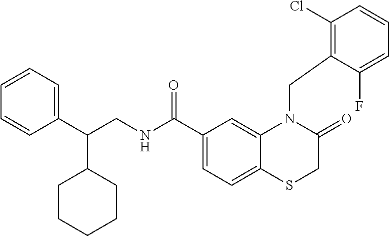 [SMILES: O=C(NCC(c1ccccc1)C1CCCCC1)c1ccc2c(c1)N(Cc1c(F)cccc1Cl)C(=O)CS2] | 4-(2-chloro-6- fluorobenzyl)- N-(2- cyclohexyl-2- phenylethyl)- 3-oxo-3,4- dihydro-2H- benzo[b][1,4] thiazine-6- carboxamide | (400 MHz; DMSO-d₆): 0.73 (m, 1H), 0.94-1.08 (m, 3H), 1.08-1.18 (m, 2H), 1.46 (m, 1H), 1.50- 1.55 (m, 3H), 1.56-1.59 (m, 1H), 1.67-1.81 (m, 1H), 3.40-3.45 (m, 1H), 3.54 (s, 2H), 3.68 (m, 1H), 5.33 (d, J = 6.08 Hz, 2H), 7.06-7.71 (m, 4H), 7.22-7.29 (m, 5H), 7.39 (d, J = 8.04 Hz, 1H), 7.59 (s, 1H), 8.17 (bs, 1H). | 537 |
| 44 | 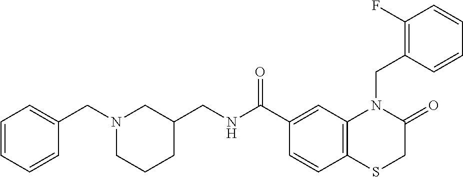 [SMILES: O=C(NCC1CCCN(Cc2ccccc2)C1)c1ccc2c(c1)N(Cc1ccccc1F)C(=O)CS2] | N-((1- benzylpiperidin- 3-yl)methyl)-4- (2- fluorobenzyl)- 3-oxo-3,4- dihydro-2H- benzo[b][1,4] thiazine-6- carboxamide | (500 MHz; DMSO-d₆): δ 0.84-0.91 (m, 1H), 1.24 (s, 2H), 1.43-1.48 (m, 1H), 1.60-1.68 (m, 2H), 1.91 (s, 1H), 2.69 (s, 4H), 3.09 (bs, 2H), 3.73 (s, 2H), 5.28 (s, 2H), 7.07-7.13 (m, 2H), 7.20- 7.57 (m, 10H), 8.50- 8.65 (m, 1H). | 504.42 |
| 45 | 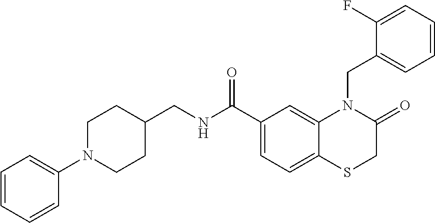 [SMILES: O=C(NCC1CCN(c2ccccc2)CC1)c1ccc2c(c1)N(Cc1ccccc1F)C(=O)CS2] | 4-(2- fluorobenzyl)- 3-oxo-N-((1- phenyl- piperidin-4- yl)methyl)- 3,4-dihydro- 2H- benzo[b][1,4] thiazine-6- carboxamide | (500 MHz; DMSO-d₆): δ 1.22-1.24 (m, 2H), 1.65- 1.70 (m, 3H), 2.56-2.60 (m, 2H), 3.14 (t, J = 5.95 Hz, 2H), 3.65-3.73 (m, 4H), 5.29 (s, 2H), 6.74 (t, J = 7.2 Hz, 1H), 6.92 (d, J = 8.15 Hz, 2H), 7.11-7.12 (m, 2H), 7.17- 7.25 (m, 3H), 7.31-7.32 (m, 1H), 7.53-7.57 (m, 3H), 8.55 (t, J = 5.5 Hz, 1H). | 490.40 |
| 46 | 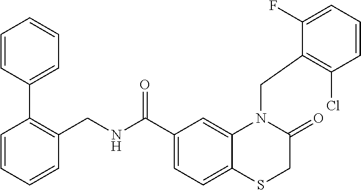 [SMILES: O=C(NCc1ccccc1-c1ccccc1)c1ccc2c(c1)N(Cc1c(F)cccc1Cl)C(=O)CS2] | N-([1,1′- biphenyl]-2- ylmethyl)-4- (2-chloro-6- fluorobenzyl)- 3-oxo-3,4- dihyddro-2H- benzo[b][1,4] thiazine-6- carboxamide | (500 MHz; DMSO-d₆): δ 3.60 (s, 2H), 4.41 (d, J = 5.50 Hz, 2H), 5.39 (s, 2H), 7.13 (t, J = 9.35 Hz, 1H), 6.25 (d, J = 7.7 Hz, 2H), 7.28-7.31 (m, 1H), 7.32-7.42 (m, 6H), 7.46- 7.48 (m, 4H), 7.77 (bs, 1H), 8.91 (t, J = 5.5 Hz, 1H). | 517.34 |
| 47 | 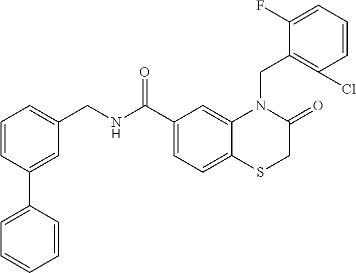 [SMILES: O=C(NCc1cccc(-c2ccccc2)c1)c1ccc2c(c1)N(Cc1c(F)cccc1Cl)C(=O)CS2] | N-([1,1′- biphenyl]-3- ylmethyl)-4- (2-chloro-6- fluorobenzyl)- 3-oxo-3,4- dihydro-2H- benzo[b][1,4] thiazine-6- carboxamide | (500 MHz; DMSO-d₆): δ 3.59 (s, 2H), 4.55 (d, J = 5.80 Hz, 2H), 5.39 (s, 2H), 7.04 (t, J = 9.50 Hz, 1H), 7.19 (d, J = 7.9 Hz, 1H), 7.22-7.26 (m, 1H), 7.30 (d, J = 7.50 Hz, 1H), 7.38 (t, J = 7.20 Hz, 1H), 7.43-7.51 (m, 6H), 7.59 (s, 1H), 7.64 (d, J = 7.4 Hz, 2H), 7.81 (bs, 1H), 9.07 (t, J = 5.85 Hz, 1H). | 517.31 |
| 48 | 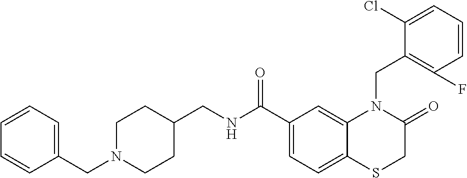 [SMILES: O=C(NCC1CCN(Cc2ccccc2)CC1)c1ccc2c(c1)N(Cc1c(F)cccc1Cl)C(=O)CS2] | N-((1- benzylpiperidin- 4-yl)methyl)-4- (2-chloro-6- fluorobenzyl)- 3-oxo-3,4- dihydro-2H- benzo[b][1,4] thiazine-6- carboxamide | (500 MHz; DMSO-d₆): δ 1.24-1.30 (bs, 2H), 1.76 (bs, 5H), 2.08 (s, 1H), 2.89 (bs, 2H), 3.14 (bs, 2H), 3.59 (s, 2H), 4.28 (bs, 1H), 5.38 (s, 2H), 7.11 (t, J = 9.3 Hz, 1H), 7.22-7.49 (m, 9H), 7.72 (s, 1H), 8.50 (bs, 1H). | 538.37 |
| 49 | 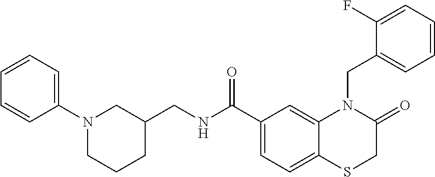 [SMILES: O=C(NCC1CCCN(c2ccccc2)C1)c1ccc2c(c1)N(Cc1ccccc1F)C(=O)CS2] | 4-(2- fluorobenzyl)- 3-oxo-N-((1- phenyl- piperidin-3- yl)methyl)- 3,4-dihydro- 2H benzo[b][1,4] thiazine-6- carboxamide | (500 MHz; DMSO-d₆): δ 1.05-1.12 (m, 1H), 1.50- 1.55 (m, 1H), 1.71 (d, J = 10.25 Hz, 2H), 1.84 (bs, 1H), 2.41 (t, J = 10.65 Hz, 1H), 2.64 (t, J = 10.75 Hz, 1H), 3.13-3.24 (m, 2H), 3.52-3.56 (m, 2H), 3.73 (s, 2H), 5.29 (s, 2H), 6.74 (t, J = 7.2 Hz, 1H), 6.89 (d, J = | 490.42 |
| | | | 8.20 Hz, 2H), 7.07-7.12 | |
| | | | (m, 2H), 7.16-7.21 (m, | |
| | | | 3H), 7.26-7.30 (m, 1H), | |
| | | | 7.53-7.58 (m, 3H), 8.58 | |
| | | | (t, J = 5.60 Hz, 1H). | |

### Examples 50-105

Examples 50-105 were prepared using Library general procedure 1 or 2 as indicated in the table below.

| | | | Method | | Purity |
|---|---|---|---|---|---|
| Exam- | | | of | LCMS | (%) by |
| ple | Structure | IUPAC Name | synthesis | [M + H] | UPLC |
| 50 | 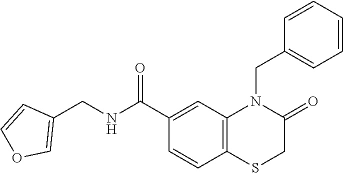 [SMILES: O=C(NCc1ccoc1)c1ccc2c(c1)N(Cc1ccccc1)C(=O)CS2] | 4-benzyl-N-(furan- 3-ylmethyl)-3-oxo- 3,4-dihydro-2H- benzo[b- 1,4]thiazine-6- carboxamide | Library general procedure 1, Method 1 | 379 | 95.39 |
| 51 | 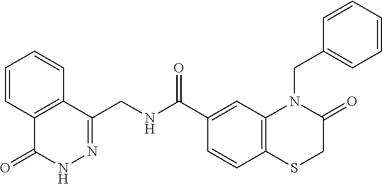 [SMILES: O=C(NCc1n[nH]c(=O)c2ccccc12)c1ccc2c(c1)N(Cc1ccccc1)C(=O)CS2] | 4-benzyl-3-oxo-N- ((4-oxo-3,4- dihydrophthalazin- 1-yl)methyl)-3,4- dihydro-2H- benzo[b- 1,4]thiazine-6- carboxamide | Library general procedure 1, Method 1 | 457 | 99.17 |
| 52 | 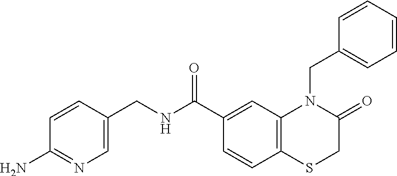 [SMILES: Nc1ccc(CNC(=O)c2ccc3c(c2)N(Cc2ccccc2)C(=O)CS3)cn1] | N-((6- aminopyridin-3- yl)methyl)-4- benzyl-3-oxo-3,4- dihydro-2H- benzo[b- 1,4]thiazine-6- carboxamide | Library general procedure 1, Method 1 | 405 | 98.95 |
| 53 | 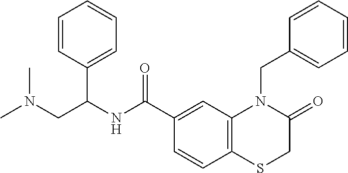 [SMILES: CN(C)CC(NC(=O)c1ccc2c(c1)N(Cc1ccccc1)C(=O)CS2)c1ccccc1] | 4-benzyl-N-(2- (dimethylamino)-1- phenylethyl)-3-oxo- 3,4-dihydro-2H- benzo[b- 1,4]thiazine-6- carboxamide | Library general procedure 1, Method 3 | 446 | 98.96 |
| 54 | 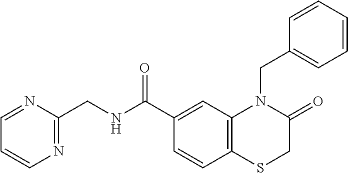 [SMILES: O=C(NCc1ncccn1)c1ccc2c(c1)N(Cc1ccccc1)C(=O)CS2] | 4-benzyl-3-oxo-N- (pyrimidin-2- ylmethyl)-3,4- dihydro-2H- benzo[b- 1,4]thiazine-6- carboxamide | Library general procedure 1, Method 1 | 391 | 98.95 |
| 55 | 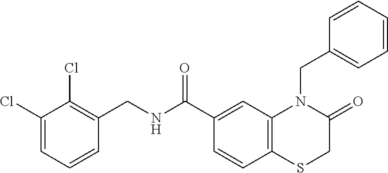 [SMILES: O=C(NCc1cccc(Cl)c1Cl)c1ccc2c(c1)N(Cc1ccccc1)C(=O)CS2] | 4-benzyl-N-(2,3- dichlorobenzyl)-3- oxo-3,4-dihydro- 2H-benzo[b- 1,4]thiazine-6- carboxamide | Library general procedure 1, Method 1 | 457 | 100 |
| 56 | 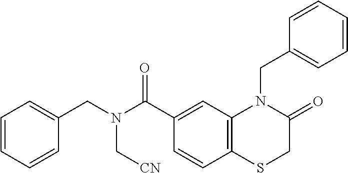 [SMILES: N#CCN(Cc1ccccc1)C(=O)c1ccc2c(c1)N(Cc1ccccc1)C(=O)CS2] | N,4-dibenzyl-N- (cyanomethyl)-3- oxo-3,4-dihydro- 2H-benzo[b- 1,4]thiazine-6- carboxamide | Library general procedure 1, Method 3 | 428 | 93.19 |
| 57 | 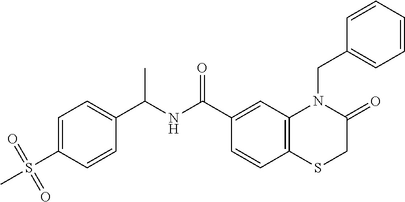 [SMILES: CC(NC(=O)c1ccc2c(c1)N(Cc1ccccc1)C(=O)CS2)c1ccc(S(C)(=O)=O)cc1] | 4-benzyl-N-(1-(4- (methylsulfonyl) phenyl)ethyl)-3-oxo- 3,4-dihydro-2H- benzo[b- 1,4]thiazine-6- carboxamide | Library general procedure 1, Method 2 | 481 | 99.79 |
| 58 | 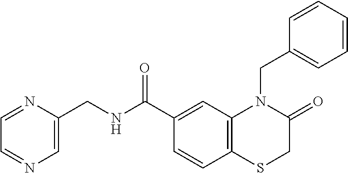 [SMILES: O=C(NCc1cnccn1)c1ccc2c(c1)N(Cc1ccccc1)C(=O)CS2] | 4-benzyl-3-oxo-N- (pyrazin-2- ylmethyl)-3,4- dihydro-2H- benzo[b- 1,4]thiazine-6- carboxamide | Library general procedure 1, Method 1 | 391 | 98.59 |
| 59 | 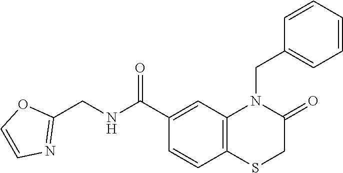 [SMILES: O=C(NCc1ncco1)c1ccc2c(c1)N(Cc1ccccc1)C(=O)CS2] | 4-benzyl-N-(oxazol- 2-ylmethyl)-3-oxo- 3,4-dihydro-2H- benzo[b- 1,4]thiazine-6- carboxamide | Library general procedure 1, Method 1 | 380 | 100 |
| 60 | 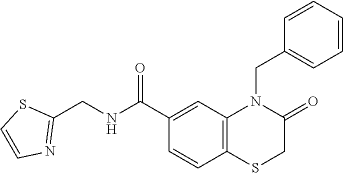 [SMILES: O=C(NCc1nccs1)c1ccc2c(c1)N(Cc1ccccc1)C(=O)CS2] | 4-benzyl-3-oxo-N- (thiazol-2- ylmethyl)-3,4- dihydro-2H- benzo[b- 1,4]thiazine-6- carboxamide | Library general procedure 1, Method 1 | 396 | 99.5 |
| 61 | 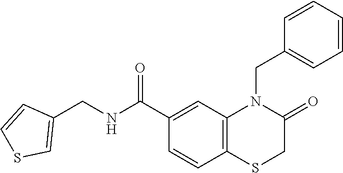 [SMILES: O=C(NCc1ccsc1)c1ccc2c(c1)N(Cc1ccccc1)C(=O)CS2] | 4-benzyl-3-oxo-N- (thiophen-3- ylmethyl)-3,4- dihydro-2H- benzo[b- 1,4]thiazine-6- carboxamide | Library general procedure 1, Method 1 | 395 | 98.32 |
| 62 | 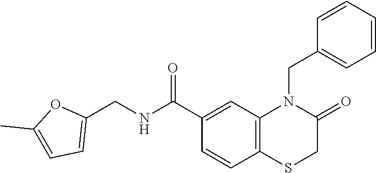 [SMILES: Cc1ccc(CNC(=O)c2ccc3c(c2)N(Cc2ccccc2)C(=O)CS3)o1] | 4-benzyl-N-((5- methylfuran-2- yl)methyl)-3-oxo- 3,4-dihydro-2H- benzo[b- 1,4]thiazine-6- carboxamide | Library general procedure 1, Method 1 | 393 | 99.05 |
| 63 | 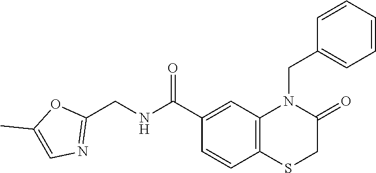 [SMILES: Cc1cnc(CNC(=O)c2ccc3c(c2)N(Cc2ccccc2)C(=O)CS3)o1] | 4-benzyl-N-((5- methyloxazol-2- yl)methyl)-3-oxo- 3,4-dihydro-2H- benzo[b- 1,4]thiazine-6- carboxamide | Library general procedure 1, Method 1 | 394 | 100 |
| 64 | 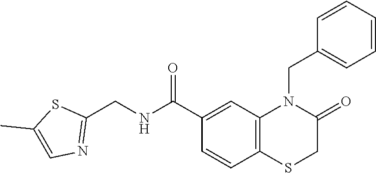 [SMILES: Cc1cnc(CNC(=O)c2ccc3c(c2)N(Cc2ccccc2)C(=O)CS3)s1] | 4-benzyl-N-((5- methylthiazol-2- yl)methyl)-3-oxo- 3,4-dihydro-2H- benzo[b- 1,4]thiazine-6- carboxamide | Library general procedure 1, Method 1 | 410 | 100 |
| 65 | 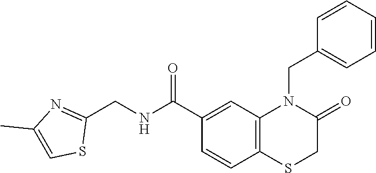 [SMILES: Cc1csc(CNC(=O)c2ccc3c(c2)N(Cc2ccccc2)C(=O)CS3)n1] | 4-benzyl-N-((4- methylthiazol-2- yl)methyl)-3-oxo- 3,4-dihydro-2H- benzo[b- 1,4]thiazine-6- carboxamide | Library general procedure 1, Method 1 | 410 | 99.21 |
| 66 | 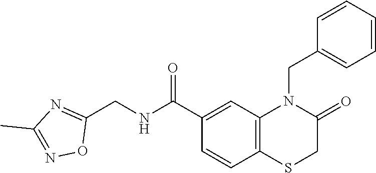 [SMILES: Cc1noc(CNC(=O)c2ccc3c(c2)N(Cc2ccccc2)C(=O)CS3)n1] | 4-benzyl-N-((3- methyl-1,2,4- oxadiazol-5- yl)methyl)-3-oxo- 3,4-dihydro-2H- benzo[b- 1,4]thiazine-6- carboxamide | Library general procedure 1, Method 1 | 395 | 95.74 |
| 67 | 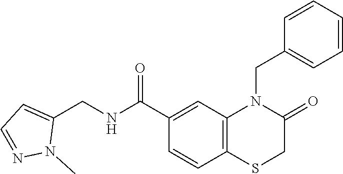 [SMILES: Cn1nccc1CNC(=O)c1ccc2c(c1)N(Cc1ccccc1)C(=O)CS2] | 4-benzyl-N-((1- methyl-1H-pyrazol- 5-yl)methyl)-3-oxo- 3,4-dihydro-2H- benzo[b- 1,4]thiazine-6- carboxamide | Library general procedure 1, Method 1 | 393 | 100 |
| 68 | 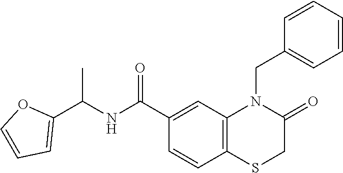 [SMILES: CC(NC(=O)c1ccc2c(c1)N(Cc1ccccc1)C(=O)CS2)c1ccco1] | 4-benzyl-N-(1- (furan-2-yl)ethyl)-3- oxo-3,4-dihydro- 2H-benzo[b- 1,4]thiazine-6- carboxamide | Library general procedure 1, Method 1 | 391 | 99.2 |
| 69 | 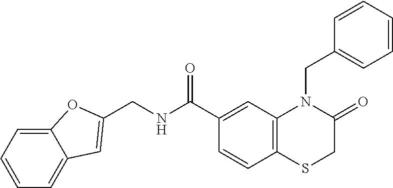 [SMILES: O=C(NCc1cc2ccccc2o1)c1ccc2c(c1)N(Cc1ccccc1)C(=O)CS2] | N-(benzofuran-2- ylmethyl)-4-benzyl- 3-oxo-3,4-dihydro- 2H-benzo[b- 1,4]thiazine-6- carboxamide | Library general procedure 1, Method 1 | 429 | 100 |
| 70 | 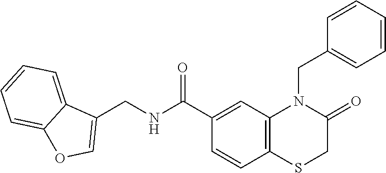 [SMILES: O=C(NCc1coc2ccccc12)c1ccc2c(c1)N(Cc1ccccc1)C(=O)CS2] | N-(benzofuran-3- ylmethyl)-4-benzyl- 3-oxo-3,4-dihydro- 2H-benzo[b- 1,4]thiazine-6- carboxamide | Library general procedure 1, Method 1 | 429 | 100 |
| 71 | 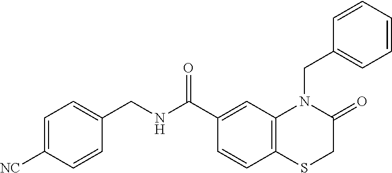 [SMILES: N#Cc1ccc(CNC(=O)c2ccc3c(c2)N(Cc2ccccc2)C(=O)CS3)cc1] | 4-benzyl-N-(4- cyanobenzyl)-3-oxo- 3,4-dihydro-2H- benzo[b- 1,4]thiazine-6- carboxamide | Library general procedure 1, Method 1 | 412 [M − H] | 100 |
| 72 | 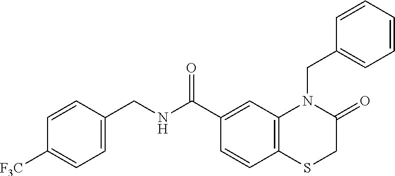 [SMILES: O=C(NCc1ccc(C(F)(F)F)cc1)c1ccc2c(c1)N(Cc1ccccc1)C(=O)CS2] | 4-benzyl-3-oxo-N- (4- (trifluoromethyl) benzyl)-3,4-dihydro- 2H-benzo[b- 1,4]thiazine-6- carboxamide | Library general procedure 1, Method 1 | 457 | 100 |
| 73 | 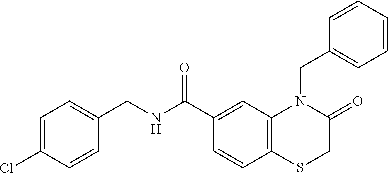 [SMILES: O=C(NCc1ccc(Cl)cc1)c1ccc2c(c1)N(Cc1ccccc1)C(=O)CS2] | 4-benzyl-N-(4- chlorobenzyl)-3- oxo-3,4-dihydro- 2H-benzo[b- 1,4]thiazine-6- carboxamide | Library general procedure 1, Method 1 | 423 | 100 |
| 74 | 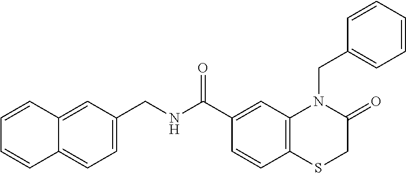 [SMILES: O=C(NCc1ccc2ccccc2c1)c1ccc2c(c1)N(Cc1ccccc1)C(=O)CS2] | 4-benzyl-N- (naphthalen-2- ylmethyl)-3-oxo- 3,4-dihydro-2H- benzo[b- 1,4]thiazine-6- carboxamide | Library general procedure 1, Method 1 | 439 | 100 |
| 75 | 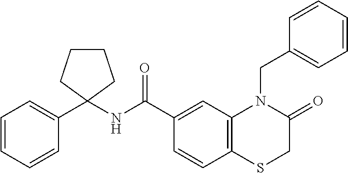 [SMILES: O=C(NC1(c2ccccc2)CCCC1)c1ccc2c(c1)N(Cc1ccccc1)C(=O)CS2] | 4-benzyl-3-oxo-N- (1- phenylcyclopentyl)- 3,4-dihydro-2H- benzo[b- 1,4]thiazine-6- carboxamide | Library general procedure 1, Method 1 | 443 | 91.82 |
| 76 | 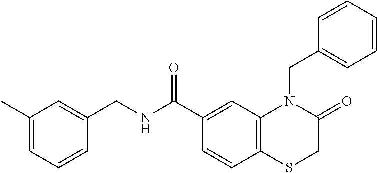 [SMILES: Cc1cccc(CNC(=O)c2ccc3c(c2)N(Cc2ccccc2)C(=O)CS3)c1] | 4-benzyl-N-(3- methylbenzyl)-3- oxo-3,4-dihydro- 2H-benzo[b- 1,4]thiazine-6- carboxamide | Library general procedure 1, Method 1 | 403 | 100 |
| 77 | 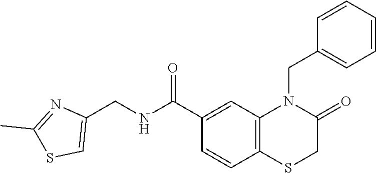 [SMILES: Cc1nc(CNC(=O)c2ccc3c(c2)N(Cc2ccccc2)C(=O)CS3)cs1] | 4-benzyl-N-((2- methylthiazol-4- yl)methyl)-3-oxo- 3,4-dihydro-2H- benzo[b- 1,4]thiazine-6- carboxamide | Library general procedure 1, Method 1 | 410 | 100 |
| 78 | 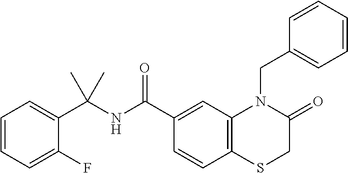 [SMILES: CC(C)(NC(=O)c1ccc2c(c1)N(Cc1ccccc1)C(=O)CS2)c1ccccc1F] | 4-benzyl-N-(2-(2- fluorophenyl) propan-2-yl)-3-oxo- 3,4-dihydro-2H- benzo[b- 1,4]thiazine-6- carboxamide | Library general procedure 1, Method 1 | 435 | 100 |
| 79 | 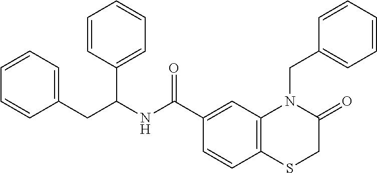 [SMILES: O=C(NC(Cc1ccccc1)c1ccccc1)c1ccc2c(c1)N(Cc1ccccc1)C(=O)CS2] | 4-benzyl-N-(1,2- diphenylethyl)-3- oxo-3,4-dihydro- 2H-benzo[b- 1,4]thiazine-6- carboxamide | Library general procedure 1, Method 1 | 479 | 100 |
| 80 | 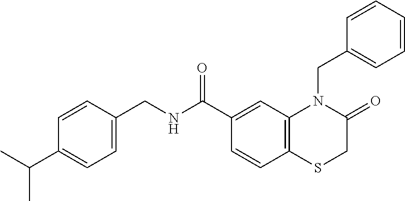 [SMILES: CC(C)c1ccc(CNC(=O)c2ccc3c(c2)N(Cc2ccccc2)C(=O)CS3)cc1] | 4-benzyl-N-(4- isopropylbenzyl)-3- oxo-3,4-dihydro- 2H-benzo[b- 1,4]thiazine-6- carboxamide | Library general procedure 1, Method 1 | 431 | 100 |
| 81 | 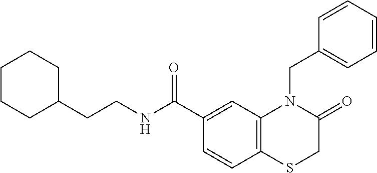 [SMILES: O=C(NCCC1CCCCC1)c1ccc2c(c1)N(Cc1ccccc1)C(=O)CS2] | 4-benzyl-N-(2- cyclohexylethyl)-3- oxo-3,4-dihydro- 2H-benzo[b- 1,4]thiazine-6- carboxamide | Library general procedure 1, Method 1 | 409 | 100 |
| 82 | 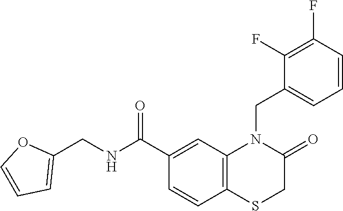 [SMILES: O=C(NCc1ccco1)c1ccc2c(c1)N(Cc1cccc(F)c1F)C(=O)CS2] | 4-(2,3- difluorobenzyl)-N- (furan-2-ylmethyl)- 3-oxo-3,4-dihydro- 2H-benzo[b- 1,4]thiazine-6- carboxamide | Library general procedure 2, Method 2 | 415 | 100 |
| 83 | 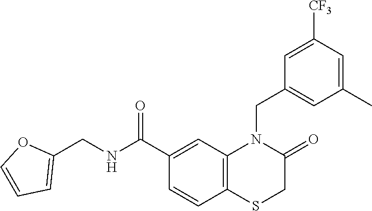 [SMILES: Cc1cc(CN2C(=O)CSc3ccc(C(=O)NCc4ccco4)cc32)cc(C(F)(F)F)c1] | N-(furan-2- ylmethyl)-4-(3- methyl-5- (trifluoromethyl) benzyl)-3-oxo-3,4- dihydro-2H- benzo[b- 1,4]thiazine-6- carboxamide | Library general procedure 2, Method 1 | 459 [M − H] | 98.65 |
| 84 | 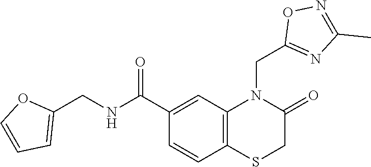 [SMILES: Cc1noc(CN2C(=O)CSc3ccc(C(=O)NCc4ccco4)cc32)n1] | N-(furan-2- ylmethyl)-4-((3- methyl-1,2,4- oxadiazol-5- yl)methyl)-3-oxo- 3,4-dihydro-2H- benzo[b- 1,4]thiazine-6- carboxamide | Library general procedure 2, Method 2 | 385 | 100 |
| 85 | 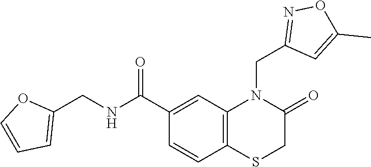 [SMILES: Cc1cc(CN2C(=O)CSc3ccc(C(=O)NCc4ccco4)cc32)no1] | N-(furan-2- ylmethyl)-4-((5- methylisoxazol-3- yl)methyl)-3-oxo- 3,4-dihydro-2H- benzo[b- 1,4]thiazine-6- carboxamide | Library general procedure 2, Method 2 | 384 | 100 |
| 86 | 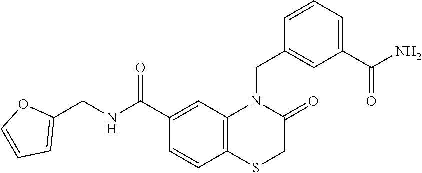 [SMILES: NC(=O)c1cccc(CN2C(=O)CSc3ccc(C(=O)NCc4ccco4)cc32)c1] | 4-(3- carbamoylbenzyl)- N-(furan-2- ylmethyl)-3-oxo- 3,4-dihydro-2H- benzo[b- 1,4]thiazine-6- carboxamide | Library general procedure 2, Method 1 | 422 | 90.6 |
| 87 | 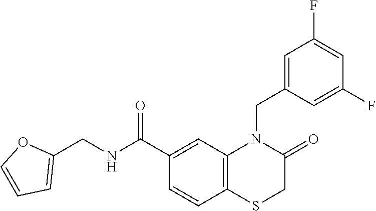 [SMILES: O=C(NCc1ccco1)c1ccc2c(c1)N(Cc1cc(F)cc(F)c1)C(=O)CS2] | 4-(3,5- difluorobenzyl)-N- (furan-2-ylmethyl)- 3-oxo-3,4-dihydro- 2H- benzo[b][1,4] thiazine-6- carboxamide | Library general procedure 2, Method 1 | 415 | 98.88 |
| 88 | 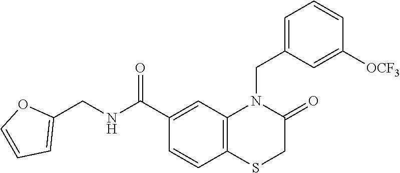 [SMILES: O=C(NCc1ccco1)c1ccc2c(c1)N(Cc1cccc(OC(F)(F)F)c1)C(=O)CS2] | N-(furan-2- ylmethyl)-3-oxo-4- (3- (trifluoromethoxy) benzyl)-3,4-dihydro- 2H-benzo[b- 1,4]thiazine-6- carboxamide | Library general procedure 2, Method 2 | 463 | 98.28 |
| 89 | 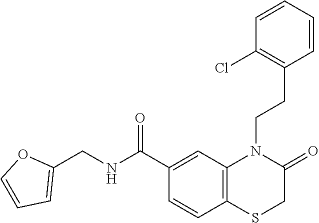 [SMILES: O=C(NCc1ccco1)c1ccc2c(c1)N(CCc1ccccc1Cl)C(=O)CS2] | 4-(2- chlorophenethyl)-N- (furan-2-ylmethyl)- 3-oxo-3,4-dihydro- 2H-benzo[b- 1,4]thiazine-6- carboxamide | Library general procedure 2, Method 2 | 427 | 94.24 |
| 90 | 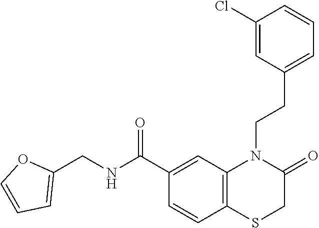 [SMILES: O=C(NCc1ccco1)c1ccc2c(c1)N(CCc1cccc(Cl)c1)C(=O)CS2] | 4-(3- chlorophenethyl)-N- (furan-2-ylmethyl)- 3-oxo-3,4-dihydro- 2H-benzo[b- 1,4]thiazine-6- carboxamide | Library general procedure 2, Method 2 | 427 | 100 |
| 91 | 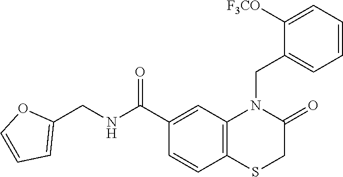 [SMILES: O=C(NCc1ccco1)c1ccc2c(c1)N(Cc1ccccc1OC(F)(F)F)C(=O)CS2] | N-(furan-2- ylmethyl)-3-oxo-4- (2- (trifluoromethoxy) benzyl)-3,4-dihydro- 2H-benzo[b- 1,4]thiazine-6- carboxamide | Library general procedure 2, Method 1 | 463 | 100 |
| 92 | 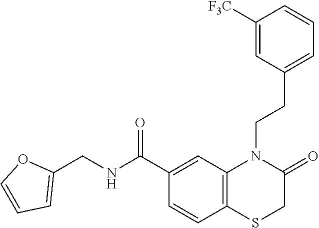 [SMILES: O=C(NCc1ccco1)c1ccc2c(c1)N(CCc1cccc(C(F)(F)F)c1)C(=O)CS2] | N-(furan-2- ylmethyl)-3-oxo-4- (3- (trifluoromethyl) phenethyl)-3,4- dihydro-2H- benzo[b- 1,4]thiazine-6- carboxamide | Library general procedure 2, Method 2 | 461 | 100 |
| 93 | 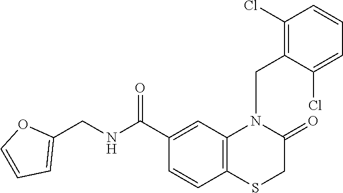 [SMILES: O=C(NCc1ccco1)c1ccc2c(c1)N(Cc1c(Cl)cccc1Cl)C(=O)CS2] | 4-(2,6- dichlorobenzyl)-N- (furan-2-ylmethyl)- 3-oxo-3,4-dihydro- 2H-benzo[b- 1,4]thiazine-6- carboxamide | Library general procedure 2, Method 1 | 447 | 100 |
| 94 | 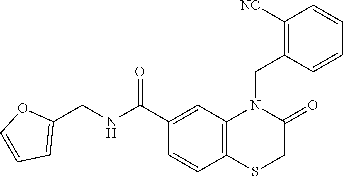 [SMILES: N#Cc1ccccc1CN1C(=O)CSc2ccc(C(=O)NCc3ccco3)cc21] | 4-(2-cyanobenzyl)- N-(furan-2- ylmethyl)-3-oxo- 3,4-dihydro-2H- benzo[b- 1,4]thiazine-6- carboxamide | Library general procedure 2, Method 1 | 404 | 100 |
| 95 | 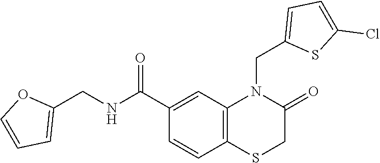 [SMILES: O=C(NCc1ccco1)c1ccc2c(c1)N(Cc1ccc(Cl)s1)C(=O)CS2] | 4-((5- chlorothiophen-2- yl)methyl)-N- (furan-2-ylmethyl)- 3-oxo-3,4-dihydro- 2H-benzo[b- 1,4]thiazine-6- carboxamide | Library general procedure 2, Method 2 | 419 | 100 |
| 96 | 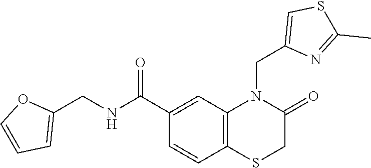 [SMILES: Cc1nc(CN2C(=O)CSc3ccc(C(=O)NCc4ccco4)cc32)cs1] | N-(furan-2- ylmethyl)-4-((2- methylthiazol-4- yl)methyl)-3-oxo- 3,4-dihydro-2H- benzo[b- 1,4]thiazine-6- carboxamide | Library general procedure 2, Method 2 | 400 | 100 |
| 97 | 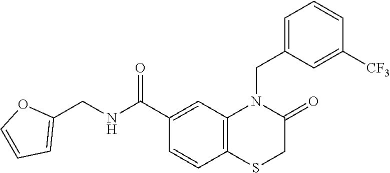 [SMILES: O=C(NCc1ccco1)c1ccc2c(c1)N(Cc1cccc(C(F)(F)F)c1)C(=O)CS2] | N-(furan-2- ylmethyl)-3-oxo-4- (3- (trifluoromethyl) benzyl)-3,4-dihydro- 2H-benzo[b- 1,4]thiazine-6- carboxamide | Library general procedure 2, Method 1 | 447 | 99.71 |
| 98 | 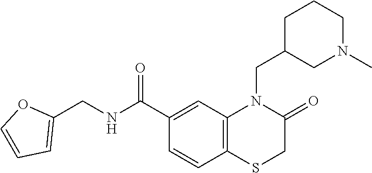 [SMILES: CN1CCCC(CN2C(=O)CSc3ccc(C(=O)NCc4ccco4)cc32)C1] | N-(furan-2- ylmethyl)-4-((1- methylpiperidin-3- yl)methyl)-3-oxo- 3,4-dihydro-2H- benzo[b- 1,4]thiazine-6- carboxamide | Library general procedure 2, Method 2 | 400 | 100 |
| 99 | 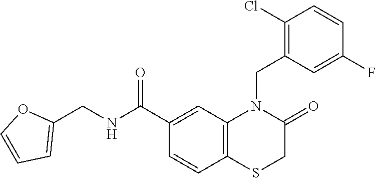 [SMILES: O=C(NCc1ccco1)c1ccc2c(c1)N(Cc1cc(F)ccc1Cl)C(=O)CS2] | 4-(2-chloro-5- fluorobenzyl)-N- (furan-2-ylmethyl)- 3-oxo-3,4-dihydro- 2H-benzo[b- 1,4]thiazine-6- carboxamide | Library general procedure 2, Method 1 | 431 | 100 |
| 100 | 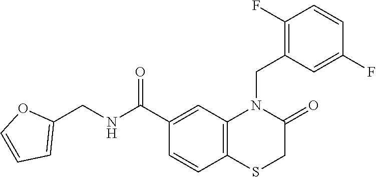 [SMILES: O=C(NCc1ccco1)c1ccc2c(c1)N(Cc1cc(F)ccc1F)C(=O)CS2] | 4-(2,5- difluorobenzyl)-N- (furan-2-ylmethyl)- 3-oxo-3,4-dihydro- 2H-benzo[b- 1,4]thiazine-6- carboxamide | Library general procedure 2, Method 2 | 415 | 98.39 |
| 101 | 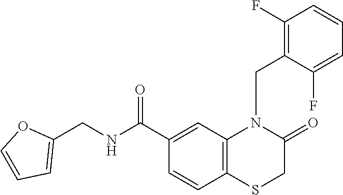 [SMILES: O=C(NCc1ccco1)c1ccc2c(c1)N(Cc1c(F)cccc1F)C(=O)CS2] | 4-(2,6- difluorobenzyl)-N- (furan-2-ylmethyl)- 3-oxo-3,4-dihydro- 2H-benzo[b- 1,4]thiazine-6- carboxamide | Library general procedure 2, Method 1 | 415 | 100 |
| 102 | 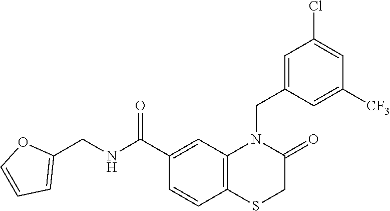 [SMILES: O=C(NCc1ccco1)c1ccc2c(c1)N(Cc1cc(Cl)cc(C(F)(F)F)c1)C(=O)CS2] | 4-(3-chloro-5- (trifluoromethyl) benzyl)-N-(furan-2- ylmethyl)-3-oxo- 3,4-dihydro-2H- benzo[b- 1,4]thiazine-6- carboxamide | Library general procedure 2, Method 2 | 481 | 100 |
| 103 | 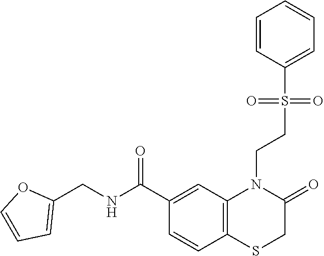 [SMILES: O=C(NCc1ccco1)c1ccc2c(c1)N(CCS(=O)(=O)c1ccccc1)C(=O)CS2] | N-(furan-2- ylmethyl)-3-oxo-4- (2- (phenylsulfonyl) ethyl)-3,4-dihydro- 2H-benzo[b- 1,4]thiazine-6- carboxamide | Library general procedure 2, Method 1 | 457 | 100 |
| 104 | 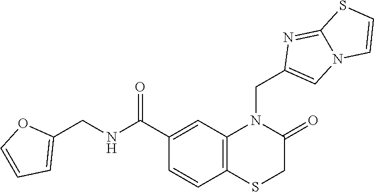 [SMILES: O=C(NCc1ccco1)c1ccc2c(c1)N(Cc1cn3ccsc3n1)C(=O)CS2] | N-(furan-2- ylmethyl)-4- (imidazo[2,1- b]thiazol-6- ylmethyl)-3-oxo- 3,4-dihydro-2H- benzo[b- 1,4]thiazine-6- carboxamide | Library general procedure 2, Method 2 | 425 | 100 |
| 105 | 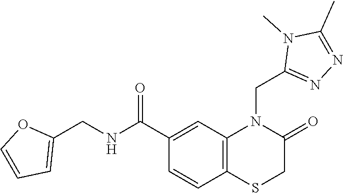 [SMILES: Cc1nnc(CN2C(=O)CSc3ccc(C(=O)NCc4ccco4)cc32)n1C] | 4-((4,5-dimethyl- 4H-1,2,4-triazol-3- yl)methyl)-N- (furan-2-ylmethyl)- 3-oxo-3,4-dihydro- 2H-benzo[b- 1,4]thiazine-6- carboxamide | Library general procedure 2, Method 2 | 398 | 100 |

### Examples 106-121

Examples 106-115 were prepared according to the above methods used to make Example 1 as described in General Procedures 1-3 using the appropriate amines and acids. Purification was as stated in the aforementioned methods.

Examples 116-121 were prepared using Library general procedure 1 or 2 as indicated in the table below.

| Ex- | | | | | LCMS |
|---|---|---|---|---|---|
| ample | Structure | IUPAC Name | ¹H-NMR | | [M + H] |
| 106 | 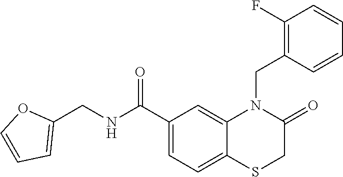 [SMILES: O=C(NCc1ccco1)c1ccc2c(c1)N(Cc1ccccc1F)C(=O)CS2] | 4-(2- fluorobenzyl)- N-(furan-2- ylmethyl)-3- oxo-3,4- dihydro-2H- benzo[b- 1,4]thiazine-6- carboxamide | (500 MHz; DMSO-d₆): δ 3.73 (s, 2H), 443 (d, J = 5.45 Hz, 2H), 5.29 (s, 2H), 6.21 (bs, 1H), 6.38 (bs, 1H), 7.10-7.11 (m, 2H), 7.22 (m, 1H), 7.30- 7.32 (m, 1H), 7.55-7.56 (m, 3H), 7.61 (s, 1H), 9.00 (bs, 1H). | | 397.21 |
| 107 | 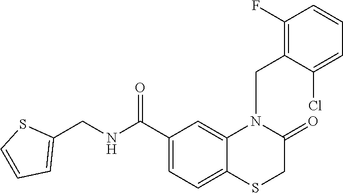 [SMILES: O=C(NCc1cccs1)c1ccc2c(c1)N(Cc1c(F)cccc1Cl)C(=O)CS2] | 4-(2-chloro-6- fluorobenzyl)- 3-oxo-N- (thiophen-2- ylmethyl)-3,4- dihydro-2H- benzo[b- 1,4]thiazine-6- carboxamide | (500 MHz; DMSO-d₆): δ 3.59 (s, 2H), 4.62 (d, J = 5.8 Hz, 2H), 5.38 (s, 2H), 6.97-7.01 (m, 2H), 7.11 (t, J = 9.1 Hz, 1H), 7.23-7.29 (m, 2H), 7.40- 7.41 (dd, J₁ = 1.15 Hz, J₂ = 5.05 Hz, 1H), 7.48 (s, 2H), 7.78 (s 1H), 9.09-9.11 (m, 1H). | | 447.07 |
| 108 | 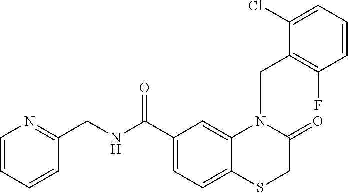 [SMILES: O=C(NCc1ccccn1)c1ccc2c(c1)N(Cc1c(F)cccc1Cl)C(=O)CS2] | 4-(2-chloro-6- fluorobenzyl)- 3-oxo-N- (pyridin-2- ylmethyl)-3,4- dihydro-2H- benzo[b- 1,4]thiazine-6- carboxamide | 500 MHz; DMSO-d₆): δ 3.60 (s, 2H), 4.56 (d, J = 5.8 Hz, 2H), 5.39 (s, 2H), 7.13 (t, J = 9.15 Hz, 1H), 7.24-7.31 (m, 4H), 7.50- 7.55 (m, 2H), 7.79-7.82 (m, 2H), 8.52-8.53 (m, 1H), 9.12 (bs, 1H). | | 442.14 |
| 109 | 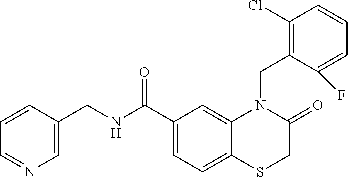 [SMILES: O=C(NCc1cccnc1)c1ccc2c(c1)N(Cc1c(F)cccc1Cl)C(=O)CS2] | 4-(2-chloro-6- fluorobenzyl)- 3-oxo-N- (pyridin-3- ylmethyl)-3,4- dihydro-2H- benzo[b- 1,4]thiazine-6- carboxamide | (500 MHz; DMSO-d₆): δ 3.65 (s, 2H), 4.45 (d, J = 5.5 Hz, 2H), 4.95 (s, 2H), 6.24 (d, J = 2.7 Hz, 1H), 6.37 (s, 1H), 7.11 (t, J = 9.1 Hz, 1H), 7.23-7.29 (m, 2H), 7.40-7.41 (dd, J₁ = 1.15 Hz, J₂ = 5.05 Hz, 1H), 7.48 (s, 2H), 7.78 (s 1H), 9.09-9.11 (m, 1H). | | 442.15 |
| 110 | 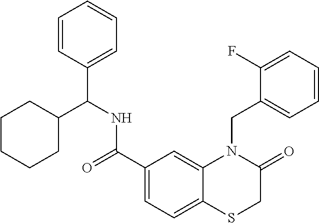 [SMILES: O=C(NC(c1ccccc1)C1CCCCC1)c1ccc2c(c1)N(Cc1ccccc1F)C(=O)CS2] | N- (cyclohexyl (phenyl) methyl)-4-(2- fluorobenzyl)- 3-oxo-3,4- dihydro-2H- benzo[b][1,4] thiazine-6- carboxamide | (500 MHz; DMSO-d₆): δ 0.79-0.86 (m, 1H), 0.90- 0.97 (m, 1H), 1.04-1.20 (m, 4H), 1.57-1.59 (m, 2H), 1.67-1.75 (m, 2H), 1.84-1.86 (m, 1H), 3.73 (bs, 2H), 4.61 (t, J = 9.2 Hz, 1H), 5.30 (bs, 2H), 7.07 (d, J = 4.5 Hz, 2H), 7.19-7.23 (m, 2H), 7.28- 7.30 (m, 5H), 7.50 (s, 1H), 7.53 (s, 2H), 8.70 (d, J = 8.75 Hz, 1H). | | 489.04 |
| 111 | 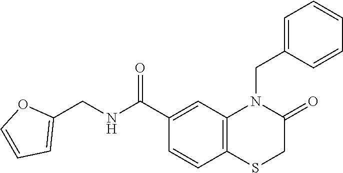 [SMILES: O=C(NCc1ccco1)c1ccc2c(c1)N(Cc1ccccc1)C(=O)CS2] | 4-benzyl-N- (furan-2- ylmethyl)-3- oxo-3,4- dihydro-2H- benzo[b- 1,4]thiazine-6- carboxamide | (500 MHz; DMSO-d₆): δ 3.72 (s, 2H), 4.42 (d, J = 5.5 Hz, 2H), 5.28 (s, 2H), 6.22 (d, J = 2.8 Hz, 1H), 6.39 (s, 1H), 7.20-7.24 (m, 3H), 7.29-7.32 (m, 2H), 7.51-7.54 (m, 2H), 7.57 (s, 1H), 7.65 (s, 1H), 8.99 (t, J = 5.45 Hz, 1H). | | 379.18 |
| 112 | 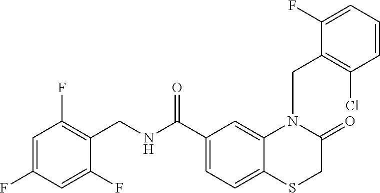 [SMILES: O=C(NCc1c(F)cc(F)cc1F)c1ccc2c(c1)N(Cc1c(F)cccc1Cl)C(=O)CS2] | 4-(2-chloro-6- fluorobenzyl)- 3-oxo-N-(2,4,6- trifluorobenzyl)- 3,4-dihydro- 2H-benzo[b- 1,4]thiazine-6- carboxamide | (500 MHz; DMSO-d₆): δ 3.58 (s, 2H), 4.45 (d, J = 4.9 Hz, 2H), 5.36 (s, 2H), 7.10 (t, J = 9.2 Hz, 1H), 7.20-7.31 (m, 4H), 7.42- 7.47 (m, 2H), 7.73 (s, 1H), 8.85 (t, J = 5.1 Hz, 1H). | | 495.24 |
| 113 | 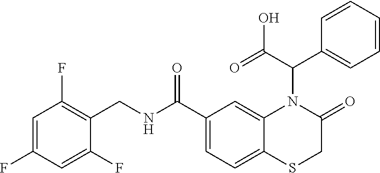 [SMILES: O=C(NCc1c(F)cc(F)cc1F)c1ccc2c(c1)N(C(C(=O)O)c1ccccc1)C(=O)CS2] | 2-(3-oxo-6- ((2,4,6- trifluorobenzyl) carbamoyl)-2H- benzo[b][1,4] thiazin-4(3H)-yl)- 2-phenylacetic acid | (400 MHz; DMSO-d₆): δ 3.61 (s, 2H), 4.37 (s, 2H), 6.17 (s, 1H), 7.33-7.14 (m, 9H), 7.57 (s, 1H), 8.78 (s, 1H), 13.0 (bs, 1H). | | 487.3 |
| 114 | 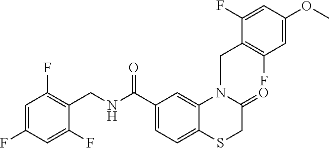 [SMILES: COc1cc(F)c(CN2C(=O)CSc3ccc(C(=O)NCc4c(F)cc(F)cc4F)cc32)c(F)c1] | 4-(2,6-difluoro- 4- methoxybenzyl)- 3-oxo-N- (2,4,6- trifluorobenzyl)- 3,4-dihydro- 2H- benzo[b][1,4] | (500 MHz; DMSO-d₆): δ 3.57 (s, 2H), 3.71 (s, 3H), 4.46 (d, J = 4.05 Hz, 2H), 5.26 (s, 2H), 6.61 (d, J = 9.85 Hz, 2H), 7.21 (t, J = 8.4 Hz, 2H), 7.45 (s, 2H), 7.78 (s, 1H), 8.85 (bs, 1H) | | 509.16 |
| | | thiazine-6- | | | |
| | | carboxamide | | | |
| 115 | 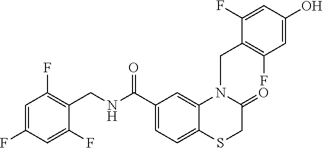 [SMILES: O=C(NCc1c(F)cc(F)cc1F)c1ccc2c(c1)N(Cc1c(F)cc(O)cc1F)C(=O)CS2] | 4-(2,6-difluoro- 4- hydroxybenzyl)- 3-oxo-N-(2,4,6- trifluorobenzyl)- 3,4-dihydro- 2H- benzo[b][1,4] thiazine-6- | (500 MHz; DMSO-d₆): δ 3.56 (s, 2H), 4.46 (d, J = 4.3 Hz, 2H), 5.22 (s, 2H), 6.31 (d, J = 9.75 Hz, 2H), 7.20 (t, J = 8.4 Hz, 2H), 7.44 (s, 2H), 7.76 (s, 1H), 8.84 (bs, 1H), 10.32 (s, 1H) | | 495.16 |
| | | carboxamide | | | |
| | | | Method | | Purity |
| Ex- | | | of | LCMS | (%) by |
| ample | Structure | IUPAC Name | synthesis | [M + H] | UPLC |
| 116 | 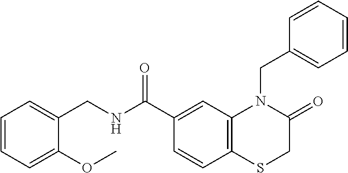 [SMILES: COc1ccccc1CNC(=O)c1ccc2c(c1)N(Cc1ccccc1)C(=O)CS2] | 4-benzyl-N-(2- methoxybenzyl)-3- oxo-3,4-dihydro- 2H-benzo[b- 1,4]thiazine-6- carboxamide | Library general procedure 1, Method 1 | 419 | 100 |
| 117 | 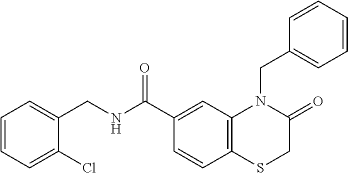 [SMILES: O=C(NCc1ccccc1Cl)c1ccc2c(c1)N(Cc1ccccc1)C(=O)CS2] | 4-benzyl-N-(2- chlorobenzyl)-3- oxo-3,4-dihydro- 2H-benzo[b- 1,4]thiazine-6- carboxamide | Library general procedure 1, Method 1 | 423 | 100 |
| 118 | 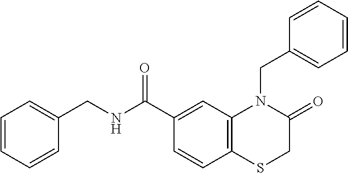 [SMILES: O=C(NCc1ccccc1)c1ccc2c(c1)N(Cc1ccccc1)C(=O)CS2] | N,4-dibenzyl-3-oxo- 3,4-dihydro-2H- benzo[b- 1,4]thiazine-6- carboxamide | Library general procedure 1, Method 1 | 389 | 100 |
| 119 | 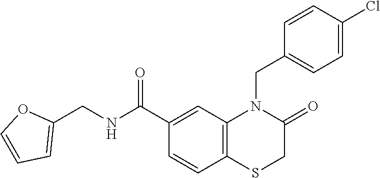 [SMILES: O=C(NCc1ccco1)c1ccc2c(c1)N(Cc1ccc(Cl)cc1)C(=O)CS2] | 4-(4-chlorobenzyl)- N-(furan-2- ylmethyl)-3-oxo- 3,4-dihydro-2H- benzo[b- 1,4]thiazine-6- carboxamide | Library general procedure 2, Method 1 | 413 | 99.31 |
| 120 | 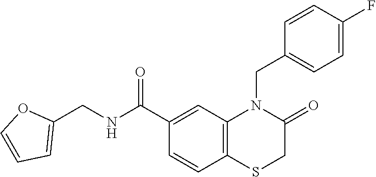 [SMILES: O=C(NCc1ccco1)c1ccc2c(c1)N(Cc1ccc(F)cc1)C(=O)CS2] | 4-(4-fluorobenzyl)- N-(furan-2- ylmethyl)-3-oxo- 3,4-dihydro-2H- benzo[b- 1,4]thiazine-6- carboxamide | Library general procedure 2, Method 2 | 397 | 100 |
| 121 | 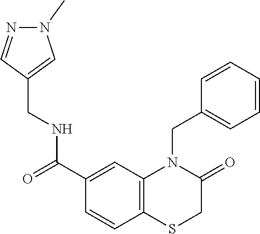 [SMILES: Cn1cc(CNC(=O)c2ccc3c(c2)N(Cc2ccccc2)C(=O)CS3)cn1] | 4-benzyl-N-((1- methyl-1H-pyrazol- 4-yl)methyl)-3-oxo- 3,4-dihydro-2H- benzo[b][1,4] thiazine-6- carboxamide | Library general procedure 1, Method 3 | 393 | 98.9 |

### Examples 122-131

Certain analogous (Examples 122-131) derivative compounds of formula (I) may also be obtained from commercial suppliers, which are also incorporated herein.

| Ex- | | | LCMS |
|---|---|---|---|
| ample | Structure | IUPAC Name | [M + H] |
| 122 | 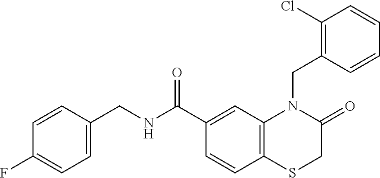 [SMILES: O=C(NCc1ccc(F)cc1)c1ccc2c(c1)N(Cc1ccccc1Cl)C(=O)CS2] | 4-(2-chlorobenzyl)-N-(4- fluorobenzyl)-3-oxo-3,4-dihydro-2H- benzo[b-1,4]thiazine-6-carboxamide | 441.17 |
| 123 | 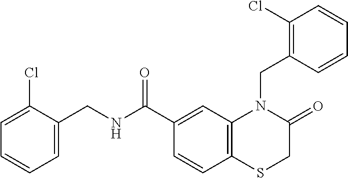 [SMILES: O=C(NCc1ccccc1Cl)c1ccc2c(c1)N(Cc1ccccc1Cl)C(=O)CS2] | N,4-bis(2-chlorobenzyl)-3-oxo-3,4- dihydro-2H-benzo[b-1,4]thiazine-6- carboxamide | 457.13 |
| 124 | 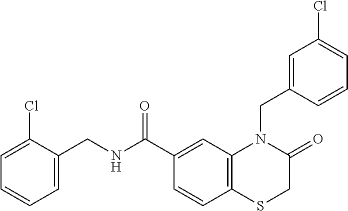 [SMILES: O=C(NCc1ccccc1Cl)c1ccc2c(c1)N(Cc1cccc(Cl)c1)C(=O)CS2] | N-(2-chlorobenzyl)-4-(3- chlorobenzyl)-3-oxo-3,4-dihydro- 2H-benzo[b-1,4]thiazine-6- carboxamide | 457.18 |
| 125 | 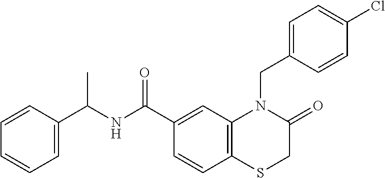 [SMILES: CC(NC(=O)c1ccc2c(c1)N(Cc1ccc(Cl)cc1)C(=O)CS2)c1ccccc1] | 4-(4-chlorobenzyl)-3-oxo-N-(1- phenylethyl)-3,4-dihydro-2H- benzo[b-1,4]thiazine-6-carboxamide | 437.2 |
| 126 | 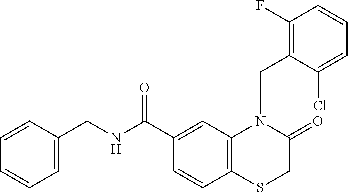 [SMILES: O=C(NCc1ccccc1)c1ccc2c(c1)N(Cc1c(F)cccc1Cl)C(=O)CS2] | N-benzyl-4-(2-chloro-6- fluorobenzyl)-3-oxo-3,4-dihydro-2H- benzo[b-1,4]thiazine-6-carboxamide | 441.19 |
| 127 | 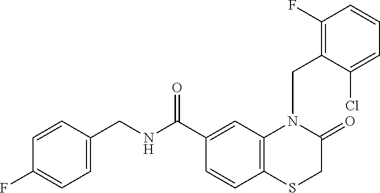 [SMILES: O=C(NCc1ccc(F)cc1)c1ccc2c(c1)N(Cc1c(F)cccc1Cl)C(=O)CS2] | 4-(2-chloro-6-fluorobenzyl)-N-(4- fluorobenzyl)-3-oxo-3,4-dihydro-2H- benzo[b-1,4]thiazine-6-carboxamide | 459.2 |
| 128 | 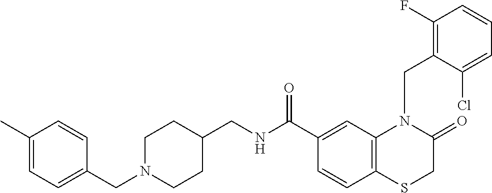 [SMILES: Cc1ccc(CN2CCC(CNC(=O)c3ccc4c(c3)N(Cc3c(F)cccc3Cl)C(=O)CS4)CC2)cc1] | 4-(2-chloro-6-fluorobenzyl)-N-((1- (4-methylbenzyl)piperidin-4- yl)methyl)-3-oxo-3,4-dihydro-2H- benzo[b][1,4]thiazine-6-carboxamide | 552.2 |
| 129 | 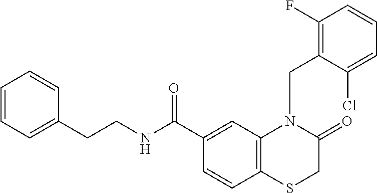 [SMILES: O=C(NCCc1ccccc1)c1ccc2c(c1)N(Cc1c(F)cccc1Cl)C(=O)CS2] | 4-(2-chloro-6-fluorobenzyl)-3-oxo- N-phenethyl-3,4-dihydro-2H- benzo[b-1,4]thiazine-6-carboxamide | 455.2 |
| 130 | 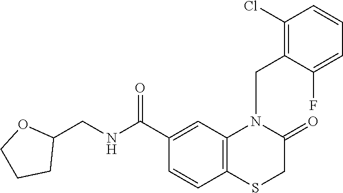 [SMILES: O=C(NCC1CCCO1)c1ccc2c(c1)N(Cc1c(F)cccc1Cl)C(=O)CS2] | 4-(2-chloro-6-fluorobenzyl)-3-oxo- N-((tetrahydrofuran-2-yl)methyl)- 3,4-dihydro-2H-benzo[b- 1,4]thiazine-6-carboxamide | 435.19 |
| 131 | 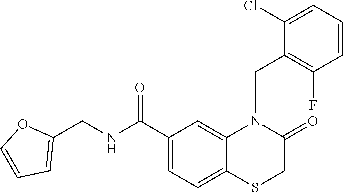 [SMILES: O=C(NCc1ccco1)c1ccc2c(c1)N(Cc1c(F)cccc1Cl)C(=O)CS2] | 4-(2-chloro-6-fluorobenzyl)-N- (furan-2-ylmethyl)-3-oxo-3,4- dihydro-2H-benzo[b][1,4]thiazine-6- carboxamide | 431.05 |

### Example 132: 4-Benzyl-N-(furan-2-ylmethyl)-2,2-dimethyl-3-oxo-3,4-dihydro-2H-benzo[b-1,4]thiazine-6-carboxamide

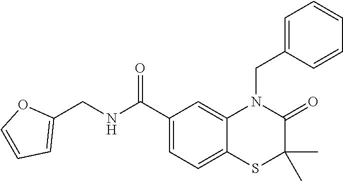 [SMILES: CC1(C)Sc2ccc(C(=O)NCc3ccco3)cc2N(Cc2ccccc2)C1=O]

### Preparation 7: Methyl benzyl-3-oxo-3,4-dihydro-2H-benzo[b-1,4]thiazine-6-carboxylate

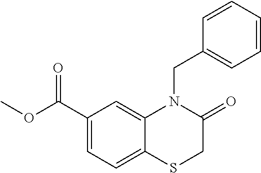 [SMILES: COC(=O)c1ccc2c(c1)N(Cc1ccccc1)C(=O)CS2]

To a stirred solution of methyl 3-oxo-3,4-dihydro-2H-benzo[b-1,4]thiazine-6-carboxylate (Preparation 1) (1.0 g, 4.48 mmol) in EDC (20 mL) was added NaOH solution (0.358 g, 5.38 mmol in 20 mL H₂O) portionwise followed by TBAB (0.105 g, 0.33 mmol) at RT. The whole was stirred for 30 min at RT and then benzyl bromide (0.643 mL, 5.38 mmol) was added dropwise. The reaction mixture was further stirred at RT for 2.5 h. Progress of the reaction was monitored by TLC and after completion; the reaction mixture was diluted with DCM, washed with water and brine solution. The combined organics were dried over anhydrous Na₂SO₄ and evaporated under reduced pressure to dryness to give crude material which was purified by column chromatography eluting with 20% EtOAc in hexanes to afford methyl benzyl-3-oxo-3,4-dihydro-2H-benzo[b-1,4]thiazine-6-carboxylate (0.75 g, 2.396 mmol, 54% yield) as a white solid. LCMS m/z: 314.09 [M+H].

### Preparation 8: Methyl 4-benzyl-2.2-dimethyl-3-oxo-3,4-dihydro-2H-1,4-benzothiazine-6-carboxylate

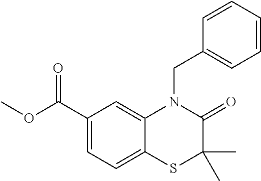 [SMILES: COC(=O)c1ccc2c(c1)N(Cc1ccccc1)C(=O)C(C)(C)S2]

To a stirred solution of methyl benzyl-3-oxo-3,4-dihydro-2H-benzo[b-1,4]thiazine-6-carboxylate (Preparation 7) (0.15 g, 0.48 mmol) in THF (2 mL) at −78° C. was added LiHMDS (0.52 mL, 1M solution in THF) drop wise. The mixture was stirred at −78 OC for 15 min. then Mel (0.032 mL, 0.527 mmol) added and the whole allowed to stir for another 30 min. at the same temperature then the reaction mixture was slowly brought to RT and further stirred for 3 h. The reaction mixture was quenched with aqueous NH₄Cl solution and extracted with DCM. The combined organics were washed with brine, dried over anhydrous Na₂SO₄ and evaporated under reduced pressure to give a crude product which was purified by column chromatography using 10% EtOAc in hexanes as eluent to afford methyl 4-benzyl-2,2-dimethyl-3-oxo-3,4-dihydro-2H-1,4-benzothiazine-6-carboxylate (0.13 g, 0.381 mmol, 80% yield) as a viscous oil. LCMS m/z: 342.29 [M+H].

### Preparation 9: 4-Benzyl-2,2-dimethyl-3-oxo-3,4-dihydro-2H-benzo[b-1,4]thiazine-6-carboxylic acid

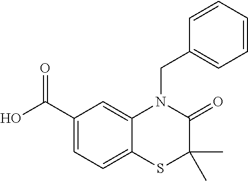 [SMILES: CC1(C)Sc2ccc(C(=O)O)cc2N(Cc2ccccc2)C1=O]

To a stirred solution of methyl 4-benzyl-2,2-dimethyl-3-oxo-3,4-dihydro-2H-1,4-benzothiazine-6-carboxylate (Preparation 8) (0.13 g, 0.381 mmol) in a mixture of MeOH, water and THF (1:1:2; 1.7 mL) was added LiOH.H₂O (0.032 g, 0.761 mmol) at RT. The reaction was allowed to stir at RT for 3 h. On completion of the reaction, the solvents were evaporated under reduced pressure and the residue was dissolved in water, washed with EtOAc and then acidified to ˜pH 5 with dilute HCl and the resulting aqueous solution was extracted with EtOAc. The combined organic extracts were washed with brine, dried over anhydrous Na₂SO₄ and evaporated to dryness to give 4-benzyl-2,2-dimethyl-3-oxo-3,4-dihydro-2H-benzo[b-1,4]thiazine-6-carboxylic acid (0.075 g. 0.229 mmol, 60% yield) as a colourless solid which was used in the next step without any further purification. LCMS m/z: 326.13 [M−H].

### Preparation 10: 4-Benzyl-N-(furan-2-ylmethyl)-2,2-dimethyl-3-oxo-3,4-dihydro-2H-benzo[b-1,4]thiazine-6-carboxamide

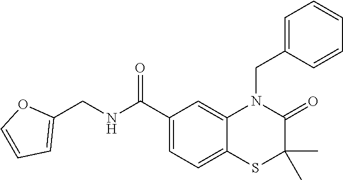 [SMILES: CC1(C)Sc2ccc(C(=O)NCc3ccco3)cc2N(Cc2ccccc2)C1=O]

To a stirred solution of 4-benzyl-2,2-dimethyl-3-oxo-3,4-dihydro-2H-benzo[b-1,4]thiazine-6-carboxylic acid (Preparation 9) (0.07 g, 0.21 mmol) in DMF (2 mL) was added HATU (0.098 g, 0.26 mmol) followed by TEA (0.066 mL, 0.47 mmol) at RT. The resulting reaction mixture was stirred at RT for 15 min. then 1-(furan-2-yl)-methanamine (0.021 mL, 0.24 mmol) was added and the whole further stirred at RT for 3 h. After complete consumption of the starting material, the reaction mixture was diluted with EtOAc and washed with chilled water and brine. The organics were dried over anhydrous Na₂SO₄ and evaporated under reduced pressure to dryness. The crude material obtained was purified by prep-HPLC to afford 4-Benzyl-N-(furan-2-ylmethyl)-2,2-dimethyl-3-oxo-3,4-dihydro-2H-benzo[b-1,4]thiazine-6-carboxamide (Example 132) (0.015 g, 0.036 mmol, 18% yield) as a white solid. LCMS m/z: 407.25 [M+H]; ¹H NMR (500 MHz; DMSO-d₆): δ 1.43 (s, 6H), 4.42 (d, J=5.55 Hz, 2H), 5.28 (s, 2H), 6.23 (bs, 1H), 6.39 (s, 1H), 7.17 (d, J=7.75 Hz, 2H), 7.23-7.25 (m, 1H), 7.31-7.34 (m, 2H), 7.51 (d, J=8.05 Hz, 1H), 7.56 (d, J=8.35 Hz, 2H), 7.64 (s, 1H), 9.02 (t, J=5.5 Hz, 1H).

### Examples 133-176

### Preparation 2: Methyl 2-bromo-3-oxo-3,4-dihydro-2H-benzo[b-1,4]thiazine-6-carboxylate

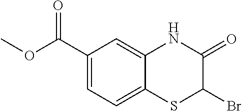 [SMILES: COC(=O)c1ccc2c(c1)NC(=O)C(Br)S2]

To a stirred solution of methyl 3-oxo-3,4-dihydro-2H-1,4-benzothiazine-6-carboxylate (Preparation 1) (1.0 g, 4.48 mmol) in CCl₄ was added NBS (0.797 g, 4.48 mmol) and benzoyl peroxide (0.0035 g. 0.14 mmol) at ice cold temperature. The reaction was allowed to warm to RT and then refluxed at 70° C. for 3 h. After completion of the reaction, the reaction mixture was cooled to room temperature and the solid obtained was filtered off. It was washed with water and dried under reduced pressure to obtain pure methyl 2-bromo-3-oxo-3,4-dihydro-2H-benzo[b][1,4]thiazine-6-carboxylate (0.8 g, 2.65 mmol, 60% yield) as a white solid. LCMS m/z: 238.05 [M−H] in MeCN.

### Preparation 3: Methyl 2-(dimethylamino)-3-oxo-3,4-dihydro-2H-1,4-benzo[b-1,4]thiazine-6-carboxylate

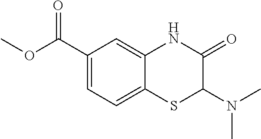

To a stirred solution of methyl 2-bromo-3-oxo-3,4-dihydro-2H-benzo[b][1,4]thiazine-6-carboxylate (Preparation 2) (0.25 g, 0.82 mmol) in THF (2.5 mL) was added dimethyl amine.HCl (0.202 g, 2.4 mmol) followed by TEA (0.7 mL, 4.92 mmol) at RT. The resulting reaction mixture was stirred at RT for 2 h. After completion of the reaction, the reaction mixture was diluted with EtOAc and washed with water and brine. The combined organics were dried over anhydrous Na₂SO₄ and evaporated under reduced pressure to afford the methyl 2-(dimethylamino)-3-oxo-3,4-dihydro-2H-1,4-benzo[b-1,4]thiazine-6-carboxylate (0.2 g, 0.75 mmol, 90% yield) as a crude sticky residue which was used as such without any further purification. LCMS m/z: 267.10 [M+H].

Examples 133-176 were prepared according to the above method used to make Example 132 and Preparation 3 as described in General Procedure 11 using the appropriate reagents. Purification was as stated in the aforementioned methods.

| | | | | LCMS |
|---|---|---|---|---|
| Example | Structure | IUPAC Name | ¹H-NMR | [M + H] |
| 133 | 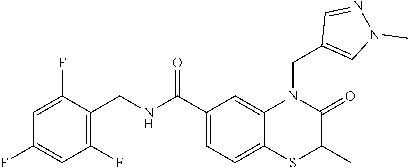 [SMILES: CC1Sc2ccc(C(=O)NCc3c(F)cc(F)cc3F)cc2N(Cc2cnn(C)c2)C1=O] | 2-methyl-N-2,4,6- trifluorobenzyl- 4-[(1-methyl- 1H-pyrazol-4- yl)methyl]-3- oxo-3,4- dihydro-2H- 1,4- benzothiazine- 6-carboxamide | (500 MHz; DMSO- d₆): δ 1.35 (d, J = 6.0 Hz, 3H), 3.76-3.78 (m, 4H), 4.47 (s, 2H), 5.05 (s, 2H), 7.19-7.22 (m, 2H), 7.28 (s, 1H), 7.47-7.53 (m, 3H), 7.77 (s, 1H), 8.98 (s, 1H) | 461.56 |
| 134 | 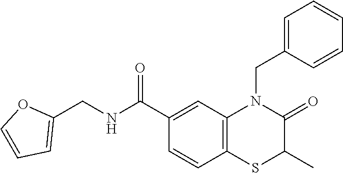 [SMILES: CC1Sc2ccc(C(=O)NCc3ccco3)cc2N(Cc2ccccc2)C1=O] | 4-benzyl-N- (furan-2- ylmethyl)-2- methyl-3-oxo- 3,4-dihydro- 2H-benzo[b- 1,4]thiazine-6- carboxamide | (500 MHz; DMSO- d₆): δ 1.39 (d, J = 6.95 Hz, 3H), 3.89-3.93 (q, J = 6.85 Hz, 1H), 4.42 (d, J = 5.65 Hz, 2H), 5.28 (s, 2H), 6.22 (d, J = 2.85 Hz, 1H), 6.39 (s, 1H), 7.19 (d, J = 7.55 Hz, 2H), 7.21- 7.24 (m, 1H), 7.30- | 393.23 |
| | | | 7.33 (m, 2H), 7.51-7.57 | |
| | | | (m, 3H), 7.66 (s, 1H), | |
| | | | 9.00 (t, J = 5.6 Hz, 1H). | |
| 135 | 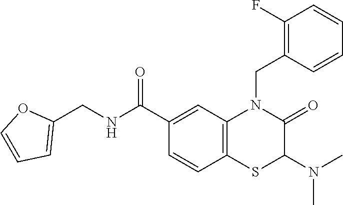 [SMILES: CN(C)C1Sc2ccc(C(=O)NCc3ccco3)cc2N(Cc2ccccc2F)C1=O] | 2- (dimethylamino)- 4-(2- fluorobenzyl)- N-(furan-2- ylmethyl)-3- oxo-3,4- dihydro-2H- benzo[b- 1,4]thiazine-6- carboxamide | (500 MHz; DMSO- d₆): δ 2.26 (s, 6H), 4.43 (bs, 2H), 4.92 (s, 1H), 5.32 (bs, 2H), 6.22 (s, 1H), 6.39 (s, 1H), 7.06-7.13 (m, 2H), 7.23-7.30 (m, 2H), 7.56-7.66 (m, 4H), 9.02 (bs, lH). | 440.24 |
| 136 | 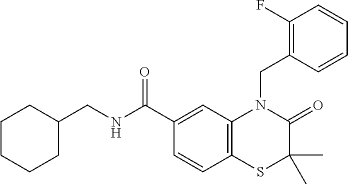 [SMILES: CC1(C)Sc2ccc(C(=O)NCC3CCCCC3)cc2N(Cc2ccccc2F)C1=O] | N- (cyclohexylmethyl)- 4-(2- fluorobenzyl)- 2,2-dimethyl-3- oxo-3,4- dihydro-2H- benzo[b- 1,4]thiazine-6- carboxamide | (500 MHz; DMSO- d₆): δ 1.13-1.16 (m, 2H), 1.24-1.30 (m, 4H), 1.43-1.47 (m, 6H), 1.59-1.65 (m, 5H), 3.04 (t, J = 6.25 Hz, 2H), 5.29 (s, 2H), 6.95 (t, J = 7.65 Hz, 0H), 7.13 (t, J = 7.5 Hz, 1H), 7.22-7.26 (m, 1H), 7.30-7.32 (m, | 441.38 |
| | | | 1H), 7.50-7.55 (m, | |
| | | | 3H), 8.49-8.51 (m, 1H). | |
| 137 | 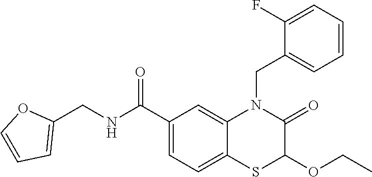 [SMILES: CCOC1Sc2ccc(C(=O)NCc3ccco3)cc2N(Cc2ccccc2F)C1=O] | 2-ethoxy-4-(2- fluorobenzyl)- N-(furan-2- ylmethyl)-3- oxo-3,4- dihydro-2H- benzo[b- 1,4]thiazine-6- carboxamide | (500 MHz; DMSO- d₆): δ 1.03 (t, J = 7.0 Hz, 3H), 3.51-3.57 (m, 1H), 3.59-3.61 (m, 1H), 3.66-3.72 (m, 1H), 4.43 (d, J = 5.55 Hz, 2H), 5.25 (d, J = 16.95 Hz, 1H), 5.44 (d, J = 16.95 Hz, 1H), 6.22 (d, J = 2.4 Hz, 1H), 6.39 (s, 1H), 6.96 | 441.26 |
| | | | (t, J = 7.55 Hz, 1H), | |
| | | | 7.12 (t, J = 7.55 Hz, | |
| | | | 1H), 7.24 (t, J = 9.85 | |
| | | | Hz, 1H), 7.30-7.34 (m, | |
| | | | 1H), 7.55-7.59 (m, | |
| | | | 3H), 7.66 (s, 1H), 9.04 | |
| | | | (t, J = 5.4 Hz, 1H). | |
| 138 | 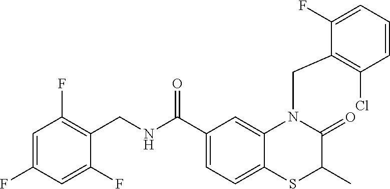 [SMILES: CC1Sc2ccc(C(=O)NCc3c(F)cc(F)cc3F)cc2N(Cc2c(F)cccc2Cl)C1=O] | 4-(2-chloro-6- fluorobenzyl)- 2-methyl-3- oxo-N-(2,4,6- trifluorobenzyl)- 3,4-dihydro- 2H-benzo[b- 1,4]thiazine-6- carboxamide | (500 MHz; DMSO- d₆): δ 1.31 (d, J = 6.95 Hz, 3H), 3.72-3.76 (q, J = 6.35 Hz, 1H), 4.45 (d, J = 4.7 Hz, 2H), 5.29 (d, J = 15.7 Hz, 1H), 5.45 (d, J = 15.75 Hz, 1H), 7.10 (t, J = 9.5 Hz, 1H), 7.20-7.30 (m, 4H), 7.46 (s, 2H), 7.72 (s, 1H), 8.86 (bs, 1H). | 509.26 |
| 139 | 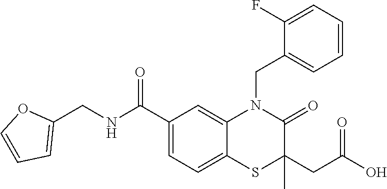 [SMILES: CC1(CC(=O)O)Sc2ccc(C(=O)NCc3ccco3)cc2N(Cc2ccccc2F)C1=O] | 2-(4-(2- fluorobenzyl)- 6-((furan-2- ylmethyl)carba moyl)-2- methyl-3-oxo- 3,4-dihydro- 2H-benzo[b- 1,4]thiazin-2- yl)acetic acid | (500 MHz; DMSO- d₆ + D₂O): δ 1.51 (s, 3H), 2.64 (s, 2H), 4.40- (d, J = 5.45 Hz, 2H), 5.22-5.30 (m, 2H), 6.21 (d, J = 3.05 Hz, 1H), 6.36-6.37 (m, 1H), 6.99 (t, J = 7.4 Hz, 1H), 7.06-7.12 (m, 1H), 7.19 (t, J = 10.05 Hz, 1H), 7.27-7.32 (m, | 469.31 |
| | | | 1H), 7.49-7.54 (m, | |
| | | | 3H), 7.60 (s, 1H), 9.06 | |
| | | | (t, J = 5.5 Hz, 1H). | |
| 140 | 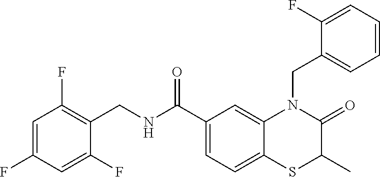 [SMILES: CC1Sc2ccc(C(=O)NCc3c(F)cc(F)cc3F)cc2N(Cc2ccccc2F)C1=O] | 4-(2- fluorobenzyl)- 2-methyl-3- oxo-N-(2,4,6- trifluorobenzyl)- 3,4-dihydro- 2H-benzo[b- 1,4]thiazine-6- carboxamide | (500 MHz; DMSO- d₆): δ 1.38 (bs, 3H), 3.91 (bs, 1H), 4.42 (bs, 2H), 5.28 (s, 2H), 7.03-7.30 (m, 6H), 7.53-7.59 (m, 3H), 8.93 (bs, 1H). | 475.26 |
| 141 | 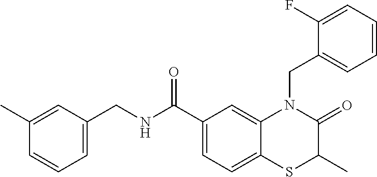 [SMILES: Cc1cccc(CNC(=O)c2ccc3c(c2)N(Cc2ccccc2F)C(=O)C(C)S3)c1] | 4-(2- fluorobenzyl)- 2-methyl-N-(3- methylbenzyl)- 3-oxo-3,4- dihydro-2H- benzo[b- 1,4]thiazine-6- carboxamide | (500 MHz; DMSO- d₆): δ 1.40 (d, J = 6.95 Hz, 3H), 2.26 (s, 3H), 3.91-3.95 (m, 1H), 4.41 (d, J = 5.85 Hz, 2H), 5.29-5.31 (m, 2H), 7.03-7.06 (m, 4H), 7.10-7.16 (m, 1H), 7.17-7.33 (m, 3H), 7.55-7.58 (m, 2H), 7.65 (s, 1H), 9.06 | 435.32 |
| | | | (t, J = 5.8 Hz, 1H). | |
| 142 | 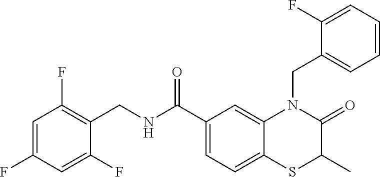 [SMILES: CC1Sc2ccc(C(=O)NCc3c(F)cc(F)cc3F)cc2N(Cc2ccccc2F)C1=O] | 4-(2- fluorobenzyl)- 2-methyl-3- oxo-N-(2,4,6- trifluorobenzyl)- 3,4-dihydro- 2H-benzo[b- 1,4]thiazine-6- carboxamide | (500 MHz; DMSO- d₆): δ 1.38 (d, J = 6.95 Hz, 3H), 3.89-3.93 (m, 1H), 4.41 (d, J = 5.0 Hz, 2H), 5.24-5.32 (m, 2H), 7.02 (t, J = 7.6 Hz, 1H), 7.11 (t, J = 7.6 Hz, 1H), 7.16-7.24 (m, 3H), 7.29-7.33 (m, 1H), 7.52 (s, 2H), 7.59 (s, 1H), 8.93 (t, J = | 475.34 |
| | | | 5.05 Hz, 1H). | |
| 143 | 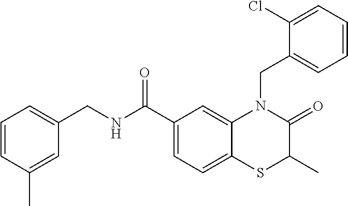 [SMILES: Cc1cccc(CNC(=O)c2ccc3c(c2)N(Cc2ccccc2Cl)C(=O)C(C)S3)c1] | 4-(2- chlorobenzyl)- 2-methyl-N-(3- methylbenzyl)- 3-oxo-3,4- dihydro-2H- benzo[b- 1,4]thiazine-6- carboxamide | 500 MHz; DMSO-d₆): δ 1.40 (d, J = 6.95 Hz, 3H), 2.26 (s, 3H), 3.95-3.99 (m, 1H), 4.39 (d, J = 5.8 Hz, 2H), 5.20 (d, J = 17.4 Hz, 1H), 5.31 (d, J = 17.35 Hz, 1H), 7.00- 7.05 (m, 4H), 7.18 (t, J = 7.45 Hz, 1H), 7.26- 7.33 (m, 2H), 7.52 (d, J = 8.25 Hz, 2H), 7.57- | 451.30 |
| | | | 7.62 (m, 2H), 9.07 (t, | |
| | | | J = 5.8 Hz, 1H). | |
| 144 | 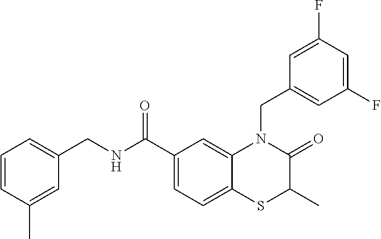 [SMILES: Cc1cccc(CNC(=O)c2ccc3c(c2)N(Cc2cc(F)cc(F)c2)C(=O)C(C)S3)c1] | 4-(3,5- difluorobenzyl)- 2-methyl-N-(3- methylbenzyl)- 3-oxo-3,4- dihydro-2H- benzo[b- 1,4]thiazine-6- carboxamide | 500 MHz; DMSO-d₆): δ 1.40 (d, J = 6.95 Hz, 3H), 2.27 (s, 3H), 3.96-4.01 (q, J = 6.8 Hz, 1H), 4.41 (d, J = 5.8 Hz, 2H), 5.22 (d, J = 17.1 Hz, 1H), 5.36 (d, J = 17.1 Hz, 1H), 6.92 (d, J = 6.7 Hz, 2H), 7.04-7.07 (m, 3H), 7.12-7.20 (m, 2H), 7.55-7.60 (m, 3H), 9.06 (t, J = 5.9 Hz, 1H). | 453.27 |
| 145 | 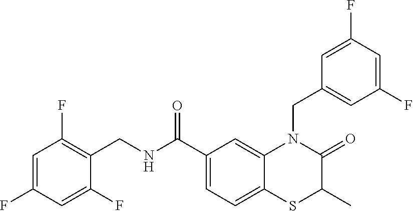 [SMILES: CC1Sc2ccc(C(=O)NCc3c(F)cc(F)cc3F)cc2N(Cc2cc(F)cc(F)c2)C1=O] | 4-(3,5- difluorobenzyl)- 2-methyl-3- oxo-N-(2,4,6- trifluorobenzyl)- 3,4-dihydro- 2H-benzo[b- 1,4]thiazine-6- carboxamide | (500 MHz; DMSO-d₆): δ 1.39 (d, J = 6.95 Hz, 3H), 3.97 (d, J = 6.95 Hz, 1H), 4.42 (s, 2H), 5.20 (d, J = 17.05 Hz, 1H), 5.34 (d, J = 17.05 Hz, 1H), 6.89 (d, J = 6.7 Hz, 2H), 7.14- 7.18 (m, 3H), 7.53 (s, 3H), 8.94 (t, J = 5.1 Hz, 1H). | 493.29 |
| 146 | 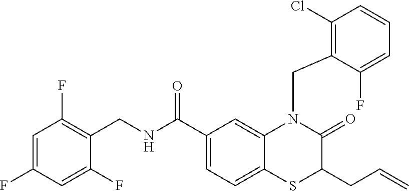 [SMILES: C=CCC1Sc2ccc(C(=O)NCc3c(F)cc(F)cc3F)cc2N(Cc2c(F)cccc2Cl)C1=O] | 2-allyl-4-(2- chloro-6- fluorobenzyl)- 3-oxo-N-(2,4,6- trifluorobenzyl)- 3,4-dihydro- 2H-benzo[b- 1,4]thiazine-6- carboxamide | (500 MHz; DMSO- d₆): δ 2.24-2.30 (m, 1H), 2.50-2.56 (m, 1H), 3.74-3.77 (m, 1H), 4.45 (d, J = 4.95 Hz, 2H), 5.05-5.08 (m, 2H), 5.34-5.42 (m 2H), 5.71-5.79 (m, 1H), 7.10 (t, J = 9.6 Hz, 1H), 7.19-7.24 (m, 3H), 7.27-7.31 (m, 1H), 7.46 (s, 2H), 7.74 | 535.31 |
| | | | (s, 1H), 8.85 (t, J = | |
| | | | 5.05 Hz, 1H). | |
| 147 | 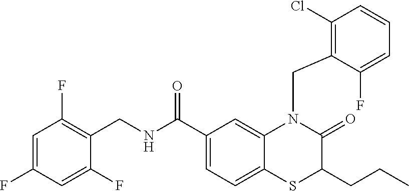 [SMILES: CCCC1Sc2ccc(C(=O)NCc3c(F)cc(F)cc3F)cc2N(Cc2c(F)cccc2Cl)C1=O] | 4-(2-chloro-6- fluorobenzyl)- 3-oxo-2-propyl- N-(2,4,6- trifluorobenzyl)- 3,4-dihydro- 2H-benzo[b- 1,4]thiazine-6- carboxamide | (500 MHz; DMSO- d₆): δ 0.83 (t, J = 7.15 Hz, 3H), 1.29-1.44 (m, 3H), 1.65-1.69 (m, 1H), 3.66 (t, J = 6.95 Hz, 1H), 4.41-4.49 (m, 2H), 5.32 (d, J = 15.75 Hz, 1H), 5.42 (d, J = 15.75 Hz, 1H), 7.11 (t, J = 9.65 Hz, 1H), 7.19- 7.31 (m, 4H), 7.46 (bs, 2H), 7.72 (s, 1H), 8.85 | 537.34 |
| | | | (t, J = 5.0 Hz, 1H). | |
| 148 | 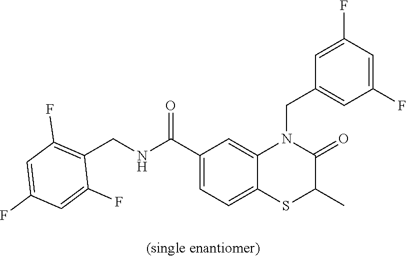 [SMILES: CC1Sc2ccc(C(=O)NCc3c(F)cc(F)cc3F)cc2N(Cc2cc(F)cc(F)c2)C1=O] | 4-(3,5- difluorobenzyl)- 2-methyl-3- oxo-N-(2,4,6- trifluorobenzyl)- 3,4-dihydro- 2H- benzo[b][1,4] thiazine-6- carboxamide | (500 MHz; DMSO- d₆): δ 1.39 (d, J = 6.8 Hz, 3H), 3.98-3.94 (m, 1H), 4.42 (s, 2H), 5.35-5.19 (m, 2H), 6.89 (d, J = 6.8 Hz, 2H), 7.18-7.13 (m, 3H), 7.53 (s, 3H), 8.92 (s, 1H). | 493.37 |
| 149 | 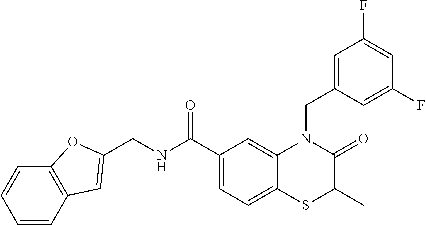 [SMILES: CC1Sc2ccc(C(=O)NCc3cc4ccccc4o3)cc2N(Cc2cc(F)cc(F)c2)C1=O] | N-(benzofuran- 2-ylmethyl)-4- (3,5- difluorobenzyl)- 2-methyl-3- oxo-3,4- dihydro-2H- benzo[b][1,4] thiazine-6- carboxamide | (500 MHz; DMSO- d₆): δ 1.41 (d, J = 6.9 Hz, 3H), 4.00-3.96 (m, 1H), 4.61 (d, J = 5.6 Hz, 2H), 5.38-5.21 (m, 2H), 6.70 (s, 1H), 6.92 (d, J = 6.7 Hz, 2H), 7.13 (t, J = 9.3 Hz, 1H), 7.28-7.20 (m, 2H), 7.62-7.51 (m, 5H), 9.17 (t, J = 5.6 Hz, 1H). | 479.33 |
| 150 | 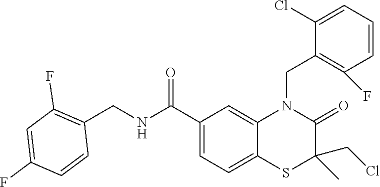 [SMILES: CC1(CCl)Sc2ccc(C(=O)NCc3ccc(F)cc3F)cc2N(Cc2c(F)cccc2Cl)C1=O] | 4-(2-chloro-6- fluorobenzyl)-2- (chloromethyl)- N-(2,4- difluorobenzyl)- 2-methyl-3- oxo-3,4- dihydro-2H- benzo[b][1,4] thiazine-6- carboxamide | (400 MHz; DMSO- d₆): δ 1.43 (s, 3H), 3.81-3.67 (m, 2H), 4.44 (d, J = 3.2 Hz, 2H), 5.50-5.31 (m, 2H), 7.11-7.07 (m, 2H), 7.31-7.19 (m, 3H), 7.40-7.34 (m, 1H), 7.55-7.48 (m, 2H), 7.81 (s, 1H), 9.00 (t, J = 5.6 Hz, 1H). | 539.28 |
| 151 | 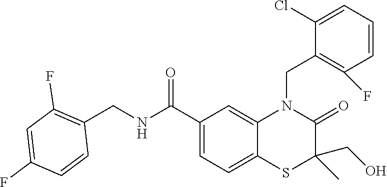 [SMILES: CC1(CO)Sc2ccc(C(=O)NCc3ccc(F)cc3F)cc2N(Cc2c(F)cccc2Cl)C1=O] | 4-(2-chloro-6- fluorobenzyl)- N-(2,4- difluorobenzyl)-2- (hydroxymethyl)- 2-methyl-3- oxo-3,4- dihydro-2H- benzo[b][1,4] thiazine-6- carboxamide | (400 MHz; DMSO- d₆): δ 1.31 (s, 3H), 3.43 (d, J = 6 Hz, 2H), 4.43 (d, J = 5.6 Hz, 2H), 5.27-5.20 (m, 2H), 5.49 (d, J = 15.8 Hz, 1H), 7.11-7.04 (m, 2H), 7.50-7.18 (m, 6H), 7.72 (bs, 1H), 8.96 (t, J = 5.6 Hz, 1H). | 521.30 |
| 152 | 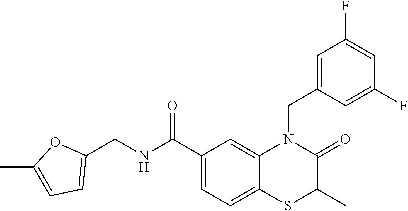 [SMILES: Cc1ccc(CNC(=O)c2ccc3c(c2)N(Cc2cc(F)cc(F)c2)C(=O)C(C)S3)o1] | 4-(3,5- difluorobenzyl)- 2-methyl-N-((5- methylfuran-2- yl)methyl)-3- oxo-3,4- dihydro-2H- benzo[b][1,4] thiazine-6- carboxamide | (500 MHz; DMSO-d₆): δ 1.40 (d, J = 6.9 Hz, 3H), 2.21 (s, 3H), 4.00-3.95 (s, 1H), 4.37 (d, J = 5.5 Hz, 2H), 5.37-5.20 (m, 2H), 5.97 (d, J = 1.9 Hz, 1H), 6.08 (d, J = 2.8 Hz, 1H), 6.91 (d, J = 6.6 Hz, 2H), 7.14 (t, J = 9.3 Hz, 1H), 7.57- 7.54 (m, 3H), 8.97 (t, J = 5.6 Hz, 1H). | 443.27 |
| 153 | 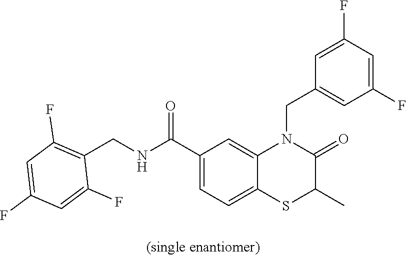 [SMILES: CC1Sc2ccc(C(=O)NCc3c(F)cc(F)cc3F)cc2N(Cc2cc(F)cc(F)c2)C1=O] | 4-(3,5- difluorobenzyl)- 2-methyl-3- oxo-N-(2,4,6- trifluorobenzyl)- 3,4-dihydro- 2H- benzo[b][1,4] thiazine-6- carboxamide | (500 MHz; DMSO- d₆): δ 1.39 (d, J = 6.8 Hz, 3H), 3.98-3.94 (m, 1H), 4.42 (s, 2H), 5.35-5.19 (m, 2H), 6.89 (d, J = 6.9 Hz, 2H), 7.18-7.13 (m, 3H), 7.53 (s, 3H), 8.92 (s, 1H). | 493.37 |
| 154 | 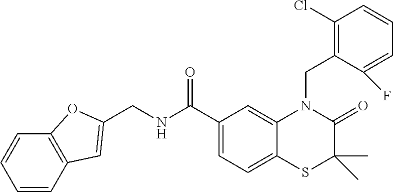 [SMILES: CC1(C)Sc2ccc(C(=O)NCc3cc4ccccc4o3)cc2N(Cc2c(F)cccc2Cl)C1=O] | N-(benzofuran- 2-ylmethyl)-4- (2-chloro-6- fluorobenzyl)- 2,2-dimethyl-3- oxo-3,4- dihydro-2H- benzo[b][1,4] thiazine-6- carboxamide | (400 MHz; DMSO- d₆): δ 1.32 (s, 6H), 4.60 (d, J = 5.5 Hz, 2H), 5.37 (S, 2H), 6.72 (s, 1H), 7.11-7.06 (m, lH), 7.28-7.18 (m, 4H), 7.58-7.46 (m, 4H), 7.78 (s, 1H), 9.11 (t, J = 5.6 Hz, 1H). | 509.22 |
| 155 | 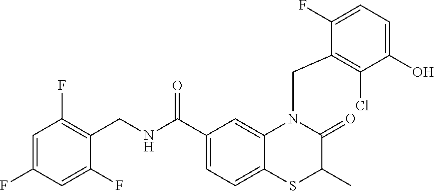 [SMILES: CC1Sc2ccc(C(=O)NCc3c(F)cc(F)cc3F)cc2N(Cc2c(F)ccc(O)c2Cl)C1=O] | 4-(2-chloro-6- fluoro-3- hydroxybenzyl)- 2-methyl-3- oxo-N-(2,4,6- trifluorobenzyl)- 3,4-dihydro- 2H- benzo[b][1,4] thiazine-6- carboxamide | (500 MHz; DMSO- d₆): δ 1.31 (d, J = 6.85 Hz, 3H), 3.72 (d, J = 6.9 Hz, 1H), 4.44 (d, J = 4.25 Hz, 2H), 5.26 (d, J = 15.65 Hz, 1H), 5.40 (d, J = 15.60 Hz, 1H), 6.81-6.92 (m, 2H), 7.18-7.21 (m, 2H), 7.46 (s, 2H), 7.69 (s, 1H), 8.38 (s, 1H), 10.09 (s, 1H). | 525.13 |

| | | | LCMS |
|---|---|---|---|
| Example | Structure | IUPAC Name | [M + H]⁺ |
| 156 | 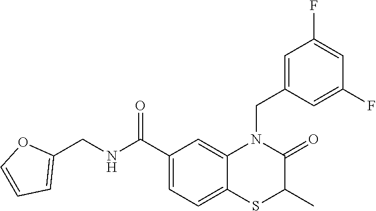 [SMILES: CC1Sc2ccc(C(=O)NCc3ccco3)cc2N(Cc2cc(F)cc(F)c2)C1=O] | 4-(3,5- difluorobenzyl)- N-(furan-2- ylmethyl)-2-methyl- 3- oxo-3,4- dihydro-2H-benzo [b][1,4]thiazine-6- carboxamide | 429.33 |
| 157 | 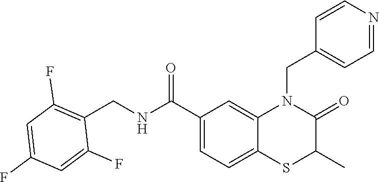 [SMILES: CC1Sc2ccc(C(=O)NCc3c(F)cc(F)cc3F)cc2N(Cc2ccncc2)C1=O] | 2-methyl-3-oxo-4- (pyridin-4- ylmethyl)-N-(2,4,6- trifluorobenzyl)- 3,4-dihydro-2H- benzo[b][1,4] thiazine-6- carboxamide | 458.35 |
| 158 | 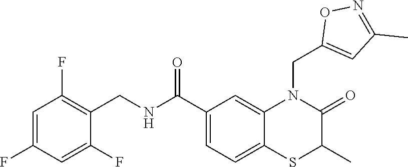 [SMILES: Cc1cc(CN2C(=O)C(C)Sc3ccc(C(=O)NCc4c(F)cc(F)cc4F)cc32)on1] | 2-methyl-4-((3- methylisoxazol- 5-yl)methyl)-3-oxo- N-(2,4,6- trifluorobenzyl)- 3,4-dihydro-2H- benzo[b][1,4] thiazine- 6-carboxamide | 461.56 |
| 159 | 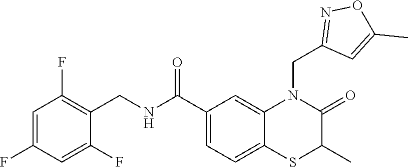 [SMILES: Cc1cc(CN2C(=O)C(C)Sc3ccc(C(=O)NCc4c(F)cc(F)cc4F)cc32)no1] | 2-methyl-4-((5- methylisoxazol- 3-yl)methyl)-3- oxo-N-(2,4,6- trifluorobenzyl)- 3,4-dihydro-2H- benzo[b][1,4] thiazine-6- carboxamide | 474.56 |
| 160 | 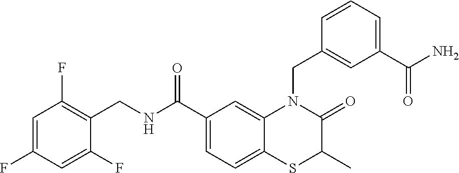 [SMILES: CC1Sc2ccc(C(=O)NCc3c(F)cc(F)cc3F)cc2N(Cc2cccc(C(N)=O)c2)C1=O] | 4-(3- carbamoylbenzyl)- 2-methyl-3- oxo-N- (2,4,6- trifluorobenzyl)- 3,4-dihydro-2H- benzo[b][1,4] thiazine-6- carboxamide | 500.17 |
| 161 | 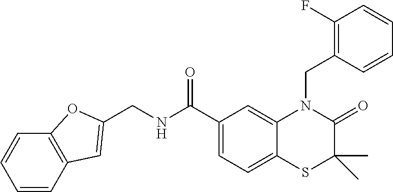 [SMILES: CC1(C)Sc2ccc(C(=O)NCc3cc4ccccc4o3)cc2N(Cc2ccccc2F)C1=O] | N-(benzofuran-2- ylmethyl)-4-(2- fluorobenzyl)- 2,2-dimethyl-3-oxo- 3,4-dihydro-2H- benzo[b][1,4] thiazine-6- carboxamide | 475.36 |
| 162 | 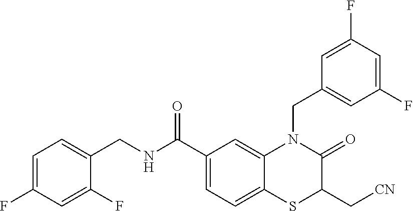 [SMILES: N#CCC1Sc2ccc(C(=O)NCc3ccc(F)cc3F)cc2N(Cc2cc(F)cc(F)c2)C1=O] | 2-(cyanomethyl)- N-(2,4- difluorobenzyl)- 4-(3,5- difluorobenzyl)-3- oxo-3,4-dihydro- 2H-benzo[b][1,4] thiazine-6- carboxamide | 500.32 |
| 163 | 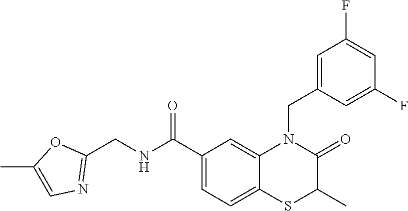 [SMILES: Cc1cnc(CNC(=O)c2ccc3c(c2)N(Cc2cc(F)cc(F)c2)C(=O)C(C)S3)o1] | 4-(3,5- difluorobenzyl)- 2-methyl-N- ((5-methyloxazol- 2-yl)methyl)-3- oxo-3,4-dihydro- 2H-benzo[b][1,4] thiazine-6- carboxamide | 444.34 |
| 164 | 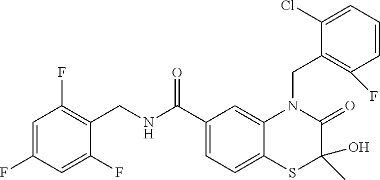 [SMILES: CC1(O)Sc2ccc(C(=O)NCc3c(F)cc(F)cc3F)cc2N(Cc2c(F)cccc2Cl)C1=O] | 4-(2-chloro- 6-fluorobenzyl)- 2-hydroxy-2- methyl-3-oxo-N- (2,4,6- trifluorobenzyl)- 3,4-dihydro-2H- benzo[b][1,4] thiazine-6- carboxamide | 525.31 |
| 165 | 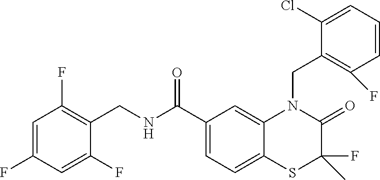 [SMILES: CC1(F)Sc2ccc(C(=O)NCc3c(F)cc(F)cc3F)cc2N(Cc2c(F)cccc2Cl)C1=O] | 4-(2- chloro-6- fluorobenzyl)-2- fluoro-2-methyl- 3-oxo-N-(2,4,6- trifluorobenzyl)- 3,4-dihydro-2H- benzo[b][1,4] thiazine-6- carboxamide | 527.28 |
| 166 | 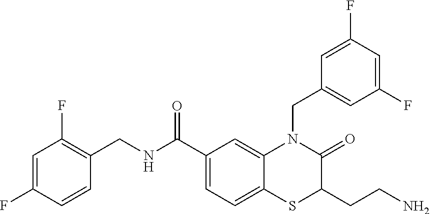 [SMILES: NCCC1Sc2ccc(C(=O)NCc3ccc(F)cc3F)cc2N(Cc2cc(F)cc(F)c2)C1=O] | 2-(2- aminoethyl)- N-(2,4- difluorobenzyl)- 4-(3,5- difluorobenzyl)- 3-oxo-3,4- dihydro- 2H-benzo[b] [1,4]thiazine- 6- carboxamide | 504.32 |
| 167 | 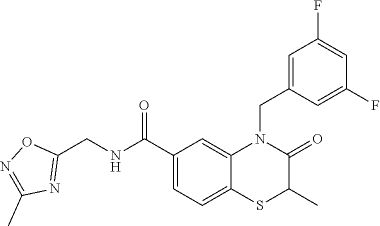 [SMILES: Cc1noc(CNC(=O)c2ccc3c(c2)N(Cc2cc(F)cc(F)c2)C(=O)C(C)S3)n1] | 4-(3,5- difluorobenzyl)- 2-methyl-N- ((3-methyl-1,2,4- oxadiazol-5- yl)methyl)-3- oxo-3,4-dihydro- 2H- benzo[b][1,4] thiazine-6- carboxamide | 445.38 |
| 168 | 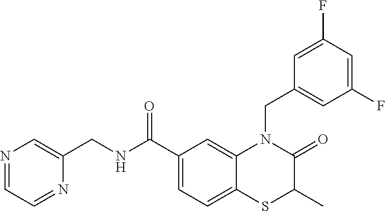 [SMILES: CC1Sc2ccc(C(=O)NCc3cnccn3)cc2N(Cc2cc(F)cc(F)c2)C1=O] | 4-(3,5- difluorobenzyl)- 2-methyl-3- oxo-N-(pyrazin- 2-ylmethyl)- 3,4- dihydro- 2H-benzo [b][1,4]thiazine- 6- carboxamide | 441.54 |
| 169 | 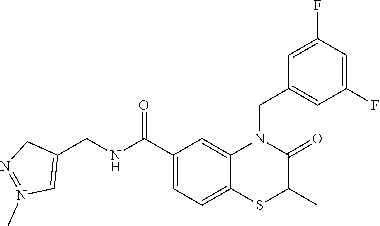 | 4-(3,5- difluorobenzyl)- 2-methyl-N- ((1-methyl-1H- pyrazol-4-yl) methyl)- 3-oxo-3,4- dihydro-2H- benzo[b][1,4] thiazine-6- carboxamide | 443.55 |
| 170 | 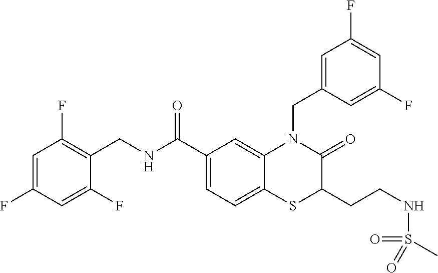 [SMILES: CS(=O)(=O)NCCC1Sc2ccc(C(=O)NCc3c(F)cc(F)cc3F)cc2N(Cc2cc(F)cc(F)c2)C1=O] | 4-(3,5- difluorobenzyl)- 2-(2- (methyl- sulfonamido) ethyl)-3-oxo-N- (2,4,6- trifluorobenzyl)- 3,4-dihydro- 2H-benzo[b] [1,4]thiazine- 6- carboxamide | 600.59 |
| 171 | 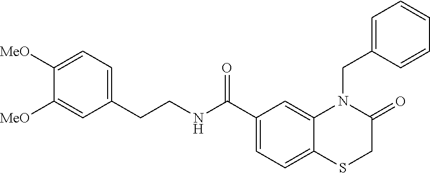 [SMILES: COc1ccc(CCNC(=O)c2ccc3c(c2)N(Cc2ccccc2)C(=O)CS3)cc1OC] | 4-benzyl-N- (3,4- dimethoxy- phenethyl)- 3-oxo-3,4- dihydro-2H- benzo[b][1,4] thiazine-6- carboxamide | 463.2 |
| 172 | 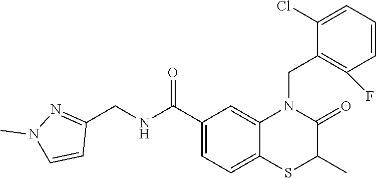 [SMILES: CC1Sc2ccc(C(=O)NCc3ccn(C)n3)cc2N(Cc2c(F)cccc2Cl)C1=O] | 4-(2-chloro- 6-fluorobenzyl)- 2-methyl-N- ((1-methyl-1H- pyrazol-3- yl)methyl)-3- oxo-3,4-dihydro- 2H-benzo[b] [1,4]thiazine-6- carboxamide | 459.16 |
| 173 | 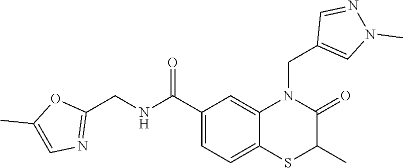 | 2-methyl-4- ((1-methyl-1H- pyrazol-4- yl)methyl)-N- ((5-methyloxazol- 2-yl)methyl)- 3-oxo-3,4-dihydro- 2H-benzo[b][1,4] thiazine-6- carboxamide | 412.14 |
| 174 | 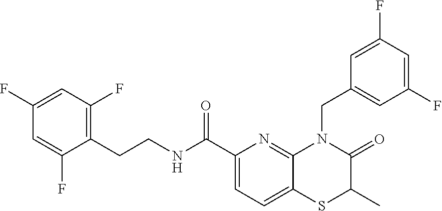 [SMILES: CC1Sc2ccc(C(=O)NCCc3c(F)cc(F)cc3F)nc2N(Cc2cc(F)cc(F)c2)C1=O] | 4-(3,5- difluorobenzyl)- 2-methyl-3- oxo-N-(2,4,6- trifluoro- phenethyl)- 3,4-dihydro-2H- pyrido[3,2- b][1,4]thiazine- 6-carboxamide | 508.3 |
| 175 | 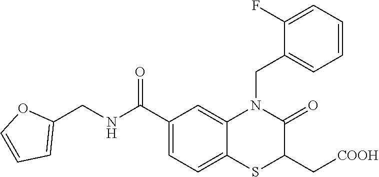 [SMILES: O=C(O)CC1Sc2ccc(C(=O)NCc3ccco3)cc2N(Cc2ccccc2F)C1=O] | 2-(4-(2- fluorobenzyl)- 6-((furan-2- ylmethyl) carbamoyl)- 3-oxo-3,4- dihydro-2H- benzo[b][1,4] thiazin-2- yl)acetic acid | 455.3 |
| 176 | 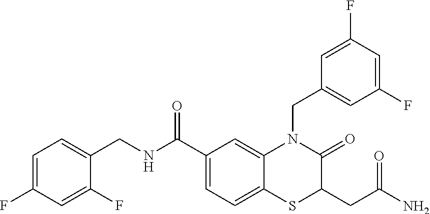 [SMILES: NC(=O)CC1Sc2ccc(C(=O)NCc3ccc(F)cc3F)cc2N(Cc2cc(F)cc(F)c2)C1=O] | 2-(2-amino-2- oxoethyl)-N- (2,4- difluorobenzyl)- 4-(3,5- difluorobenzyl)- 3-oxo-3,4- dihydro- 2H-benzo[b] [1,4]thiazine-6- carboxamide | 518.31 |

### Example 177: 1-(2-Chloro-6-fluorobenzyl)-N-(furan-2-ylmethyl)-2-oxo-2,3-dihydro-1H-pyrido[2,3-b-1,4]thiazine-7-carboxamide

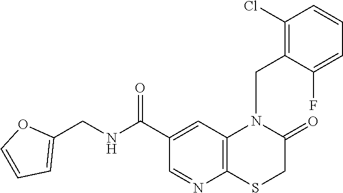 [SMILES: O=C(NCc1ccco1)c1cnc2c(c1)N(Cc1c(F)cccc1Cl)C(=O)CS2]

### Preparation 11: Methyl 6-((2-ethoxy-2-oxoethyl)thio)-5-nitronicotinate

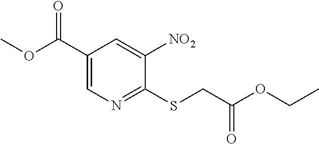 [SMILES: CCOC(=O)CSc1ncc(C(=O)OC)cc1[N+](=O)[O-]]

To a stirred solution of methyl-6-chloro-5-nitronicotinate (0.8 g, 3.70 mmol) in acetonitrile (10 mL) was added TEA (1.03 mL, 7.41 mmol) followed by ethyl thioglycolate (0.50 mL, 5.63 mmol) dropwise at RT. The reaction mixture was stirred for 15 min. and then diluted with EtOAc and washed with water and brine. The organic layer was dried over anhydrous Na₂SO₄ and evaporated under reduced pressure to dryness to give methyl 6-((2-ethoxy-2-oxoethyl)thio)-5-nitronicotinate (1.1 g, 3.66 mmol, 99% yield) as a yellowish solid, which was pure enough to be used in the next step without any further purification. LCMS m/z: 301.07 [M+H].

### Preparation 12: Methyl 2-oxo-2,3-dihydro-1H-pyrido[2,3-b][1,4]thiazine-7-carboxylate

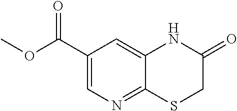

To a stirred solution of methyl 6-((2-ethoxy-2-oxoethyl)thio)-5-nitronicotinate (Preparation 11) (1.0 g, 3.66 mmol) in AcOH (10 mL) was added iron powder (0.819 g, 14.66 mmol) at RT. The resulting reaction mixture was stirred at 90° C. for 2.5 h, after which time the reaction mixture was diluted with EtOAc and washed with water. The organic layer was dried over anhydrous Na₂SO₄ and evaporated under reduced pressure to dryness to give methyl 2-oxo-2,3-dihydro-1H-pyrido[2,3-b][1,4]thiazine-7-carboxylate (1.0 g, 4.46 mmol, 99% yield) as a brown solid, which was pure enough to be used in the next step without any further purification. LCMS m/z: 225.03 [M+H].

### Preparation 13: 1-(2-Chloro-6-fluorobenzyl)-N-(furan-2-ylmethyl)-2-oxo-2,3-dihydro-1H-pyrido[2,3-b-1,4]thiazine-7-carboxamide

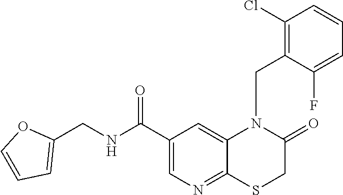 [SMILES: O=C(NCc1ccco1)c1cnc2c(c1)N(Cc1c(F)cccc1Cl)C(=O)CS2]

1-(2-Chloro-6-fluorobenzyl)-N-(furan-2-ylmethyl)-2-oxo-2,3-dihydro-1H-pyrido[2,3-b-1,4]thiazine-7-carboxamide (Example 177) was made from methyl 2-oxo-2,3-dihydro-1H-pyrido[2,3-b][1,4]thiazine-7-carboxylate (Preparation 12) using the methods described in Preparation 4, Preparation 5 and Preparation 6, using 2-chloro-6-fluoro-benzyl chloride and 1-(furan-2-yl)-methanamine. LCMS m/z: 432.05 [M+H]; ¹H NMR (500 MHz; DMSO-d₆): δ 3.77 (s, 2H), 4.48 (d, J=5.5 Hz, 2H), 5.34 (s, 2H), 6.29 (d, J=2.35 Hz, 1H), 6.42 (s, 1H), 7.14 (t, J=9.25 Hz, 1H), 7.25-7.33 (m, 2H), 7.61 (s, 1H), 8.03 (s, 1H), 8.54 (s, 1H), 9.14 (t, J=5.5 Hz, 1H).

### Examples 178-218

Examples 178-218 were prepared according to the methods described for the synthesis of Example 177 as described in General Procedures 4 and 5 and 1-3 starting from an appropriate pyridine and using the appropriate benzyl halides and amines.

| | | | | LCMS |
|---|---|---|---|---|
| Example | Structure | IUPAC Name | ¹H-NMR | [M + H] |
| 178 | 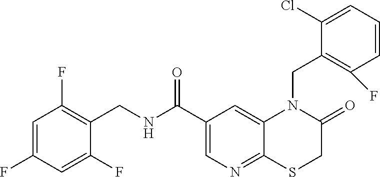 [SMILES: O=C(NCc1c(F)cc(F)cc1F)c1cnc2c(c1)N(Cc1c(F)cccc1Cl)C(=O)CS2] | 1-(2-chloro-6- fluorobenzyl)- 2-oxo-N-(2,4,6- trifluorobenzyl)- 2,3-dihydro- 1H-pyrido[2,3- b-1,4]thiazine- 7-carboxamide | (500 MHz; DMSO- d₆): δ 3.76 (s, 2H), 4.47 (d, J = 4.85 Hz, 2H), 5.32 (s, 2H), 7.11- 7.33 (m, 5H), 7.98 (s, 1H), 8.49 (s, 1H), 9.03 (t, J = 4.95 Hz, lH). | 469.19 |
| 179 | 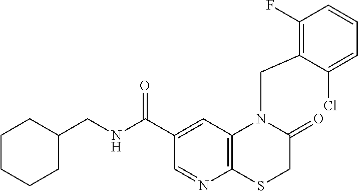 [SMILES: O=C(NCC1CCCCC1)c1cnc2c(c1)N(Cc1c(F)cccc1Cl)C(=O)CS2] | 1-(2-chloro-6- fluorobenzyl)- N- (cyclohexyl- methyl)- 2-oxo-2,3- dihydro-1H- pyrido[2,3-b- 1,4]thiazine-7- carboxamide | (500 MHz; DMSO- d₆): δ 0.87-0.94 (m, 2H), 1.13-1.23 (m, 3H), 1.51-1.70 (m, 6H), 3.08-3.11 (m, 2H), 3.77 (s, 2H), 5.35 (s, 2H), 7.13 (t, J = 9.05 Hz, 1H), 7.24- 7.31 (m, 2H), 7.96 (s, 1H), 8.49 (s, 1H), 8.59 (bs, 1H). | 448.24 |
| 180 | 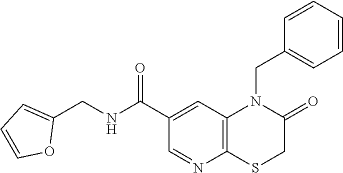 [SMILES: O=C(NCc1ccco1)c1cnc2c(c1)N(Cc1ccccc1)C(=O)CS2] | 1-benzyl-N- (furan-2- ylmethyl)-2- oxo-2,3- dihydro-1H- pyrido[2,3-b- 1,4]thiazine-7- carboxamide | (500 MHz; DMSO- d₆): δ 3.92 (s, 2H), 4.45 (d, J = 5.35 Hz, 2H), 5.24 (s, 2H), 6.26 (d, J = 2.15 Hz, 1H), 6.40 (s, 1H), 6.20-6.25 (m, 3H), 7.31-7.34 (m, 2H), 7.59 (s, 1H), 7.86 (s, 1H), 8.57 (s, 1H), 9.16 (t, J = 5.15 1H). | 380.18 |
| 181 | 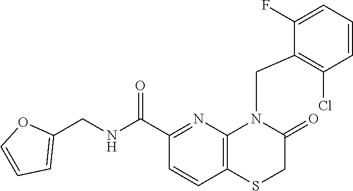 [SMILES: O=C(NCc1ccco1)c1ccc2c(n1)N(Cc1c(F)cccc1Cl)C(=O)CS2] | 4-(2-chloro-6- fluorobenzyl)- N-(furan-2- ylmethyl)-3- oxo-3,4- dihydro-2H- pyrido[3,2-b- 1,4]thiazine-6- carboxamide | (400 MHz; DMSO- d₆): δ 3.70 (s, 2H), 4.49 (d, J = 5.76 Hz, 2H), 5.61 (s, 2H), 6.25 (s, 1H), 6.40 (s, 1H), 7.06 (t, J = 8.52 Hz, 1H), 7.21-7.28 (m, 2H), 7.58 (s, 1H), 7.68 (d, J = 7.88 Hz, 1H), 8.04 (d, J = 7.96 Hz, 1H), 8.83 (bs, 1H). | 432.0 |
| 182 | 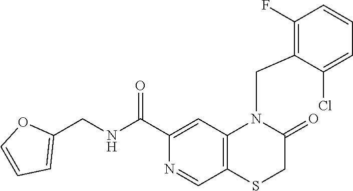 [SMILES: O=C(NCc1ccco1)c1cc2c(cn1)SCC(=O)N2Cc1c(F)cccc1Cl] | 1-(2-chloro-6- fluorobenzyl)- N-(furan-2- ylmethyl)-2- oxo-2,3- dihydro-1H- pyrido[3,4-b- 1,4]thiazine-7- carboxamide | (400 MHz; DMSO- d₆): δ 3.70 (s, 2H), 4.43 (d, J = 6.08 Hz, 2H), 5.41 (s, 2H), 6.21 (d, J = 3.0 Hz, 1H), 6.37 (t, J = 2.88 Hz, 1H), 7.15-7.17 (m, 1H), 7.26-7.32 (m, 2H), 7.54 (s, 1H), 7.81 (s, 1H), 8.56 (s, 1H), 9.04-9.06 (m, 1H). | 432.0 |
| 183 | 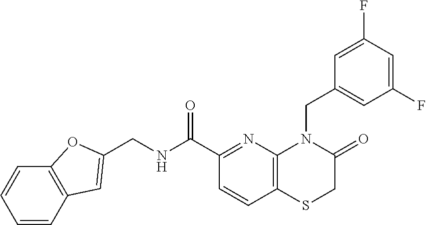 [SMILES: O=C(NCc1cc2ccccc2o1)c1ccc2c(n1)N(Cc1cc(F)cc(F)c1)C(=O)CS2] | N-(benzofuran- 2-ylmethyl)-4- (3,5- difluorobenzyl)- 3-oxo-3,4- dihydro-2H- pyrido[3,2-b- 1,4]thiazine-6- carboxamide | (400 MHz; DMSO- d₆): δ 3.85 (s, 2H), 4.65 (d, J = 5.32 Hz, 2H), 5.57 (s, 2H), 6.58 (s, 1H), 6.95-7.01 (m, 3H), 7.19-7.27 (m, 2H), 7.49-7-55 (m, 2H), 7.69 (d, J = 7.88 Hz, 1H), 8.06 (d, J = 7.80 Hz, 1H), 9.09 (bs, 1H). | 466.1 |
| 184 | 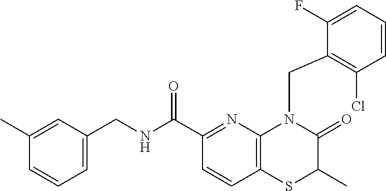 [SMILES: Cc1cccc(CNC(=O)c2ccc3c(n2)N(Cc2c(F)cccc2Cl)C(=O)C(C)S3)c1] | 4-(2-chloro-6- fluorobenzyl)- 2-methyl-N-(3- methylbenzyl)- 3-oxo-3,4- dihydro-2H- pyrido[3,2-b- 1,4]thiazine-6- carboxamide | (400 MHz; DMSO- d₆): δ 1.33 (d, J = 7.0 Hz, 3H), 2.26 (s, 3H), 3.88-3.91 (m, 1H), 4.46 (t, J = 5.56 Hz, 2H), 5.54 (d, J = 15.08 Hz, 1H), 5.75 (d, J = 15.16 Hz, 1H), 7.02- 7.09 (m, 4H), 7.18- 7.27 (m, 3H), 7.71 (d, J = 7.88 Hz, 1H), 8.06 (d, J = 7.88 Hz, 1H), | 470.3 |
| | | | 8.93-8.95 (m, 1H). | |
| 185 | 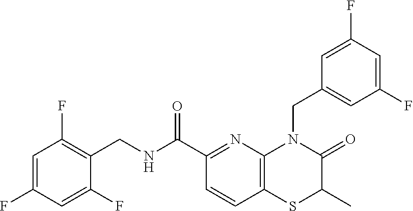 [SMILES: CC1Sc2ccc(C(=O)NCc3c(F)cc(F)cc3F)nc2N(Cc2cc(F)cc(F)c2)C1=O] | 4-(3,5- difluorobenzyl)- 2-methyl-3- oxo-N-(2,4,6- trifluorobenzyl)- 3,4-dihydro- 2H-pyrido[3,2- b-1,4]thiazine- 6-carboxamide | (400 MHz; DMSO-d₆): δ 1.40 (d, J = 6.96 Hz, 3H), 4.07-4.09 (m, 1H), 4.49 (t, J = 3.84 Hz, 2H), 4.99 (d, J = 15.44 Hz, 1H), 5.75 (d, J = 15.4 Hz, 1H), 6.88 (d, J = 6.48 Hz, 2H), 7.02-7.03 (m, 1H), 7.11 (t, J = 8.72 Hz, 2H), 7.65 (d, J = 7.88 Hz, 1H), 8.04 (d, J = 7.88 Hz, 1H), 8.76 (m, 1H). | 494.1 |
| 186 | 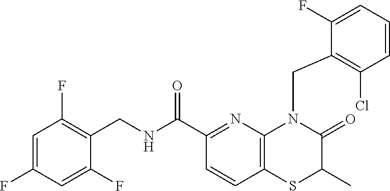 [SMILES: CC1Sc2ccc(C(=O)NCc3c(F)cc(F)cc3F)nc2N(Cc2c(F)cccc2Cl)C1=O] | 4-(2-chloro-6- fluorobenzyl)- 2-methyl-3- oxo-N-(2,4,6- trifluorobenzyl)- 3,4-dihydro- 2H-pyrido[3,2- b-1,4]thiazine- 6-carboxamide | (400 MHz; DMSO-d₆): δ 1.33 (d, J = 6.88 Hz, 3H), 3.88-3.93 (q, J = 7.16 Hz, 1H), 4.52 (t, J = 6.04 Hz, 2H), 5.51 (d, J = 15.32 Hz, 1H), 5.66 (d, J = 15.24 Hz, 1H), 7.03 (t, J = 9.56 Hz, 1H), 7.17-7.27 (m, 4H), 7.66 (d, J = 7.84 Hz, H), 8.04 (d, J = 7.80 Hz, 1H), 8.65 (bs, 1H). | 510.2 |
| 187 | 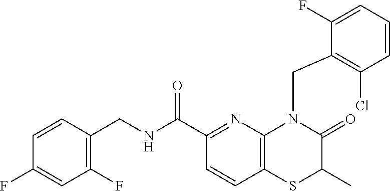 [SMILES: CC1Sc2ccc(C(=O)NCc3ccc(F)cc3F)nc2N(Cc2c(F)cccc2Cl)C1=O] | 4-(2-chloro-6- fluorobenzyl)- N-(2,4- difluorobenzyl)- 2-methyl-3- oxo-3,4- dihydro-2H- pyrido[3,2-b- 1,4]thiazine-6- carboxamide | (400 MHz; DMSO-d₆): δ 1.33 (d, J = 6.88 Hz, 3H), 3.90-3.91 (m, 1H), 4.51 (s, 2H), 5.55 (d, J = 15.40 Hz, 1H), 5.75 (d, J = 15.08 Hz, 1H), 7.04-7.06 (m, 2H), 7.21-7.28 (m, 3H), 7.32-7-36 (m, 1H), 7.70 (d, J = 7.76 Hz, 1H), 8.06 (d, J = 7.68 Hz, 1H), 8.98 (bs, 1H). | 492.2 |
| 188 | 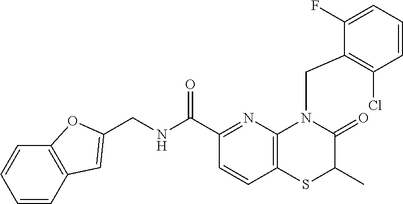 [SMILES: CC1Sc2ccc(C(=O)NCc3cc4ccccc4o3)nc2N(Cc2c(F)cccc2Cl)C1=O] | N-(benzofuran- 2-ylmethyl)-4- (2-chloro-6- fluorobenzyl)- 2-methyl-3- oxo-3,4- dihydro-2H- pyrido[3,2-b- 1,4]thiazine-6- carboxamide | (400 MHz; DMSO-d₆): δ 1.32 (d, J = 6.68 Hz, 3H), 3.89-3.91 (m, 1H), 4.66 (s, 2H), 5.56 (d, J = 15.24 Hz, 1H), 5.76 (d, J = 14.88 Hz, 1H), 6.72 (bs, 1H), 7.05-7.08 (m, 1H), 7.19-7.26 (m, 4H), 7.52 (d, J = 6.76 Hz, 1H), 7.58 (d, J = 8.08 Hz, 1H), 7.72 (d, J = | 496.2 |
| | | | 8.0 Hz, 1H), 8.07 (d, J = | |
| | | | 7.88 Hz, 1H), 9.05 | |
| | | | (bs, 1H). | |
| 189 | 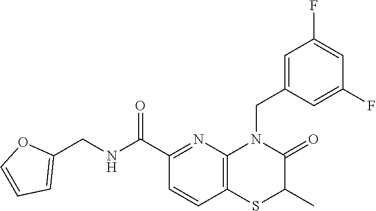 [SMILES: CC1Sc2ccc(C(=O)NCc3ccco3)nc2N(Cc2cc(F)cc(F)c2)C1=O] | 4-(3,5- difluorobenzyl)- N-(furan-2- ylmethyl)-2- methyl-3-oxo- 3,4-dihydro- 2H-pyrido[3,2- b-1,4]thiazine- 6-carboxamide | (400 MHz; DMSO- d₆): δ 1.40 (d, J = 6.76 Hz, 3H), 4.08 (d, J = 7.0 Hz, 1H), 4.48 (d, J = 4.92 Hz, 2H), 5.49 (d, J = 15.48 Hz, 1H), 5.61 (d, J = 15.28 Hz, 1H), 6.16 (s, 1H), 6.37 (s, 1H), 6.92 (d, J = 6.24 Hz, 2H), 7.05- 7.07 (m, 1H), 7.53 (s, 1H), 7.69 (d, J = 7.84 Hz, 1H), 8.04 (d, J = | 429.9 |
| | | | 7.88 Hz, 1H), 8.95 (bs, | |
| | | | 1H). | |
| 190 | 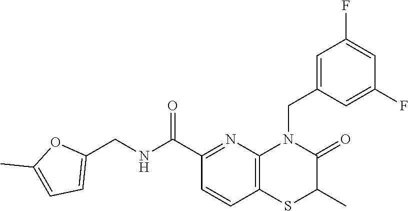 [SMILES: Cc1ccc(CNC(=O)c2ccc3c(n2)N(Cc2cc(F)cc(F)c2)C(=O)C(C)S3)o1] | 4-(3,5- difluorobenzyl)- 2-methyl-N-((5- methylfuran-2- yl)methyl)-3- oxo-3,4- dihydro-2H- pyrido[3,2-b- 1,4]thiazine-6- carboxamide | (400 MHz; DMSO- d₆): δ 1.40 (d, J = 6.64 Hz, 3H), 2.20 (s, 3H), 4.08 (d, J = 6.56 Hz, 1H), 4.40 (s, 2H), 5.49 (d, J = 15.12 Hz, 1H), 5.61 (d, J = 15.16 Hz, 1H), 5.95 (s, 1H), 6.02 (s, 1H), 6.92 (d, J = 6.84 Hz, 2H), 7.05 (m, 1H), 7.69 (d, J = 7.56 Hz, 1H), 8.05 (d, J = 7.80 Hz, 1H), 8.88 (bs, 1H). | 444.2 |
| 191 | 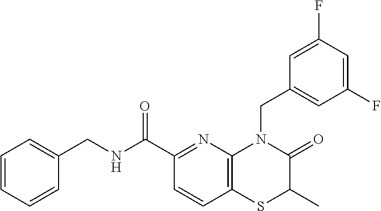 [SMILES: CC1Sc2ccc(C(=O)NCc3ccccc3)nc2N(Cc2cc(F)cc(F)c2)C1=O] | N-benzyl-4- (3,5- difluorobenzyl)- 2-methyl-3- oxo-3,4- dihydro-2H- pyrido[3,2-b- 1,4]thiazine-6- carboxamide | (400 MHz; DMSO- d₆): δ 1.41 (d, J = 6.80 Hz, 3H), 4.08-4.11 (m, 1H), 4.50 (s, 2H), 5.52 (d, J = 15.44 Hz, 1H), 5.63 (d, J = 15.32 Hz, 1H), 6.94 (d, J = 6.2 Hz, 2H), 7.03-7.05 (m, 1H), 6.23-7.28 (m, 5H), 7.69 (d, J = 7.80 Hz, 1H), 8.06 (d, J = 7.84 Hz, 1H), 9.09 (bs, 1H). | 437.9 [M − H] |
| 192 | 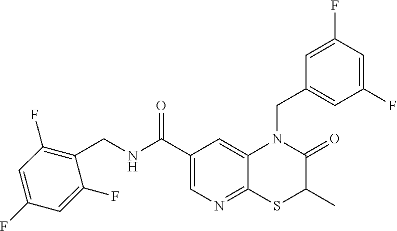 [SMILES: CC1Sc2ncc(C(=O)NCc3c(F)cc(F)cc3F)cc2N(Cc2cc(F)cc(F)c2)C1=O] | 1-(3,5- difluorobenzyl)- 3-methyl-2- oxo-N-(2,4,6- trifluorobenzyl)- 2,3-dihydro- 1H-pyrido[2,3- b][1,4]thiazine- 7-carboxamide | (400 MHz; DMSO- d₆): δ 1.42 (d, J = 7.0 Hz, 3H), 4.19-4.14 (m, 1H), 4.42 (d, J = 5.0 Hz, 2H), 5.31-5.11 (m, 2H), 6.89 (d, J = 6.4 Hz, 2H), 7.17-7.09 (m, 3H), 7.73 (d, J = 1.7 Hz, 1H), 8.55 (d, J = 1.6 Hz, 1H), 9.06 (t, J = 5.0 Hz, 1H). | 494.31 |
| 193 | 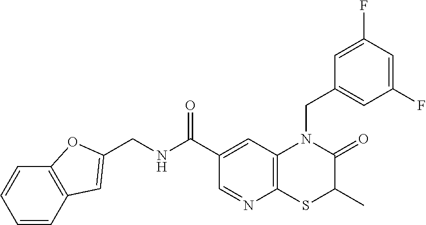 [SMILES: CC1Sc2ncc(C(=O)NCc3cc4ccccc4o3)cc2N(Cc2cc(F)cc(F)c2)C1=O] | N-(benzofuran- 2-ylmethyl)-1- (3,5- difluorobenzyl)- 3-methyl-2- oxo-2,3- dihydro-1H- pyrido[2,3- b][1,4]thiazine- 7-carboxamide | (500 MHz; DMSO- d₆): δ 1.46 (d, J = 7.0 Hz, 3H), 4.23-4.19 (m, 1H), 4.64 (d, J = 5.5 Hz, 2H), 5.36-5.19 (m, 2H), 6.75 (s, 1H), 6.94 (d, J = 6.6 Hz, 2H), 7.29-7.13 (m, 3H), 7.52 (d, J = 8.0 Hz, 1H), 7.58 (d, J = 7.5 Hz, 1H), 7.84 (s, 1H), 8.66 (d, J = 1.2 Hz, 1H), 9.33 (t, J = 5.5 Hz, 1H). | 480.27 |
| 194 | 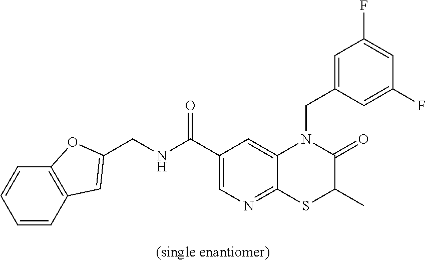 [SMILES: CC1Sc2ncc(C(=O)NCc3cc4ccccc4o3)cc2N(Cc2cc(F)cc(F)c2)C1=O] | N-(benzofuran- 2-ylmethyl)-1- (3,5- difluorobenzyl)- 3-methyl-2- oxo-2,3- dihydro-1H- pyrido[2,3- b][1,4]thiazine- 7-carboxamide | (400 MHz; DMSO- d₆): δ 1.45 (d, J = 7.0 Hz, 3H), 4.22-4.17 (m, 1H), 4.63 (d, J = 5.5 Hz, 2H), 5.33-5.15 (m, 2H), 6.74 (s, 1H), 6.94 (d, J = 6.7 Hz, 2H), 7.28-7.11 (m, 3H), 7.58-7.50 (m, 2H), 7.82 (s, 1H), 8.66 (s, 1H), 9.30 (t, J = 5.5 Hz, 1H). | 479.9 |
| 195 | 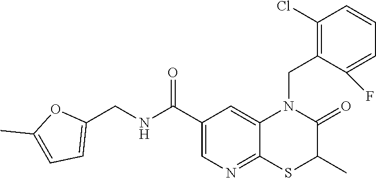 [SMILES: Cc1ccc(CNC(=O)c2cnc3c(c2)N(Cc2c(F)cccc2Cl)C(=O)C(C)S3)o1] | 1-(2-chloro-6- fluorobenzyl)- 3-methyl-N-((5- methylfuran-2- yl)methyl)-2- oxo-2,3- dihydro-1H- pyrido[2,3- b][1,4]thiazine- 7-carboxamide | (500 MHz; DMSO-d₆): δ 1.36 (d, J = 7.0 Hz, 3H), 2.24 (s, 3H), 3.96-3.92 (m, 1H), 4.42 (d, J = 5.3 Hz, 2H), 544-5.26 (m, 2H), 6.01 (s, 1H), 6.16 (d, J = 2.2 Hz, 1H), 7.14 (t, J = 9.7 Hz, 1H), 3.34-7.26 (m, 2H), 8.04 (s, 1H), 8.58 (s, 1H), 9.08 (t, J = 5.3 Hz, 1H). | 460.33 |
| 196 | 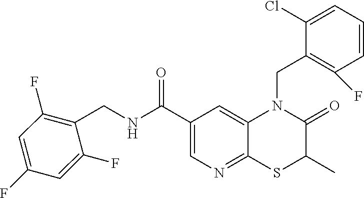 [SMILES: CC1Sc2ncc(C(=O)NCc3c(F)cc(F)cc3F)cc2N(Cc2c(F)cccc2Cl)C1=O] | 1-(2-chloro-6 fluorobenzyl) 3-methyl-2- oxo-N-(2,4,6- trifluorobenzyl)- 2,3-dihydro- 1H-pyrido[2,3- b][1,4]thiazine- 7-carboxamide | (500 MHz; DMSO- d₆): δ 1.37 (d, J = 6.9 Hz, 3H), 3.96-3.92 (m, 1H), 4.48 (d, J = 4.7 Hz, 2H), 5.44-5.23 (m, 2H), 7.13 (t, J = 9.2 Hz, 1H), 7.33-7.20 (m, 4H), 7.99 (s, 1H), 8.52 (s, 1H), 9.02 (bs, 1H). | 510.26 |
| 197 | 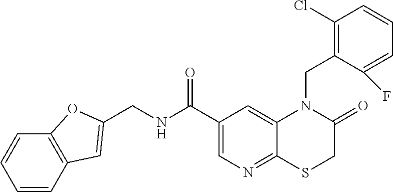 [SMILES: O=C(NCc1cc2ccccc2o1)c1cnc2c(c1)N(Cc1c(F)cccc1Cl)C(=O)CS2] | N-(benzofuran- 2-ylmethyl)-1- (2-chloro-6- fluorobenzyl)- 2-oxo-2,3- dihydro-1H- pyrido[2,3- b][1,4]thiazine- 7-carboxamide | (400 MHz; DMSO- d₆): δ 3.31 (s, 2H), 4.63 (d, J = 5.5 Hz, 2H), 5.32 (s, 2H), 6.75 (s, 1H), 7.09 (t, J = 9.0 Hz, 1H), 7.30-7.19 (m, 4H), 7.59-7.51 (dd, 2H), 8.02 (d, J = 1.3 Hz, 1H), 8.56 (d, J = 1.4 Hz, 1H), 9.25 (t, J = 5.6 Hz, 1H). | 482.52 |

| | | | LCMS |
|---|---|---|---|
| Example | Structure | IUPAC Name | [M + H] |
| 198 | 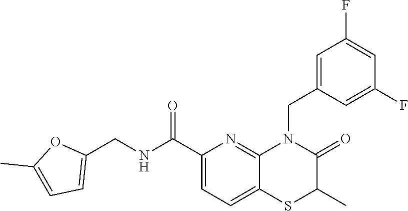 [SMILES: Cc1ccc(CNC(=O)c2ccc3c(n2)N(Cc2cc(F)cc(F)c2)C(=O)C(C)S3)o1] | 4-(3,5- difluorobenzyl)- 2-methyl-N- ((5-methylfuran- 2-yl)methyl)-3- oxo-3,4-dihydro- 2H-pyrido[3,2- b][1,4]thiazine- 6-carboxamide | 444.0 |
| 199 | 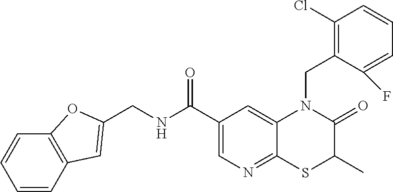 [SMILES: CC1Sc2ncc(C(=O)NCc3cc4ccccc4o3)cc2N(Cc2c(F)cccc2Cl)C1=O] | N-(benzofuran- 2-ylmethyl)-1- (2-chloro-6- fluorobenzyl)- 3-methyl-2- oxo-2,3-dihydro- 1H-pyrido[2,3- b][1,4]thiazine- 7-carboxamide | 496.33 |
| 200 | 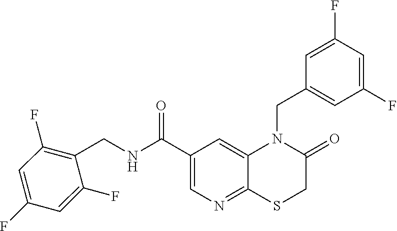 [SMILES: O=C(NCc1c(F)cc(F)cc1F)c1cnc2c(c1)N(Cc1cc(F)cc(F)c1)C(=O)CS2] | 1-(3,5- difluorobenzyl)- 2-oxo-N- (2,4,6- trifluorobenzyl)- 2,3-dihydro- 1H-pyrido[2,3-b] [1,4]thiazine-7- carboxamide | 480.31 |
| 201 | 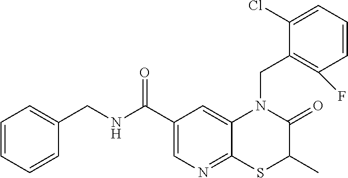 [SMILES: CC1Sc2ncc(C(=O)NCc3ccccc3)cc2N(Cc2c(F)cccc2Cl)C1=O] | N-benzyl-1- (2-chloro-6- fluorobenzyl)- 3-methyl-2- oxo-2,3- dihydro-1H- pyrido[2,3- b][1,4]thiazine- 7-carboxamide | 456.33 |
| 202 | 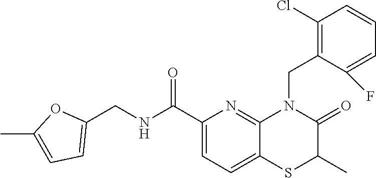 [SMILES: Cc1ccc(CNC(=O)c2ccc3c(n2)N(Cc2c(F)cccc2Cl)C(=O)C(C)S3)o1] | 4-(2-chloro-6- fluorobenzyl)-2- methyl-N-((5- methylfuran-2- yl)methyl)-3- oxo-3,4-dihydro- 2H- pyrido[3,2-b] [1,4]thiazine-6- carboxamide | 460.1 |
| 203 | 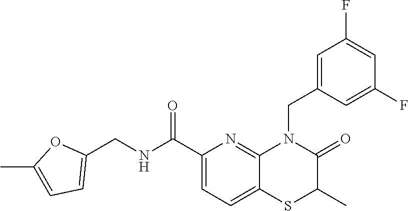 [SMILES: Cc1ccc(CNC(=O)c2ccc3c(n2)N(Cc2cc(F)cc(F)c2)C(=O)C(C)S3)o1] | 4-(3,5- difluorobenzyl)- 2-methyl-N- ((5-methylfuran- 2-yl)methyl)-3- oxo-3,4-dihydro- 2H-pyrido[3,2- b][1,4]thiazine- 6-carboxamide | 444.2 |
| 204 | 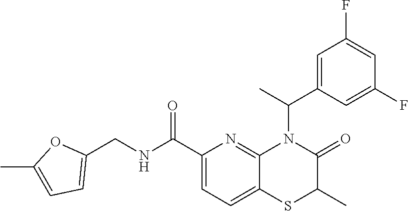 [SMILES: Cc1ccc(CNC(=O)c2ccc3c(n2)N(C(C)c2cc(F)cc(F)c2)C(=O)C(C)S3)o1] | 4-(1-(3,5- difluorophenyl) ethyl)-2- methyl-N-((5- methylfuran-2- yl)methyl)-3-oxo- 3,4-dihydro-2H- pyrido[3,2-b] [1,4] thiazine-6- carboxamide | 458.0 |
| 205 | 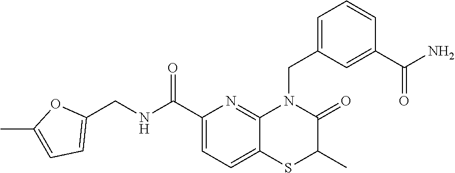 [SMILES: Cc1ccc(CNC(=O)c2ccc3c(n2)N(Cc2cccc(C(N)=O)c2)C(=O)C(C)S3)o1] | 4-(3-carbamoyl- benzyl)- 2-methyl-N- ((5-methylfuran-2- yl)methyl)-3-oxo- 3,4-dihydro-2H- pyrido[3,2- b][1,4]thiazine- 6-carboxamide | 451.0 |
| 206 | 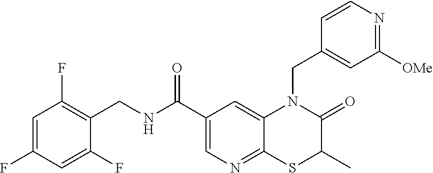 [SMILES: COc1cc(CN2C(=O)C(C)Sc3ncc(C(=O)NCc4c(F)cc(F)cc4F)cc32)ccn1] | 1-((2- methoxypyridin- 4-yl)methyl)- 3-methyl-2-oxo- N-(2,4,6- trifluorobenzyl)- 2,3-dihydro-1H- pyrido[2,3-b][1,4] thiazine-7- carboxamide | 489.32 |
| 207 | 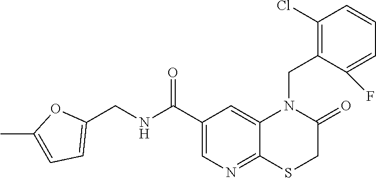 [SMILES: Cc1ccc(CNC(=O)c2cnc3c(c2)N(Cc2c(F)cccc2Cl)C(=O)CS3)o1] | 1-(2-chloro-6- fluorobenzyl)- N-((5- methylfuran- 2-yl)methyl)-2 -oxo-2,3- dihydro-1H-pyrido [2,3-b][1,4] thiazine-7- carboxamide | 446.28 |
| 208 | 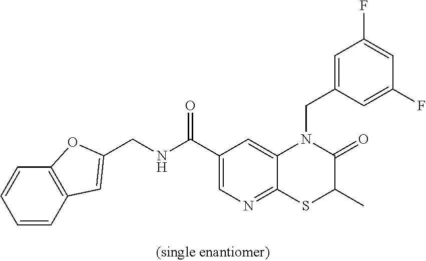 [SMILES: CC1Sc2ncc(C(=O)NCc3cc4ccccc4o3)cc2N(Cc2cc(F)cc(F)c2)C1=O] | N-(benzofuran- 2-ylmethyl)-1- (3,5- difluorobenzyl)- 3-methyl-2-oxo- 2,3- dihydro-1H- pyrido[2,3- b][1,4] thiazine-7- carboxamide | 479.8 |
| 209 | 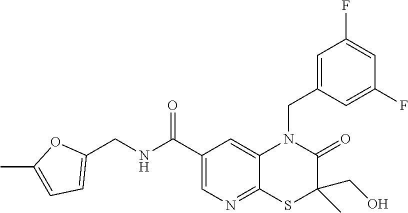 [SMILES: Cc1ccc(CNC(=O)c2cnc3c(c2)N(Cc2cc(F)cc(F)c2)C(=O)C(C)(CO)S3)o1] | 1-(3,5- difluorobenzyl)- 3- (hydroxymethyl)- 3-methyl-N-((5- methylfuran-2-yl) methyl)-2-oxo- 2,3-dihydro- 1H-pyrido [2,3-b] [1,4]thiazine- 7-carboxamide | 474.56 |
| 210 | 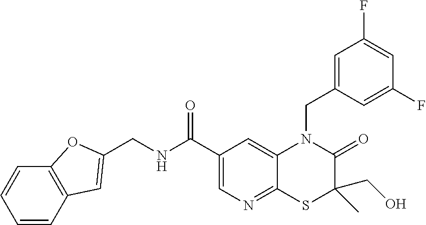 [SMILES: CC1(CO)Sc2ncc(C(=O)NCc3cc4ccccc4o3)cc2N(Cc2cc(F)cc(F)c2)C1=O] | N-(benzofuran-2- ylmethyl)-1-(3,5- difluorobenzyl)- 3- (hydroxymethyl)- 3-methyl-2-oxo- 2,3- dihydro-1H- pyrido[2,3-b] [1,4] thiazine-7- carboxamide | 510.57 |
| 211 | 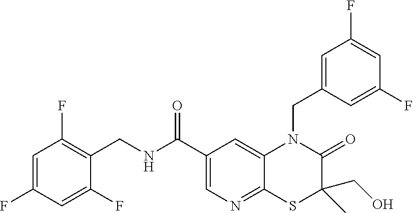 [SMILES: CC1(CO)Sc2ncc(C(=O)NCc3c(F)cc(F)cc3F)cc2N(Cc2cc(F)cc(F)c2)C1=O] | 1-(3,5- difluorobenzyl)- 3- (hydroxymethyl)- 3-methyl-2-oxo- N-(2,4,6- trifluorobenzyl)- 2,3-dihydro- 1H-pyrido [2,3-b] [1,4]thiazine-7- carboxamide | 524.55 |
| 212 | 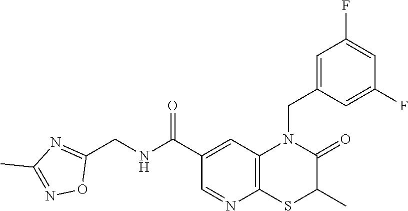 [SMILES: Cc1noc(CNC(=O)c2cnc3c(c2)N(Cc2cc(F)cc(F)c2)C(=O)C(C)S3)n1] | 1-(3,5- difluorobenzyl)- 3-methyl-N- ((3-methyl-1,2,4- oxadiazol-5- yl)methyl)-2- oxo-2,3- dihydro-1H- pyrido[2,3-b] [1,4] thiazine-7- carboxamide | 446.36 |
| 213 | 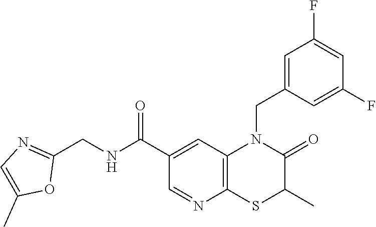 [SMILES: Cc1cnc(CNC(=O)c2cnc3c(c2)N(Cc2cc(F)cc(F)c2)C(=O)C(C)S3)o1] | 1-(3,5- difluorobenzyl)- 3-methyl-N- ((5- methyloxazol- 2-yl)methyl)-2- oxo-2,3-dihydro- 1H-pyrido[2,3- b][1,4]thiazine- 7- carboxamide | 445.38 |
| 214 | 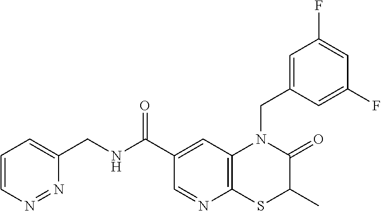 [SMILES: CC1Sc2ncc(C(=O)NCc3cccnn3)cc2N(Cc2cc(F)cc(F)c2)C1=O] | 1-(3,5- difluorobenzyl)- 3-methyl-2- oxo-N- (pyridazin- 3-ylmethyl)-2,3- dihydro-1H- pyrido [2,3-b][1,4] thiazine-7- carboxamide | 442.40 |
| 215 | 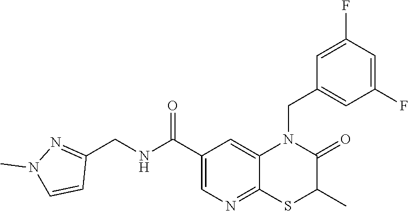 [SMILES: CC1Sc2ncc(C(=O)NCc3ccn(C)n3)cc2N(Cc2cc(F)cc(F)c2)C1=O] | 1-(3,5- difluorobenzyl)- 3-methyl-N- ((1-methyl-1H- pyrazol-3-yl) methyl)- 2-oxo-2,3- dihydro- 1H-pyrido[2,3- b][1,4]thiazine- 7-carboxamide | 444.15 |
| 216 | 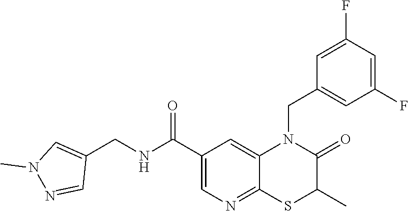 [SMILES: CC1Sc2ncc(C(=O)NCc3cnn(C)c3)cc2N(Cc2cc(F)cc(F)c2)C1=O] | 1-(3,5- difluorobenzyl)- 3-methyl-N- ((1-methyl-1H- pyrazol-4-yl) methyl)- 2-oxo-2,3- dihydro-1H- pyrido[2,3- b][1,4] thiazine-7- carboxamide | 444.56 |
| 217 | 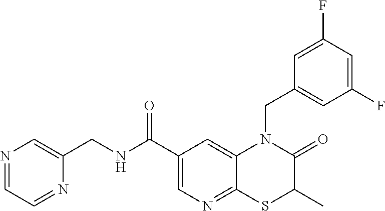 [SMILES: CC1Sc2ncc(C(=O)NCc3cnccn3)cc2N(Cc2cc(F)cc(F)c2)C1=O] | 1-(3,5- difluorobenzyl)- 3-methyl oxo-N-(pyrazin- 2-ylmethyl)-2,3 dihydro-1H- pyrido[2,3- b][1,4]thiazine- 7-carboxamide | 442.29 |
| 218 | 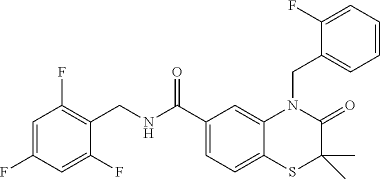 [SMILES: CC1(C)Sc2ccc(C(=O)NCc3c(F)cc(F)cc3F)cc2N(Cc2ccccc2F)C1=O] | 4-(2- fluorobenzyl)- 2,2-dimethyl-3- oxo-N-(2,4,6- trifluorobenzyl)- 3,4-dihydro-2H- benzo[b][1,4] thiazine-6- carboxamide | 489.29 |

### Example 219: 4-Benzyl-8-cyano-N-(furan-2-ylmethyl)-3-oxo-3,4-dihydro-2H-benzo[b-1,4]thiazine-6-carboxamide

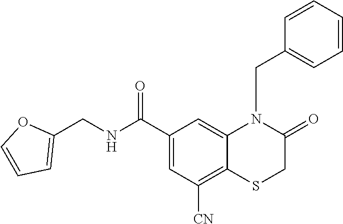 [SMILES: N#Cc1cc(C(=O)NCc2ccco2)cc2c1SCC(=O)N2Cc1ccccc1]

### Preparation 14: Methyl 3-bromo-4-((2-ethoxy-2-oxoethyl)thio)-5-nitrobenzoate

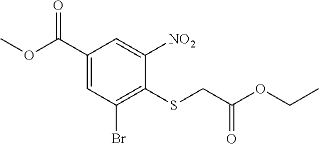 [SMILES: CCOC(=O)CSc1c(Br)cc(C(=O)OC)cc1[N+](=O)[O-]]

To a stirred solution of commercially available methyl 3-bromo-4-fluoro-5-nitrobenzoate (0.25 g, 0.89 mmol) in MeCN (10 mL) was added ethyl thioglycolate (0.118 mL, 0.98 mmol) followed by TEA (0.188 mL, 1.85 mmol) at 0-5° C. and further stirred at the same temperature for 10 min. The reaction performance was monitored by TLC and after completion the reaction mixture was diluted with EtOAc and washed with water followed by brine. The combined organics were dried over anhydrous Na₂SO₄ and concentrated under reduced pressure to dryness to afford methyl 3-bromo-4-((2-ethoxy-2-oxoethyl)thio)-5-nitrobenzoate (0.30 g, 0.79 mmol, 89% yield) which was used in the next step as such. LCMS m/z: 378.09 [M+H].

### Preparation 15: Methyl 8-bromo-1-oxo-3,4-dihydro-2H-1,4-benzothiazine-6-carboxylate

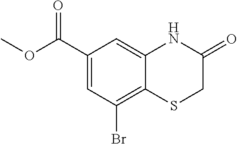 [SMILES: COC(=O)c1cc(Br)c2c(c1)NC(=O)CS2]

Methyl 8-bromo-3-oxo-3,4-dihydro-2H-1,4-benzothiazine-6-carboxylate was made from methyl 3-bromo-4-((2-ethoxy-2-oxoethyl)thio)-5-nitrobenzoate (Preparation 14) using the method described for Preparation 1. LCMS m/z: 300.01 [M−H].

### Preparation 16: 8-Bromo-4-benzyl-N-(furan-2-ylmethyl)-3-oxo-, 4-dihydro-2H-benzo[b-1,4]thiazine-6-carboxamide

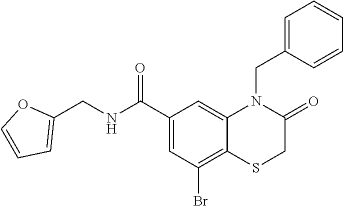 [SMILES: O=C(NCc1ccco1)c1cc(Br)c2c(c1)N(Cc1ccccc1)C(=O)CS2]

8-Bromo-4-benzyl-N-(furan-2-ylmethyl)-3-oxo-3,4-dihydro-2H-benzo[b-1,4]thiazine-6-carboxamide was made from methyl 8-bromo-3-oxo-3,4-dihydro-2H-1,4-benzothiazine-6-carboxylate (Preparation 15) using the methods described in Preparation 4, Preparation 5 and Preparation 6, using benzyl chloride and 1-(furan-2-yl)-methanamine. ¹H NMR (500 MHz; DMSO-d₆): δ 3.82 (s, 2H), 4.42 (d, J=5.55 Hz, 2H), 5.27 (s, 2H), 6.24 (d, J=3.05 Hz, 1H), 6.39-6.40 (m, 1H), 7.11-7.12 (m, 2H), 7.21-7.25 (m, 1H), 7.30-7.33 (m, 1H), 7.58-7.61 (m, 2H), 7.89 (d, J=1.20 Hz, 1H), 9.11 (t, J=5.5 Hz, 1H).

### Preparation 17: 4-Benzyl-8-cyano-N-(furan-2-ylmethyl)-3-oxo-3,4-dihydro-2H-benzo[b-1,4]thiazine-6-carboxamide

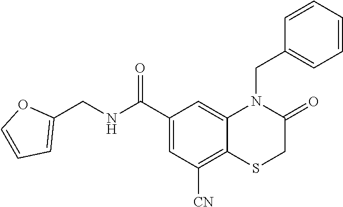 [SMILES: N#Cc1cc(C(=O)NCc2ccco2)cc2c1SCC(=O)N2Cc1ccccc1]

A stirred solution of 4-benzyl-8-bromo-N-(furan-2-ylmethyl)-3-oxo-3,4-dihydro-2H-benzo[b-1,4]thiazine-6-carboxamide (Preparation 16) (0.095 g, 0.20 mmol) in NMP (20 mL) was degassed thoroughly with Ar. To the mixture was added CuCN (0.093 g, 1.03 mmol) under an Ar atmosphere and the resulting reaction mixture was heated at 145-150° C. for 16 h. After completion of the reaction (monitored by TLC/LCMS), the reaction mass was cooled to RT, diluted with EtOAc and washed with an aqueous FeCl₃ solution followed by water and brine. The organic layer was dried over anhydrous Na₂SO₄ and evaporated under reduced pressure. The crude product obtained was purified by column chromatography using 30% EtOAc in hexanes as eluent to afford 4-benzyl-8-cyano-N-(furan-2-ylmethyl)-3-oxo-3,4-dihydro-2H-benzo[b-1,4]thiazine-6-carboxamide (Example 219) (0.02 g, 0.049 mmol, 25% yield) as an off white solid. LCMS m/z: 402.28 [M−H]; ¹H NMR (500 MHz; DMSO-d₆): δ 3.91 (s, 2H), 4.43 (d, J=5.35 Hz, 2H), 5.30 (s, 2H), 6.26 (d, J=2.6 Hz, 1H), 6.40 (s, 1H), 7.20-7.26 (m, 3H), 7.31-7.34 (m, 2H), 7.59 (s, 1H), 7.85 (s, 1H), 8.04 (s, 1H), 9.16 (t, J=5.25 Hz, 1H).

### Examples 220-225

Examples 220-225 were prepared in an entirely analogous manner to Example 219 using the methods described in Preparations 15 and 16, as described in General Procedures 4, 5 and 1-3 starting with an appropriate nitroarene and using the appropriate benzyl amine and either 1-(furan-2-yl)-methanamine or 2,4,6-trifluorobenzyl-amine.

| | | | | LCMS |
|---|---|---|---|---|
| Example | Structure | IUPAC Name | ¹H-NMR | [M+ H] |
| 220 | 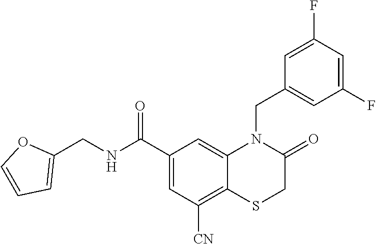 [SMILES: N#Cc1cc(C(=O)NCc2ccco2)cc2c1SCC(=O)N2Cc1cc(F)cc(F)c1] | 8-cyano-4- (3,5- difluorobenzyl)- N-(furan-2- ylmethyl)-3- oxo-3,4- dihydro-2H- benzo[b- 1,4]thiazine-6- carboxamide | (500 MHz; DMSO-d₆): δ 3.95 (s, 2H), 4.44 (d, J = 5.45 Hz, 2H), 5.29 (s, 2H), 6.26 (d, J = 2.75 Hz, 1H), 6.39 (s, 1H), 6.98 (d, J = 6.75 Hz, 2H), 7.16 (t, J = 9.2 Hz, 1H), 7.58 (s, 1H), 7.71 (s, 1H), 8.08 (s, 1H), 9.17 (t, J = 5.45 Hz, 1H). | 440.26 |
| 221 | 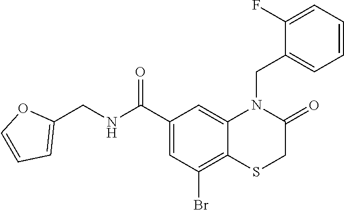 [SMILES: O=C(NCc1ccco1)c1cc(Br)c2c(c1)N(Cc1ccccc1F)C(=O)CS2] | 8-bromo-4-(2- fluorobenzyl)- N-(furan-2- ylmethyl)-3- oxo-3,4- dihydro-2H- benzo[b- 1,4]thiazine-6- carboxamide | 500 MHz; DMSO-d₆): δ 3.82 (s, 2H), 4.42 (d, J = 5.55 Hz, 2H), 5.27 (s, 2H), 6.24 (d, J = 3.05 Hz, 1H), 6.39-6.40 (m, 1H), 7.11-7.12 (m, 2H), 7.21-7.25 (m, 1H), 7.30- 7.33 (m, 1H), 7.58-7.61 (m, 2H), 7.89 (d, J = 1.20 Hz, 1H), 9.11 (t, J = 5.5 Hz, 1H). | 474.88 |
| 222 | 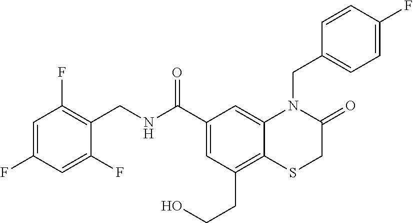 [SMILES: O=C(NCc1c(F)cc(F)cc1F)c1cc(CCO)c2c(c1)N(Cc1ccc(F)cc1)C(=O)CS2] | 4-(4- fluorobenzyl)- 8-(2- hydroxyethyl)- 3-oxo-N- (2,4,6- trifluorobenzyl)- 3,4-dihydro- 2H- benzo[b][1,4] thiazine-6- carboxamide | (500 MHz; DMSO-d₆): δ 2.87 (t, J = 6.90 Hz, 2H), 3.56-3.60 (q, J = 6.70 Hz, 2H), 3.64 (s, 2H), 4.41 (d, J = 4.8 Hz, 2H), 4.78 (t, J = 5.20 Hz, 1H), 5.22 (s, 2H), 7.13 (t, J = 8.75 Hz, 2H), 7.17-7.25 (m, 4H), 7.47 (d, J = 3.7 Hz, 2H), 8.86 (t, J = 4.75 Hz, 1H). | 505.31 |
| 223 | 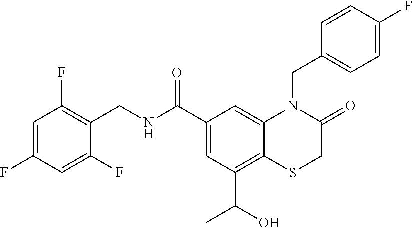 [SMILES: CC(O)c1cc(C(=O)NCc2c(F)cc(F)cc2F)cc2c1SCC(=O)N2Cc1ccc(F)cc1] | 4-(4- fluorobenzyl)- 8-(1- hydroxyethyl)- 3-oxo-N- (2,4,6- trifluorobenzyl)- 3,4-dihydro- 2H- benzo[b][1,4] thiazine-6- carboxamide | (500 MHz; DMSO-d₆): δ 1.33 (d, J = 6.3 Hz, 3H), 3.58-3.65 (m, 2H), 4.40-4.47 (m, 2H), 4.96-4.98 (m, 1H), 5.18- 5.30 (m, 2H), 5.48 (d, J = 3.9 Hz, 1H), 7.13 (t, J = 8.80 Hz, 2H), 7.17- 7.25 (m, 4H), 7.53 (bs, 1H), 7.72 (bs, 1H), 8.95- 8.96 (m, 1H). | 505.30 |
| 224 | 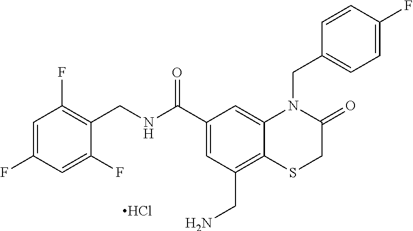 [SMILES: NCc1cc(C(=O)NCc2c(F)cc(F)cc2F)cc2c1SCC(=O)N2Cc1ccc(F)cc1 ; Cl] | 8- (aminomethyl)- 4-(4- fluorobenzyl)- 3-oxo-N- (2,4,6- trifluorobenzyl)- 3,4-dihydro- 2H- benzo[b][1,4] thiazine-6- carboxamide hydrochloride | (500 MHz; DMSO-d₆): δ 3.69 (s, 2H), 4.11 (s, 2H), 4.46 (d, J = 4.8 Hz, 2H), 5.32 (s, 2H), 7.11 (t, J = 8.8 Hz, 2H), 7.19- 7.25 (m, 4H), 7.75 (d, J = 5.95 Hz, 2H), 8.41 (bs, 3H), 8.01 (t, J = 4.8 Hz, 1H). | 490.32 |
| 225 | 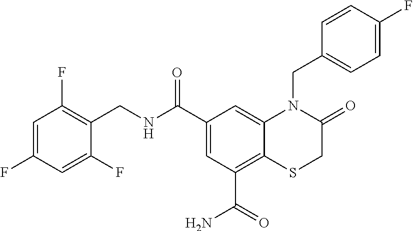 [SMILES: NC(=O)c1cc(C(=O)NCc2c(F)cc(F)cc2F)cc2c1SCC(=O)N2Cc1ccc(F)cc1] | 4-(4- fluorobenzyl)- 3-oxo-N6- (2,4,6- trifluorobenzyl)- 3,4-dihydro- 2H- benzo[b][1,4] thiazine-6,8- dicarboxamide | (400 MHz; DMSO-d₆): δ 3.47 (s, 2H), 4.42 (d, J = 5.0 Hz, 2H), 5.23 (s, 2H), 7.12-7.08 (m, 2H), 7.21-7.18 (m, 4H), 7.64 (bs, 1H), 7.67 (d, J = 1.3 Hz, 1H), 7.74 (d, J = 1.3 Hz, 1H), 8.00 (s, 1H), 8.86 (t, J = 5.1 Hz, 1H). | 504.30 |

### Example 226: 4-(2-Fluorobenzyl)-N-(furan-2-ylmethyl)-8-meth yl-3-oxo-3,4-dihydro-2H-benzo[b-1,4]thiazine-6-carboxamide

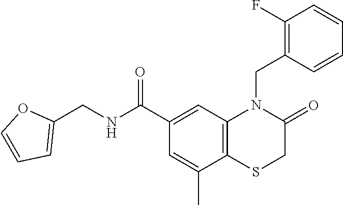 [SMILES: Cc1cc(C(=O)NCc2ccco2)cc2c1SCC(=O)N2Cc1ccccc1F]

### Preparation 18

To a stirred solution of 8-bromo-4-(2-fluorobenzyl)-N-(furan-2-ylmethyl)-3-oxo-3,4-dihydro-2H-benzo[b-1,4]thiazine-6-carboxamide (Example 221) (0.08 g, 0.20 mmol) in a mixture of 1,4-dioxane and water (10:1; 9 mL) was added trimethylboroxin (0.07 mL, 0.50 mmol) and K₂CO₃ (0.056 g, 0.40 mmol) at RT in a sealed tube. The resulting reaction mixture was degassed with Ar for 15 min. then Pd(PPh₃)₄(0.047 g, 0.040 mmol, 20 mol %) was added at RT. Finally, the reaction mixture was heated at 110° C. for 16 h. The progress of reaction was monitored by TLC/LCMS and after completion; the reaction mixture was cooled down to RT, diluted with EtOAc, washed with water and brine. The organic layer was dried over anhydrous Na₂SO₄ and concentrated under reduced pressure to afford crude material which was purified by prep-HPLC to give 4-(2-Fluorobenzyl)-N-(furan-2-ylmethyl)-8-methyl-3-oxo-3,4-dihydro-2H-benzo[b-1,4]thiazine-6-carboxamide (Example 226) (0.02 g, 0.048 mmol, 25% yield) as an off white solid. LCMS m/z: 411.01 [M+H]; ¹H NMR (500 MHz; DMSO-d₆): δ 2.36 (s, 3H), 3.70 (s, 2H), 4.42 (d, J=5.20 Hz, 2H), 5.28 (s, 2H), 6.21 (S, 1H), 6.39 (s, 1H), 7.09-7.12 (m, 2H), 7.22 (t, J=9.7 Hz, 1H), 7.30-7.33 (m, 1H), 7.49-7.61 (m, 3H), 8.97 (bs, 1H).

### Example 227

Example 227 was prepared in an entirely analogous manner to Examples 226 and 219 (below) starting from Example 221.

| | | | | LCMS |
|---|---|---|---|---|
| Example | Structure | IUPAC Name | ¹H-NMR | [M + H] |
| 227 | 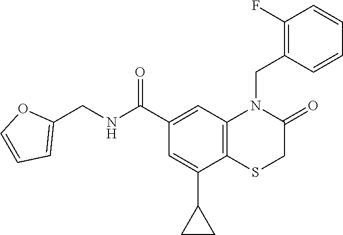 [SMILES: O=C(NCc1ccco1)c1cc(C2CC2)c2c(c1)N(Cc1ccccc1F)C(=O)CS2] | 8-cyclopropyl-4- (2-fluorobenzyl)- N-(furan-2- ylmethyl)-3-oxo- 3,4-dihydro-2H- benzo[b][1,4]thi- azine-6- carboxamide | (500 MHz; DMSO- d₆): δ 0.74-0.86 (m, 2H), 0.99-1.02 (m, 2H), 2.05-2.11 (m, 1H), 3.72 (s,2H), 4.41 (d, J = 5.65 Hz, 2H), 5.24 (s,2H), 6.21 (d, J = 3.05 Hz, 1H), 6.38-6.39 (m, 1H), 7.08-7.13 (m, 2H), 7.21-7.25 (m, 2H), 7.29-7.33 (m, 1H), 7.44 (d, J = 1.15 Hz, 1H), 7.57 (d, J = 0.9 Hz, 1H), 8.99 (t, J = 5.65 Hz, 1H). | 436.94 |

### Example 228: 7-bromo-4-(2-fluorobenzyl)-N-(furan-2-ylmethyl)-3-oxo-3,4-dihydro-2H-benzo[b][1,4]thiazine-6-carboxamide

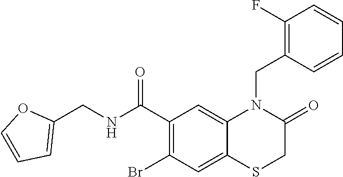 [SMILES: O=C(NCc1ccco1)c1cc2c(cc1Br)SCC(=O)N2Cc1ccccc1F]

### Preparation 19: Methyl 2-bromo-4-fluoro-5-nitrobenzoate

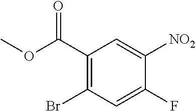

Methyl 2-bromo-4-fluorobenzoate (1.0 g, 4.31 mmol) was dissolved in H₂SO₄ (10 mL) and cooled to 0-5° C., then potassium nitrite (0.43 g, 4.31 mmol) was added portionwise and the reaction mixture was stirred at room temperature for 1 h. After completion, the reaction mass was poured into crushed ice and stirred for 30 min. The solid obtained was filtered, washed with cold water and dried under reduced pressure to obtain methyl 2-bromo-4-fluorobenzoate (0.86 g, 3.09 mmol, 72% yield) as a yellow solid which was pure enough to be used in the next step. LCMS m/z: 276.07 [M−H].

### Preparation 20: Methyl 7-bromo-3-oxo-3,4-dihydro-2H-benzo[b][1,4]thiazine-6-carboxylate

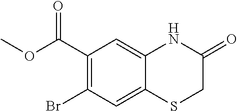 [SMILES: COC(=O)c1cc2c(cc1Br)SCC(=O)N2]

Methyl 7-bromo-3-oxo-3,4-dihydro-2H-benzo[b][1,4]thiazine-6-carboxylate was made from methyl 2-bromo-4-fluoro-5-nitrobenzoate (Preparation 19) using the method described in Preparation 1. LCMS m/z: 300.03 [M−H].

### Preparation 21: 7-bromo-4-(2-fluorobenzyl)-N-(furan-2-ylmethyl)-3-oxo-3,4-dihydro-2H-benzo[b][1,4]thiazine-6-carboxamide

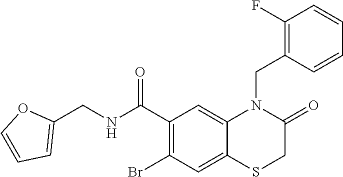 [SMILES: O=C(NCc1ccco1)c1cc2c(cc1Br)SCC(=O)N2Cc1ccccc1F]

4-Benzyl-8-bromo-N-(furan-2-ylmethyl)-3-oxo-3,4-dihydro-2H-1,4-benzo[b-1,4]thiazine-6-carboxamide (Example 228) was made from methyl-7-bromo-3-oxo-3,4-dihydro-2H-benzo[b][1,4]thiazine-6-carboxylate (Preparation 20) using the methods described in Preparations 4 to 6, according to the General Procedures 1-3 using 2-fluorobenzyl chloride and 1-(furan-2-yl)-methanamine. LCMS m/z: 474.85 [M+H]; ¹H NMR (500 MHz; DMSO-d₆): δ 3.74 (s, 2H), 4.37 (d, J=5.7 Hz, 2H), 5.24 (s, 2H), 6.15 (d, J=2.65 Hz, 1H), 6.38 (s, 1H), 7.09-7.15 (m, 3H), 7.24 (t, J=9.8 Hz, 1H), 7.32-7.36 (m, 1H), 7.55 (s, 1H), 7.78 (s, 1H), 8.91 (bs, 1H).

### Example 229

### Preparation 22: 7-Cyclopropyl-4-(2-fluorobenzyl)-N-(furan-2-ylmethyl)-3-oxo-3,4-dihydro-2H-benzo[b-1,4]thiazine-6-carboxamide

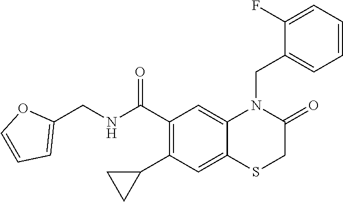 [SMILES: O=C(NCc1ccco1)c1cc2c(cc1C1CC1)SCC(=O)N2Cc1ccccc1F]

To a stirred solution of 4-benzyl-8-bromo-N-(furan-2-ylmethyl)-3-oxo-3,4-dihydro-2H-1,4-benzo[b-1,4]thiazine-6-carboxamide (Example 228/Preparation 21) (0.14 g, 0.29 mmol) in a mixture of 1,4-dioxane and water (10:1; 11 mL) was added cyclopropylboronic acid (0.076 g, 0.88 mmol) and sodium tert-butoxide (0.056 g, 0.58 mmol) at RT in a sealed tube. The resulting reaction mixture was degassed with Ar for 15 min. then tricyclohexylphosphine (0.016 g, 0.058 mmol) and PdCl₂(PPh₃)2 (0.020 g, 0.029 mmol, 10 mol %) was added at RT. Finally, the reaction mixture was heated at 110° C. for 16 h. The progress of reaction was monitored by TLC/LCMS and after completion; the reaction mixture was cooled down to RT, diluted with EtOAc, washed with water and brine. The organic layer was dried over anhydrous Na₂SO₄ and concentrated under reduced pressure to afford crude material which was purified by prep-HPLC to give 7-cyclopropyl-4-(2-fluorobenzyl)-N-(furan-2-ylmethyl)-3-oxo-3,4-dihydro-2H-benzo[b-1,4]thiazine-6-carboxamide (Example 229) (0.01 g, 0.022 mmol, 8% yield) as an off white solid. LCMS m/z: 437.36 [M+H]; ¹H NMR (500 MHz; DMSO-d₆): δ 0.64 (d, J=4.1 Hz, 2H), 0.83 (d, J=7.85 Hz, 2H), 2.11 (bs, 1H), 3.68 (s, 2H), 4.38 (d, J=5.3 Hz, 2H), 5.22 (s, 2H), 6.07 (s, 1H), 6.37 (s, 1H), 6.98 (d, J=15.15 Hz, 2H), 7.08-7.15 (m, 2H), 7.25 (t, J=9.55 Hz, 1H), 7.33-7.34 (m, 1H), 7.53 (s, 1H), 8.80 (bs, 1H).

### Example 230: 4-Benzyl-N-(furan-2-ylmethyl)-7-methyl-3-oxo-3,4-dihydro-2H-benzo[b-1,4]thiazine-6-carboxamide

Example 230 was prepared in an entirely analogous manner to Example 229 from Example 228.

| | | | | LCMS |
|---|---|---|---|---|
| Example | Structure | IUPAC Name | ¹H-NMR | [M + H] |
| 230 | 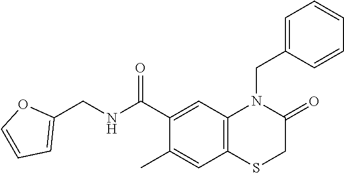 [SMILES: Cc1cc2c(cc1C(=O)NCc1ccco1)N(Cc1ccccc1)C(=O)CS2] | 4-benzyl-N- (furan-2- ylmethyl)-7- methyl-3-oxo- 3,4-dihydro- 2H-benzo[b- 1,4]thiazine-6- carboxamide | (500 MHz; DMSO-d₆): δ 2.23 (s, 3H), 3.68 (s, 2H), 4.37 (d, J = 5.65 Hz, 2H), 5.22 (s, 2H), 6.10 (d, J = 2.5 Hz, 1H), 6.39 (s, 1H), 7.08 (s, 1H), 7.20-7.26 (m, 3H), 7.31-7.34 (m, 3H), 7.57 (s, 1H), 8.73 (t, J = 5.6 Hz, 1H). | 393.26 |

### Example 231: 4-(2-Chloro-6-fluorobenzyl)-N-(furan-2-ylmethyl)-3-oxo-3,4-dihydro-2H-benzo[b-1,4]oxazine-6-carboxamide

Example 231 was prepared in an entirely analogous manner to Example 1, as described in General procedures 1-6, starting with an appropriate alcohol in place of the thiol.

| | | | | LCMS |
|---|---|---|---|---|
| Example | Structure | IUPAC Name | ¹H-NMR | [M + H] |
| 231 | 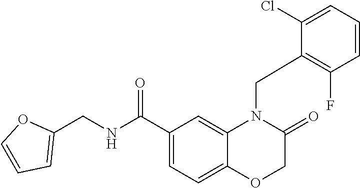 [SMILES: O=C(NCc1ccco1)c1ccc2c(c1)N(Cc1c(F)cccc1Cl)C(=O)CO2] | 4-(2-chloro-6- fluorobenzyl)- N-(furan-2- ylmethyl)-3- oxo-3,4- dihydro-2H- benzo[b- 1,4]oxazine-6- carboxamide | (500 MHz; DMSO-d₆): δ 4.43 (d, J = 5.6 Hz, 2H), 4.75 (s, 2H), 5.33 (s, 2H), 6.24 (d, J = 2.9 Hz, 1H), 6.40-6.41 (m, 1H), 7.07 (d, J = 8.35 Hz, 1H), 7.17 (t, J = 9.8 Hz, 1H), 7.31-7.35 (m, 2H), 7.52-7.54 (dd, J₁= 1.65 Hz, J₂= 8.35 Hz, 1H), 7.58 (s, 1H), 7.68 (d, J = 1.5 Hz, 1H), 8.85 (t, J = 5.6 Hz, 1H). | 414.99 |

### Preparation 23: Methyl 4-((1-ethoxy-1-oxopropan-2-yl)oxy)-3-nitrobenzoate

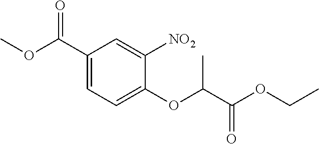 [SMILES: CCOC(=O)C(C)Oc1ccc(C(=O)OC)cc1[N+](=O)[O-]]

Methyl-4-fluoro-3-nitrobenzoate (0.5 g, 2.5 mmol) and 2-hydroxy methyl propionate (0.356 g, 0.30 mmol) were dissolved in 1,4-dioxane (5 mL) at RT. In to the resulting reaction mixture, NaH (0.0726 g, 3.01 mmol; 60% suspension in oil) was added portionwise with ice bath cooling and allowed to stir at room temperature for 16 h. On completion of the reaction it was quenched with aqueous NH₄Cl and extracted with EtOAc. The organic layer was washed with brine, dried over anhydrous Na₂SO₄ and evaporated under reduced pressure to afford methyl 4-((1-ethoxy-1-oxopropan-2-yl)oxy)-3-nitrobenzoate (0.6 g, 2.02 mmol, 81% yield) as a colourless oil which was used directly in the next step without further purification. LCMS m/z: 298.16 [M+H].

### Preparation 24: Methyl 2-methyl-3-oxo-3,4-dihydro-2H-1,4-benzoxazine-6-carboxylate

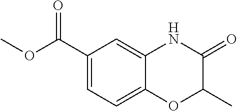 [SMILES: COC(=O)c1ccc2c(c1)NC(=O)C(C)O2]

Methyl 2-methyl-3-oxo-3,4-dihydro-2H-1,4-benzoxazine-6-carboxylate was prepared from Preparation 23 using the method described in Preparation 1. LCMS m/z: 222.06 [M+H].

### Example 232

### Preparation 25: 2-Methyl-4-(3,5-difluorobenzvyl)-3-oxo-N-(2,4,6-trifluorobenzyl)-3,4-dihydro-2H-1,4-benzoxazine-6-carboxamide

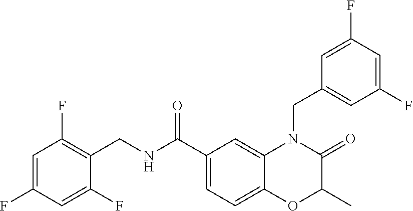 [SMILES: CC1Oc2ccc(C(=O)NCc3c(F)cc(F)cc3F)cc2N(Cc2cc(F)cc(F)c2)C1=O]

Example 232 was made from Preparation 24 using the methods described in Preparations 4 to 6, using 3,5-difluorobenzyl chloride and 1-(2,4,6-trifluorophenyl)-methanamine. LCMS m/z: 477.28 [M+H]; ¹H NMR (500 MHz; DMSO-d₆): δ 1.53 (d, J=6.65 Hz, 3H), 4.41 (s, 2H), 5.02-5.10 (m, 2H), 5.29 (d, J=16.8 Hz, 1H), 6.99 (d, J=6.5 Hz, 2H), 7.11-7.17 (m, 4H), 7.41 (s, 1H), 7.53 (d, J=8.25 Hz, 1H), 8.73 (bs, 1H).

### Examples 233-235

Examples 233-235 were prepared in an analogous manner to Example 232, following the methods described in Preparations 23 to 25, starting with an appropriate nitroarene and alcohol.

| | | | | LCMS |
|---|---|---|---|---|
| Example | Structure | IUPAC Name | ¹H-NMR | [M + H] |
| 233 | 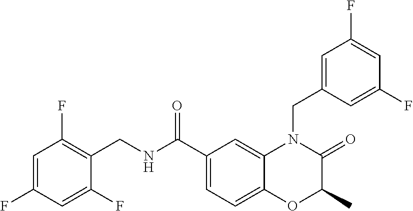 [SMILES: C[C@H]1Oc2ccc(C(=O)NCc3c(F)cc(F)cc3F)cc2N(Cc2cc(F)cc(F)c2)C1=O] | (R)-4-(3,5- difluorobenzyl)- 2-methyl-3- oxo-N-(2,4.,6- trifluorobenzyl)- 3,4-dihydro- 2H-benzo[b- 1,4]oxazine-6- carboxamide | (500 MHz; DMSO-d₆): δ 1.52 (d, J = 6.75 Hz, 3H), 4.37-4.44 (m, 2H), 5.01- 5.05 (m, 1H), 5.08 (d, J = 16.9 Hz, 1H), 5.28 (d, J = 16.85 Hz, 1H), 6.99 (d, J = 6.35 Hz, 2H), 7.11 (d, J = 8.35 Hz, 1H), 7.14-7.18 (m, 3H), 7.40 (d, J = 1.7 Hz, 1H), 7.52-7.54 (dd, J₁ = 8.35 Hz, J₂ = 1.8 Hz, 1H), 8.83 (t, J = 5.05 Hz, 1H). | 477.30 |
| 234 | 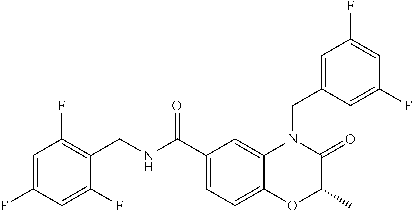 [SMILES: C[C@@H]1Oc2ccc(C(=O)NCc3c(F)cc(F)cc3F)cc2N(Cc2cc(F)cc(F)c2)C1=O] | (S)-4-(3,5- difluorobenzyl)- 2-methyl-3- oxo-N-(2,4,6- trifluorobenzyl)- 3,4-dihydro- 2H-benzo[b- 1,4]oxazine-6- carboxamide | (500 MHz; DMSO-d₆): δ 1.51-1.53 (m, 3H), 4.37- 4.44 (m, 2H), 4.98-5.05 (m, 1H), 5.08 (d, J = 16.8 Hz, 1H), 5.28 (d, J = 16.85 Hz, 1H), 6.99 (d, J = 6.8 Hz, 2H), 7.09-7.18 (m, 4H), 7.40 (s, 1H), 7.52-7.54 (m, 1H), 8.83 (t, J = 5.0 Hz, 1H). | 476.9 |
| 235 | 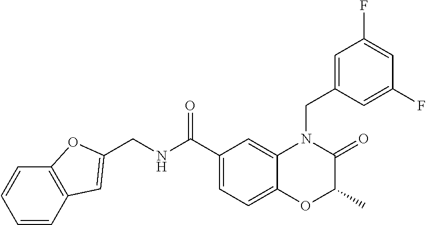 [SMILES: C[C@@H]1Oc2ccc(C(=O)NCc3cc4ccccc4o3)cc2N(Cc2cc(F)cc(F)c2)C1=O] | (S)-N- (benzofuran-2- ylmethyl)-4- (3,5- difluorobenzyl)- 2-methyl-3- oxo-3,4- dihydro-2H- benzo[b- 1,4]oxazine-6- carboxamide | (500 MHz; DMSO-d₆): δ 1.54 (d, J = 6.7 Hz, 3H), 4.59 (d, J = 5.5Hz, 2H), 5.01-5.07 (m, 1H), 5.11 (d, J = 16.85 Hz, 1H), 5.31 (d, J = 16.9 Hz, 1H), 6.68 (s, 1H), 7.02 (d, J = 6.7 Hz, 2H), 7.14-7.28 (m, 4H), 7.49-7.52 (m, 2H), 7.57 (d, J = 7.45 Hz, 1H), 7.61 (d, J = 8.4 Hz, 1H), 9.06 (t, J = 5.45 Hz, 1H). | 463.32 |

### Examples 236-243

Examples 236-243 were prepared in an entirely analogous manner to Examples 233-235 as described in General Procedures 1-5, starting with an appropriate alcohol in place of the thiol.

| | | | | LCMS |
|---|---|---|---|---|
| Example | Structure | IUPAC Name | ¹H-NMR | [M + H] |
| 236 | 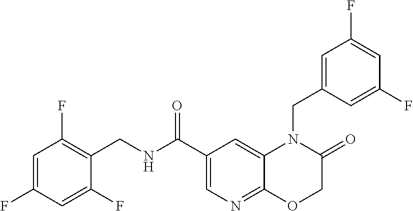 [SMILES: O=C(NCc1c(F)cc(F)cc1F)c1cnc2c(c1)N(Cc1cc(F)cc(F)c1)C(=O)CO2] | 1-(3,5- difluorobenzyl)- 2-oxo-N-(2,4,6- trifluorobenzyl)- 2,3-dihydro- 1H-pyrido[2,3- b-1,4]oxazine-7- carboxamide | (500 MHz; DMSO-d₆): δ 4.42 (d, J = 4.7 Hz, 2H), 5.10 (s, 2H), 5.17 (s, 2H), 7.09-7.19 (m, 5H), 7.57 (s, 1H), 8.32 (s, 1H), 8.98 (bs, 1H). | 464.24 |
| 237 | 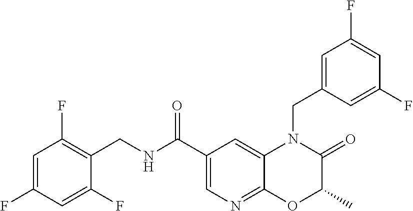 [SMILES: C[C@@H]1Oc2ncc(C(=O)NCc3c(F)cc(F)cc3F)cc2N(Cc2cc(F)cc(F)c2)C1=O] | (S)-1-(3,5- difluorobenzyl)- 3-methyl-2- oxo-N-(2,4,6- trifluorobenzyl)- 2,3-dihydro- 1H-pyrido[2,3- b-1,4]oxazine-7- carboxamide | (400 MHz; DMSO-d₆): δ 1.55 (d, J = 6.8 Hz, 3H), 4.40 (d, J = 4.88 Hz, 2H), 5.03 (d, J = 16.96 Hz, 1H), 5.22- 5.26 (m 2H), 7.01 (d, J = 6.44 Hz, 2H), 7.11- 7.16 (m, 3H), 7.61 (d, J = 1.88 Hz, 1H), 8.32 (d, J = 1.92 Hz, 1H), 8.94 (t, J = 4.92 Hz, 1H). | 478.32 |
| 238 | 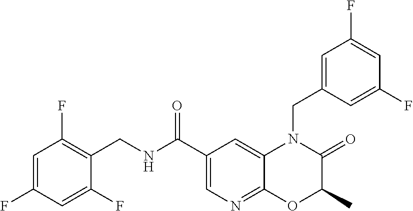 [SMILES: C[C@H]1Oc2ncc(C(=O)NCc3c(F)cc(F)cc3F)cc2N(Cc2cc(F)cc(F)c2)C1=O] | (R)-1-(3,5- difluorobenzyl)- 3-methyl-2- oxo-N-(2,4,6- trifluorobenzyl)- 2,3-dihydro- 1H-pyrido[2,3- b-1,4]oxazine-7- carboxamide | (400 MHz; DMSO-d₆): δ 1.55 (d, J = 6.76 Hz, 3H), 4.40 (d, J = 4.72 Hz, 2H), 5.03 (d, J = 16.84 Hz, 1H), 5.22- 5.26 (m 2H), 7.01 (d, J = 6.56 Hz, 2H), 7.12- 7.16 (m, 3H), 7.61 (d, J = 1.88 Hz, 1H), 8.31 (d, J = 1.88 Hz, 1H), 8.94 (t, J = 5.12 Hz, 1H). | 478.34 |
| 239 | 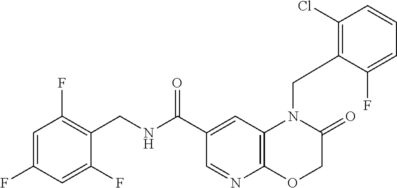 [SMILES: O=C(NCc1c(F)cc(F)cc1F)c1cnc2c(c1)N(Cc1c(F)cccc1Cl)C(=O)CO2] | 1-(2-chloro-6- fluorobenzyl)- 2-oxo-N-(2,4,6- trifluorobenzyl)- 2,3-dihydro- 1H-pyrido[2,3- b-1,4]oxazine-7- carboxamide | (500 MHz; DMSO-d₆): δ 4.43 (d, J = 4.64 Hz, 2H), 4.92 (s, 2H), 5.25 (s 2H), 7.16-7.20 (m, 3H), 7.30-7.33 (m, 2H), 7.86 (d, J = 1.68 Hz, 1H), 8.26 (d, J = 1.76 Hz, 1H), 8.89 (t, J = 4.92 Hz, 1H). | 480.26 |
| 240 | 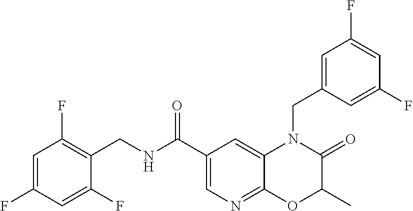 [SMILES: CC1Oc2ncc(C(=O)NCc3c(F)cc(F)cc3F)cc2N(Cc2cc(F)cc(F)c2)C1=O] | 1-(3,5- difluorobenzyl)- 3-methyl-2- oxo-N-(2,4,6- trifluorobenzyl)- 2,3-dihydro- 1H-pyrido[2,3- b-1,4]oxazine-7- carboxamide | (500 MHz; DMSO-d₆): δ 1.58 (d, J = 6.75 Hz, 3H), 4.43 (bs, 2H), 5.25-5.29 (m, 2H), 7.05 (d, J = 6.65 Hz, 2H), 7.17 (t, J = 8.65 Hz, 3H), 7.64 (s, 1H), 8.35 (s, 1H), 8.89 (bs, 1H). | 478.30 |
| 241 | 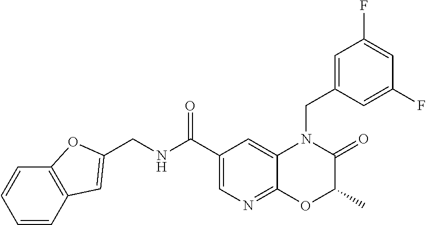 [SMILES: C[C@@H]1Oc2ncc(C(=O)NCc3cc4ccccc4o3)cc2N(Cc2cc(F)cc(F)c2)C1=O] | (S)-N- (benzofuran-2- ylmethyl)-1- (3,5- difluorobenzyl)- 3-methyl-2- oxo-2,3- dihydro-1H- pyrido[2,3- b][1,4]oxazine- 7-carboxamide | (500 MHz; DMSO-d₆): δ 1.59 (d, J = 6.75 Hz, 3H), 4.62 (d, J = 5.55Hz, 2H), 5.07 (d, J = 17 Hz, 1H), 5.27-5.32 (m, 2H), 6.73 (s, 1H), 7.07 (d, J = 6.6 Hz, 2H), 7.15-7.28 (m, 3H), 7.51-7.58 (m, 2H), 7.71 (d, J = 1.8 Hz, 1H), 8.43 (d, J = 1.85 Hz, 1H), 9.22 (t, J = 5.6 Hz, 1H). | 464.33 |
| 242 | 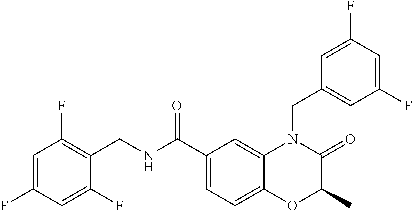 [SMILES: C[C@H]1Oc2ccc(C(=O)NCc3c(F)cc(F)cc3F)cc2N(Cc2cc(F)cc(F)c2)C1=O] | (R)-4-(3,5- difluorobenzyl)- 2-methyl-3- oxo-N-(2,4,6- trifluorobenzyl)- 3,4-dihydro- 2H- benzo[b][1,4] oxazine-6- carboxamide | (400 MHz; DMSO-d₆): δ 1.52 (d, J = 6.7 Hz, 3H), 4.40 (d, J = 4.9 Hz, 2H), 5.29-5.00 (m, 3H), 6.98 (d, J = 6.5 Hz, 2H), 7.16-7.08 (m, 4H), 7.40 (d, J = 1.5 Hz, 1H), 7.53-7.51 (m, 1H), 8.79 (bs, 1H). | 477.1 |
| 243 | 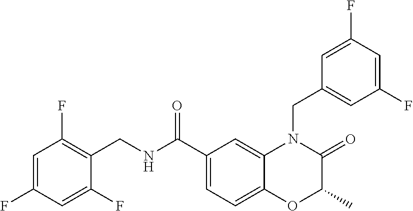 [SMILES: C[C@@H]1Oc2ccc(C(=O)NCc3c(F)cc(F)cc3F)cc2N(Cc2cc(F)cc(F)c2)C1=O] | (S)-4-(3,5- difluorobenzyl)- 2-methyl-3- oxo-N-(2,4,6- trifluorobenzyl)- 3,4-dihydro- 2H- benzo[b][1,4] oxazine-6- carboxamide | (400 MHz; DMSO-d₆): δ 1.52 (d, J = 6.7 Hz, 3H), 4.40 (d, J = 4.1 Hz, 2H), 5.29-5.00 (m, 3H), 6.98 (d, J = 6.3 Hz, 2H), 7.16-7.08 (m, 4H), 7.40 (s, 1H), 7.52 (d, J = 7Hz, 1H), 8.79 (bs, 1H). | 477.1 |
| 244 | 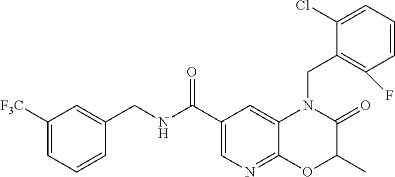 [SMILES: CC1Oc2ncc(C(=O)NCc3cccc(C(F)(F)F)c3)cc2N(Cc2c(F)cccc2Cl)C1=O] | 1-(2-chloro-6- fluorobenzyl)- 3-methyl-2- oxo-N-(3- (trifluoromethyl) benzyl)-2,3- dihydro-1H- pyrido[2,3- b][1,4]oxazine- 7-carboxamide | (500 MHz; DMSO-d₆): δ 1.51 (d, J = 5.3 Hz, 3H), 4.56 (bs, 2H), 5.08 (d, J = 5.8 Hz, 1H), 5.42-5.20 (m, 2H), 7.18 (t, J = 8.6 Hz, 1H), 7.35-7.32 (m, 2H), 7.66-7.59 (m, 4H), 7.95 (s, 1H), 8.40 (s, 1H), 9.20 (s, 1H). | 508.13 |

### Example 245

### Preparation 26: 4-(2-chloro-6-fluorobenzyl)-N-(furan-2-ylmethyl)-3-oxo-3,4-dihydro-2H-benzo[b][1,4]thiazine-6-carboxamide 1-oxide

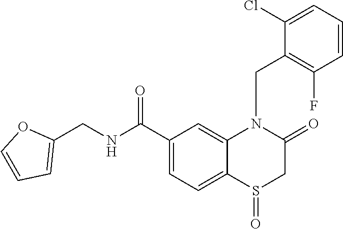 [SMILES: O=C(NCc1ccco1)c1ccc2c(c1)N(Cc1c(F)cccc1Cl)C(=O)CS2=O]

To a stirred solution of 4-(2-chloro-6-fluorobenzyl)-N-(furan-2-ylmethyl)-3-oxo-3,4-dihydro-2H-benzo[b-1,4]thiazine-6-carboxamide (Example 131) (0.03 g 0.069 mmol) in dry DCM (2 mL) was added mCPBA (0.012 g, 0.069 mmol) at 0-5° C. The resulting reaction mixture was stirred at room temperature for 1 h. It was then quenched with saturated Na₂S₂O₃ solution and extracted with EtOAc. The combined organics were washed with NaHCO₃ solution followed by water and brine. The organic layer was dried over anhydrous Na₂SO₄ and evaporated under reduced pressure. The crude product obtained was purified by prep-HPLC to obtain 4-(2-chloro-6-fluorobenzyl)-N-(furan-2-ylmethyl)-3-oxo-3,4-dihydro-2H-benzo-[b-1,4 thiazine-6-carboxamide-1-oxide (Example 245) (0.008 g, 0.017 mmol, 26% yield) as an off white solid. LCMS m/z: 447.09 [M+H]. ¹H NMR (500 MHz; DMSO-d₆): δ 4.28 (s, 2H), 4.48 (t, J=4.75 Hz, 2H), 5.36 (d, J=15.9 Hz, 1H), 5.45 (d, J=15.9 Hz, 1H), 6.29 (d, J=2.8 Hz, 1H), 6.41-6.42 (m, 1H), 7.15 (t, J=9.05 Hz, 1H), 7.30-7.35 (m, 2H), 7.60 (d, J=0.8 Hz, 1H), 7.73 (d, J=7.9 Hz, 1H), 7.92 (d, J=8.1 Hz, 2H), 9.18 (t, J=5.5 Hz, 1H).

### Example 246: 4-Benzyl-N-(furan-2-ylmethyl)-3,4-dihydro-2H-benzo[b-1,4]thiazine-6-carboxamide

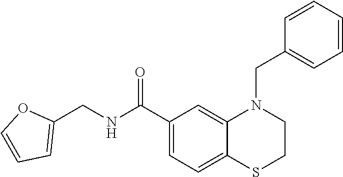 [SMILES: O=C(NCc1ccco1)c1ccc2c(c1)N(Cc1ccccc1)CCS2]

### Preparation 27: Methyl-4-benzyl-3,4-dihydro-2H-benzo[b][1,4]thiazine-6-carboxylat

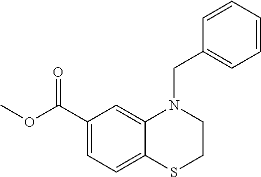 [SMILES: COC(=O)c1ccc2c(c1)N(Cc1ccccc1)CCS2]

To a stirred suspension of methyl 4-benzyl-3-oxo-3,4-dihydro-2H-benzo[b-1,4]thiazine-6-carboxylate (Preparation 7) (0.25 g, 0.80 mmol) in THF (2.5 mL) was added borane-THF solution (5.6 mL, 0.9 mol/L solution in THF) at 0-5° C. The resulting reaction mixture was stirred at RT for 16 h. After completion of the reaction, the reaction mixture was quenched by dropwise addition of MeOH (15 mL) at 0-5° C. The solvent was evaporated under reduced pressure. The residue obtained was partitioned between EtOAc and water; the organic layer was washed with brine, dried over anhydrous Na₂SO₄ and evaporated to dryness. The crude product obtained of methyl 4-benzyl-3,4-dihydro-2H-benzo[b][1,4]thiazine-6-carboxylate (0.25 g, 0.84 mmol) was used in next step without any further purification. LCMS m/z: 300.19 [M+H].

### Preparation 28: 4-Benzyl-N-(furan-2-ylmethyl)-3,4-dihydro-2H-benzo[b-1,4]thiazine-6-carboxamide

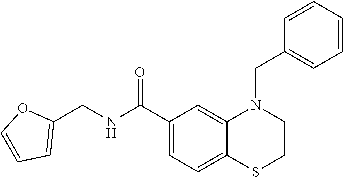 [SMILES: O=C(NCc1ccco1)c1ccc2c(c1)N(Cc1ccccc1)CCS2]

4-Benzyl-N-(furan-2-ylmethyl)-3,4-dihydro-2H-benzo[b-1,4]thiazine-6-carboxamide (Example 246) was prepared from methyl 4-benzyl-3,4-dihydro-2H-1,4-benzothiazine-6-carboxylate (Preparation 27) using an identical procedure to that described for Preparations 5 and 6. LCMS m/z: 365.22 [M+H]; ¹H NMR (500 MHz; DMSO-d₆): δ 3.13 (t, J=5.0 Hz, 2H), 3.63-3.65 (m, 2H), 4.38 (d, J=5.65 Hz, 2H), 4.62 (s, 2H), 6.18 (d, J=2.85 Hz, 1H), 6.37 (s, 1H), 7.04-7.09 (m, 2H), 7.17 (s, 1H), 7.24-7.30 (m, 3H), 7.34-7.37 (m, 2H), 7.55 (s, 1H), 8.76 (t, J=5.6 Hz, 1H).

### Examples 247-249

Examples 247-249 were prepared in an analogous manner to Example 246 as described in General Procedure 6 starting from the appropriate lactam.

| | | IUPAC | | LCMS |
|---|---|---|---|---|
| Example | Structure | Name | ¹H-NMR | [M + H] |
| 247 | 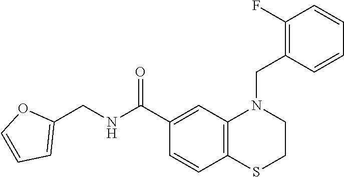 [SMILES: O=C(NCc1ccco1)c1ccc2c(c1)N(Cc1ccccc1F)CCS2] | 4-(2- fluorobenzyl)- N-(furan-2- ylmethyl)- 3,4-dihydro- 2H-benzo[b- 1,4]thiazine- 6- carboxamide | (500 MHz; DMSO-d₆): δ 3.12-3.14 (m, 2H), 3.65-3.67 (m, 2H), 4.37 (d, J = 5.65 Hz, 2H), 4.68 (s, 2H), 6.17 (d, J = 2.8 Hz, 1H), 6.37 (s, 1H), 7.05-7.27 (m, 6H), 7.31-7.35 (m, 1H), 7.55 (s, 1H), 8.78 (t, J = 5.45 Hz, 1H). | 383.26 |
| 248 | 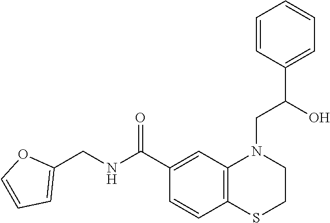 [SMILES: O=C(NCc1ccco1)c1ccc2c(c1)N(CC(O)c1ccccc1)CCS2] | N-(furan-2- ylmethyl)-4- (2-hydroxy- 2- phenylethyl)- 3,4-dihydro- 2H-benzo[b- 1,4]thiazine- 6- carboxamide | (500 MHz; DMSO-d₆): δ 2.89-3.01 (m, 2H), 3.47- 3.54 (m, 3H), 3.68-3.72 (m, 1H), 4.41-4.51 (m, 2H), 4.89-4.91 (m, 1H), 5.55 (d, J = 4.05 Hz, 1H), 6.28 (d, J = 2.15 Hz, 1H), 6.42 (s, 1H), 7.00 (d, J = 7.95 Hz, 1H), 7.05 (d, J = 7.9 Hz, 1H), 7.29-7.30 (m, 2H), 7.34- 7.40 (m, 4H), 7.60 (s, 1H), 8.84 (t, J = 5.4 Hz, 1H). | 395.24 |
| 249 | 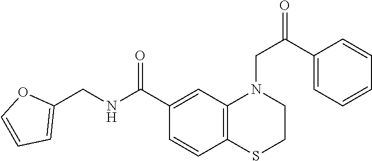 [SMILES: O=C(CN1CCSc2ccc(C(=O)NCc3ccco3)cc21)c1ccccc1] | N-(furan-2- ylmethyl)-4- (2-oxo-2- phenylethyl)- 3,4-dihydro- 2H- benzo[b][1,4] thiazine-6- carboxamide | (500 MHz; DMSO-d₆): δ 3.13-3.15 (m, 2H), 3.66- 3.68 (m, 2H), 4.34 (d, J = 5.6 Hz, 2H), 5.09 (s, 2H), 6.13 (d, J = 2.6 Hz, 1H), 6.33 (s, 1H), 6.88 (s, 1H), 7.03-7.09 (q, J = 8.1 Hz, 2H), 7.51 (s, 1H), 7.60 (t, J = 7.6 Hz, 2H), 7.71-7.74 (m, 1H), 8.06 (d, J = 7.7 Hz, 2H), 8.73 (t, J = 5.65 Hz, 1H). | 393.25 |

### Example 250: 4-(4-Chlorobenzyl)-N-(furan-2-ylmethyl)-3,4-dihydro-2H-benzo[b-1,4]thiazine-6-carboxamide

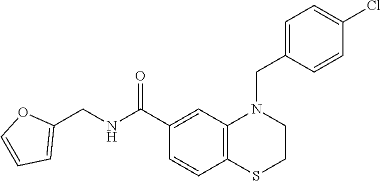 [SMILES: O=C(NCc1ccco1)c1ccc2c(c1)N(Cc1ccc(Cl)cc1)CCS2]

4-(4-Chlorobenzyl)-N-(furan-2-ylmethyl)-3,4-dihydro-2H-benzo[b-1,4]thiazine-6-carboxamide (Example 250) was purchased from a commercial supplier. LCMS m/z: 399.24 [M+H].

### Example 251

### Preparation 29: 4-Benzyl-N-(furan-2-ylmethyl)-, 4-dihydro-2H-benzo[b-1,4]thiazine-6-carboxamide-1-oxide

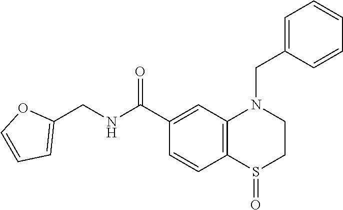 [SMILES: O=C(NCc1ccco1)c1ccc2c(c1)N(Cc1ccccc1)CCS2=O]

4-Benzyl-N-(furan-2-ylmethyl)-3,4-dihydro-2H-benzo[b-1,4]thiazine-6-carboxamide 1-oxide (Example 251) was prepared from 4-benzyl-N-(furan-2-ylmethyl)-3,4-dihydro-2H-benzo[b-1,4]thiazine-6-carboxamide (Example 246) using an identical procedure to that described in Preparation 26 and described in General Procedure 16. LCMS m/z: 381.29 [M+H]; ¹H NMR (500 MHz; DMSO-d₆): δ 2.93-2.99 (m, 1H), 3.16-3.19 (m, 1H), 3.66-3.68 (m, 1H), 3.95 (t, J=13 Hz, 1H), 4.41 (d, J=5.60 Hz, 2H), 4.78-4.88 (q, J=17.2 Hz, 2H), 6.20 (d, J=2.7 Hz, 1H), 6.38 (s, 1H), 7.13 (d, J=8.3 Hz, 1H), 7.29 (t, J=6.95 Hz, 4H), 7.34-7.37 (m, 2H), 7.57-7.61 (m, 2H), 9.00 (t, J=5.6 Hz, 1H).

### Example 252

### Preparation 30: 4-Benzyl-N-(furan-2-ylmethyl)-3,4-dihydro-2H-benzo[b-1,4]thiazine-6-carboxamide 1,1-dioxide

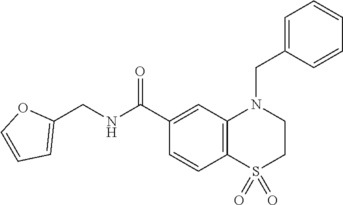 [SMILES: O=C(NCc1ccco1)c1ccc2c(c1)N(Cc1ccccc1)CCS2(=O)=O]

To a stirred solution of 4-benzyl-N-(furan-2-ylmethyl)-3,4-dihydro-2H-benzo[b-1,4]thiazine-6-carboxamide (Example 246) (0.1 g 0.27 mmol) in dry DCM (2 mL) was added mCPBA (0.142 g, 0.82 mmol) in 3 equal portions at an interval of 6 h between portions at 0-5° C. under an inert atmosphere. The resulting reaction mixture was then stirred at room temperature for 20 h. It was quenched with saturated Na₂S₂O₃ solution and extracted with EtOAc. The combined organics were washed with NaHCO₃ solution followed by water and brine. The organic layer was dried over anhydrous Na₂SO₄ and evaporated under reduced pressure. The crude product obtained was purified by prep-HPLC to obtain 4-benzyl-N-(furan-2-ylmethyl)-3,4-dihydro-2H-benzo[b-1,4]thiazine-6-carboxamide 1,1-dioxide (Example 252) (0.03 g, 0.075 mmol, 28% yield) as an off white solid. LCMS m/z: 397.21 [M+H]; ¹H NMR (500 MHz; DMSO-d₆): δ 3.65-3.67 (m, 2H), 3.97-3.99 (m, 2H), 4.41 (d, J=5.60 Hz, 2H), 4.78 (s, 2H), 6.21 (d, J=2.9 Hz, 1H), 6.38 (s, 1H), 7.21-7.24 (m, 2H), 7.27-7.30 (m, 3H), 7.35-7.38 (m, 2H), 7.57 (s, 1H), 7.71 (d, J=8.15 Hz, 1H), 9.04 (t, J=5.6 Hz, 1H).

### Example 253: 4-(2-Fluorobenzoyl)-N-(furan-2-ylmethyl)-3,4-dihydro-2H-benzo[b-1,4]thiazine-6-carboxamide

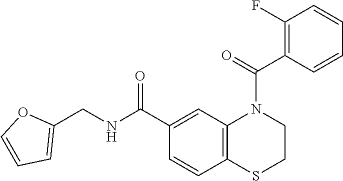 [SMILES: O=C(NCc1ccco1)c1ccc2c(c1)N(C(=O)c1ccccc1F)CCS2]

### Preparation 31: Methyl-3,4-dihydro-2H-benzo[b-1,4]thiazine-6-carboxylate

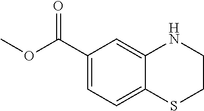

Methyl-3,4-dihydro-2H-benzo[b-1,4]thiazine-6-carboxylate was prepared from methyl 3-oxo-3,4-dihydro-2H-benzo[b-1,4]-thiazine-6-carboxylate (Preparation 1, Step 2) using an identical procedure to that described for Preparation 27. LCMS m/z: 210.08 [M+H].

### Preparation 32: Methyl 4-(2-fluorobenzoyl)-3,4-dihydro-2H-benzo[b-1,4]thiazine-6-carboxylate

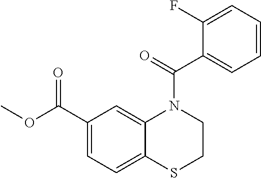 [SMILES: COC(=O)c1ccc2c(c1)N(C(=O)c1ccccc1F)CCS2]

To a stirred solution of methyl 3,4-dihydro-2H-benzo[b-1,4]thiazine-6-carboxylate (Preparation 31) (0.2 g, 0.96 mmol) in dry DCM (5 mL) was added TEA (0.33 mL, 2.39 mmol) at RT, then 2-fluorobenzoyl chloride (0.227, 1.44 mmol) was added dropwise with ice cooling and the reaction mixture was stirred at room temperature for 3 h. After completion of the reaction it was diluted with DCM and washed successively with 1N HCl, saturated NaHCO₃ solution and then brine. The organic layer was dried over anhydrous Na₂SO₄ and evaporated to dryness. The crude product of methyl 4-(2-fluorobenzoyl)-3,4-dihydro-2H-benzo[b-1,4]thiazine-6-carboxylate (0.25 g, 0.76 mmol, 79% yield) obtained was used in the next step with no further purification. LCMS m/z: 332.15 [M+H].

### Preparation 33: 4-(2-Fluorobenzoyl)-3,4-dihydro-2H-benzo[b][1,4]thiazine-6-carboxylic acid

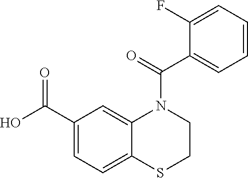 [SMILES: O=C(O)c1ccc2c(c1)N(C(=O)c1ccccc1F)CCS2]

To a stirred solution of methyl 4-(2-fluorobenzoyl)-3,4-dihydro-2H-benzo[b-1,4]thiazine-6-carboxylate (Preparation 32) (0.25 g, 0.76 mmol) in a mixture of MeOH, water and THF (1:1:2; 20 mL) was added LiOH.H₂O (0.064 g, 1.51 mmol) at RT. The reaction was allowed to stir at room temperature for 16 h. Upon completion of the reaction, the solvents were evaporated under reduced pressure and the residue was dissolved in water, washed with EtOAc and then acidified to ˜pH 5 with dilute HCl. The acidified aqueous solution was then extracted with EtOAc. The combined organic extracts were washed with brine, dried over anhydrous Na₂SO₄ and evaporated to dryness to give 4-(2-fluorobenzoyl)-3,4-dihydro-2H-benzo[b][1,4]thiazine-6-carboxylic acid (0.2 g. 0.63 mmol, 84% yield) as an off white solid which was used in the next step without any further purification. LCMS m/z: 316.12 [M+H].

### Preparation 34: 4-(2-Fluorobenzoyl)-N-(furan-2-ylmethyl)-3,4-dihydro-2H-benzo[b-1,4]thiazine-6-carboxamide

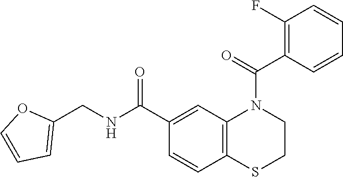 [SMILES: O=C(NCc1ccco1)c1ccc2c(c1)N(C(=O)c1ccccc1F)CCS2]

4-(2-Fluorobenzoyl)-N-(furan-2-ylmethyl)-3,4-dihydro-2H-benzo[b-1,4]thiazine-6-carboxamide (Example 253) was prepared from 4-(2-fluorobenzoyl)-3,4-dihydro-2H-benzo[b-1,4]-thiazine-6-carboxylic acid (Preparation 33) following the method described for Example 1. LCMS m/z: 397.23 [M+H]; ¹H NMR (500 MHz; DMSO-d₆): δ 3.32 (bs, 2H), 3.37 (s, 2H), 4.34 (bs, 2H), 6.12 (bs, 1H), 6.39 (s, 1H), 7.05-7.23 (m, 3H), 7.32 (d, J=8.25 Hz, 2H), 7.33-7.45 (m, 2H), 7.52-7.53 (m, 1H), 7.57 (s, 1H), 8.70 (bs, 1H).

### Example 254

### Preparation 35: 4-(2-Fluorobenzoyl)-N-(furan-2-ylmethyl)-3,4-dihydro-2H-benzo[b-1,4]thiazine-6-carboxamide 1,1-dioxide

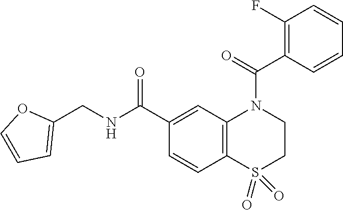 [SMILES: O=C(NCc1ccco1)c1ccc2c(c1)N(C(=O)c1ccccc1F)CCS2(=O)=O]

4-(2-fluorobenzoyl)-N-(furan-2-ylmethyl)-3,4-dihydro-2H-benzo[b-1,4]thiazine-6-carboxamide 1,1-dioxide (Example 254) was prepared from 4-(2-fluorobenzoyl)-N-(furan-2-ylmethyl)-3,4-dihydro-2H-benzo[b-1,4]thiazine-6-carboxamide (Example 253) following the method described for Preparation 30/Example 249. LCMS m/z: 429.23 [M+H]; ¹H NMR (500 MHz; DMSO-d₆): δ 3.92 (s, 2H), 4.38 (bs, 4H), 6.18 (s, 1H), 6.41 (s, 1H), 7.22-7.29 (m, 2H), 7.54-7.59 (m, 3H), 7.69 (bs, 1H), 7.84 (d, J=8.05 Hz, 1H), 7.98 (d, J=7.8 Hz, 1H), 9.11 (bs, 1H).

### Examples 255-258

Examples 255-258 were prepared in an analogous manner to that described for Example 253 starting from Preparation 31 using the appropriate acyl halides and amines.

| | | IUPAC | LCMS | |
|---|---|---|---|---|
| Example | Structure | Name | ¹H-NMR | [M + H] |
| 255 | 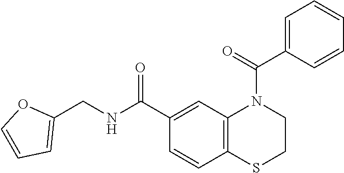 [SMILES: O=C(NCc1ccco1)c1ccc2c(c1)N(C(=O)c1ccccc1)CCS2] | 4-benzoyl-N- (furan-2- ylmethyl)-3,4- dihydro-2H- benzo[b][1,4] thiazine-6- carboxamide | (500 MHz; DMSO-d₆): δ 3.33-3.36 (m, 2H), 4.04 (bs, 2H), 4.33 (d, J = 5.4 Hz, 2H), 6.10 (s, 1H), 6.38 (s, 1H), 7.33-7.36 (m, 6H), 7.40-7.41 (m, 1H), 7.53-7.55 (m, 2H), 8.71-8.73 (m, 1H). | 379.23 |
| 256 | 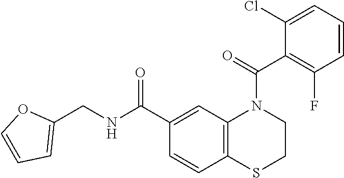 [SMILES: O=C(NCc1ccco1)c1ccc2c(c1)N(C(=O)c1c(F)cccc1Cl)CCS2] | 4-(2-chloro-6- fluorobenzoyl)- N-(furan-2- ylmethyl)-3,4- dihydro-2H- benzo[b][1,4] thiazine-6- carboxamide | (500 MHz; DMSO-d₆): δ 3.95 (bs, 1H), 4.33 (d, J = 5.3 Hz, 3H), 6.13 (d, J = 2.5 Hz, 1H), 6.40 (s, 1H), 7.19-7.70 (m, 9H), 8.77 (t, J =5.5 Hz, 1H). | 431.17 |
| 257 | 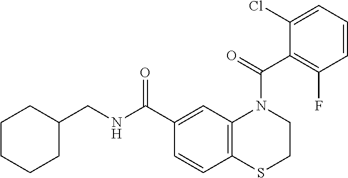 [SMILES: O=C(NCC1CCCCC1)c1ccc2c(c1)N(C(=O)c1c(F)cccc1Cl)CCS2] | 4-(2-chloro-6- fluorobenzoyl)- N- (cyclohexyl methyl)-3,4- dihydro-2H- benzo[b][1,4] thiazine-6- carboxamide | (500 MHz; DMSO-d₆): 0.83 (bs, 1H), 1.16 (bs, 4H), 1.40 (bs, 1H), 1.59- 1.68 (m, 5H), 2.95 (bs, 2H), 3.11 (bs, 1H), 3.76 (bs, 1H), 3.97 (bs, 1H), 4.33 (bs, 1H), 7.16-7.46 (m, 6H), 8.19 (bs, 1H). | 447.25 |
| 258 | 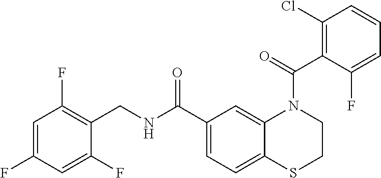 [SMILES: O=C(NCc1c(F)cc(F)cc1F)c1ccc2c(c1)N(C(=O)c1c(F)cccc1Cl)CCS2] | 4-(2-chloro-6- fluorobenzoyl)- N-(2,4,6- trifluorobenzyl)- 3,4- dihydro-2H- benzo[b][1,4] thiazine-6- carboxamide | (500 MHz; DMSO-d₆): δ 3.73-3.76 (m, 1H), 3.92 (bs, 1H), 4.32-4.35 (m, 3H), 7.14 (s, 1H), 7.17- 7.22 (m, 4H), 7.29 (d, J = 8.25 Hz, 1H), 7.34- 7.41 (m, 1H), 7.45-7.48 (m, 1H), 7.53-7.67 (m, 1H), 8.66 (t, J = 4.5 Hz, 1H). | 495.24 |
| 259 | 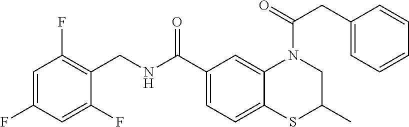 [SMILES: CC1CN(C(=O)Cc2ccccc2)c2cc(C(=O)NCc3c(F)cc(F)cc3F)ccc2S1] | 2-methyl-4- (2- phenylacetyl)- N-(2,4,6- trifluorobenzyl)- 3,4- dihydro-2H- benzo[b][1,4] thiazine-6- carboxamide | (400 MHz; DMSO-d₆): δ 1.24 (d, J = 6.6 Hz, 3H), 3.19 (bs, 1H), 3.52 (bs, 1H), 3.85 (s, 2H), 4.31 (bs, 1H), 4.44 (s, 2H), 7.24-7.14 (m, 8H), 7.58 (d, J = 7.6 Hz, 1H), 7.89 (s, 1H), 8.81 (s, 1H). | 471.26 |
| 260 | 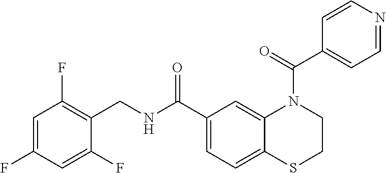 [SMILES: O=C(NCc1c(F)cc(F)cc1F)c1ccc2c(c1)N(C(=O)c1ccncc1)CCS2] | 4- isonicotinoyl- N-(2,4,6- trifluorobenzyl)- 3,4- dihydro-2H- benzo[b][1,4] thiazine-6- carboxamide | (500 MHz; DMSO-d₆): δ 3.30 (s, 2H), 4.00 (bs, 2H), 4.34 (s, 2H), 7.17 (t, J = 8.5 Hz, 2H), 7.29 (bs, 2H), 7.34 (d, J = 8.2 Hz, 2H), 7.54 (d, 8.1 Hz, 1H), 8.58 (s, 2H), 8.64 (s, 1H). | 444.30 |

### Example 261: 4-(Phenylsulfonyl)-N-(2,4,6-trifluorobenzyl)-3,4-dihydro-2H-benzo[b-1,4]thiazine-6-carboxamide

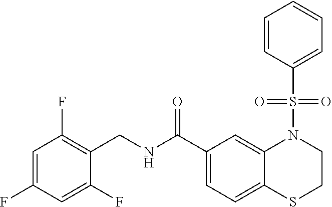 [SMILES: O=C(NCc1c(F)cc(F)cc1F)c1ccc2c(c1)N(S(=O)(=O)c1ccccc1)CCS2]

### Preparation 36: Methyl-4-(phenylsulfonyl)-3,4-dihydro-2H-benzo[b-1,4]thiazine-6-carboxylate

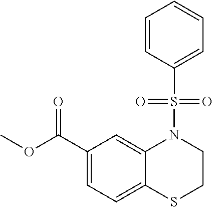 [SMILES: COC(=O)c1ccc2c(c1)N(S(=O)(=O)c1ccccc1)CCS2]

To a stirred solution of methyl 3,4-dihydro-2H-benzo[b-1,4]thiazine-6-carboxylate (Preparation 31) (0.15 g, 0.72 mmol) in dry pyridine (6 mL) was added DMAP (0.01 g, 0.082 mmol) at RT, then benzenesulfonyl chloride (0.142 mL, 1.1 mmol) was added dropwise with ice cooling, then the reaction mixture was stirred at 60° C. for 4 h. After this time, the reaction mixture was acidified with 3N HCl and the resulting solution was extracted with EtOAc. The organic layer was washed with saturated NaHCO₃ solution and then brine and then dried over anhydrous Na₂SO₄ and evaporated to dryness. The crude product of methyl-4-(phenylsulfonyl)-3,4-dihydro-2H-benzo[b-1,4]thiazine-6-carboxylate (0.2 g, 0.57 mmol, 80% yield) obtained was used in the next step with no further purification. LCMS m/z: 350.16 [M+H].

### Preparation 37: 4-(Phenylsulfonyl)-N-(2,4,6-trifluorobenzyl)-3,4-dihydro-2H-benzo[b-1,4]thiazine-6-carboxamide

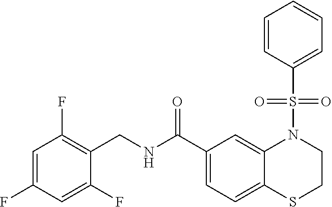 [SMILES: O=C(NCc1c(F)cc(F)cc1F)c1ccc2c(c1)N(S(=O)(=O)c1ccccc1)CCS2]

4-(Phenylsulfonyl)-N-(2,4,6-trifluorobenzyl)-3,4-dihydro-2H-benzo[b-1,4]thiazine-6-carboxamide (Example 261) was prepared using the method described in Preparation 36 and Example 1 using the General Procedures 1-3. LCMS m/z: 479.27 [M+H]; ¹H NMR (500 MHz; DMSO-d₆): δ 2.90 (t, J=5.4 Hz, 2H), 3.93 (t, J=5.35 Hz, 2H), 4.46 (d, J=4.85 Hz, 2H), 7.20-7.23 (m, 3H), 7.51-7.64 (m, 5H), 7.70 (t, J=6.9 Hz, 1H), 8.04 (d, J=1.25 Hz, 1H), 8.92-8.94 (m, 1H).

### Example 262: 4-((1,5-Difluorophenyl)sulfonyl)-N-(2,4,6-trifluorobenzyl)-3,4-dihydro-2H-benzo[b-1,4]thiazine-6-carboxamide

Example 262 was prepared in an analogous manner to Example 261 using 3,5-difluorobenzene sulfonyl chloride and 2,4,6-trifluorobenzyl amine.

| | | IUPAC | | LCMS |
|---|---|---|---|---|
| Example | Structure | Name | ¹H-NMR | [M + H] |
| 262 | 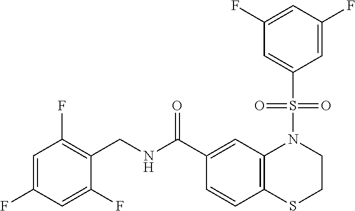 [SMILES: O=C(NCc1c(F)cc(F)cc1F)c1ccc2c(c1)N(S(=O)(=O)c1cc(F)cc(F)c1)CCS2] | 4-((3,5- difluorophenyl) sulfonyl-N- (2,4,6- trifluorobenzyl)- 3,4- dihydro-2H- benzo[b- 1,4]thiazine- 6- carboxamide | (400 MHz; DMSO-d₆): δ 3.00 (t, J = 5.72 Hz, 2H), 3.95 (t, J = 5.52 Hz, 2H), 4.44 (d, J = 4.88 Hz, 2H), 7.17 (t, J = 8.56 Hz, 2H), 7.25 (d, J = 8.28 Hz, 1H), 7.31-7.33 (m, 2H), 7.62- 7.64 (m, 1H), 7.68-7.73 (m, 1H), 7.91 (d, J = 1.72 Hz, 1H), 8.90 (t, J = 5.12 Hz, 1H). | 515.27 |

### Examples 263-26t

Examples 263-265 were prepared in an entirely analogous manner to Example 253, starting with the appropriate lactam and using the appropriate acyl halide, alkyl halide and amine as described in General Procedures 1-3 and 6.

| | | IUPAC | | LCMS |
|---|---|---|---|---|
| Example | Structure | Name | ¹H-NMR | [M + H] |
| 263 | 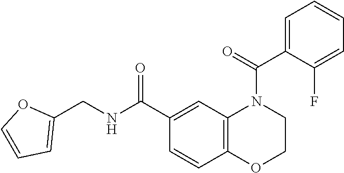 [SMILES: O=C(NCc1ccco1)c1ccc2c(c1)N(C(=O)c1ccccc1F)CCO2] | 4-(2- fluorobenzoyl)- N-(furan- 2-ylmethyl)- 3,4-dihydro- 2H-benzo[b- 1,4]oxazine- 6- carboxamide | (500 MHz; DMSO-d₆): δ 3.78-3.79 (m, 2H), 4.34- 4.39 (m, 5H), 6.20 (bs, 1H), 6.40 (s, 1H), 6.99 (d, J = 8.4 Hz, 1H), 7.23- 7.33 (m, 1H), 7.57-7.60 (m, 5H), 8.66-8.85 (m, 1H). | 381.19 |
| 264 | 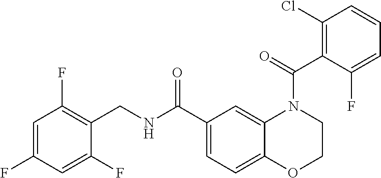 [SMILES: O=C(NCc1c(F)cc(F)cc1F)c1ccc2c(c1)N(C(=O)c1c(F)cccc1Cl)CCO2] | 4-(2-chloro- 6- fluorobenzoyl)- N-(2,4,6- trifluorobenzyl)- 3,4- dihydro-2H- benzo[b- 1,4]oxazine- 6- carboxamide | (500 MHz; DMSO-d₆): δ 3.68 (bs, 1H), 3.94-3.98 (m, 0.5H), 4.32 (bs, 2H), 4.45 (s, 2H), 6.94-7.03 (m, 2H), 7.20-7.29 (m, 3H), 7.45-7.53 (m, 2H), 7.60-7.66 (m, 1H), 8.54- 8.58 (m, 0.5H), 8.75- 8.86 (m, 1H). | 479.23 |
| 265 | 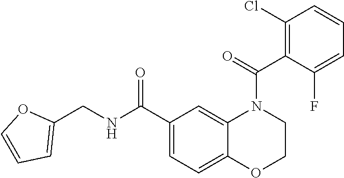 [SMILES: O=C(NCc1ccco1)c1ccc2c(c1)N(C(=O)c1c(F)cccc1Cl)CCO2] | 4-(2-chloro- 6- fluorobenzoyl)- N-(furan- 2-ylmethyl)- 3,4-dihydro- 2H-benzo[b- 1,4]oxazine- 6- carboxamide | (500 MHz; DMSO-d₆): δ 3.60-3.69 (m, 1.5H), 3.98-4.00 (m, 0.5H), 4.32 (bs, 2H), 4.46-4.47 (m, 2H), 6.13-6.27 (m, 1H), 6.40 (s, 1H), 6.96- 7.10 (m, 1H), 7.27-7.31 (m, 1H), 7.44-7.70 (m, 4H), 8.69-8.96 (m, 2H). | 415.24 |

### Example 266: 4-Benzyl-N-(furan-2-ylmethyl)-3-thioxo-3,4-dihydro-2H-benzo[b-1,4]thiazine-6-carboxamide

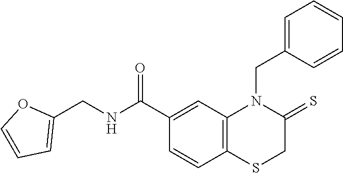 [SMILES: O=C(NCc1ccco1)c1ccc2c(c1)N(Cc1ccccc1)C(=S)CS2]

### Preparation 38: Methyl-4-benzyl-3-thioxo-3,4-dihydro-2H-benzo[b][1,4]thiazine-6-carboxylate

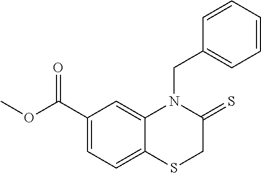 [SMILES: COC(=O)c1ccc2c(c1)N(Cc1ccccc1)C(=S)CS2]

To a stirred solution of methyl 4-benzyl-3-oxo-3,4-dihydro-2H-benzo[b-1,4]thiazine-6-carboxylate (Preparation 7) (0.24 g, 0.80 mmol) in xylene (8 mL) was added Lawesson's reagent (0.258 g, 0.64 mmol) at RT. The resulting reaction mixture was heated at 115-120° C. for 24 h. TLC and LCMS showed product formation along with some starting material. The reaction mixture was diluted with water, extracted with EtOAc; the combined organics were washed with brine, dried over anhydrous Na₂SO₄ and concentrated under reduced pressure to give the crude product which was purified by column chromatography using 5-10% EtOAc in hexanes to afford methyl 4-benzyl-3-thioxo-3,4-dihydro-2H-benzo[b][1,4]thiazine-6-carboxylate (0.12 g, 0.36 mmol, 46% yield) as a yellow solid. LCMS m/z: 330.2 [M+H].

### Preparation 39: 4-Benzyl-N-(furan-2-ylmethyl)-3-thioxo-3,4-dihydro-2H-benzo[b-1,4]thiazine-6-carboxamide

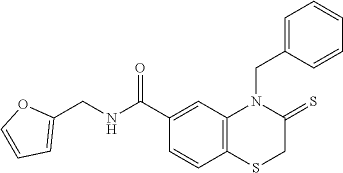 [SMILES: O=C(NCc1ccco1)c1ccc2c(c1)N(Cc1ccccc1)C(=S)CS2]

4-benzyl-N-(furan-2-ylmethyl)-3-thioxo-3,4-dihydro-2H-benzo[b-1,4]thiazine-6-carboxamide (Example 266) was prepared from methyl 4-benzyl-3-thioxo-3,4-dihydro-2H-benzo[b-1,4]thiazine-6-carboxylate (Preparation 38) using identical methods described in Preparations 5 and 6. LCMS m/z: 393.0 [M−H]; ¹H NMR (400 MHz; CDCl₃): δ 0.86-0.88 (m, 1H), 4.03 (s, 2H), 4.55 (d, J=4.84 Hz, 2H), 5.91 (s, 2H), 6.25 (s, 1H), 6.14 (s, 1H), 6.24 (s, 1H), 6.33 (s, 1H), 7.19-7.20 (m, 1H), 7.25-7.33 (m, 3H), 7.36-7.39 (m, 2H), 7.68 (bs, 1H).

### Example 267: 1-(2-Chloro-6-fluorobenzyl)-3-methyl-2-oxo-N-(2,4,6-trifluorobenzyl)-1,2,3,4-tetrahydroquinoline-7-carboxamide

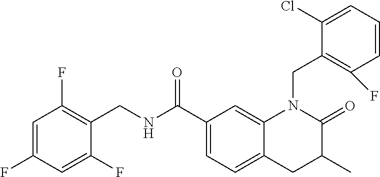 [SMILES: CC1Cc2ccc(C(=O)NCc3c(F)cc(F)cc3F)cc2N(Cc2c(F)cccc2Cl)C1=O]

### Preparation 40: Methyl-1-(2-chloro-6-fluorobenzyl)-2-oxo-1,2,3,4-tetrahydro quinoline-7-carboxylate

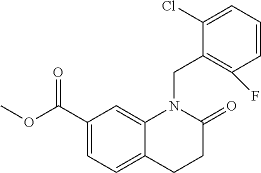 [SMILES: COC(=O)c1ccc2c(c1)N(Cc1c(F)cccc1Cl)C(=O)CC2]

Methyl-1-(2-chloro-6-fluorobenzyl)-2-oxo-1,2,3,4-tetrahydroquinoline-7-carboxylate was prepared from commercially available methyl 2-oxo-1,2,3,4-tetrahydroquinoline-7-carboxylate and 2-chloro-6-fluoro-benzyl chloride using an identical method to that described in Preparation 4. LCMS m/z: 348.14 [M+H].

### Preparation 41: Methyl-1-(2-chloro-6-fluorobenzyl)-3-methyl-2-oxo-1,2,3,4-tetrahydroquinoline-7-carboxylate

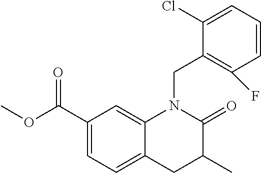 [SMILES: COC(=O)c1ccc2c(c1)N(Cc1c(F)cccc1Cl)C(=O)C(C)C2]

To a stirred solution of methyl 1-(2-chloro-6-fluorobenzyl)-2-oxo-1,2,3,4 tetrahydroquinoline-7-carboxylate (Preparation 40) (0.6 g, 0.28 mmol) in dry THF (20 mL) was added LiHMDS (1.6 mL, 2.07 mmol) at −78° C. and allowed to stir for 15 min. at −78 C followed by addition of Mel (0.16 mL, 2.07 mmol) and maintained at −78° C. for 30 min. After completion of reaction (monitored by TLC or and LCMS), the reaction mixture was quenched with water, extracted with EtOAc, dried and evaporated to obtain the crude which was purified by Combi-flash (4.0 g column) and eluted with 32% EtOAc in hexanes to afford methyl 1-(2-chloro-6-fluorobenzyl)-3-methyl-2-oxo-1,2,3,4-tetrahydroquinoline-7-carboxylate (0.11 g, 0.35 mmol, 17% yield) as a colourless oil. LCMS m/z: 362.21 [M+H].

### Preparation 42: 1-(2-Chloro-6-fluorobenzyl)-3-methyl-2-oxo-N-(2,4,6-trifluorobenzyl)-1,2,3,4-tetrahydroquinoline-7-carboxamide

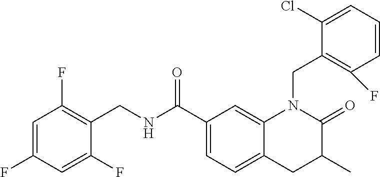 [SMILES: CC1Cc2ccc(C(=O)NCc3c(F)cc(F)cc3F)cc2N(Cc2c(F)cccc2Cl)C1=O]

1-(2-chloro-6-fluorobenzyl)-3-methyl-2-oxo-N-(2,4,6-trifluorobenzyl)-1,2,3,4-tetrahydroquinoline-7-carboxamide (Example 267) was prepared from methyl-1-(2-chloro-6-fluorobenzyl)-3-methyl-2-oxo-1,2,3,4-tetrahydroquinoline-7-carboxylate (Preparation 41) using identical methods described in Preparations 5 and 6. LCMS m/z: 491.27 [M+H]; ¹H NMR (500 MHz; DMSO-d₆): δ 1.14 (d, J=6.4 Hz, 3H), 2.56-2.68 (m, 2H), 2.93-2.96 (m, 1H), 4.39-4.48 (m, 2H), 5.16 (d, J=15.85 Hz, 1H), 5.45 (d, J=15.9 Hz, 1H), 7.10-7.14 (m, 1H), 7.21 (t, J=8.7 Hz, 2H), 7.27-7.32 (m, 3H), 7.41-7.43 (dd, J₁=7.7 Hz, J₂=0.95 Hz, 1H), 7.50 (s, 1H), 8.78 (t, J=5.1 Hz, 1H).

### Examples 268-275

Examples 268-275 were prepared in an identical manner to that described for Example 267 starting from an appropriate nitroarene and using the appropriate benzyl halides and amines as described for General procedures 7-10.

| | | IUPAC | | LCMS |
|---|---|---|---|---|
| Example | Structure | Name | ¹H-NMR | [M + H] |
| 268 | 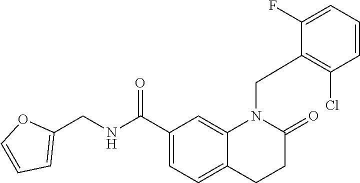 [SMILES: O=C(NCc1ccco1)c1ccc2c(c1)N(Cc1c(F)cccc1Cl)C(=O)CC2] | 1-(2-chloro- 6- fluorobenzyl)- N-(furan-2- ylmethyl)-2- oxo-1,2,3,4- tetrahydro quinoline-7- carboxamide | (500 MHz; DMSO-d₆): δ 2.60 (t, J = 7.6 Hz, 2H), 2.88 (t, J = 6.55 Hz, 2H), 4.43 (d, J = 5.65 Hz, 2H), 5.31 (s, 2H), 6.24 (d, J = 2.8 Hz, 1H), 6.40-6.41 (m, 1H), 7.12-7.16 (m, 1H), 7.27-7.33 (m, 3H), 7.46-7.48 (m, 1H), 7.57- 7.59 (m, 2H), 8.90 (t, J = 5.5 Hz, 1H). | 412.94 |
| 269 | 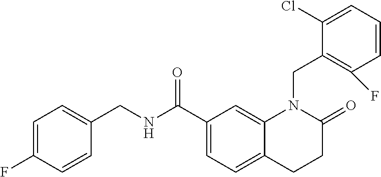 [SMILES: O=C(NCc1ccc(F)cc1)c1ccc2c(c1)N(Cc1c(F)cccc1Cl)C(=O)CC2] | 1-(2-chloro- 6- fluorobenzyl)- N-(4- fluorobenzyl)- 2-oxo- 1,2,3,4- tetrahydro quinoline-7- carboxamide | (500 MHz; DMSO-d₆): δ 2.59-2.64 (m, 2H), 2.88 (t, J = 6.6 Hz, 2H), 4.43 (d, J = 5.85 Hz, 2H), 5.32 (s, 2H), 7.13-7.57 (m, 10H), 8.98 (t, J = 5.8 Hz, 1H). | 441.25 |
| 270 | 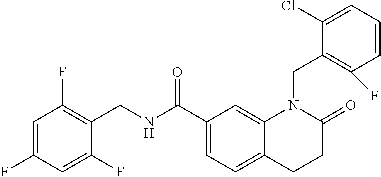 [SMILES: O=C(NCc1c(F)cc(F)cc1F)c1ccc2c(c1)N(Cc1c(F)cccc1Cl)C(=O)CC2] | 1-(2-chloro- 6- fluorobenzyl)- 2-oxo-N- (2,4,6- trifluorobenzyl)- 1,2,3,4- tetrahydro quinoline-7- carboxamide | (500 MHz; DMSO-d₆): δ 2.59 (t, J = 7.65 Hz, 2H), 2.86 (t, J = 6.75 Hz, 2H), 4.43 (d, J = 4.95 Hz, 2H), 5.30 (s, 2H), 7.13-7.15 (m, 1H), 7.21 (t, J = 8.55 Hz, 2H), 7.26-7.31 (m, 3H), 7.42 (d, J = 7.75 Hz, 1H), 7.51 (s, 1H), 8.78 (bs, 1H). | 477.42 |
| 271 | 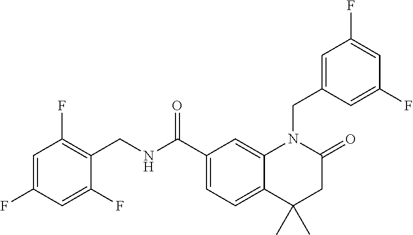 [SMILES: CC1(C)CC(=O)N(Cc2cc(F)cc(F)c2)c2cc(C(=O)NCc3c(F)cc(F)cc3F)ccc21] | 1-(3,5- difluorobenzyl)- 4,4- dimethyl-2- oxo-N-(2,4,6- trifluorobenzyl)- 1,2,3,4- tetrahydro quinoline-7- carboxamide | (400 MHz; DMSO-d₆): δ 1.26 (s, 6H), 2.66 (s, 2H), 4.41 (d, J = 4.92 Hz, 2H), 5.20 (s, 2H), 6.93-6.94 (m, 2H), 7.10-7.16 (m, 3H), 7.32 (s, 1H), 7.43 (d, J = 8.0 Hz, 1H), 7.53 (d, J = 7.84 Hz, 1H), 8.85 (t, J = 4.88 Hz, 1H). | 489.1 |
| 272 | 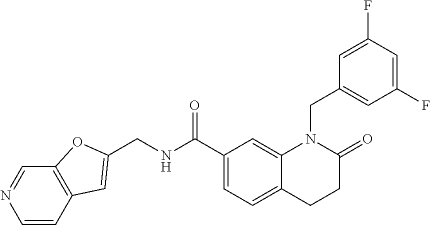 [SMILES: O=C(NCc1cc2ccncc2o1)c1ccc2c(c1)N(Cc1cc(F)cc(F)c1)C(=O)CC2] | 1-(3,5- difluorobenzyl)- N- (furo[2,3- c]pyridin-2- ylmethyl)-2- oxo-1,2,3,4- tetrahydro quinoline-7- carboxamide | (400 MHz; DMSO-d₆): δ 2.74 (t, J = 7.4 Hz, 2H), 3.02 (t, J = 7.4 Hz, 2H), 4.62 (d, J = 5.5 Hz, 2H), 5.19 (s, 2H), 6.80 (s, 1H), 6.96 (d, J = 6.5 Hz, 2H), 7.1 (bs, 1H), 7.36 (bs, 2H), 7.60-7.53 (m, 2H), 8.41 (d, J = 5.6 Hz, 1H), 8.86 (s, 1H), 9.09 (bs, 1H). | 448.2 |
| 273 | 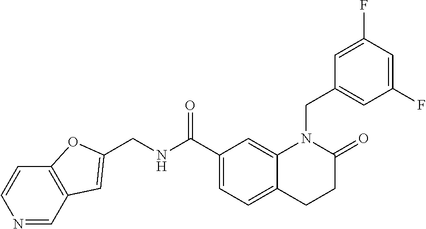 [SMILES: O=C(NCc1cc2cnccc2o1)c1ccc2c(c1)N(Cc1cc(F)cc(F)c1)C(=O)CC2] | 1-(3,5- difluorobenzyl)- N- (furo[3,2- c]pyridin-2- ylmethyl)-2- oxo-1,2,3,4- tetrahydro quinoline-7- carboxamide | (400 MHz; DMSO-d₆): δ 2.74 (t, J = 7.3 Hz, 2H), 3.02 (t, J = 7.3 Hz, 2H), 4.62 (d, J = 5.5 Hz, 2H), 5.19 (s, 2H), 6.81 (s, 1H), 6.96 (d, J = 6.9 Hz, 2H), 7.11 (bs, 1H), 7.36 (bs, 2H), 7.60-7.54 (m, 2H), 8.41 (d, J = 5.7 Hz, 1H), 8.86 (s, 1H), 9.09 (bs, 1H). | 448.1 |
| 274 | 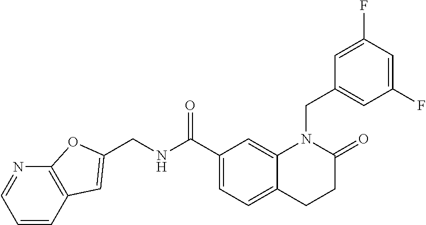 [SMILES: O=C(NCc1cc2cccnc2o1)c1ccc2c(c1)N(Cc1cc(F)cc(F)c1)C(=O)CC2] | 1-(3,5- difluorobenzyl)- N- (furo[2,3- b]pyridin-2- ylmethyl)-2- oxo-1,2,3,4- tetrahydro quinoline-7- carboxamide | (400 MHz; DMSO-d₆): δ 2.71 (t, J = 7.3 Hz, 2H), 2.99 (t, J = 7.2 Hz, 2H), 4.58 (d, J = 5.5 Hz, 2H), 5.16 (s, 2H), 6.68 (s, 1H), 6.93 (d, J = 6.6 Hz, 2H), 7.07 (t, J = 9.3 Hz, 1H), 7.34-7.26 (m, 3H), 7.52 (d, J = 7.8 Hz, 1H), 8.00- 7.98 (dd, 1H), 8.21-8.19 (dd, 1H), 9.07 (t, J = 5.6 Hz, 1H). | 447.9 |
| 275 | 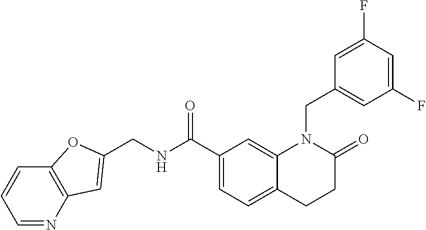 [SMILES: O=C(NCc1cc2ncccc2o1)c1ccc2c(c1)N(Cc1cc(F)cc(F)c1)C(=O)CC2] | 1-(3,5- difluorobenzyl)- N- (furo[3,2- b]pyridin-2- ylmethyl)-2- oxo-1,2,3,4- tetrahydro quinoline-7- carboxamide | (400 MHz; DMSO-d₆): δ 2.74 (t, J = 7.3 Hz, 2H), 3.02 (t, J = 7.3 Hz, 2H), 4.64 (d, J = 5.5 Hz, 2H), 5.19 (s, 2H), 6.85 (s, 1H), 6.96 (d, J = 8.8 Hz, 2H), 7.09 (t, J = 9.1 Hz, 1H), 7.31-7.26 (m, 3H), 7.56 (d, J = 8.1 Hz, 1H), 7.93 (d, J = 8.2 Hz, 1H), 8.45 (d, J = 4.6 Hz, 1H), 9.1 (t, J = 5.6 Hz, 1H). | 448.2 |

### Example 276: 5-(3,5-Difluorobenzyl)-6-oxo-N-(2,4,6-trifluorobenzyl)-5,6,7,8-tetrahydro-1,5-naphthyridine-3-carboxamide

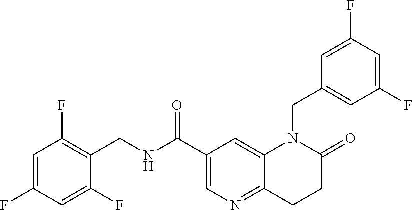 [SMILES: O=C(NCc1c(F)cc(F)cc1F)c1cnc2c(c1)N(Cc1cc(F)cc(F)c1)C(=O)CC2]

### Preparation 43: (E)-Methyl-5-amino-6-(3-ethoxy-3-oxoprop-1-en-1-yl)nicotinate

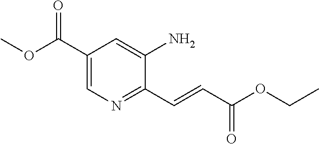 [SMILES: CCOC(=O)/C=C/c1ncc(C(=O)OC)cc1N]

A sealed tube was charged with commercially available 5-amino-6-bromo-nicotinic acid methyl ester (2.0 g, 8.66 mmol), DMF (1.0 mL), ethyl acrylate (2.16 mL, 20.30 mmol) and then TEA (2.19 mL, 15.7 mmol) and the whole degassed with N₂ gas for 15 min. ATAPHOS (0.307 g, 0.44 mmol, 50 mol %) was then added at RT. The resulting reaction mixture was heated at 140° C. for 12 h. After completion of the reaction, the reaction mixture was poured into water and extracted with EtOAc. The combined organics were washed with brine, dried over anhydrous Na₂SO₄ and concentrated under reduced pressure to obtain the crude product which was purified by column chromatography to afford (E)-methyl 5-amino-6-(3-ethoxy-3-oxoprop-1-en-1-yl)nicotinate (0.95 g, 3.8 mmol, 44% yield). LCMS m/z: 251.2 [M+H].

### Preparation 44: Methyl-5-amino-6-(3-ethoxy-3-oxopropyl)nicotinate

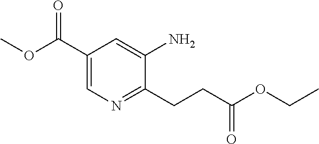 [SMILES: CCOC(=O)CCc1ncc(C(=O)OC)cc1N]

To a stirred solution of (E)-methyl-5-amino-6-(3-ethoxy-3-oxoprop-1-en-1-yl)nicotinate (Preparation 43) (0.5 g, 2.0 mmol) in MeOH (15 mL) was added Pd/C (0.55 g, 10% w/w) under a N₂ gas atmosphere at RT. The resulting reaction mixture was stirred under H₂ gas balloon pressure at 0-5° C. for 2 h. The reaction was monitored by TLC and after completion the whole was filtered through a celite bed and washed with MeOH. The filtrate was concentrated under reduced pressure to give methyl 5-amino-6-(3-ethoxy-3-oxopropyl)nicotinate (0.45 g, 1.79 mmol, 89% yield) as a crude solid which was used as such in the next step without further purification.

LCMS m/z: 252.9 [M+H].

### Preparation 45: Methyl-6-oxo-5,6,7,8-tetrahydro-1,5-naphthyridine-3-carboxylate

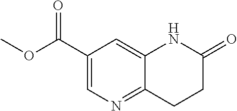

To a stirred solution of methyl-5-amino-6-(3-ethoxy-3-oxopropyl)nicotinate (Preparation 44) (0.4 g, 1.59 mmol) in toluene (5 mL) was added formic acid (3.0 mL) at RT. The resulting mixture was heated to reflux for 1h. After completion of the reaction, the reaction mixture was evaporated under reduced pressure to obtain a yellow residue, which was diluted with water and extracted with EtOAc. The combined organics were dried over anhydrous Na₂SO₄ and concentrated under reduced pressure to give crude product which was purified by column chromatography to afford methyl-6-oxo-5,6,7,8-tetrahydro-1,5-naphthyridine-3-carboxylate (0.25 g, 1.21 mmol, 76% yield) as an off white solid. LCMS m/z: 207.1 [M+H].

### Preparation 46: 5-(3,5-Difluorobenzyl)-6-oxo-N-(2,4,6-trifluorobenzyl)-5,6,7,8-tetrahydro-1,5-naphthyridine-3-carboxamide

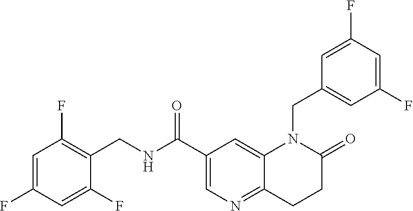 [SMILES: O=C(NCc1c(F)cc(F)cc1F)c1cnc2c(c1)N(Cc1cc(F)cc(F)c1)C(=O)CC2]

5-(3,5-Difluorobenzyl)-6-oxo-N-(2,4,6-trifluorobenzyl)-5,6,7,8-tetrahydro-1,5-naphthyridine-3-carboxamide (Example 276) was prepared from methyl-6-oxo-5,6,7,8-tetrahydro-1,5-naphthyridine-3-carboxylate (Preparation 45) using identical methods described in Preparations 5 and 6. LCMS m/z: 462.1 [M+H]; ¹H NMR (400 MHz; DMSO-d₆): δ 2.87 (t, J=7.76 Hz, 2H), 3.18 (t, J=7.12 Hz, 2H), 4.43 (d, J=4.24 Hz, 2H), 5.15 (s, 2H), 6.68 (d, J=6.24 Hz, 2H), 7.09-7.18 (m, 3H), 7.51 (s, 1H), 8.56 (s, 1H), 9.02 (bs, 1H).

### Examples 277-283

Examples 274-280 were made in an analogous manner to Example 273 starting from the appropriate pyridine and using the appropriate benzyl halides and amines as described for General procedures 7-10.

| | | IUPAC | | LCMS |
|---|---|---|---|---|
| Example | Structure | Name | ¹H-NMR | [M + H] |
| 277 | 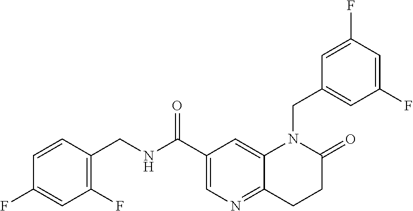 [SMILES: O=C(NCc1ccc(F)cc1F)c1cnc2c(c1)N(Cc1cc(F)cc(F)c1)C(=O)CC2] | N-(2,4- difluorobenzyl)- 5-(3,5- difluorobenzyl)- 6-oxo- 5,6,7,8- tetrahydro- 1,5- naphthyridine- 3- carboxamide | (400 MHz; CDCl₃): δ 2.91 (t, J = 7.92 Hz, 2H), 3.25 (t, J = 7.28 Hz, 2H), 4.60 (d, J = 5.8 Hz, 2H), 5.12 (s, 2H), 6.43 (bs, 1H), 6.66-6.71 (m, 3H), 6.80- 6.87 (m, 2H), 7.35-7.37 (m, 1H), 7.56 (s, 1H), 8.44 (s, 1H). | 444.2 |
| 278 | 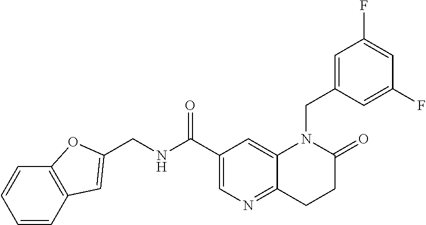 [SMILES: O=C(NCc1cc2ccccc2o1)c1cnc2c(c1)N(Cc1cc(F)cc(F)c1)C(=O)CC2] | N- (benzofuran- 2-ylmethyl)-5- (3,5- difluorobenzyl)- 6-oxo- 5,6,7,8- tetrahydro- 1,5- naphthyridine- 3- carboxamide | (400 MHz; CDCl₃): δ 2.91 (t, J = 7.76 Hz, 2H), 3.25 (t, J = 7.72 Hz, 2H), 4.750 (s, 2H), 5.13 (s, 2H), 6.48 (bs, 1H), 6.67-6.70 (m, 4H), 7.21-7.25 (m, 1H), 7.43 (d, J = 8.12 Hz, 1H), 7.52 (d, J = 6.92 Hz, 1H), 7.60 (s, 1H), 8.4 (s, 1H). | 448.4 |
| 279 | 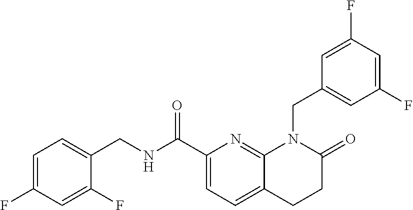 [SMILES: O=C(NCc1ccc(F)cc1F)c1ccc2c(n1)N(Cc1cc(F)cc(F)c1)C(=O)CC2] | N-(2,4- difluorobenzyl)- 8-(3,5- difluorobenzyl)- 7-oxo- 5,6,7,8- tetrahydro- 1,8- naphthyridine- 2- carboxamide | (400 MHz; DMSO-d₆): δ 2.77 (t, J = 7.60 Hz, 2H), 3.03 (t, J = 6.20 Hz, 2H), 4.49 (d, J = 6.08 Hz, 2H), 5.43 (s, 2H), 6.96-7.03 (m, 4H), 7.20-7.22 (m, 2H), 7.65 (d, J = 7.76 Hz, 1H), 7.83 (d, J = 7.0 Hz, 1H), 8.93 (bs, 1H). | 444.2 |
| 280 | 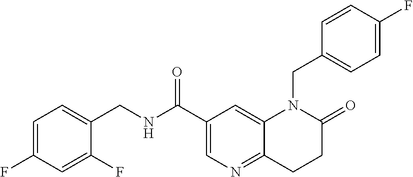 [SMILES: O=C(NCc1ccc(F)cc1F)c1cnc2c(c1)N(Cc1ccc(F)cc1)C(=O)CC2] | N-(2,4- difluorobenzyl)- 5-(4- fluorobenzyl)- 6-oxo-5,6,7,8- tetrahydro- 1,5- naphthyridine- 3- carboxamide | (400 MHz; DMSO-d₆): δ 2.85 (t, J = 7.96 Hz, 2H), 3.17 (t, J = 7.28 Hz, 2H), 4.44 (d, J = 5.48 Hz, 2H), 5.14 (s, 2H), 7.04 (t, J = 9.28 Hz, 1H), 7.13 (t, J = 8.76 Hz, 2H), 7.22-7.28 (m, 3H), 7.30-7.36 (m, 1H), 7.64 (s, 1H), 8.60 (s, 1H), 9.13 (t, J = 5.64 Hz, 1H). | 426.1 |
| 281 | 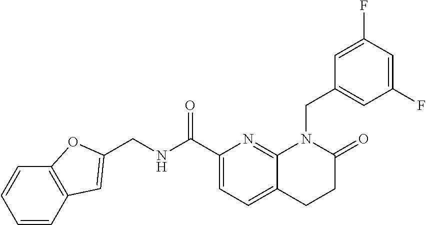 [SMILES: O=C(NCc1cc2ccccc2o1)c1ccc2c(n1)N(Cc1cc(F)cc(F)c1)C(=O)CC2] | N- (benzofuran- 2-ylmethyl)- 8-(3,5- difluorobenzyl)- 7-oxo- 5,6,7,8- tetrahydro- 1,8- naphthyridine- 2- carboxamide | (400 MHz; DMSO-d₆): δ 2.77 (t, J = 7.80 Hz, 2H), 3.03 (t, J = 7.0 Hz, 2H), 4.65 (d, J = 5.84 Hz, 2H), 5.45 (s, 2H), 6.56 (s, 1H), 6.98-7.06 (m, 3H), 7.19- 7.27 (m, 2H), 7.49-7.55 (m, 2H), 7.68 (d, J = 7.44 Hz, 1H), 7.84 (d, J = 7.44 Hz, 1H), 9.04 (bs, 1H). | 448.3 |
| 282 | 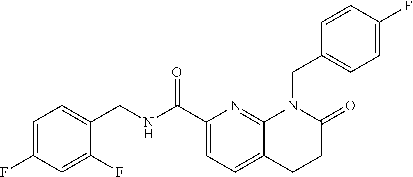 [SMILES: O=C(NCc1ccc(F)cc1F)c1ccc2c(n1)N(Cc1ccc(F)cc1)C(=O)CC2] | N-(2,4- difluorobenzyl)- 8-(4- fluorobenzyl)- 7-oxo-5,6,7,8- tetrahydro- 1,8- naphthyridine- 2- carboxamide | (400 MHz; DMSO-d₆): δ 2.74 (t, J = 7.28 Hz, 2H), 2.96 (t, J = 7.92 Hz, 2H), 4.51 (d, J = 5.52 Hz, 2H), 5.40 (s, 2H), 6.98-7.06 (m, 3H), 7.23-7.37 (m, 4H), 7.64 (d, J = 7.8 Hz, 1H), 7.80 (d, J =6.96 Hz, 1H), 8.93 (bs, 1H). | 426.3 |
| 283 | 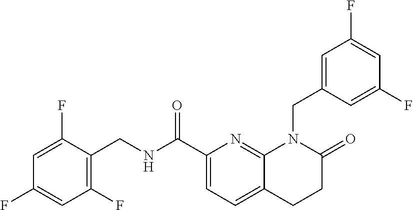 [SMILES: O=C(NCc1c(F)cc(F)cc1F)c1ccc2c(n1)N(Cc1cc(F)cc(F)c1)C(=O)CC2] | (3,5- difluorobenzyl)- 7-oxo-N- (2,4,6- trifluorobenzyl)- 5,6,7,8- tetrahydro- 1,8- naphthyridine- 2- carboxamide | (400 MHz; DMSO-d₆): δ 2.76 (t, J = 7.60 Hz, 2H), 3.01 (t, J = 7.08 Hz, 2H), 4.49 (d, J = 5.56 Hz, 2H), 5.37 (s, 2H), 6.96-7.00 (m, 3H), 7.11 (t, J = 8.32 Hz, 2H), 7.63 (d, J = 7.76 Hz, 1H), 7.81 (d, J = 7.28 Hz, 1H), 8.63 (bs, 1H). | 462.2 |

### Example 284: 1-(3,5-Difluorobenzyl)-N-(2,4,6-trifluorobenzyl)-3,4-dihydro-1H-benzo[c][1,2]thiazine-7-carboxamide-2,2-dioxide

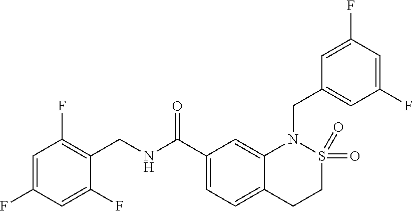 [SMILES: O=C(NCc1c(F)cc(F)cc1F)c1ccc2c(c1)N(Cc1cc(F)cc(F)c1)S(=O)(=O)CC2]

Example 284 was prepared according to the methods described in General Procedures 1-3, and the methods described below.

### Preparation 47: Methyl-3-amino-4-formylbenzoate

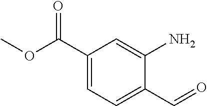

To a stirred solution of commercially available methyl 4-formyl-3-nitrobenzoate (2.0 g, 9.57 mmol) in EtOH (30 mL) was added Fe powder (1.60 g, 28.70 mmol) followed by 0.15 M HCl (816 mL) and resulting reaction mixture was refluxed for 2 h. The progress of reaction was monitored by TLC and LCMS and after completion the mixture was filtered, washed with water, extracted with EtOAc and washed with brine. The organic layer was dried over anhydrous Na₂SO₄ and concentrated under reduced pressure to give the title compound (1.7 g, 99% yield and purity >97%) as a brown oily liquid which was used in the next step without any further purification. LCMS m/z: 180.08 [M+H].

### Preparation 48: Methyl 4-formyl-3-(methylsulfonamido)benzoate

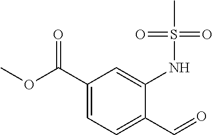 [SMILES: COC(=O)c1ccc(C=O)c(NS(C)(=O)=O)c1]

To a stirred solution of methyl 3-amino-4-formylbenzoate (Preparation 47) (1.5 g, 8.38 mmol) in pyridine (31 mL) was added DMAP (1.54 g, 12.57 mmol) followed by methanesulfonyl chloride (0.713 mL, 9.21 mmol) dropwise and the whole was allowed to stir at RT for 48 h. After complete consumption of the starting material (monitored by TLC and LCMS) the mixture was diluted with EtOAc and successively washed with 1N aqueous hydrochloric acid, water, saturated aqueous sodium bicarbonate, and saturated brine solutions. The organic layer was dried over anhydrous Na₂SO₄, filtered, and concentrated under reduced pressure to give a crude product which was purified by Combi-flash column chromatography using a 4 g silica column using 50% EtOAc in hexane as eluent to afford the title compound (0.6 g, 40% yield and purity 97%) as a yellow solid. LCMS m/z: 258.04 [M+H].

### Preparation 49: Methyl 1-(3,5-difluorobenzyl)-1H-benzo[1,2]thiazine-7-carboxylate 2,2-dioxide

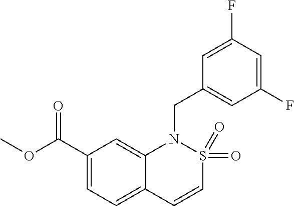 [SMILES: COC(=O)c1ccc2c(c1)N(Cc1cc(F)cc(F)c1)S(=O)(=O)C=C2]

To a stirred solution of methyl 4-formyl-3-(methylsulfonamido)benzoate (Preparation 48) (0.15 g, 0.58 mmol) in DMF (5 mL) was added Cs₂CO₃ (0.38 g, 1.17 mmol) followed by 3,5-difluorobenzyl bromide (0.15 mL, 1.17 mmol) under an inert atmosphere and the whole was refluxed at 50° C. for 12 h. The course of reaction was monitored by TLC and LCMS and after completion the mixture was diluted with EtOAc and successively washed with ice cold water, brine, dried over anhydrous Na₂SO₄ and evaporated under reduced pressure to give a crude residue which was purified by column chromatography in Combi-flash eluting with 25-30% EtOAc/hexane to give the title compound (0.1 g, 84% yield and purity >98%) as a yellow solid. LCMS m/z: 366.1 [M+H].

### Preparation 50: Methyl 1-(3,5-difluorobenzyl)-3,4-dihydro-1H-benzo[c][1,2]thiazine-7-carboxylate 2,2-dioxide

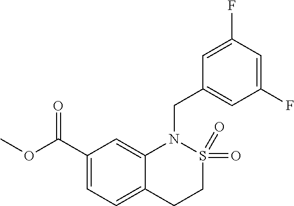 [SMILES: COC(=O)c1ccc2c(c1)N(Cc1cc(F)cc(F)c1)S(=O)(=O)CC2]

To a stirred solution of methyl 1-(3,5-difluorobenzyl)-1H-benzo[c][1,2]thiazine-7-carboxylate 2,2-dioxide (Preparation 49) (0.1 g, 0.032 mmol) in MeOH (5 mL) was added 10% Pd—C (10 mg, 50% w/w in water) under a N₂ gas atmosphere. The resulting reaction mixture was then stirred under H₂ gas balloon pressure at RT for 1 h. After completion of the reaction the mixture was filtered carefully through a celite bed. The filtrate was dried over anhydrous Na₂SO₄ and evaporated under reduced pressure to give the title compound (0.1 g, 95% yield and purity 99%) as a white solid. LCMS m/z: 368.07 [M+H].

### Example 284: 1-(3,5-Difluorobenzyl)-N-(2,4,6-trifluorobenzyl)-3,4-dihydro-1H-benzo[c][1,2]thiazine-7-carboxamide 2,2-dioxide

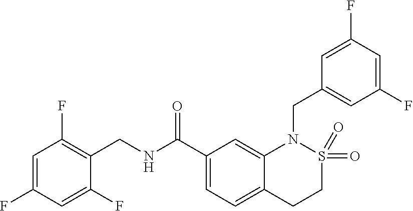 [SMILES: O=C(NCc1c(F)cc(F)cc1F)c1ccc2c(c1)N(Cc1cc(F)cc(F)c1)S(=O)(=O)CC2]

1-(3,5-Difluorobenzyl)-N-(2,4,6-trifluorobenzyl)-3,4-dihydro-1H-benzo[c][1,2]thiazine-7-carboxamide 2,2-dioxide (Example 284) was prepared from methyl 1-(3,5-difluorobenzyl)-3,4-dihydro-1H-benzo[c][1,2]thiazine-7-carboxylate 2,2-dioxide (Preparation 50) using identical methods described in Preparation 5 and 6. White solid; Yield 56%; Purity 98%; LCMS m/z: 497.56 [M+H]; ¹H NMR (400 MHz; DMSO-d₆): δ 3.41 (t, J=6.9 Hz, 2H), 3.67 (t, J=6.9 Hz, 2H), 4.38 (d, J=4.9 Hz, 2H), 5.00 (s, 2H), 7.19-7.05 (m, 6H), 7.32 (d, J=8 Hz, 1H), 7.46 (d, J=8 Hz, 1H), 8.81 (t, J=5.1 Hz, 1H).

### Example 285: 1-(3,5-difluorobenzyl)-3-methyl-N-(2,4,6-trifluorobenzyl)-3,4-dihydro-1H-benzo[c][1,2]thiazine-7-carboxamide 2,2-dioxide

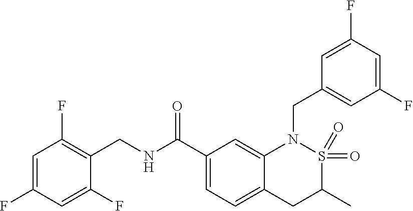 [SMILES: CC1Cc2ccc(C(=O)NCc3c(F)cc(F)cc3F)cc2N(Cc2cc(F)cc(F)c2)S1(=O)=O]

### Preparation 51: Methyl 3-methyl-1H-benzo[c][1,2]thiazine-7-carboxylate 2,2-dioxide

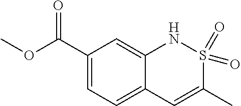 [SMILES: COC(=O)c1ccc2c(c1)NS(=O)(=O)C(C)=C2]

To a stirred solution of methyl 3-amino-4-formylbenzoate (Preparation 47) (0.5 g, 2.79 mmol) in DCM (10 mL) was added TEA (2.2 mL, 22.34 mmol) followed by ethanesulfonyl chloride (0.064 mL, 6.70 mmol) in portion wise and allowed to stir at RT for 48 h. The progress of reaction was monitored by TLC and LCMS and after completion the mixture was diluted with EtOAc and washed with water. The organics were dried over anhydrous Na₂SO₄ and evaporated under reduced pressure to give a crude product which was purified by column chromatography using 50% EtOAc/hexane as eluent to give the title compound (0.2 g, 48% yield and purity >96%) as a white solid. LCMS m/z: 254.04 [M+H].

### Preparation 52: Methyl 1-(3,5-difluorobenzyl)-3-methyl-1H-benzo[c][1,2]thiazine-7-carboxylate 2,2-dioxide

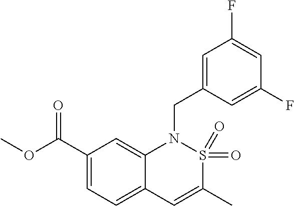 [SMILES: COC(=O)c1ccc2c(c1)N(Cc1cc(F)cc(F)c1)S(=O)(=O)C(C)=C2]

To a stirred solution of methyl 3-methyl-1H-benzo[c][1,2]thiazine-7-carboxylate 2,2-dioxide (Preparation 51) (0.1 g, 0.40 mmol) in DMF (5 mL) was added NaH (22 mg, 0.47 mmol) followed by 3,5-difluorobenzyl bromide (0.056 mL, 0.43 mmol) under an inert atmosphere at 0-5° C. The resulting reaction mixture was allowed to stir for 15 min. at RT. After completion of the reaction the mixture was diluted with EtOAc and washed with ice cold water and brine. The organics were dried over anhydrous Na₂SO₄ and evaporated under reduced pressure to give a crude product which was purified by Combi-flash using 10% EtOAc/hexane as eluent to afford the title compound (0.1 g, 67% yield and purity >97%) as a yellow solid. LCMS m/z: 380.07 [M+H].

### Example 285: 1-(3,5-Difluorobenzyl)-3-methyl-N-(2,4,6-trifluorobenzyl)-3,4-dihydro-1H-benzo[c][1,2]thiazine-7-carboxamide 2,2-dioxide

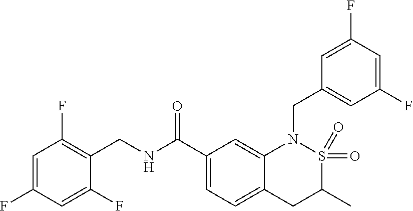 [SMILES: CC1Cc2ccc(C(=O)NCc3c(F)cc(F)cc3F)cc2N(Cc2cc(F)cc(F)c2)S1(=O)=O]

1-(3,5-Difluorobenzyl)-3-methyl-N-(2,4,6-trifluorobenzyl)-3,4-dihydro-1H-benzo[c][1,2]thiazine-7-carboxamide 2,2-dioxide (Example 285) was prepared from methyl 1-(3,5-difluorobenzyl)-3-methyl-1H-benzo[c][1,2]thiazine-7-carboxylate 2,2-dioxide (Preparation 52) using identical methods described in Preparation 50, 5 and 6. White solid; Yield 60%; Purity 99%; LCMS m/z: 511.1 [M+H]; ¹H NMR (500 MHz; DMSO-d₆): δ 1.36 (d, J=4.9 Hz, 3H), 3.20-3.15 (m, 1H), 3.54 (d, J=16.6 Hz, 1H), 3.83 (bs, 1H), 4.41 (s, 2H), 5.07 (s, 2H), 7.20-7.11 (m, 6H), 7.32 (d, J=7.2 Hz, 1H), 7.49 (d, J=6.8 Hz, 1H), 8.84 (s, 1H).

### Biological Assays

### Stable Cell Line Generation

a) Stable STING expressing cells—Stable HEK293T STING-expressing cell lines were generated using plasmids purchased from Invivogen, Calif., USA, that contain STING cDNA cloned into the pUNO-1 vector under hEF1-HTLV promoter and containing the Blasticidin selection cassette. The plasmids hSTING(R232), hSTING(H232), hSTING(HAQ) were directly procured from Invivogen while hSTING (AQ) and hSTING (Q) were derived from hSTING(HAQ) and hSTING (R232) plasmids respectively by using a PCR based site directed mutagenesis method. These vectors were individually transfected into HEK293T cells using Lipofectamine (Invitrogen) and transfected cells were selected under Blasticidin selection. These transfected cells were further subjected to clonal selection using the limiting dilution method to obtain clonally pure populations of HEK cells transfected with each of the above mentioned human STING variants. Only those clones were selected in which ligand independent activation of STING was minimal.
b) Stable Luciferase reporter gene expressing cells—Stable HEK293T Luciferase reporter gene expressing cell lines were generated using pCDNA4 plasmids under an IRF-inducible promoter. This promoter is comprised of five tandem interferon-stimulated response elements (ISRE) fused to an ISG54 minimal promoter. This vector was transfected into HEK293T cells using Lipofectamine (Invitrogen) and transfected cells were selected under Zeocin selection. These transfected cells were further subjected to clonal selection using the limiting dilution method to obtain clonally pure populations of HEK cells transfected the Luciferase reporter construct. Only those clones were selected in which ligand independent induction of luciferase was minimal.

### LuciferaseAssay

5×10⁵ clonally selected HEK293T-hSTING-Luciferase cells were seeded in 384-well plates in growth medium and stimulated with novel compounds. After 20 hr of stimulation supernatant were removed and secretary reporter gene activity were measured using the Quanti-Luc detection system (Invivogen) on a Spectramax i3X luminometer.

In the tables below, EC₅₀ value ranges for exemplary compounds are given. The EC₅₀ ranges are indicated as “A” for values less than or equal to 1 μM, “B” for values greater than 1 μM and less than or equal to 10 μM, and “C” for values greater than 10 μM.

All compounds were first tested in a primary screen using WT/R232 STING protein to obtain a ‘fold-induction’ over baseline levels of protein activity. Only those compounds that had a fold induction >1 have been included in the table and all are considered ‘active’. These active compounds were then further investigated to obtain an EC₅₀ value.

R232 activity

| | R232 |
|---|---|
| Example | Activity |
| 1 | C |
| 2 | C |
| 3 | C |
| 4 | C |
| 5 | C |
| 6 | C |
| 7 | C |
| 8 | C |
| 9 | C |
| 10 | C |
| 11 | B |
| 12 | B |
| 13 | B |
| 14 | B |
| 15 | C |
| 16 | B |
| 17 | C |
| 18 | C |
| 19 | C |
| 20 | C |
| 21 | B |
| 22 | C |
| 23 | C |
| 24 | C |
| 25 | C |
| 26 | C |
| 27 | C |
| 28 | C |
| 29 | C |
| 30 | C |
| 31 | A |
| 32 | B |
| 33 | C |
| 34 | A |
| 35 | C |
| 36 | C |
| 37 | A |
| 38 | B |
| 39 | B |
| 40 | A |
| 41 | C |
| 42 | C |
| 43 | C |
| 44 | C |
| 45 | C |
| 46 | C |
| 47 | C |
| 48 | C |
| 49 | C |
| 50 | C |
| 51 | C |
| 52 | C |
| 53 | C |
| 54 | C |
| 55 | C |
| 56 | C |
| 57 | C |
| 58 | C |
| 59 | C |
| 6o | C |
| 61 | B |
| 62 | B |
| 63 | C |
| 64 | C |
| 65 | C |
| 66 | B |
| 67 | C |
| 68 | C |
| 69 | B |
| 70 | C |
| 71 | B |
| 72 | C |
| 73 | B |
| 74 | B |
| 75 | C |
| 76 | C |
| 77 | C |
| 78 | C |
| 79 | C |
| 8o | C |
| 81 | C |
| 82 | B |
| 83 | B |
| 84 | C |
| 85 | C |
| 86 | C |
| 87 | A |
| 88 | C |
| 89 | C |
| 90 | C |
| 91 | C |
| 92 | C |
| 93 | B |
| 94 | C |
| 95 | C |
| 96 | C |
| 97 | C |
| 98 | C |
| 99 | B |
| 100 | B |
| 101 | B |
| 102 | B |
| 103 | C |
| 104 | C |
| 105 | C |
| 106 | C |
| 107 | B |
| 108 | C |
| 109 | C |
| 110 | C |
| 111 | C |
| 112 | A |
| 113 | C |
| 114 | A |
| 115 | A |
| 116 | C |
| 117 | C |
| 118 | B |
| 119 | B |
| 120 | B |
| 121 | C |
| 122 | B |
| 123 | C |
| 124 | C |
| 125 | C |
| 126 | B |
| 127 | B |
| 128 | C |
| 129 | C |
| 130 | C |
| 131 | B |
| 132 | C |
| 133 | B |
| 134 | B |
| 135 | C |
| 136 | B |
| 137 | C |
| 138 | A |
| 139 | C |
| 140 | A |
| 141 | A |
| 142 | A |
| 143 | B |
| 144 | A |
| 145 | A |
| 146 | A |
| 147 | A |
| 148 | A |
| 149 | A |
| 150 | A |
| 151 | A |
| 152 | A |
| 153 | A |
| 154 | A |
| 155 | A |
| 156 | A |
| 157 | A |
| 158 | A |
| 159 | A |
| 160 | A |
| 161 | B |
| 162 | B |
| 163 | B |
| 164 | B |
| 165 | C |
| 166 | C |
| 167 | B |
| 168 | C |
| 169 | C |
| 170 | C |
| 171 | C |
| 172 | C |
| 173 | C |
| 174 | C |
| 175 | C |
| 176 | C |
| 177 | C |
| 178 | A |
| 179 | C |
| 180 | C |
| 181 | B |
| 182 | C |
| 183 | B |
| 184 | C |
| 185 | B |
| 186 | B |
| 187 | B |
| 188 | A |
| 189 | A |
| 190 | A |
| 191 | A |
| 192 | A |
| 193 | A |
| 194 | A |
| 195 | A |
| 196 | A |
| 197 | B |
| 198 | A |
| 199 | A |
| 200 | A |
| 201 | B |
| 202 | B |
| 203 | B |
| 204 | B |
| 205 | B |
| 206 | B |
| 207 | B |
| 208 | C |
| 209 | C |
| 210 | C |
| 211 | C |
| 212 | C |
| 213 | C |
| 214 | C |
| 215 | C |
| 216 | C |
| 217 | C |
| 218 | A |
| 219 | C |
| 220 | C |
| 221 | C |
| 222 | C |
| 223 | C |
| 224 | C |
| 225 | C |
| 226 | C |
| 227 | C |
| 228 | C |
| 229 | C |
| 230 | C |
| 231 | C |
| 232 | A |
| 233 | A |
| 234 | A |
| 235 | A |
| 236 | C |
| 237 | B |
| 238 | B |
| 239 | C |
| 240 | B |
| 241 | B |
| 242 | A |
| 243 | A |
| 244 | C |
| 245 | C |
| 246 | C |
| 247 | C |
| 248 | C |
| 249 | C |
| 250 | C |
| 251 | C |
| 252 | C |
| 253 | C |
| 254 | C |
| 255 | C |
| 256 | C |
| 257 | B |
| 258 | B |
| 259 | C |
| 260 | C |
| 261 | B |
| 262 | C |
| 263 | C |
| 264 | C |
| 265 | C |
| 266 | C |
| 267 | A |
| 268 | C |
| 269 | A |
| 270 | A |
| 271 | A |
| 272 | C |
| 273 | C |
| 274 | C |
| 275 | C |
| 276 | B |
| 277 | B |
| 278 | C |
| 279 | B |
| 280 | C |
| 281 | B |
| 282 | B |
| 283 | B |
| 284 | A |
| 285 | B |

### STING Polymorphisms

Single nucleotide polymorphisms of human STING have been described, which can affect the functional potency of compounds that modulate the activity of the STING protein (see Yi et. al., PLoS One, October 2013, 8(10), e77846). The 5 major polymorphisms of human STING are shown in FIG. 1, with their prevalence in human populations indicated.

The tables below show the potency of selected compounds of the invention against the most common polymorphisms.

H232 activity

| | H232 | | H232 | | H232 |
|---|---|---|---|---|---|
| Example | activity | Example | activity | Example | activity |
| 145 | A | 192 | A | 136 | C |
| 269 | C | 151 | B | 258 | C |
| 270 | A | 150 | B | 138 | A |
| 178 | C | 157 | B | 140 | A |
| 267 | A | 156 | B | 87 | B |
| 40 | B | 242 | C | 83 | C |
| 233 | B | 243 | A | 181 | C |
| 234 | B | 196 | A | 69 | B |
| 183 | B | 203 | B | 118 | B |
| 271 | A | 201 | B | 120 | B |
| 187 | C | 199 | A | 82 | B |
| 188 | B | 195 | B | 93 | B |
| 146 | B | 158 | B | 31 | A |
| 147 | B | 197 | B | 141 | B |
| 235 | B | 284 | C | 142 | A |
| 153 | B | 202 | B | 34 | A |
| 148 | A | 198 | A | 37 | B |
| 232 | B | 133 | B | 39 | B |
| 218 | B | 160 | C | 144 | B |
| 279 | C | 159 | B | 152 | C |
| 161 | C | 285 | B | 149 | A |
| 189 | B | 154 | B | 193 | B |
| 281 | B | 131 | B | 200 | B |
| 190 | B | 127 | B | 194 | A |
| 191 | B | 112 | A | | |

HAQ activity

| | HAQ | | HAQ | | HAQ |
|---|---|---|---|---|---|
| Example | activity | Example | activity | Example | activity |
| 145 | A | 192 | A | 136 | C |
| 269 | B | 151 | B | 258 | C |
| 270 | B | 150 | B | 138 | A |
| 178 | C | 157 | B | 140 | A |
| 267 | A | 156 | B | 87 | C |
| 40 | B | 242 | B | 83 | C |
| 233 | B | 243 | A | 181 | C |
| 234 | B | 196 | A | 69 | B |
| 183 | B | 203 | B | 118 | C |
| 271 | A | 201 | B | 120 | C |
| 187 | C | 199 | A | 82 | B |
| 188 | B | 195 | B | 93 | C |
| 146 | B | 158 | B | 31 | B |
| 147 | B | 197 | B | 141 | A |
| 235 | B | 284 | C | 142 | A |
| 153 | B | 202 | B | 34 | B |
| 148 | A | 198 | A | 37 | C |
| 232 | B | 133 | B | 39 | B |
| 218 | B | 160 | C | 144 | B |
| 279 | C | 159 | B | 152 | C |
| 161 | B | 285 | B | 149 | A |
| 189 | B | 154 | A | 193 | B |
| 281 | B | 131 | C | 200 | B |
| 190 | B | 127 | B | 194 | A |
| 191 | B | 112 | A | | |

### Reporter Gene Expression Assay for IRF & NFkB Axis in THP-1 Cells

THP1-Dual™ cells (Invivogen) were derived from the human THP-1 monocyte cell line by stable integration of two inducible reporter constructs. As a result, THP1-Dual™ cells allow the simultaneous study of the NF-κB pathway, by monitoring the activity of secreted SEAP, and the IRF pathway, by assessing the activity of a secreted luciferase (Lucia). 5×10⁵ THP1-Dual™ cells were seeded in 384-well plates in growth medium and stimulated with novel compounds. After 2 ohr of stimulation supernatant were removed and reporter proteins were readily measured in the cell culture supernatant using QUANTI-Blue™ (Invivogen), a SEAP detection reagent, and QUANTI-Luc™ (Invivogen), a luciferase detection reagent on a Spectramax i3X luminometer. EC₅₀ value ranges for exemplary compounds are given. The EC₅₀ ranges are indicated as “A” for values less than or equal to 1 μM, “B” for values greater than 1 μM and less than or equal to 10 μM, and “C” for values greater than 10 μM.

IRF/NF_{K}B activity

| | THP- | THP- | | | THP- |
|---|---|---|---|---|---|
| | IRF | NF_{K}B | | THP-IRF | NF_{K}B |
| Example | activity | activity | Example | activity | activity |
| 145 | B | B | 158 | B | B |
| 269 | C | C | 197 | C | C |
| 270 | C | B | 284 | C | C |
| 178 | C | C | 202 | C | C |
| 267 | B | B | 198 | B | B |
| 40 | B | B | 133 | C | B |
| 233 | C | C | 160 | C | C |
| 234 | C | C | 159 | B | B |
| 183 | C | B | 285 | C | C |
| 271 | B | B | 154 | B | B |
| 187 | C | C | 131 | C | C |
| 188 | C | B | 127 | C | C |
| 146 | C | C | 112 | C | C |
| 147 | C | C | 136 | C | C |
| 235 | B | B | 258 | C | C |
| 153 | B | B | 138 | B | B |
| 148 | B | A | 140 | B | B |
| 232 | C | C | 87 | C | C |
| 218 | B | B | 83 | C | C |
| 279 | C | C | 181 | C | C |
| 161 | C | C | 69 | C | C |
| 189 | C | C | 118 | C | C |
| 281 | C | C | 120 | C | C |
| 190 | B | B | 82 | C | C |
| 191 | B | B | 93 | C | C |
| 192 | B | B | 31 | C | C |
| 151 | B | B | 141 | B | B |
| 150 | B | B | 142 | B | B |
| 157 | B | B | 34 | C | C |
| 156 | B | B | 37 | C | C |
| 242 | C | C | 39 | C | C |
| 243 | C | C | 144 | B | B |
| 196 | B | B | 152 | C | C |
| 203 | C | C | 149 | B | A |
| 201 | C | C | 193 | C | C |
| 199 | B | B | 200 | C | C |
| 195 | C | B | 194 | B | B |

### Western Blot Assay

5×10⁵ clonally selected HEK293T-hSTING-Luciferase cells were seeded in 24-well plates in 500 μl growth medium and stimulated with novel compounds or a vehicle control (VC), i.e. the solvent with no compound. After 2 hr of stimulation cells were harvested through centrifugation and cells pellet were lysed in RIPA buffer (20 mM tris-Cl, 150 mM NaCl, 0.5 mM EDTA, 1% NP40, 0.05% SDS) containing Ix phosphatase inhibitor cocktail 3 (Sigma) and Ix protease inhibitor (Roche) to extract the soluble fraction of protein. 10 μg of extracted protein was electrophoresed in 10% SDS-PAGE gels and transferred onto Immobilon-P membranes (Millipore). Blots were incubated with antibodies specific for phosphorylated STING (Ser366), phosphorylated IRF3 (Ser396), total STING, ACTIN (Cell Signaling) and IRF3 (Abcam). Anti-rabbit HRP label secondary antibody (Abcam) and Clarity Max™ western ECL substrate (Biorad) were used for visualization of bands with help of the BioRad XRS plus imager. The Western blot assays are shown in FIG. 2.

### Analysis of Cytokines by ELISA

Freshly isolated 2×10⁵ human PBMCs using Histopaque (Sigma) from different healthy donors were stimulated with novel compounds (10 μM) in 200 μl growth medium for 6 hr. Post treatment supernatant media was harvested and stored at −80° C. in different aliquots for secreted Cytokine analysis. Key cytokines like IFNβ, IFNα, IL6, CXCL10, and TNFα were measured using respective manufacturers recommendations. IFNβ, IFNα were purchased from PBL Assay science, IL6, CXCL10 were procured from Abcam and TNFα was purchased from R&D systems. The results are shown in FIG. 3.

### In Vivo Tumor Experiments

1×10⁶ CT26 tumor cells stably expressing R232.hSTING were injected subcutaneously in 100 μl RPMI on the right side of the flank of Balb/C mice. Following tumor implantation, when the average tumors size was around 50 mm³ to 70 mm³, mice were randomized into different groups. Total number of animals per group is around 5 to 8. New chemical entity which was tested in this tumor model was formulated in 100% PEG400. For the treatment groups compounds were dosed intra-tumorally thrice in a week. Control animals were injected vehicle by same route as per same scheduled of compound dosing, and are identified as vehicle controls (VC). Growth of the tumors was measured regularly during the course of the study.

### CONCLUSION

The inventors have synthesised a large number of compounds which fall within the general formula (I). They have shown that these compounds activate the STING protein, and so could be used to treat a number of diseases, including cancer.

1. A compound of formula (I): 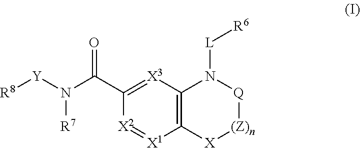
or a pharmaceutically acceptable salt or prodrug thereof, wherein:
X is CR⁹R¹⁰, O, S, S═O or SO₂;
X¹ is CR¹ or N;
X² is CR² or N;
X³ is CR³ or N;
the or each Z is CR¹¹R¹²;
n is 1 or 2;
Q is C═O, S═O, SO₂, C═S or CR⁴R⁵;
L is optionally substituted C₁-C₆ alkyl, C₁-C₃ polyfluoroalkyl, optionally substituted C₃-C₆ cycloalkyl, optionally substituted C₂-C₆ alkenyl, optionally substituted C₂-C₆ alkynyl, C═O, S═O, SO₂, —CH₂C(O)—, —CH₂CONH—, or —CONH—;
Y is an optionally substituted C₁-C₆ alkyl, C₁-C₃ polyfluoroalkyl, an optionally substituted C₂-C₆ alkenyl, an optionally substituted C₂-C₆ alkynyl, an optionally substituted C₃-C₆ cycloalkyl, an optionally substituted mono or bicyclic 3 to 8 membered heterocycle;
R¹, R² and R³ are each independently selected from the group consisting of H, halogen, CN, hydroxyl, COOH, CONR¹R², NR¹R², NHCOR, optionally substituted C₁-C₆ alkyl, C₁-C₃ polyfluoroalkyl, optionally substituted C₁-C₆ alkylsulfonyl, optionally substituted mono or bicyclic C₃-C₆ cycloalkyl, optionally substituted C₂-C₆ alkenyl, optionally substituted C₂-C₆ alkynyl, optionally substituted C₁-C₆ alkoxy, optionally substituted C₁-C₆ alkoxycarbonyl group, mono or bicyclic optionally substituted C₅-C₁₀ aryl, mono or bicyclic optionally substituted 5 to 10 membered heteroaryl, optionally substituted mono or bicyclic 3 to 8 membered heterocycle, optionally substituted aryloxy, optionally substituted heteroaryloxy, and optionally substituted heterocyclyloxy;
R⁴ and R⁵ are each independently selected from the group consisting of H, halogen, optionally substituted C₁-C₆ alkyl, optionally substituted (C₃-C₆) cycloalkyl or R⁴ and R⁵ together with the atom to which they are attached form a spirocyclic ring;
R⁶ is mono or bicyclic optionally substituted C₅-C₁₀ aryl, mono or bicyclic optionally substituted 5 to 10 membered heteroaryl, optionally substituted C₃-C₆ cycloalkyl or an optionally substituted mono or bicyclic 3 to 8 membered heterocycle;
R⁷ is H, optionally substituted C₁-C₆ alkyl, optionally substituted sulfonyl, optionally substituted C₁-C₆ alkylsulfonyl, optionally substituted C₃-C₆ cycloalkyl, optionally substituted C₂-C₆ alkenyl and optionally substituted C₂-C₆ alkynyl;
R⁸ is mono or bicyclic optionally substituted C₅-C₁₀ aryl, mono or bicyclic optionally substituted 5 to 10 membered heteroaryl, optionally substituted mono or bicyclic C₃-C₆ cycloalkyl or an optionally substituted mono or bicyclic 3 to 8 membered heterocycle;
R⁹ and R¹⁰ are each independently selected from the group consisting of optionally substituted C₁-C₆ alkyl, H, halogen, CN, hydroxyl, CO₂H, CONR¹R², azido, sulfonyl, NR¹R², NHCOR¹, C₁-C₃ polyfluoroalkyl, optionally substituted C₁-C₆ thioalkyl, optionally substituted C₁-C₆ alkylsulfonyl, optionally substituted C₃-C₆ cycloalkyl, optionally substituted C₂-C₆ alkenyl, optionally substituted C₂-C₆ alkynyl, optionally substituted C₁-C₆ alkoxy, optionally substituted C₁-C₆ alkoxycarbonyl, mono or bicyclic optionally substituted C₅-C₁₀ aryl, mono or bicyclic optionally substituted 5 to 10 membered heteroaryl, optionally substituted heterocycle, optionally substituted aryloxy, and an optionally substituted heteroaryloxy; or R⁹ and R¹⁰ together with the C atom to which they are attached can combine to form an optionally substituted spirocyclic ring; and
R¹¹ and R¹² are each independently selected from the group consisting of optionally substituted C₁-C₆ alkyl, H, halogen, CN, hydroxyl, CO₂H, CONR¹R², azido, sulfonyl, NR¹R², NHCOR¹, C₁-C₃ polyfluoroalkyl, optionally substituted C₁-C₆ thioalkyl, optionally substituted C₁-C₆ alkylsulfonyl, optionally substituted C₃-C₆ cycloalkyl, optionally substituted C₂-C₆ alkenyl, optionally substituted C₂-C₆ alkynyl, optionally substituted C₁-C₆ alkoxy, optionally substituted C₁-C₆ alkoxycarbonyl, mono or bicyclic optionally substituted C₅-C₁₀ aryl, mono or bicyclic optionally substituted 5 to 10 membered heteroaryl, optionally substituted heterocycle, optionally substituted aryloxy, and an optionally substituted heteroaryloxy; or R¹¹ and R¹² together with the C atom to which they are attached can combine to form an optionally substituted spirocyclic ring;
with the proviso that when X is S; X¹, X² and X³ are CH; n is 1; Z is CH₂; Q is C═O, L is —CH₂—; Y is —CH₂—; R⁷ is H; and R⁶ is unsubstituted phenyl, 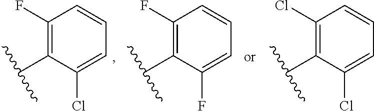
then R⁸ is not unsubstituted furanyl; and
when X is S; X¹, X² and X³ are CH; n is 1; Z is CH₂; Q is C═O, L is —CH₂—; Y is —CH₂—;
R⁷ is H; and R⁶ is 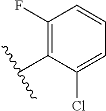 [SMILES: *c1c(F)cccc1Cl]
then R⁸ is not unsubstituted phenyl;
or a pharmaceutically acceptable complex, salt, solvate, tautomeric form or polymorphic form thereof, for use in therapy.

2. A compound according to claim 1, wherein X¹ is CR¹, X² is CR² and X³ is CR³.

3. A compound according to claim 1, wherein one or two of X¹, X² and X³ is N.

4. A compound according to any preceding claim, wherein R¹, R² and R³ are each H.

5. A compound according to any preceding claim, wherein X is O, S or CR⁹R¹⁰.

6. A compound according to claim 5, wherein X is S or CR⁹R¹⁰.

7. A compound according to any preceding claim, wherein at least one of R⁹ and R¹⁰ is an optionally substituted C₁-C₆ alkyl, halogen, H, a C₃-C₆ cycloalkyl or C₁-C₃ polyfluoroalkyl.

8. A compound according to claim 7, wherein both R⁹ and R¹⁰ are H.

9. A compound according to any preceding claim, wherein n is 1.

10. A compound according to any preceding claim, wherein at least one of R¹¹ and R¹² is H.

11. A compound according to claim 10, wherein both R¹¹ and R¹² is H.

12. A compound according to claim 10, wherein one of R¹¹ and R¹² is H and the other is an optionally substituted C₁-C₆ alkyl or an optionally substituted C₂-C₆ alkenyl.

13. A compound according to any preceding claim, wherein Q is C═O, SO₂ or CR⁴R⁵.

14. A compound according to claim 13, wherein Q is C═O.

15. A compound according to any preceding claim, wherein L is C═O or SO₂, an optionally substituted C₁-C₆ alkyl, —CH₂C(O)— or —CH₂CONH—.

16. A compound according to claim 15, wherein L is —CH₂—, —CH₂CH₂—, —CH₂CH₂CH₂—, C(Me)H, CF₂ or C(H)F.

17. A compound according to any preceding claim, wherein R⁶ is a mono or bicyclic optionally substituted C₅-C₁₀ aryl, a mono or bicyclic optionally substituted 5 to 10 membered heteroaryl, an optionally substituted C₃-C₆ cycloalkyl or an optionally substituted C₃-C₆ heterocyclyl.

18. A compound according to claim 17, wherein R⁶ is an optionally substituted phenyl, an optionally substituted pyridine, an optionally substituted pyrazole, an optionally substituted thiazole or an optionally substituted isoxazole.

19. A compound according to either claim 17 or claim 20, wherein R⁶ is a mono or bicyclic C₅-C₁₀ aryl or a mono or bicyclic 5 to 10 membered heteroaryl, wherein the aryl or heteroaryl is substituted with between 1 and 5 substituents, and the or each substituent is independently selected from the list consisting of halogen, C₁-C₆ alkyl, CN, C₁-C₆ alkoxy, C₁-C₃ polyfluoroalkyl, azido, CONR¹R² and —OH.

20. A compound according to any one of claims 17 to 19, wherein the aryl is phenyl or naphthyl.

21. A compound according to claim 20, wherein phenyl or the naphthyl is substituted by 1 or 2 halogens.

22. A compound according to any preceding claim, wherein when X¹ is CH, X² is CH and X³ is CH then R⁶ does not comprise an unsubstituted phenyl.

23. A compound according to any preceding claim, wherein R⁷ is preferably H or an optionally substituted C₁-C₆ alkyl.

24. A compound according to any one of claims 1 to 22, wherein Y is an optionally substituted C₁-C₆ alkyl.

25. A compound according to claim 24, wherein Y is —CH₂—, —CH₂CH₂—, —CH₂CH₂CH₂—, —CH(CH₃)—, —CH(F)— and —CF₂—.

26. A compound according to any preceding claim, wherein R⁸ is a mono or bicyclic optionally substituted C₅-C₁₀ aryl, a mono or bicyclic optionally substituted 5 to 10 membered heteroaryl, an optionally substituted C₃-C₆ cycloalkyl or an optionally substituted C₃-C₆ heterocyclyl.

27. A compound according to claim 26, wherein R⁸ is an optionally substituted phenyl, an optionally substituted pyridine, an optionally substituted naphthyl, an optionally substituted furanyl, an optionally substituted benzofuranyl, an optionally substituted thiophene, an optionally substituted pyridofuran, an optionally substituted benzoxazole or an optionally substituted benzothiazole.

28. A compound according to either claim 26 or claim 27, wherein R⁸ is a mono or bicyclic C₅-C₁₀ aryl or a mono or bicyclic 5 to 10 membered heteroaryl substituted with between 1 and 5 substituents, and the or each substituent is independently selected from the list consisting of C₁-C₆ alkyl, halogen, OH, C₁-C₆ alkoxy, C₁-C₃ polyfluoroalkyl, CONR¹R², CN and azido.

29. A compound according to claim 1, wherein:
X is S, O or CR⁹R¹⁰;
X² is CR²;
n is 1;
Q is C═O or CR⁴R⁵;
L is optionally substituted C₁-C₃ alkyl or C₁-C₃ polyfluoroalkyl;
Y is an optionally substituted C₁-C₆ alkyl;
R¹, R² and R³ are each independently selected from the group consisting of H, halogen, CN, optionally substituted C₁-C₆ alkyl, C₁-C₃ polyfluoroalkyl, and optionally substituted mono or bicyclic C₃-C₆ cycloalkyl;
R⁴ and R⁵ are each independently selected from the group consisting of H and C₁-C₆ alkyl;
R⁶ is a mono or bicyclic substituted C₅-C₁₀ aryl, a mono or bicyclic optionally substituted 5 to 10 membered heteroaryl or an optionally substituted C₃-C₆ cycloalkyl;
R⁷ is H;
R⁸ is a mono or bicyclic optionally substituted C₅-C₁₀ aryl, a mono or bicyclic optionally substituted 5 to 10 membered heteroaryl or an optionally substituted C₃-C₆ cycloalkyl;
R⁹ and R¹⁰ are each independently selected from the group consisting of optionally substituted C₁-C₆ alkyl, H, halogen, CN, hydroxyl, azido, NR¹R², C₁-C₃ polyfluoroalkyl, optionally substituted C₃-C₆ cycloalkyl, optionally substituted C₁-C₆ alkoxy or optionally substituted C₂-C₆ alkenyl; and
R¹¹ and R¹² are each independently selected from the group consisting of optionally substituted C₁-C₆ alkyl, H, halogen, CN, hydroxyl, azido, NR¹R², C₁-C₃ polyfluoroalkyl, optionally substituted C₃-C₆ cycloalkyl, optionally substituted C₁-C₆ alkoxy or optionally substituted C₂-C₆ alkenyl.

30. A compound according to claim 29, wherein:
L is a C₁-C₂ alkyl; and
Y is a C₁-C₂ alkyl.

31. A compound according to claim 1, wherein:
X is S or CR⁹R¹⁰;
X¹ is CH or N;
X² is CH;
X³ is CH or N;
n is 1;
Q is C═O;
L is a C₁-C₂ alkyl;
Y is a C₁-C₂ alkyl;
R⁶ is a mono or bicyclic optionally substituted C₅-C₁₀ aryl;
R⁷ is H;
R⁸ is a mono or bicyclic optionally substituted C₅-C₁₀ aryl or a mono or bicyclic optionally substituted 5 to 10 membered heteroaryl;
R⁹ and R¹⁰ are each independently selected from the group consisting of C₁-C₆ alkyl, H, halogen, CN and azido; and
R¹¹ and R¹² are each independently selected from the group consisting of C₁-C₆ alkyl, H, halogen, CN and azido.

32. A compound according to claim 31, wherein:
L is —CH₂—;
Y is —CH₂—;
R⁶ is a phenyl ring substituted with at least one chlorine and/or fluorine;
R⁸ is a phenyl ring substituted with at least one fluorine;
R⁹ and R¹⁰ are each independently selected from the group consisting of C₁-C₃ alkyl and H; and
R¹¹ and R¹² are each independently selected from the group consisting of C₁-C₃ alkyl and H.

33. A compound according to claim 1, wherein the compound is:
4-(2-chlorobenzyl)-N-(furan-2-ylmethyl)-3-oxo-3,4-dihydro-2H-benzo[b-1,4]thiazine-6-carboxamide;
N-(furan-2-ylmethyl)-3-oxo-4-(2-oxo-2-phenylethyl)-3,4-dihydro-2H-benzo[b][1,4]thiazine-6-carboxamide;
N-(furan-2-ylmethyl)-4-(2-(indolin-1-yl)-2-oxoethyl)-3-oxo-3,4-dihydro-2H-benzo[b-1,4]thiazine-6-carboxamide;
N-(furan-2-ylmethyl)-3-oxo-4-(2-oxo-2-(pyridin-4-ylamino)ethyl)-3,4-dihydro-2H-benzo[b-1,4]thiazine-6-carboxamide;
N-(furan-2-ylmethyl)-3-oxo-4-(2-oxo-2-(phenylamino)ethyl)-3,4-dihydro-2H-benzo[b-1,4]thiazine-6-carboxamide;
4-(benzo[d]thiazol-2-ylmethyl)-N-(furan-2-ylmethyl)-3-oxo-3,4-dihydro-2H-benzo[b-1,4]thiazine-6-carboxamide;
4-(benzo[d]oxazol-2-ylmethyl)-N-(furan-2-ylmethyl)-3-oxo-3,4-dihydro-2H-benzo[b-1,4]thiazine-6-carboxamide;
N-(furan-2-ylmethyl)-3-oxo-4-phenethyl-3,4-dihydro-2H-benzo[b-1,4]thiazine-6-carboxamide;
4-(2-((2-aminopyrimidin-5-yl)amino)-2-oxoethyl)-N-(furan-2-ylmethyl)-3-oxo-3,4-dihydro-2H-benzo[b-1,4]thiazine-6-carboxamide;
4-benzyl-N-((1-methyl-1H-pyrrol-2-yl)methyl)-3-oxo-3,4-dihydro-2H-benzo[b-1,4]thiazine-6-carboxamide;
4-(2,3-dichlorobenzyl)-N-(furan-2-ylmethyl)-3-oxo-3,4-dihydro-2H-benzo[b-1,4]thiazine-6-carboxamide;
4-(2,3-dimethylbenzyl)-N-(furan-2-ylmethyl)-3-oxo-3,4-dihydro-2H-benzo[b-1,4]thiazine-6-carboxamide;
4-(2,6-dimethylbenzyl)-N-(furan-2-ylmethyl)-3-oxo-3,4-dihydro-2H-benzo[b-1,4]thiazine-6-carboxamide;
N-(furan-2-ylmethyl)-4-(naphthalen-1-ylmethyl)-3-oxo-3,4-dihydro-2H-benzo[b-1,4]thiazine-6-carboxamide;
4-(cyclohexylmethyl)-N-(furan-2-ylmethyl)-3-oxo-3,4-dihydro-2H-benzo[b-1,4]thiazine-6-carboxamide;
N-(cyclohexylmethyl)-4-(2-fluorobenzyl)-3-oxo-3,4-dihydro-2H-benzo[b-1,4]thiazine-6-carboxamide;
N-(furan-2-ylmethyl)-4-(2-hydroxy-2-phenylethyl)-3-oxo-3,4-dihydro-2H-benzo[b-1,4]thiazine-6-carboxamide;
(S)—N-(1-cyclohexylethyl)-4-(2-fluorobenzyl)-3-oxo-3,4-dihydro-2H-benzo[b-1,4]thiazine-6-carboxamide;
(R)—N-(1-cyclohexylethyl)-4-(2-fluorobenzyl)-3-oxo-3,4-dihydro-2H-benzo[b-1,4]thiazine-6-carboxamide;
N-(cyclopropylmethyl)-4-(2-fluorobenzyl)-3-oxo-3,4-dihydro-2H-benzo[b-1,4]thiazine-6-carboxamide;
N,4-bis(2-chloro-6-fluorobenzyl)-3-oxo-3,4-dihydro-2H-benzo[b-1,4]thiazine-6-carboxamide;
N-((6,6-dimethylbicyclo[3.1.1]heptan-3-yl)methyl)-4-(2-fluorobenzyl)-3-oxo-3,4-dihydro-2H-benzo[b-1,4]thiazine-6-carboxamide;
N-(1-((3r,5r,7r)-adamantan-1-yl)ethyl)-4-(2-fluorobenzyl)-3-oxo-3,4-dihydro-2H-benzo[b-1,4]thiazine-6-carboxamide;
N-((3r,5r,7r)-adamantan-1-ylmethyl)-4-(2-fluorobenzyl)-3-oxo-3,4-dihydro-2H-benzo[b-1,4]thiazine-6-carboxamide;
N-(cyclohexylmethyl)-4-(2,3-dimethylbenzyl)-3-oxo-3,4-dihydro-2H-benzo[b-1,4]thiazine-6-carboxamide;
4-(2-fluorobenzyl)-N-(naphthalen-1-ylmethyl)-3-oxo-3,4-dihydro-2H-benzo[b-1,4]thiazine-6-carboxamide;
N-(furan-2-ylmethyl)-3-oxo-4-(pyridin-3-ylmethyl)-3,4-dihydro-2H-benzo[b-1,4]thiazine-6-carboxamide;
N-(furan-2-ylmethyl)-3-oxo-4-(pyridin-4-ylmethyl)-3,4-dihydro-2H-benzo[b-1,4]thiazine-6-carboxamide;
N-(furan-2-ylmethyl)-3-oxo-4-(1-phenylethyl)-3,4-dihydro-2H-benzo[b-1,4]thiazine-6-carboxamide;
N-(furan-2-ylmethyl)-3-oxo-4-(2-(trifluoromethyl)benzyl)-3,4-dihydro-2H-benzo[b-1,4]thiazine-6-carboxamide;
4-(2-chloro-6-fluorobenzyl)-N-(2,6-difluorobenzyl)-3-oxo-3,4-dihydro-2H-benzo[b-1,4]thiazine-6-carboxamide;
4-(2-chloro-6-fluorobenzyl)-N-(naphthalen-2-ylmethyl)-3-oxo-3,4-dihydro-2H-benzo[b-1,4]thiazine-6-carboxamide;
4-benzyl-N-(furan-2-ylmethyl)-N-methyl-3-oxo-3,4-dihydro-2H-benzo[b][1,4]thiazine-6-carboxamide;
4-(2-chloro-6-fluorobenzyl)-N-(2,4-difluorobenzyl)-3-oxo-3,4-dihydro-2H-benzo[b-1,4]thiazine-6-carboxamide;
4-(2-chloro-6-fluorobenzyl)-N-(cyclopentylmethyl)-3-oxo-3,4-dihydro-2H-benzo[b][1,4]thiazine-6-carboxamide;
4-(2-chloro-6-fluorobenzyl)-N-((6-methylpyridin-3-yl)methyl)-3-oxo-3,4-dihydro-2H-benzo[b-1,4]thiazine-6-carboxamide;
4-(2-chloro-6-fluorobenzyl)-N-(3-methylbenzyl)-3-oxo-3,4-dihydro-2H-benzo[b-1,4]thiazine-6-carboxamide;
4-(2-chloro-6-fluorobenzyl)-N-(3-fluorobenzyl)-3-oxo-3,4-dihydro-2H-benzo[b-1,4]thiazine-6-carboxamide;
3-oxo-4-(pyridin-4-ylmethyl)-N-(2,4,6-trifluorobenzyl)-3,4-dihydro-2H-benzo[b-1,4]thiazine-6-carboxamide;
N-(benzofuran-2-ylmethyl)-4-(2-chloro-6-fluorobenzyl)-3-oxo-3,4-dihydro-2H-benzo[b-1,4]thiazine-6-carboxamide;
4-(2-chloro-6-fluorobenzyl)-N-(3-cyano-2-phenylpropyl)-3-oxo-3,4-dihydro-2H-benzo[b-1,4]thiazine-6-carboxamide;
N-benzhydryl-4-(2-fluorobenzyl)-3-oxo-3,4-dihydro-2H-benzo[b][1,4]thiazine-6-carboxamide;
4-(2-chloro-6-fluorobenzyl)-N-(2-cyclohexyl-2-phenylethyl)-3-oxo-3,4-dihydro-2H-benzo[b][1,4]thiazine-6-carboxamide;
N-((1-benzylpiperidin-3-yl)methyl)-4-(2-fluorobenzyl)-3-oxo-3,4-dihydro-2H-benzo[b][1,4]thiazine-6-carboxamide;
4-(2-fluorobenzyl)-3-oxo-N-((1-phenylpiperidin-4-yl)methyl)-3,4-dihydro-2H-benzo[b][1,4]thiazine-6-carboxamide;
N-([1,1′-biphenyl]-2-ylmethyl)-4-(2-chloro-6-fluorobenzyl)-3-oxo-3,4-dihydro-2H-benzo[b][1,4]thiazine-6-carboxamide;
N-([1,1′-biphenyl]-3-ylmethyl)-4-(2-chloro-6-fluorobenzyl)-3-oxo-3,4-dihydro-2H-benzo[b][1,4]thiazine-6-carboxamide;
N-((1-benzylpiperidin-4-yl)methyl)-4-(2-chloro-6-fluorobenzyl)-3-oxo-3,4-dihydro-2H-benzo[b][1,4]thiazine-6-carboxamide;
4-(2-fluorobenzyl)-3-oxo-N-((1-phenylpiperidin-3-yl)methyl)-3,4-dihydro-2H-benzo[b][1,4]thiazine-6-carboxamide;
4-benzyl-N-(furan-3-ylmethyl)-3-oxo-3,4-dihydro-2H-benzo[b-1,4]thiazine-6-carboxamide;
4-benzyl-3-oxo-N-((4-oxo-3,4-dihydrophthalazin-1-yl)methyl)-3,4-dihydro-2H-benzo[b-1,4]thiazine-6-carboxamide;
N-((6-aminopyridin-3-yl)methyl)-4-benzyl-3-oxo-3,4-dihydro-2H-benzo[b-1,4]thiazine-6-carboxamide;
4-benzyl-N-(2-(dimethylamino)-1-phenylethyl)-3-oxo-3,4-dihydro-2H-benzo[b-1,4]thiazine-6-carboxamide;
4-benzyl-3-oxo-N-(pyrimidin-2-ylmethyl)-3,4-dihydro-2H-benzo[b-1,4]thiazine-6-carboxamide;
4-benzyl-N-(2,3-dichlorobenzyl)-3-oxo-3,4-dihydro-2H-benzo[b-1,4]thiazine-6-carboxamide;
N,4-dibenzyl-N-(cyanomethyl)-3-oxo-3,4-dihydro-2H-benzo[b-1,4]thiazine-6-carboxamide;
4-benzyl-N-(1-(4-(methylsulfonyl)phenyl)ethyl)-3-oxo-3,4-dihydro-2H-benzo[b-1,4]thiazine-6-carboxamide;
4-benzyl-3-oxo-N-(pyrazin-2-ylmethyl)-3,4-dihydro-2H-benzo[b-1,4]thiazine-6-carboxamide;
4-benzyl-N-(oxazol-2-ylmethyl)-3-oxo-3,4-dihydro-2H-benzo[b-1,4]thiazine-6-carboxamide;
4-benzyl-3-oxo-N-(thiazol-2-ylmethyl)-3,4-dihydro-2H-benzo[b-1,4]thiazine-6-carboxamide;
4-benzyl-3-oxo-N-(thiophen-3-ylmethyl)-3,4-dihydro-2H-benzo[b-1,4]thiazine-6-carboxamide;
4-benzyl-N-((5-methylfuran-2-yl)methyl)-3-oxo-3,4-dihydro-2H-benzo[b-1,4]thiazine-6-carboxamide;
4-benzyl-N-((5-methyloxazol-2-yl)methyl)-3-oxo-3,4-dihydro-2H-benzo[b-1,4]thiazine-6-carboxamide;
4-benzyl-N-((5-methylthiazol-2-yl)methyl)-3-oxo-3,4-dihydro-2H-benzo[b-1,4]thiazine-6-carboxamide;
4-benzyl-N-((4-methylthiazol-2-yl)methyl)-3-oxo-3,4-dihydro-2H-benzo[b-1,4]thiazine-6-carboxamide;
4-benzyl-N-((3-methyl-1,2,4-oxadiazol-5-yl)methyl)-3-oxo-3,4-dihydro-2H-benzo[b-1,4]thiazine-6-carboxamide;
4-benzyl-N-((1-methyl-1H-pyrazol-5-yl)methyl)-3-oxo-3,4-dihydro-2H-benzo[b-1,4]thiazine-6-carboxamide;
4-benzyl-N-(1-(furan-2-yl)ethyl)-3-oxo-3,4-dihydro-2H-benzo[b-1,4]thiazine-6-carboxamide;
N-(benzofuran-2-ylmethyl)-4-benzyl-3-oxo-3,4-dihydro-2H-benzo[b-1,4]thiazine-6-carboxamide;
N-(benzofuran-3-ylmethyl)-4-benzyl-3-oxo-3,4-dihydro-2H-benzo[b-1,4]thiazine-6-carboxamide;
4-benzyl-N-(4-cyanobenzyl)-3-oxo-3,4-dihydro-2H-benzo[b-1,4]thiazine-6-carboxamide;
4-benzyl-3-oxo-N-(4-(trifluoromethyl)benzyl)-3,4-dihydro-2H-benzo[b-1,4]thiazine-6-carboxamide;
4-benzyl-N-(4-chlorobenzyl)-3-oxo-3,4-dihydro-2H-benzo[b-1,4]thiazine-6-carboxamide;
4-benzyl-N-(naphthalen-2-ylmethyl)-3-oxo-3,4-dihydro-2H-benzo[b-1,4]thiazine-6-carboxamide;
4-benzyl-3-oxo-N-(1-phenylcyclopentyl)-3,4-dihydro-2H-benzo[b-1,4]thiazine-6-carboxamide;
4-benzyl-N-(3-methylbenzyl)-3-oxo-3,4-dihydro-2H-benzo[b-1,4]thiazine-6-carboxamide;
4-benzyl-N-((2-methylthiazol-4-yl)methyl)-3-oxo-3,4-dihydro-2H-benzo[b-1,4]thiazine-6-carboxamide;
4-benzyl-N-(2-(2-fluorophenyl)propan-2-yl)-3-oxo-3,4-dihydro-2H-benzo[b-1,4]thiazine-6-carboxamide;
4-benzyl-N-(1,2-diphenylethyl)-3-oxo-3,4-dihydro-2H-benzo[b-1,4]thiazine-6-carboxamide;
4-benzyl-N-(4-isopropylbenzyl)-3-oxo-3,4-dihydro-2H-benzo[b-1,4]thiazine-6-carboxamide;
4-benzyl-N-(2-cyclohexylethyl)-3-oxo-3,4-dihydro-2H-benzo[b-1,4]thiazine-6-carboxamide;
4-(2,3-difluorobenzyl)-N-(furan-2-ylmethyl)-3-oxo-3,4-dihydro-2H-benzo[b-1,4]thiazine-6-carboxamide;
N-(furan-2-ylmethyl)-4-(3-methyl-5-(trifluoromethyl)benzyl)-3-oxo-3,4-dihydro-2H-benzo[b-1,4]thiazine-6-carboxamide;
N-(furan-2-ylmethyl)-4-((3-methyl-1,2,4-oxadiazol-5-yl)methyl)-3-oxo-3,4-dihydro-2H-benzo[b-1,4]thiazine-6-carboxamide;
N-(furan-2-ylmethyl)-4-((5-methylisoxazol-3-yl)methyl)-3-oxo-3,4-dihydro-2H-benzo[b-1,4]thiazine-6-carboxamide;
4-(3-carbamoylbenzyl)-N-(furan-2-ylmethyl)-3-oxo-3,4-dihydro-2H-benzo[b-1,4]thiazine-6-carboxamide;
4-(3,5-difluorobenzyl)-N-(furan-2-ylmethyl)-3-oxo-3,4-dihydro-2H-benzo[b][1,4]thiazine-6-carboxamide;
N-(furan-2-ylmethyl)-3-oxo-4-(3-(trifluoromethoxy)benzyl)-3,4-dihydro-2H-benzo[b-1,4]thiazine-6-carboxamide;
4-(2-chlorophenethyl)-N-(furan-2-ylmethyl)-3-oxo-3,4-dihydro-2H-benzo[b-1,4]thiazine-6-carboxamide;
4-(3-chlorophenethyl)-N-(furan-2-ylmethyl)-3-oxo-3,4-dihydro-2H-benzo[b-1,4]thiazine-6-carboxamide;
N-(furan-2-ylmethyl)-3-oxo-4-(2-(trifluoromethoxy)benzyl)-3,4-dihydro-2H-benzo[b-1,4]thiazine-6-carboxamide;
N-(furan-2-ylmethyl)-3-oxo-4-(3-(trifluoromethyl)phenethyl)-3,4-dihydro-2H-benzo[b-1,4]thiazine-6-carboxamide;
4-(2,6-dichlorobenzyl)-N-(furan-2-ylmethyl)-3-oxo-3,4-dihydro-2H-benzo[b-1,4]thiazine-6-carboxamide;
4-(2-cyanobenzyl)-N-(furan-2-ylmethyl)-3-oxo-3,4-dihydro-2H-benzo[b-1,4]thiazine-6-carboxamide;
4-((5-chlorothiophen-2-yl)methyl)-N-(furan-2-ylmethyl)-3-oxo-3,4-dihydro-2H-benzo[b-1,4]thiazine-6-carboxamide;
N-(furan-2-ylmethyl)-4-((2-methylthiazol-4-yl)methyl)-3-oxo-3,4-dihydro-2H-benzo[b-1,4]thiazine-6-carboxamide;
N-(furan-2-ylmethyl)-3-oxo-4-(3-(trifluoromethyl)benzyl)-3,4-dihydro-2H-benzo[b-1,4]thiazine-6-carboxamide;
N-(furan-2-ylmethyl)-4-((1-methylpiperidin-3-yl)methyl)-3-oxo-3,4-dihydro-2H-benzo[b-1,4]thiazine-6-carboxamide;
4-(2-chloro-5-fluorobenzyl)-N-(furan-2-ylmethyl)-3-oxo-3,4-dihydro-2H-benzo[b-1,4]thiazine-6-carboxamide;
4-(2,5-difluorobenzyl)-N-(furan-2-ylmethyl)-3-oxo-3,4-dihydro-2H-benzo[b-1,4]thiazine-6-carboxamide;
4-(2,6-difluorobenzyl)-N-(furan-2-ylmethyl)-3-oxo-3,4-dihydro-2H-benzo[b-1,4]thiazine-6-carboxamide;
4-(3-chloro-5-(trifluoromethyl)benzyl)-N-(furan-2-ylmethyl)-3-oxo-3,4-dihydro-2H-benzo[b-1,4]thiazine-6-carboxamide;
N-(furan-2-ylmethyl)-3-oxo-4-(2-(phenylsulfonyl)ethyl)-3,4-dihydro-2H-benzo[b-1,4]thiazine-6-carboxamide;
N-(furan-2-ylmethyl)-4-(imidazo[2,1-b]thiazol-6-ylmethyl)-3-oxo-3,4-dihydro-2H-benzo[b-1,4]thiazine-6-carboxamide;
4-((4,5-dimethyl-4H-1,2,4-triazol-3-yl)methyl)-N-(furan-2-ylmethyl)-3-oxo-3,4-dihydro-2H-benzo[b-1,4]thiazine-6-carboxamide;
4-(2-fluorobenzyl)-N-(furan-2-ylmethyl)-3-oxo-3,4-dihydro-2H-benzo[b-1,4]thiazine-6-carboxamide;
4-(2-chloro-6-fluorobenzyl)-3-oxo-N-(thiophen-2-ylmethyl)-3,4-dihydro-2H-benzo[b-1,4]thiazine-6-carboxamide;
4-(2-chloro-6-fluorobenzyl)-3-oxo-N-(pyridin-2-ylmethyl)-3,4-dihydro-2H-benzo[b-1,4]thiazine-6-carboxamide;
4-(2-chloro-6-fluorobenzyl)-3-oxo-N-(pyridin-3-ylmethyl)-3,4-dihydro-2H-benzo[b-1,4]thiazine-6-carboxamide;
N-(cyclohexyl(phenyl)methyl)-4-(2-fluorobenzyl)-3-oxo-3,4-dihydro-2H-benzo[b][1,4]thiazine-6-carboxamide;
4-benzyl-N-(furan-2-ylmethyl)-3-oxo-3,4-dihydro-2H-benzo[b-1,4]thiazine-6-carboxamide;
4-(2-chloro-6-fluorobenzyl)-3-oxo-N-(2,4,6-trifluorobenzyl)-3,4-dihydro-2H-benzo[b-1,4]thiazine-6-carboxamide;
2-(3-oxo-6-((2,4,6-trifluorobenzyl)carbamoyl)-2H-benzo[b][1,4]thiazin-4(3H)-yl)-2-phenylacetic acid;
4-(2,6-difluoro-4-methoxybenzyl)-3-oxo-N-(2,4,6-trifluorobenzyl)-3,4-dihydro-2H-benzo[b][1,4]thiazine-6-carboxamide;
4-(2,6-difluoro-4-hydroxybenzyl)-3-oxo-N-(2,4,6-trifluorobenzyl)-3,4-dihydro-2H-benzo[b][1,4]thiazine-6-carboxamide;
4-benzyl-N-(2-methoxybenzyl)-3-oxo-3,4-dihydro-2H-benzo[b-1,4]thiazine-6-carboxamide;
4-benzyl-N-(2-chlorobenzyl)-3-oxo-3,4-dihydro-2H-benzo[b-1,4]thiazine-6-carboxamide;
N,4-dibenzyl-3-oxo-3,4-dihydro-2H-benzo[b-1,4]thiazine-6-carboxamide;
4-(4-chlorobenzyl)-N-(furan-2-ylmethyl)-3-oxo-3,4-dihydro-2H-benzo[b-1,4]thiazine-6-carboxamide;
4-(4-fluorobenzyl)-N-(furan-2-ylmethyl)-3-oxo-3,4-dihydro-2H-benzo[b-1,4]thiazine-6-carboxamide;
4-benzyl-N-((1-methyl-1H-pyrazol-4-yl)methyl)-3-oxo-3,4-dihydro-2H-benzo[b][1,4]thiazine-6-carboxamide;
4-(2-chlorobenzyl)-N-(4-fluorobenzyl)-3-oxo-3,4-dihydro-2H-benzo[b-1,4]thiazine-6-carboxamide;
N,4-bis(2-chlorobenzyl)-3-oxo-3,4-dihydro-2H-benzo[b-1,4]thiazine-6-carboxamide;
N-(2-chlorobenzyl)-4-(3-chlorobenzyl)-3-oxo-3,4-dihydro-2H-benzo[b-1,4]thiazine-6-carboxamide;
4-(4-chlorobenzyl)-3-oxo-N-(1-phenylethyl)-3,4-dihydro-2H-benzo[b-1,4]thiazine-6-carboxamide;
N-benzyl-4-(2-chloro-6-fluorobenzyl)-3-oxo-3,4-dihydro-2H-benzo[b-1,4]thiazine-6-carboxamide;
4-(2-chloro-6-fluorobenzyl)-N-(4-fluorobenzyl)-3-oxo-3,4-dihydro-2H-benzo[b-1,4]thiazine-6-carboxamide;
4-(2-chloro-6-fluorobenzyl)-N-((1-(4-methylbenzyl)piperidin-4-yl)methyl)-3-oxo-3,4-dihydro-2H-benzo[b][1,4]thiazine-6-carboxamide;
4-(2-chloro-6-fluorobenzyl)-3-oxo-N-phenethyl-3,4-dihydro-2H-benzo[b-1,4]thiazine-6-carboxamide;
4-(2-chloro-6-fluorobenzyl)-3-oxo-N-((tetrahydrofuran-2-yl)methyl)-3,4-dihydro-2H-benzo[b-1,4]thiazine-6-carboxamide;
4-(2-chloro-6-fluorobenzyl)-N-(furan-2-ylmethyl)-3-oxo-3,4-dihydro-2H-benzo[b][1,4]thiazine-6-carboxamide;
4-Benzyl-N-(furan-2-ylmethyl)-2,2-dimethyl-3-oxo-3,4-dihydro-2H-benzo[b-1,4]thiazine-6-carboxamide;
2-methyl-N-2,4,6-trifluorobenzyl-4-[(1-methyl-1H-pyrazol-4-yl)methyl]-3-oxo-3,4-dihydro-2H-1,4-benzothiazine-6-carboxamide;
4-benzyl-N-(furan-2-ylmethyl)-2-methyl-3-oxo-3,4-dihydro-2H-benzo[b-1,4]thiazine-6-carboxamide;
2-(dimethylamino)-4-(2-fluorobenzyl)-N-(furan-2-ylmethyl)-3-oxo-3,4-dihydro-2H-benzo[b-1,4]thiazine-6-carboxamide;
N-(cyclohexylmethyl)-4-(2-fluorobenzyl)-2,2-dimethyl-3-oxo-3,4-dihydro-2H-benzo[b-1,4]thiazine-6-carboxamide;
2-ethoxy-4-(2-fluorobenzyl)-N-(furan-2-ylmethyl)-3-oxo-3,4-dihydro-2H-benzo[b-1,4]thiazine-6-carboxamide;
4-(2-chloro-6-fluorobenzyl)-2-methyl-3-oxo-N-(2,4,6-trifluorobenzyl)-3,4-dihydro-2H-benzo[b-1,4]thiazine-6-carboxamide;
2-(4-(2-fluorobenzyl)-6-((furan-2-ylmethyl)carbamoyl)-2-methyl-3-oxo-3,4-dihydro-2H-benzo[b-1,4]thiazin-2-yl)acetic acid;
4-(2-fluorobenzyl)-2-methyl-3-oxo-N-(2,4,6-trifluorobenzyl)-3,4-dihydro-2H-benzo[b-1,4]thiazine-6-carboxamide;
4-(2-fluorobenzyl)-2-methyl-N-(3-methylbenzyl)-3-oxo-3,4-dihydro-2H-benzo[b-1,4]thiazine-6-carboxamide;
4-(2-fluorobenzyl)-2-methyl-3-oxo-N-(2,4,6-trifluorobenzyl)-3,4-dihydro-2H-benzo[b-1,4]thiazine-6-carboxamide;
4-(2-chlorobenzyl)-2-methyl-N-(3-methylbenzyl)-3-oxo-3,4-dihydro-2H-benzo[b-1,4]thiazine-6-carboxamide;
4-(3,5-difluorobenzyl)-2-methyl-N-(3-methylbenzyl)-3-oxo-3,4-dihydro-2H-benzo[b-1,4]thiazine-6-carboxamide;
4-(3,5-difluorobenzyl)-2-methyl-3-oxo-N-(2,4,6-trifluorobenzyl)-3,4-dihydro-2H-benzo[b-1,4]thiazine-6-carboxamide;
2-allyl-4-(2-chloro-6-fluorobenzyl)-3-oxo-N-(2,4,6-trifluorobenzyl)-3,4-dihydro-2H-benzo[b-1,4]thiazine-6-carboxamide;
4-(2-chloro-6-fluorobenzyl)-3-oxo-2-propyl-N-(2,4,6-trifluorobenzyl)-3,4-dihydro-2H-benzo[b-1,4]thiazine-6-carboxamide;
4-(3,5-difluorobenzyl)-2-methyl-3-oxo-N-(2,4,6-trifluorobenzyl)-3,4-dihydro-2H-benzo[b][1,4]thiazine-6-carboxamide;
N-(benzofuran-2-ylmethyl)-4-(3,5-difluorobenzyl)-2-methyl-3-oxo-3,4-dihydro-2H-benzo[b][1,4]thiazine-6-carboxamide;
4-(2-chloro-6-fluorobenzyl)-2-(chloromethyl)-N-(2,4-difluorobenzyl)-2-methyl-3-oxo-3,4-dihydro-2H-benzo[b][1,4]thiazine-6-carboxamide;
4-(2-chloro-6-fluorobenzyl)-N-(2,4-difluorobenzyl)-2-(hydroxymethyl)-2-methyl-3-oxo-3,4-dihydro-2H-benzo[b][1,4]thiazine-6-carboxamide;
4-(3,5-difluorobenzyl)-2-methyl-N-((5-methylfuran-2-yl)methyl)-3-oxo-3,4-dihydro-2H-benzo[b][1,4]thiazine-6-carboxamide;
4-(3,5-difluorobenzyl)-2-methyl-3-oxo-N-(2,4,6-trifluorobenzyl)-3,4-dihydro-2H-benzo[b][1,4]thiazine-6-carboxamide;
N-(benzofuran-2-ylmethyl)-4-(2-chloro-6-fluorobenzyl)-2,2-dimethyl-3-oxo-3,4-dihydro-2H-benzo[b][1,4]thiazine-6-carboxamide;
4-(2-chloro-6-fluoro-3-hydroxybenzyl)-2-methyl-3-oxo-N-(2,4,6-trifluorobenzyl)-3,4-dihydro-2H-benzo[b][1,4]thiazine-6-carboxamide;
4-(3,5-difluorobenzyl)-N-(furan-2-ylmethyl)-2-methyl-3-oxo-3,4-dihydro-2H-benzo[b][1,4]thiazine-6-carboxamide;
2-methyl-3-oxo-4-(pyridin-4-ylmethyl)-N-(2,4,6-trifluorobenzyl)-3,4-dihydro-2H-benzo[b][1,4]thiazine-6-carboxamide;
2-methyl-4-((3-methylisoxazol-5-yl)methyl)-3-oxo-N-(2,4,6-trifluorobenzyl)-3,4-dihydro-2H-benzo[b][1,4]thiazine-6-carboxamide;
2-methyl-4-((5-methylisoxazol-3-yl)methyl)-3-oxo-N-(2,4,6-trifluorobenzyl)-3,4-dihydro-2H-benzo[b][1,4]thiazine-6-carboxamide;
4-(3-carbamoylbenzyl)-2-methyl-3-oxo-N-(2,4,6-trifluorobenzyl)-3,4-dihydro-2H-benzo[b][1,4]thiazine-6-carboxamide;
N-(benzofuran-2-ylmethyl)-4-(2-fluorobenzyl)-2,2-dimethyl-3-oxo-3,4-dihydro-2H-benzo[b][1,4]thiazine-6-carboxamide;
2-(cyanomethyl)-N-(2,4-difluorobenzyl)-4-(3,5-difluorobenzyl)-3-oxo-3,4-dihydro-2H-benzo[b][1,4]thiazine-6-carboxamide;
4-(3,5-difluorobenzyl)-2-methyl-N-((5-methyloxazol-2-yl)methyl)-3-oxo-3,4-dihydro-2H-benzo[b][1,4]thiazine-6-carboxamide;
4-(2-chloro-6-fluorobenzyl)-2-hydroxy-2-methyl-3-oxo-N-(2,4,6-trifluorobenzyl)-3,4-dihydro-2H-benzo[b][1,4]thiazine-6-carboxamide;
4-(2-chloro-6-fluorobenzyl)-2-fluoro-2-methyl-3-oxo-N-(2,4,6-trifluorobenzyl)-3,4-dihydro-2H-benzo[b][1,4]thiazine-6-carboxamide;
2-(2-aminoethyl)-N-(2,4-difluorobenzyl)-4-(3,5-difluorobenzyl)-3-oxo-3,4-dihydro-2H-benzo[b][1,4]thiazine-6-carboxamide;
4-(3,5-difluorobenzyl)-2-methyl-N-((3-methyl-1,2,4-oxadiazol-5-yl)methyl)-3-oxo-3,4-dihydro-2H-benzo[b][1,4]thiazine-6-carboxamide;
4-(3,5-difluorobenzyl)-2-methyl-3-oxo-N-(pyrazin-2-ylmethyl)-3,4-dihydro-2H-benzo[b][1,4]thiazine-6-carboxamide;
4-(3,5-difluorobenzyl)-2-methyl-N-((1-methyl-1H-pyrazol-4-yl)methyl)-3-oxo-3,4-dihydro-2H-benzo[b][1,4]thiazine-6-carboxamide;
4-(3,5-difluorobenzyl)-2-(2-(methylsulfonamido)ethyl)-3-oxo-N-(2,4,6-trifluorobenzyl)-3,4-dihydro-2H-benzo[b][1,4]thiazine-6-carboxamide;
4-benzyl-N-(3,4-dimethoxyphenethyl)-3-oxo-3,4-dihydro-2H-benzo[b][1,4]thiazine-6-carboxamide;
4-(2-chloro-6-fluorobenzyl)-2-methyl-N-((1-methyl-1H-pyrazol-3-yl)methyl)-3-oxo-3,4-dihydro-2H-benzo[b][1,4]thiazine-6-carboxamide;
2-methyl-4-((1-methyl-1H-pyrazol-4-yl)methyl)-N-((5-methyloxazol-2-yl)methyl)-3-oxo-3,4-dihydro-2H-benzo[b][1,4]thiazine-6-carboxamide;
4-(3,5-difluorobenzyl)-2-methyl-3-oxo-N-(2,4,6-trifluorophenethyl)-3,4-dihydro-2H-pyrido[3,2-b][1,4]thiazine-6-carboxamide;
2-(4-(2-fluorobenzyl)-6-((furan-2-ylmethyl)carbamoyl)-3-oxo-3,4-dihydro-2H-benzo[b][1,4]thiazin-2-yl)acetic acid;
2-(2-amino-2-oxoethyl)-N-(2,4-difluorobenzyl)-4-(3,5-difluorobenzyl)-3-oxo-3,4-dihydro-2H-benzo[b][1,4]thiazine-6-carboxamide;
1-(2-Chloro-6-fluorobenzyl)-N-(furan-2-ylmethyl)-2-oxo-2,3-dihydro-1H-pyrido[2,3-b-1,4]thiazine-7-carboxamide;
1-(2-chloro-6-fluorobenzyl)-2-oxo-N-(2,4,6-trifluorobenzyl)-2,3-dihydro-1H-pyrido[2,3-b-1,4]thiazine-7-carboxamide;
1-(2-chloro-6-fluorobenzyl)-N-(cyclohexylmethyl)-2-oxo-2,3-dihydro-1H-pyrido[2,3-b-1,4]thiazine-7-carboxamide;
1-benzyl-N-(furan-2-ylmethyl)-2-oxo-2,3-dihydro-1H-pyrido[2,3-b-1,4]thiazine-7-carboxamide;
4-(2-chloro-6-fluorobenzyl)-N-(furan-2-ylmethyl)-3-oxo-3,4-dihydro-2H-pyrido[3,2-b-1,4]thiazine-6-carboxamide;
1-(2-chloro-6-fluorobenzyl)-N-(furan-2-ylmethyl)-2-oxo-2,3-dihydro-1H-pyrido[3,4-b-1,4]thiazine-7-carboxamide;
N-(benzofuran-2-ylmethyl)-4-(3,5-difluorobenzyl)-3-oxo-3,4-dihydro-2H-pyrido[3,2-b-1,4]thiazine-6-carboxamide;
4-(2-chloro-6-fluorobenzyl)-2-methyl-N-(3-methylbenzyl)-3-oxo-3,4-dihydro-2H-pyrido[3,2-b-1,4]thiazine-6-carboxamide;
4-(3,5-difluorobenzyl)-2-methyl-3-oxo-N-(2,4,6-trifluorobenzyl)-3,4-dihydro-2H-pyrido[3,2-b-1,4]thiazine-6-carboxamide;
4-(2-chloro-6-fluorobenzyl)-2-methyl-3-oxo-N-(2,4,6-trifluorobenzyl)-3,4-dihydro-2H-pyrido[3,2-b-1,4]thiazine-6-carboxamide;
4-(2-chloro-6-fluorobenzyl)-N-(2,4-difluorobenzyl)-2-methyl-3-oxo-3,4-dihydro-2H-pyrido[3,2-b-1,4]thiazine-6-carboxamide;
N-(benzofuran-2-ylmethyl)-4-(2-chloro-6-fluorobenzyl)-2-methyl-3-oxo-3,4-dihydro-2H-pyrido[3,2-b-1,4]thiazine-6-carboxamide;
4-(3,5-difluorobenzyl)-N-(furan-2-ylmethyl)-2-methyl-3-oxo-3,4-dihydro-2H-pyrido[3,2-b-1,4]thiazine-6-carboxamide;
4-(3,5-difluorobenzyl)-2-methyl-N-((5-methylfuran-2-yl)methyl)-3-oxo-3,4-dihydro-2H-pyrido[3,2-b-1,4]thiazine-6-carboxamide;
N-benzyl-4-(3,5-difluorobenzyl)-2-methyl-3-oxo-3,4-dihydro-2H-pyrido[3,2-b-1,4]thiazine-6-carboxamide;
1-(3,5-difluorobenzyl)-3-methyl-2-oxo-N-(2,4,6-trifluorobenzyl)-2,3-dihydro-1H-pyrido[2,3-b][1,4]thiazine-7-carboxamide;
N-(benzofuran-2-ylmethyl)-1-(3,5-difluorobenzyl)-3-methyl-2-oxo-2,3-dihydro-1H-pyrido[2,3-b][1,4]thiazine-7-carboxamide;
N-(benzofuran-2-ylmethyl)-1-(3,5-difluorobenzyl)-3-methyl-2-oxo-2,3-dihydro-H-pyrido[2,3-b][1,4]thiazine-7-carboxamide;
1-(2-chloro-6-fluorobenzyl)-3-methyl-N-((5-methylfuran-2-yl)methyl)-2-oxo-2,3-dihydro-1H-pyrido[2,3-b][1,4]thiazine-7-carboxamide;
1-(2-chloro-6-fluorobenzyl)-3-methyl-2-oxo-N-(2,4,6-trifluorobenzyl)-2,3-dihydro-H-pyrido[2,3-b][1,4]thiazine-7-carboxamide;
N-(benzofuran-2-ylmethyl)-1-(2-chloro-6-fluorobenzyl)-2-oxo-2,3-dihydro-H-pyrido[2,3-b][1,4]thiazine-7-carboxamide;
4-(3,5-difluorobenzyl)-2-methyl-N-((5-methylfuran-2-yl)methyl)-3-oxo-3,4-dihydro-2H-pyrido[3,2-b][1,4]thiazine-6-carboxamide;
N-(benzofuran-2-ylmethyl)-1-(2-chloro-6-fluorobenzyl)-3-methyl-2-oxo-2,3-dihydro-1H-pyrido[2,3-b][1,4]thiazine-7-carboxamide;
1-(3,5-difluorobenzyl)-2-oxo-N-(2,4,6-trifluorobenzyl)-2,3-dihydro-H-pyrido[2,3-b][1,4]thiazine-7-carboxamide;
N-benzyl-1-(2-chloro-6-fluorobenzyl)-3-methyl-2-oxo-2,3-dihydro-1H-pyrido[2,3-b][1,4]thiazine-7-carboxamide;
4-(2-chloro-6-fluorobenzyl)-2-methyl-N-((5-methylfuran-2-yl)methyl)-3-oxo-3,4-dihydro-2H-pyrido[3,2-b][1,4]thiazine-6-carboxamide;
4-(3,5-difluorobenzyl)-2-methyl-N-((5-methylfuran-2-yl)methyl)-3-oxo-3,4-dihydro-2H-pyrido[3,2-b][1,4]thiazine-6-carboxamide;
4-(1-(3,5-difluorophenyl)ethyl)-2-methyl-N-((5-methylfuran-2-yl)methyl)-3-oxo-3,4-dihydro-2H-pyrido[3,2-b][1,4]thiazine-6-carboxamide;
4-(3-carbamoylbenzyl)-2-methyl-N-((5-methylfuran-2-yl)methyl)-3-oxo-3,4-dihydro-2H-pyrido[3,2-b][1,4]thiazine-6-carboxamide;
1-((2-methoxypyridin-4-yl)methyl)-3-methyl-2-oxo-N-(2,4,6-trifluorobenzyl)-2,3-dihydro-1H-pyrido[2,3-b][1,4]thiazine-7-carboxamide;
1-(2-chloro-6-fluorobenzyl)-N-((5-methylfuran-2-yl)methyl)-2-oxo-2,3-dihydro-H-pyrido[2,3-b][1,4]thiazine-7-carboxamide;
N-(benzofuran-2-ylmethyl)-1-(3,5-difluorobenzyl)-3-methyl-2-oxo-2,3-dihydro-1H-pyrido[2,3-b][1,4]thiazine-7-carboxamide;
1-(3,5-difluorobenzyl)-3-(hydroxymethyl)-3-methyl-N-((5-methylfuran-2-yl)methyl)-2-oxo-2,3-dihydro-1H-pyrido[2,3-b][1,4]thiazine-7-carboxamide;
N-(benzofuran-2-ylmethyl)-1-(3,5-difluorobenzyl)-3-(hydroxymethyl)-3-methyl-2-oxo-2,3-dihydro-1H-pyrido[2,3-b][1,4]thiazine-7-carboxamide;
1-(3,5-difluorobenzyl)-3-(hydroxymethyl)-3-methyl-2-oxo-N-(2,4,6-trifluorobenzyl)-2,3-dihydro-1H-pyrido[2,3-b][1,4]thiazine-7-carboxamide;
1-(3,5-difluorobenzyl)-3-methyl-N-((3-methyl-1,2,4-oxadiazol-5-yl)methyl)-2-oxo-2,3-dihydro-1H-pyrido[2,3-b][1,4]thiazine-7-carboxamide;
1-(3,5-difluorobenzyl)-3-methyl-N-((5-methyloxazol-2-yl)methyl)-2-oxo-2,3-dihydro-1H-pyrido[2,3-b][1,4]thiazine-7-carboxamide;
1-(3,5-difluorobenzyl)-3-methyl-2-oxo-N-(pyridazin-3-ylmethyl)-2,3-dihydro-1H-pyrido[2,3-b][1,4]thiazine-7-carboxamide;
1-(3,5-difluorobenzyl)-3-methyl-N-((1-methyl-1H-pyrazol-3-yl)methyl)-2-oxo-2,3-dihydro-1H-pyrido[2,3-b][1,4]thiazine-7-carboxamide;
1-(3,5-difluorobenzyl)-3-methyl-N-((1-methyl-1H-pyrazol-4-yl)methyl)-2-oxo-2,3-dihydro-1H-pyrido[2,3-b][1,4]thiazine-7-carboxamide;
1-(3,5-difluorobenzyl)-3-methyl-2-oxo-N-(pyrazin-2-ylmethyl)-2,3-dihydro-1H-pyrido[2,3-b][1,4]thiazine-7-carboxamide;
4-(2-fluorobenzyl)-2,2-dimethyl-3-oxo-N-(2,4,6-trifluorobenzyl)-3,4-dihydro-2H-benzo[b][1,4]thiazine-6-carboxamide;
4-Benzyl-8-cyano-N-(furan-2-ylmethyl)-3-oxo-3,4-dihydro-2H-benzo[b-1,4]thiazine-6-carboxamide;
8-cyano-4-(3,5-difluorobenzyl)-N-(furan-2-ylmethyl)-3-oxo-3,4-dihydro-2H-benzo[b-1,4]thiazine-6-carboxamide;
8-bromo-4-(2-fluorobenzyl)-N-(furan-2-ylmethyl)-3-oxo-3,4-dihydro-2H-benzo[b-1,4]thiazine-6-carboxamide;
4-(4-fluorobenzyl)-8-(2-hydroxyethyl)-3-oxo-N-(2,4,6-trifluorobenzyl)-3,4-dihydro-2H-benzo[b][1,4]thiazine-6-carboxamide;
4-(4-fluorobenzyl)-8-(1-hydroxyethyl)-3-oxo-N-(2,4,6-trifluorobenzyl)-3,4-dihydro-2H-benzo[b][1,4]thiazine-6-carboxamide;
8-(aminomethyl)-4-(4-fluorobenzyl)-3-oxo-N-(2,4,6-trifluorobenzyl)-3,4-dihydro-2H-benzo[b][1,4]thiazine-6-carboxamide hydrochloride;
4-(4-fluorobenzyl)-3-oxo-N6-(2,4,6-trifluorobenzyl)-3,4-dihydro-2H-benzo[b][1,4]thiazine-6,8-dicarboxamide;
4-(2-Fluorobenzyl)-N-(furan-2-ylmethyl)-8-methyl-3-oxo-3,4-dihydro-2H-benzo[b-1,4]thiazine-6-carboxamide;
8-cyclopropyl-4-(2-fluorobenzyl)-N-(furan-2-ylmethyl)-3-oxo-3,4-dihydro-2H-benzo[b][1,4]thiazine-6-carboxamide;
7-bromo-4-(2-fluorobenzyl)-N-(furan-2-ylmethyl)-3-oxo-3,4-dihydro-2H-benzo[b][1,4]thiazine-6-carboxamide;
7-cyclopropyl-4-(2-fluorobenzyl)-N-(furan-2-ylmethyl)-3-oxo-3,4-dihydro-2H-benzo[b-1,4]thiazine-6-carboxamide;
4-benzyl-N-(furan-2-ylmethyl)-7-methyl-3-oxo-3,4-dihydro-2H-benzo[b-1,4]thiazine-6-carboxamide;
4-(2-chloro-6-fluorobenzyl)-N-(furan-2-ylmethyl)-3-oxo-3,4-dihydro-2H-benzo[b-1,4]oxazine-6-carboxamide;
2-Methyl-4-(3,5-difluorobenzyl)-3-oxo-N-(2,4,6-trifluorobenzyl)-3,4-dihydro-2H-1,4-benzoxazine-6-carboxamide;
(R)-4-(3,5-difluorobenzyl)-2-methyl-3-oxo-N-(2,4,6-trifluorobenzyl)-3,4-dihydro-2H-benzo[b-1,4]oxazine-6-carboxamide;
(S)-4-(3,5-difluorobenzyl)-2-methyl-3-oxo-N-(2,4,6-trifluorobenzyl)-3,4-dihydro-2H-benzo[b-1,4]oxazine-6-carboxamide;
(S)—N-(benzofuran-2-ylmethyl)-4-(3,5-difluorobenzyl)-2-methyl-3-oxo-3,4-dihydro-2H-benzo[b-1,4]oxazine-6-carboxamide;
1-(3,5-difluorobenzyl)-2-oxo-N-(2,4,6-trifluorobenzyl)-2,3-dihydro-1H-pyrido[2,3-b-1,4]oxazine-7-carboxamide;
(S)-1-(3,5-difluorobenzyl)-3-methyl-2-oxo-N-(2,4,6-trifluorobenzyl)-2,3-dihydro-1H-pyrido[2,3-b-1,4]oxazine-7-carboxamide;
(R)-1-(3,5-difluorobenzyl)-3-methyl-2-oxo-N-(2,4,6-trifluorobenzyl)-2,3-dihydro-1H-pyrido[2,3-b-1,4]oxazine-7-carboxamide;
1-(2-chloro-6-fluorobenzyl)-2-oxo-N-(2,4,6-trifluorobenzyl)-2,3-dihydro-1H-pyrido[2,3-b-1,4]oxazine-7-carboxamide;
1-(3,5-difluorobenzyl)-3-methyl-2-oxo-N-(2,4,6-trifluorobenzyl)-2,3-dihydro-1H-pyrido[2,3-b-1,4]oxazine-7-carboxamide;
(S)—N-(benzofuran-2-ylmethyl)-1-(3,5-difluorobenzyl)-3-methyl-2-oxo-2,3-dihydro-1H-pyrido[2,3-b][1,4]oxazine-7-carboxamide;
(R)-4-(3,5-difluorobenzyl)-2-methyl-3-oxo-N-(2,4,6-trifluorobenzyl)-3,4-dihydro-2H-benzo[b][1,4]oxazine-6-carboxamide;
(S)-4-(3,5-difluorobenzyl)-2-methyl-3-oxo-N-(2,4,6-trifluorobenzyl)-3,4-dihydro-2H-benzo[b][1,4]oxazine-6-carboxamide;
1-(2-chloro-6-fluorobenzyl)-3-methyl-2-oxo-N-(3-(trifluoromethyl)benzyl)-2,3-dihydro-1H-pyrido[2,3-b][1,4]oxazine-7-carboxamide;
4-(2-chloro-6-fluorobenzyl)-N-(furan-2-ylmethyl)-3-oxo-3,4-dihydro-2H-benzo[b][1,4]thiazine-6-carboxamide 1-oxide;
4-benzyl-N-(furan-2-ylmethyl)-3,4-dihydro-2H-benzo[b-1,4]thiazine-6-carboxamide;
4-(2-fluorobenzyl)-N-(furan-2-ylmethyl)-3,4-dihydro-2H-benzo[b-1,4]thiazine-6-carboxamide;
N-(furan-2-ylmethyl)-4-(2-hydroxy-2-phenylethyl)-3,4-dihydro-2H-benzo[b-1,4]thiazine-6-carboxamide;
N-(furan-2-ylmethyl)-4-(2-oxo-2-phenylethyl)-3,4-dihydro-2H-benzo[b][1,4]thiazine-6-carboxamide;
4-(4-chlorobenzyl)-N-(furan-2-ylmethyl)-3,4-dihydro-2H-benzo[b-1,4]thiazine-6-carboxamide;
4-benzyl-N-(furan-2-ylmethyl)-3,4-dihydro-2H-benzo[b-1,4]thiazine-6-carboxamide 1-oxide;
4-benzyl-N-(furan-2-ylmethyl)-3,4-dihydro-2H-benzo[b-1,4]thiazine-6-carboxamide 1,1-dioxide;
4-(2-fluorobenzoyl)-N-(furan-2-ylmethyl)-3,4-dihydro-2H-benzo[b-1,4]thiazine-6-carboxamide;
4-(2-fluorobenzoyl)-N-(furan-2-ylmethyl)-3,4-dihydro-2H-benzo[b-1,4]thiazine-6-carboxamide 1,1-dioxide;
4-benzoyl-N-(furan-2-ylmethyl)-3,4-dihydro-2H-benzo[b][1,4]thiazine-6-carboxamide;
4-(2-chloro-6-fluorobenzoyl)-N-(furan-2-ylmethyl)-3,4-dihydro-2H-benzo[b][1,4]thiazine-6-carboxamide;
4-(2-chloro-6-fluorobenzoyl)-N-(cyclohexylmethyl)-3,4-dihydro-2H-benzo[b][1,4]thiazine-6-carboxamide;
4-(2-chloro-6-fluorobenzoyl)-N-(2,4,6-trifluorobenzyl)-3,4-dihydro-2H-benzo[b][1,4]thiazine-6-carboxamide;
2-methyl-4-(2-phenylacetyl)-N-(2,4,6-trifluorobenzyl)-3,4-dihydro-2H-benzo[b][1,4]thiazine-6-carboxamide;
4-isonicotinoyl-N-(2,4,6-trifluorobenzyl)-3,4-dihydro-2H-benzo[b][1,4]thiazine-6-carboxamide;
4-(phenylsulfonyl)-N-(2,4,6-trifluorobenzyl)-3,4-dihydro-2H-benzo[b-1,4]thiazine-6-carboxamide;
4-((3,5-difluorophenyl)sulfonyl)-N-(2,4,6-trifluorobenzyl)-3,4-dihydro-2H-benzo[b-1,4]thiazine-6-carboxamide;
4-(2-fluorobenzoyl)-N-(furan-2-ylmethyl)-3,4-dihydro-2H-benzo[b-1,4]oxazine-6-carboxamide;
4-(2-chloro-6-fluorobenzoyl)-N-(2,4,6-trifluorobenzyl)-3,4-dihydro-2H-benzo[b-1,4]oxazine-6-carboxamide;
4-(2-chloro-6-fluorobenzoyl)-N-(furan-2-ylmethyl)-3,4-dihydro-2H-benzo[b-1,4]oxazine-6-carboxamide;
4-benzyl-N-(furan-2-ylmethyl)-3-thioxo-3,4-dihydro-2H-benzo[b-1,4]thiazine-6-carboxamide;
1-(2-chloro-6-fluorobenzyl)-3-methyl-2-oxo-N-(2,4,6-trifluorobenzyl)-1,2,3,4-tetrahydroquinoline-7-carboxamide;
1-(2-chloro-6-fluorobenzyl)-N-(furan-2-ylmethyl)-2-oxo-1,2,3,4-tetrahydroquinoline-7-carboxamide;
1-(2-chloro-6-fluorobenzyl)-N-(4-fluorobenzyl)-2-oxo-1,2,3,4-tetrahydroquinoline-7-carboxamide;
1-(2-chloro-6-fluorobenzyl)-2-oxo-N-(2,4,6-trifluorobenzyl)-1,2,3,4-tetrahydroquinoline-7-carboxamide;
1-(3,5-difluorobenzyl)-4,4-dimethyl-2-oxo-N-(2,4,6-trifluorobenzyl)-1,2,3,4-tetrahydroquinoline-7-carboxamide;
1-(3,5-difluorobenzyl)-N-(furo[2,3-c]pyridin-2-ylmethyl)-2-oxo-1,2,3,4-tetrahydroquinoline-7-carboxamide;
1-(3,5-difluorobenzyl)-N-(furo[3,2-c]pyridin-2-ylmethyl)-2-oxo-1,2,3,4-tetrahydroquinoline-7-carboxamide;
1-(3,5-difluorobenzyl)-N-(furo[2,3-b]pyridin-2-ylmethyl)-2-oxo-1,2,3,4-tetrahydroquinoline-7-carboxamide;
1-(3,5-difluorobenzyl)-N-(furo[3,2-b]pyridin-2-ylmethyl)-2-oxo-1,2,3,4-tetrahydroquinoline-7-carboxamide;
5-(3,5-difluorobenzyl)-6-oxo-N-(2,4,6-trifluorobenzyl)-5,6,7,8-tetrahydro-1,5-naphthyridine-3-carboxamide;
N-(2,4-difluorobenzyl)-5-(3,5-difluorobenzyl)-6-oxo-5,6,7,8-tetrahydro-1,5-naphthyridine-3-carboxamide;
N-(benzofuran-2-ylmethyl)-5-(3,5-difluorobenzyl)-6-oxo-5,6,7,8-tetrahydro-1,5-naphthyridine-3-carboxamide;
N-(2,4-difluorobenzyl)-8-(3,5-difluorobenzyl)-7-oxo-5,6,7,8-tetrahydro-1,8-naphthyridine-2-carboxamide;
N-(2,4-difluorobenzyl)-5-(4-fluorobenzyl)-6-oxo-5,6,7,8-tetrahydro-1,5-naphthyridine-3-carboxamide;
N-(benzofuran-2-ylmethyl)-8-(3,5-difluorobenzyl)-7-oxo-5,6,7,8-tetrahydro-1,8-naphthyridine-2-carboxamide;
N-(2,4-difluorobenzyl)-8-(4-fluorobenzyl)-7-oxo-5,6,7,8-tetrahydro-1,8-naphthyridine-2-carboxamide;
(3,5-difluorobenzyl)-7-oxo-N-(2,4,6-trifluorobenzyl)-5,6,7,8-tetrahydro-1,8-naphthyridine-2-carboxamide;
1-(3,5-Difluorobenzyl)-N-(2,4,6-trifluorobenzyl)-3,4-dihydro-1H-benzo[c][1,2]thiazine-7-carboxamide-2,2-dioxide; or
1-(3,5-difluorobenzyl)-3-methyl-N-(2,4,6-trifluorobenzyl)-3,4-dihydro-1H-benzo[c][1,2]thiazine-7-carboxamide 2,2-dioxide.

34. A pharmaceutical composition comprising a compound of Formula (I), as defined by any one of claims 1 to 33, or a pharmaceutically acceptable complex, salt, solvate, tautomeric form or polymorphic form thereof, and a pharmaceutically acceptable vehicle.

35. A process for making the composition of claim 34, the process comprising contacting a therapeutically effective amount of a compound according to any one of claims 1 to 37, or a pharmaceutically acceptable salt, solvate, tautomeric form or polymorphic form thereof, and a pharmaceutically acceptable vehicle.

36. A compound according to any one of claims 1 to 33 or a pharmaceutically acceptable complex, salt, solvate, tautomeric form or polymorphic form thereof, or a pharmaceutical composition according to claim 34, for use in modulating the Stimulator of Interferon Genes (STING) protein.

37. A compound or pharmaceutical composition for use according to claim 38, wherein the compound or composition is for use in activating, or agonising, the STING protein.

38. A compound according to any one of claims 1 to 33 or a pharmaceutically acceptable complex, salt, solvate, tautomeric form or polymorphic form thereof, or a pharmaceutical composition according to claim 34, for use in treating, ameliorating or preventing a disease selected from cancer, bacterial infection, viral infection, fungal infection, parasitic infection, immune-mediated disorder, central nervous system disease, peripheral nervous system disease, neurodegenerative disease, mood disorder, sleep disorder, cerebrovascular disease, peripheral artery disease or cardiovascular disease.

39. A compound or pharmaceutical composition for use according to claim 38, wherein the disease is cancer.

40. A compound or pharmaceutical composition for use according to claim 39, wherein the cancer is selected from the group consisting of colorectal cancer, aero-digestive squamous cancer, lung cancer, brain cancer, liver cancer, stomach cancer, sarcoma, leukaemia, lymphoma, multiple myeloma, ovarian cancer, uterine cancer, breast cancer, melanoma, prostate cancer, bladder cancer, pancreatic carcinoma or renal carcinoma.

41. A compound or pharmaceutical composition for use according to any one of claims 38 to 40, wherein the compound is for use with a second therapeutic agent, optionally wherein the second therapeutic agent comprises an antiviral agent, an anti-inflammation agent, conventional chemotherapy, an anti-cancer vaccine and/or hormonal therapy.

42. A compound or pharmaceutical composition for use according to claim 41, wherein the second therapeutic agent comprises a B7 costimulatory molecule, interleukin-2, interferon-g, GM-CSF, a CTLA-4 antagonist (such as Ipilimumab and tremilimumab), an IDO inhibitor or IDO/TDO inhibitor (such as Epacadostat and GDC-o919), a PD-1 inhibitor (such as Nivolumab, Pembrolizumab, Pidilizumab, AMP-224, and MDX-1106), a PD-L1 inhibitor (such as Durvalumab, Avelumab and Atezolizumab), an OX-40 ligand, a LAG3 inhibitor, a CD40 ligand, a 41BB/CD137 ligand, a CD27 ligand, Bacille Calmette-Guerin (BCG), liposomes, alum, Freund's complete or incomplete adjuvant, a TLR agonist (such as Poly I:C, MPL, LPS, bacterial flagellin, imiquimod, resiquimod, loxoribine and a CpG dinucleotide) and/or detoxified endotoxins.

43. A compound of formula (I): 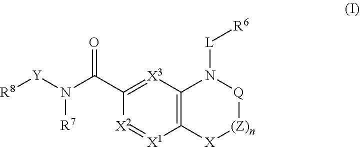
or a pharmaceutically acceptable salt or prodrug thereof, wherein:
X is CR⁹R¹⁰, O, S, S═O or SO₂;
X¹ is CR¹ or N;
X² is CR² or N;
X³ is CR³ or N;
the or each Z is CR¹¹R¹²;
n is 1 or 2;
Q is C═O, S═O, SO₂, C═S or CR⁴R⁵;
L is optionally substituted C₁-C₆ alkyl, C₁-C₃ polyfluoroalkyl, optionally substituted C₃-C₆ cycloalkyl, optionally substituted C₂-C₆ alkenyl, optionally substituted C₂-C₆ alkynyl, C═O, S═O, SO₂, —CH₂C(O)—, —CH₂CONH—, or —CONH—;
Y is an optionally substituted C₁-C₆ alkyl, C₁-C₃ polyfluoroalkyl, an optionally substituted C₂-C₆ alkenyl, an optionally substituted C₂-C₆ alkynyl, an optionally substituted C₃-C₆ cycloalkyl;
R¹, R² and R³ are each independently selected from the group consisting of H, halogen, CN, hydroxyl, COOH, CONR¹R², NR¹R², NHCOR¹, optionally substituted C₁-C₆ alkyl, C₁-C₃ polyfluoroalkyl, optionally substituted C₁-C₆ alkylsulfonyl, optionally substituted mono or bicyclic C₃-C₆ cycloalkyl, optionally substituted C₂-C₆ alkenyl, optionally substituted C₂-C₆ alkynyl, optionally substituted C₁-C₆ alkoxy, optionally substituted C₁-C₆ alkoxycarbonyl group, mono or bicyclic optionally substituted C₅-C₁₀ aryl, mono or bicyclic optionally substituted 5 to 10 membered heteroaryl, optionally substituted mono or bicyclic 3 to 8 membered heterocycle, optionally substituted aryloxy, optionally substituted heteroaryloxy, and optionally substituted heterocyclyloxy;
R⁴ and R⁵ are each independently selected from the group consisting of H, halogen, optionally substituted C₁-C₆ alkyl and optionally substituted (C₃-C₆) cycloalkyl; or R⁴ and R⁵ together with the atom to which they are attached form a spirocyclic ring;
R⁶ is mono or bicyclic optionally substituted C₅-C₁₀ aryl, mono or bicyclic optionally substituted 5 to 10 membered heteroaryl, optionally substituted C₃-C₆ cycloalkyl or an optionally substituted mono or bicyclic 3 to 8 membered heterocycle;
R⁷ is H, optionally substituted C₁-C₆ alkyl, optionally substituted sulfonyl, optionally substituted C₁-C₆ alkylsulfonyl, optionally substituted C₃-C₆ cycloalkyl, optionally substituted C₂-C₆ alkenyl and optionally substituted C₂-C₆ alkynyl;
R⁸ is mono or bicyclic optionally substituted C₅-C₁₀ aryl, mono or bicyclic optionally substituted 5 to 10 membered heteroaryl, optionally substituted mono or bicyclic C₃-C₆ cycloalkyl or an optionally substituted mono or bicyclic 3 to 8 membered heterocycle;
R⁹ and R¹⁰ are each independently selected from the group consisting of optionally substituted C₁-C₆ alkyl, H, halogen, CN, hydroxyl, CO₂H, CONR¹R², azido, sulfonyl, NR¹R², NHCOR¹, C₁-C₃ polyfluoroalkyl, optionally substituted C₁-C₆ thioalkyl, optionally substituted C₁-C₆ alkylsulfonyl, optionally substituted C₃-C₆ cycloalkyl, optionally substituted C₂-C₆ alkenyl, optionally substituted C₂-C₆ alkynyl, optionally substituted C₁-C₆ alkoxy, optionally substituted C₁-C₆ alkoxycarbonyl, mono or bicyclic optionally substituted C₅-C₁₀ aryl, mono or bicyclic optionally substituted 5 to 10 membered heteroaryl, optionally substituted heterocycle, optionally substituted aryloxy, and an optionally substituted heteroaryloxy; or R⁹ and R¹⁰ together with the C atom to which they are attached can combine to form an optionally substituted spirocyclic ring; and
R¹¹ and R¹² are each independently selected from the group consisting of optionally substituted C₁-C₆ alkyl, H, halogen, CN, hydroxyl, CO₂H, CONR¹R², azido, sulfonyl, NR¹R², NHCOR¹, C₁-C₃ polyfluoroalkyl, optionally substituted C₁-C₆ thioalkyl, optionally substituted C₁-C₆ alkylsulfonyl, optionally substituted C₃-C₆ cycloalkyl, optionally substituted C₂-C₆ alkenyl, optionally substituted C₂-C₆ alkynyl, optionally substituted C₁-C₆ alkoxy, optionally substituted C₁-C₆ alkoxycarbonyl, mono or bicyclic optionally substituted C₅-C₁₀ aryl, mono or bicyclic optionally substituted 5 to 10 membered heteroaryl, optionally substituted heterocycle, optionally substituted aryloxy, and an optionally substituted heteroaryloxy; or R¹ and R¹² together with the C atom to which they are attached can combine to form an optionally substituted spirocyclic ring;
with the proviso that when X is S; X¹, X² and X³ are CH; n is 1; Z is CH₂; Q is C═O; L is —CH₂—; Y is —CH₂—; R⁷ is H; and R⁶ is 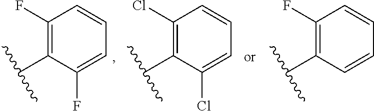
then R⁸ is not unsubstituted furanyl;
when X is S; X¹, X² and X³ are CH; n is 1; Z is CH₂; Q is C═O; L is —CH₂—; Y is —CH₂—;
R⁷ is H; and R⁶ is 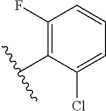 [SMILES: *c1c(F)cccc1Cl]
then R⁸ is not unsubstituted phenyl, unsubstituted thiophenyl, unsubstituted pyridinyl, unsubstituted furanyl, unsubstituted tetrahydrofuranyl, 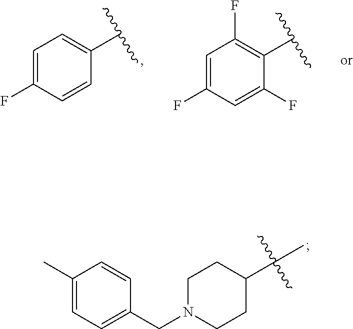 [SMILES: *c1ccc(F)cc1 ; *c1c(F)cc(F)cc1F ; *C1CCN(Cc2ccc(C)cc2)CC1]
when X is S; X¹, X² and X³ are CH; n is 1; Z is CH₂; Q is C═O; L is —CH₂—; Y is —CH₂CH₂—; R⁷ is H; and R⁶ is 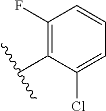 [SMILES: *c1c(F)cccc1Cl]
then R⁸ is not unsubstituted phenyl;
when X is S; X¹, X² and X³ are CH; n is 1; Z is CH₂; Q is C═O; L is —CH₂—; Y is 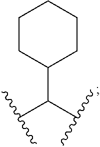 [SMILES: *C(*)C1CCCCC1]
R⁷ is H; and R⁶ is 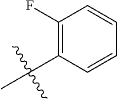 [SMILES: *c1ccccc1F]
then R⁸ is not unsubstituted phenyl;
when X is S; X¹, X² and X³ are CH; n is 1; Z is CH₂; Q is C═O; L is —CH₂—; Y is —CH₂—;
R⁷ is H; and R⁶ is unsubstituted phenyl then R⁸ is not unsubstituted furanyl, unsubstituted phenyl, 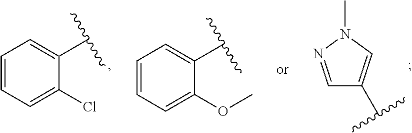
when X is S; X¹, X² and X³ are CH; n is 1; Z is CH₂; Q is C═O or CH₂; L is —CH₂—; Y is —CH₂—; R⁷ is H; and R⁶ is 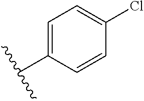 [SMILES: *c1ccc(Cl)cc1]
then R⁸ is not unsubstituted furanyl;
when X is S; X¹, X² and X³ are CH; n is 1; Z is CH₂; Q is C═O; L is —CH₂—; Y is —CH₂—;
R⁷ is H; and R⁶ is 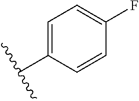 [SMILES: *c1ccc(F)cc1]
then R⁸ is not unsubstituted furanyl;
when X is S; X¹, X² and X³ are CH; n is 1; Z is CH₂; Q is C═O; L is —CH₂—; Y is —CH₂—;
R⁷ is H; and R⁶ is 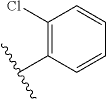 [SMILES: *c1ccccc1Cl]
then R⁸ is not unsubstituted furanyl, 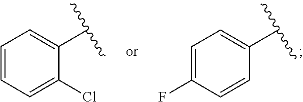 [SMILES: *c1ccccc1Cl ; *c1ccc(F)cc1]
when X is S; X¹, X² and X³ are CH; n is 1; Z is CH₂; Q is C═O; L is —CH₂—; Y is —CH₂—;
R⁷ is H; and R⁶ is 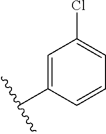 [SMILES: *c1cccc(Cl)c1]
then R⁸ is not unsubstituted furanyl or 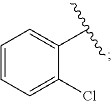 [SMILES: *c1ccccc1Cl]
and
when X is S; X¹, X² and X³ are CH; n is 1; Z is CH₂; Q is C═O; L is —CH₂—; Y is 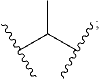 [SMILES: *C(*)C]
R⁷ is H; and R⁶ is 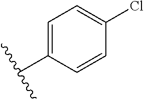 [SMILES: *c1ccc(Cl)cc1]
then R⁸ is not unsubstituted phenyl;
or a pharmaceutically acceptable complex, salt, solvate, tautomeric form or polymorphic form thereof.

44. A compound of the formula (II) or (III): 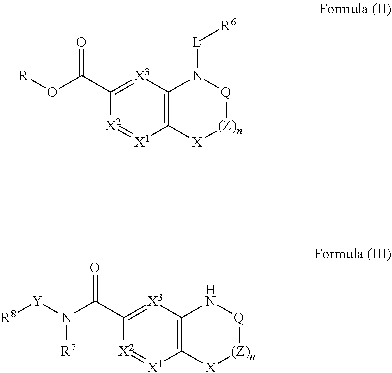
wherein, X, X¹, X², X³, n, Z, Q, L, Y, R⁶, R⁷ and R⁸ are as defined in any one of claims 1 to 35; and
R is H or a C₁-C₆ alkyl,
to or a pharmaceutically acceptable complex, salt, solvate, tautomeric form or polymorphic form thereof.

45. A compound according to claim 44, wherein the compound is selected from:

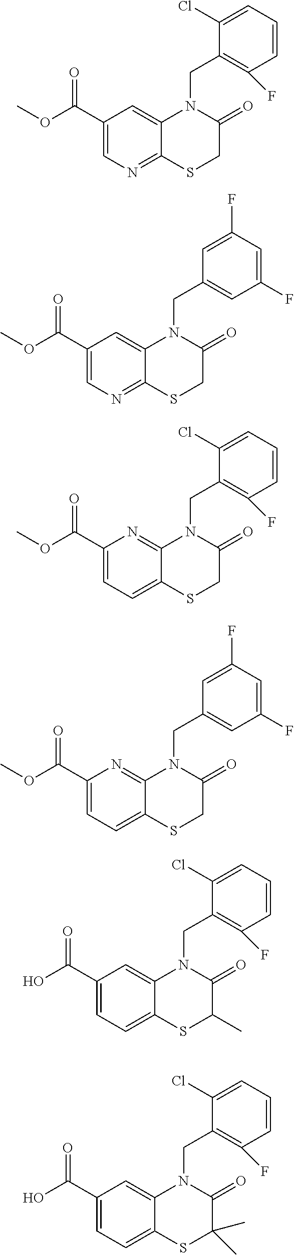

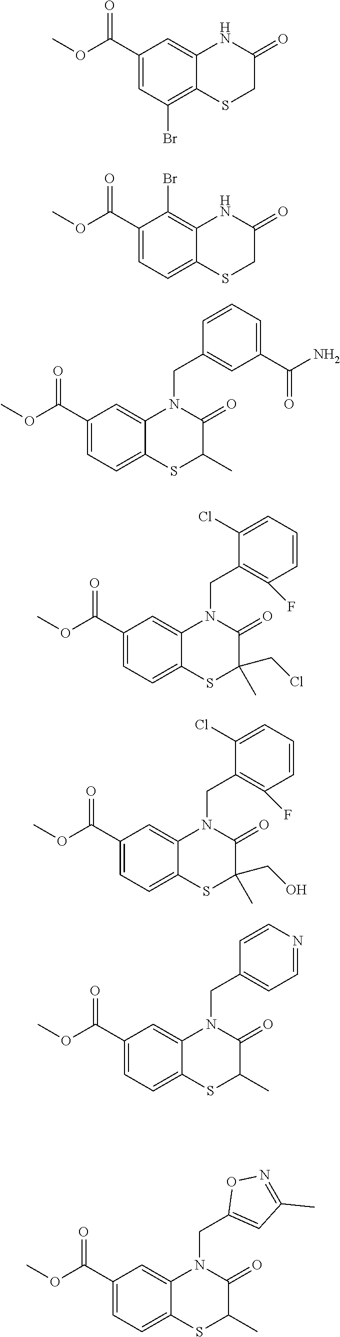

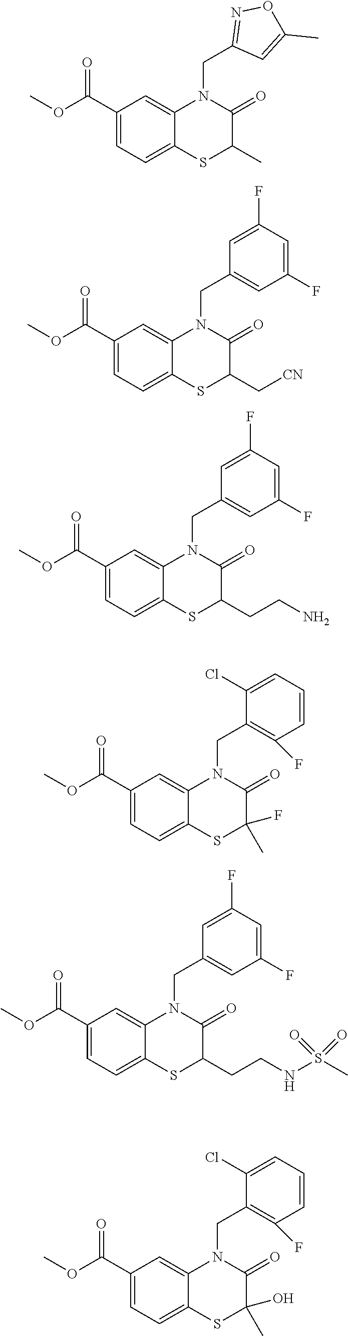

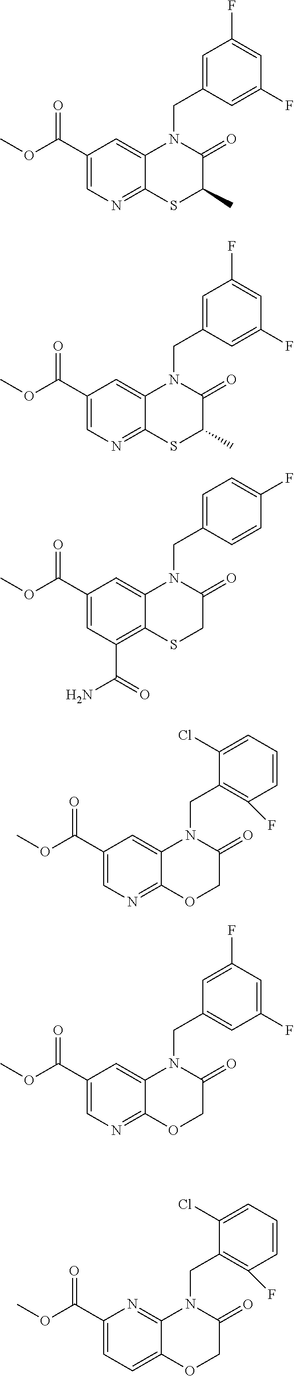

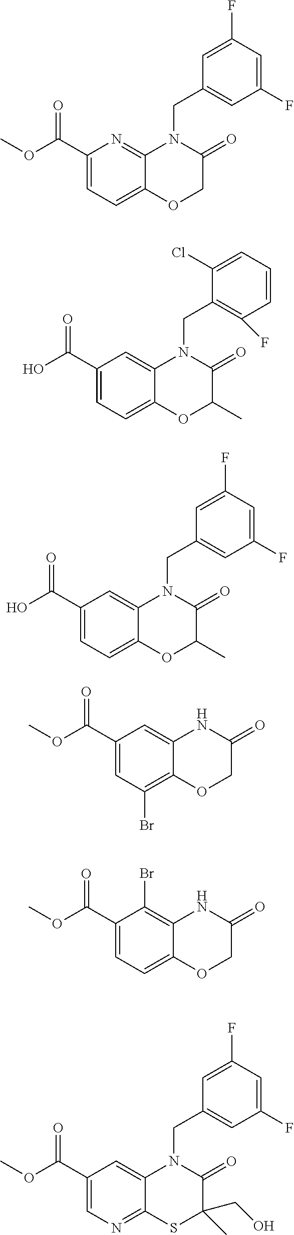

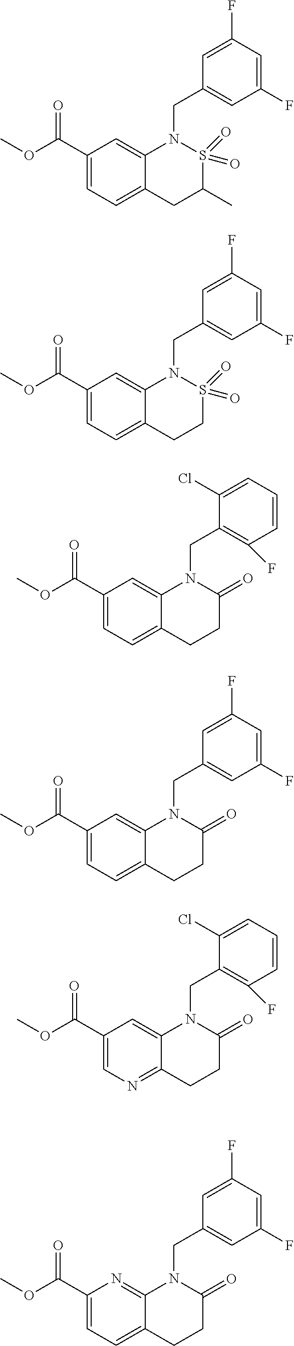

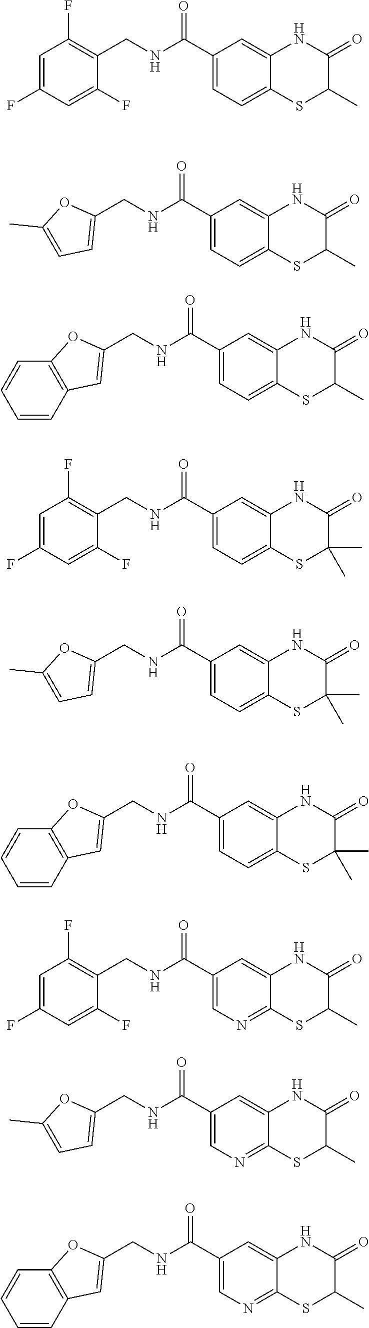

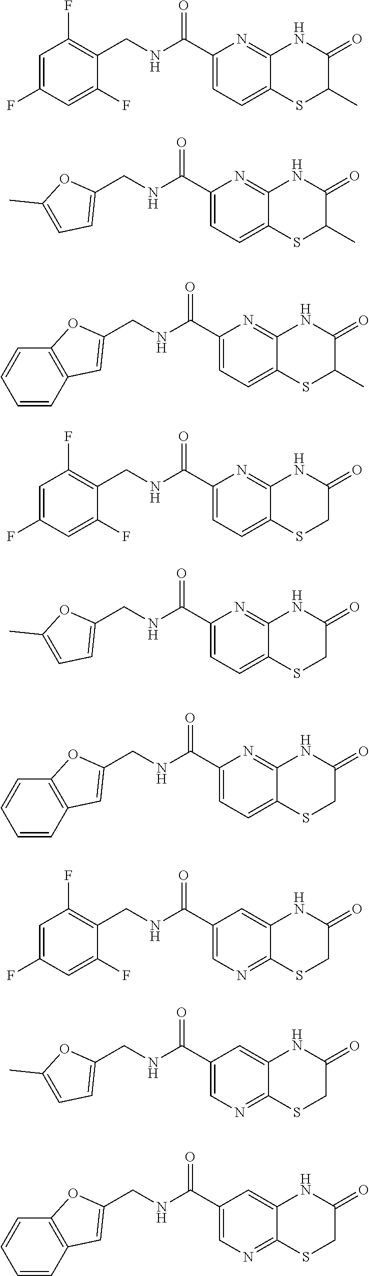

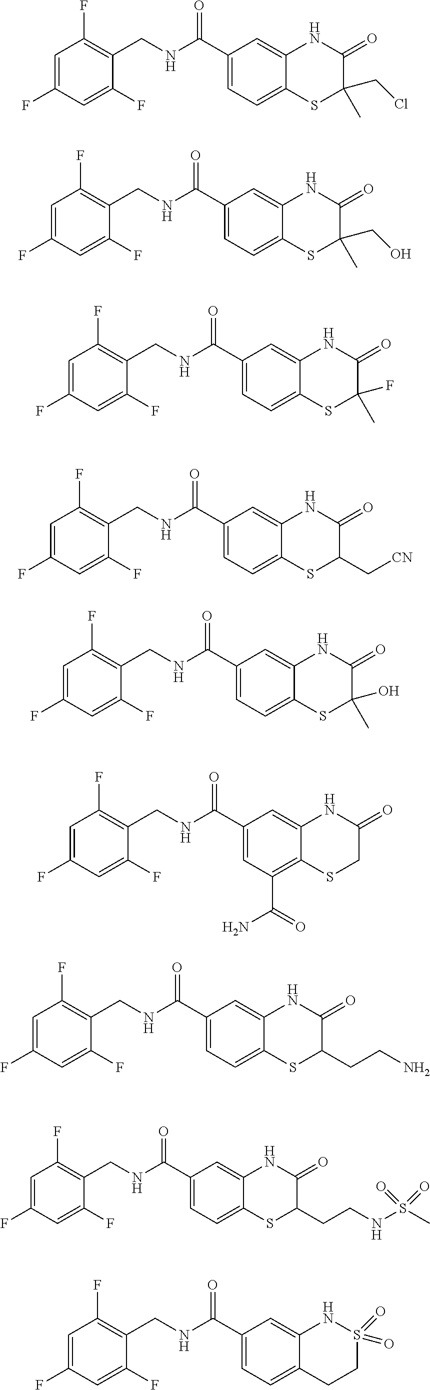

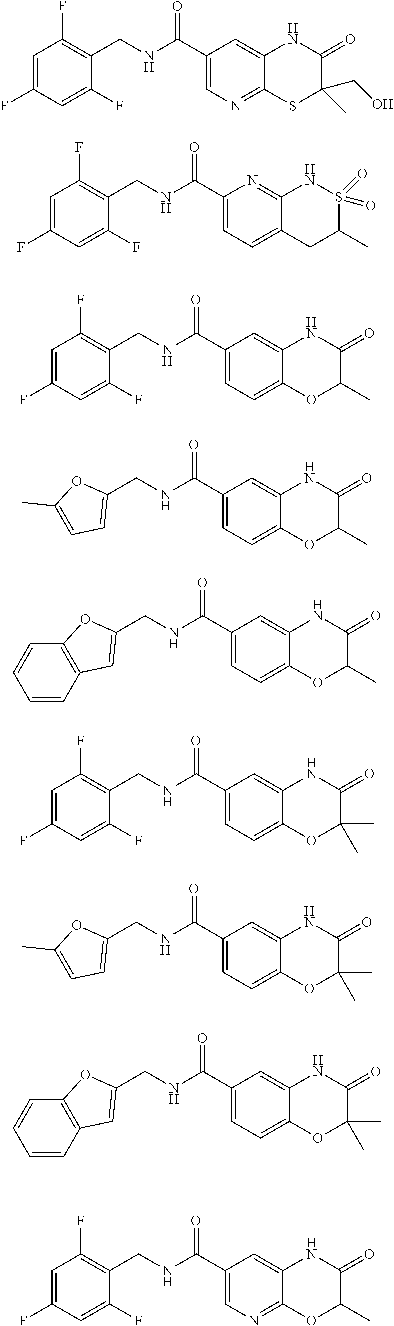

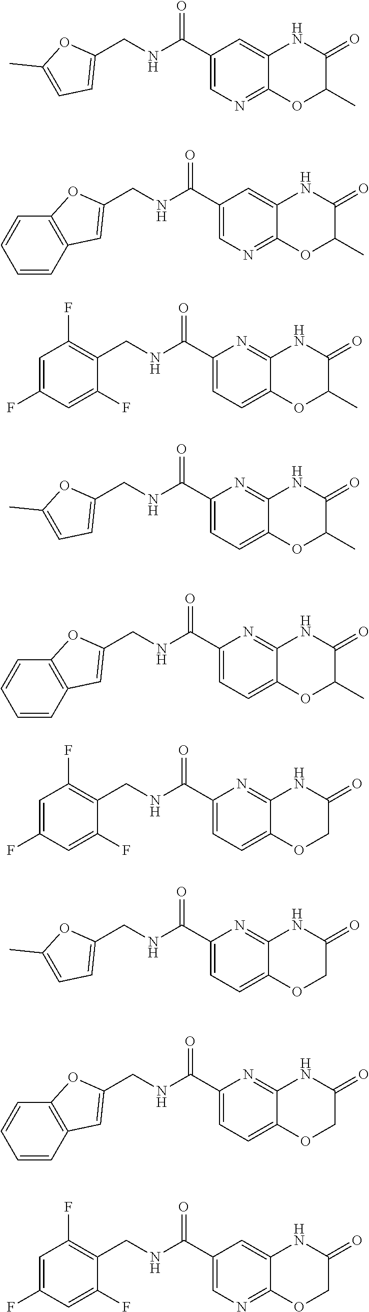

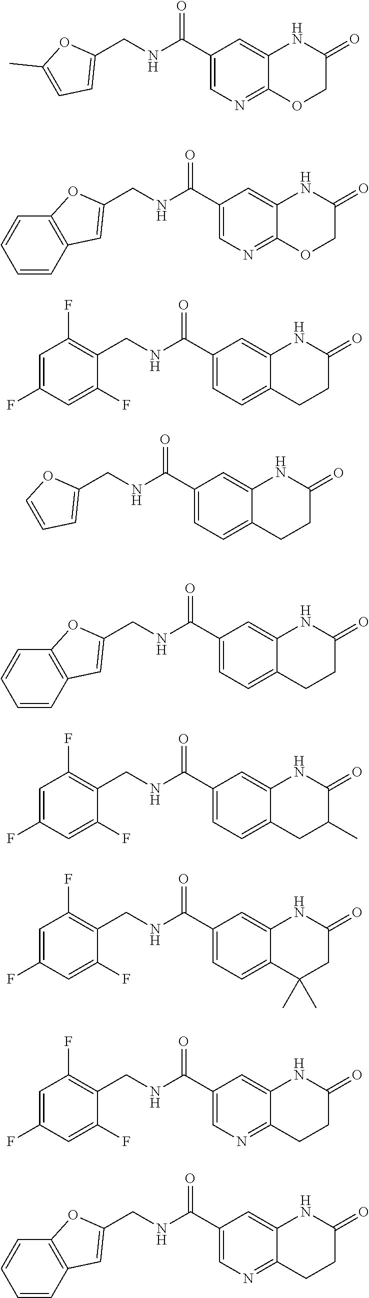

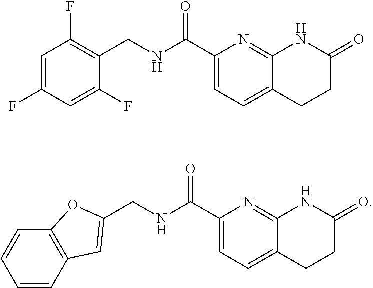 [SMILES: O=C1CCc2ccc(C(=O)NCc3c(F)cc(F)cc3F)nc2N1 ; O=C1CCc2ccc(C(=O)NCc3cc4ccccc4o3)nc2N1]

46. A conjugate of formula (IV): 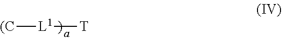
wherein C is a compound as defined by any one of claims 1 to 33;
L¹ is a linker;
T is a targeting moiety; and
a is an integer between 1 and 10.
